(19) United States
(12) Patent Application Publication
(10) Pub. No.: US 2024/0306501 A1
(43) Pub. Date: Sep. 12, 2024

(54) ORGANIC COMPOUND, OPTOELECTRONIC DEVICE INCLUDING THE SAME, AND ELECTRONIC APPARATUS AND ELECTRONIC EQUIPMENT INCLUDING THE OPTOELECTRONIC DEVICE

(71) Applicant: Samsung Display Co., Ltd., Yongin-si (KR)

(72) Inventors: Hyejin Jung, Yongin-si (KR); Hwasook Ryu, Yongin-si (KR); Seokgyu Yoon, Yongin-si (KR)

(21) Appl. No.: 18/481,157

(22) Filed: Oct. 4, 2023

(30) Foreign Application Priority Data
Feb. 23, 2023 (KR) 10-2023-0024592

(51) Int. Cl. H10K 85/60; C07D 495/04; C07D 519/00; C09K 11/06

(52) U.S. Cl. CPC H10K 85/6576; C07D 495/04; C07D 519/00; C09K 11/06; H10K 85/633; H10K 85/636; H10K 85/654; H10K 85/657; H10K 85/6572; C09K 2211/1018; H10K 50/11; H10K 85/615

(57) ABSTRACT

An optoelectronic device having excellent or suitable external quantum efficiency is provided. The optoelectronic device includes a first electrode, a second electrode facing the first electrode, an optical activation layer arranged between the first electrode and the second electrode, and a first compound. The first compound (i.e., small organic compound) has excellent or suitable deposition stability and heat resistance and the structure is described.

### CROSS-REFERENCE TO RELATED APPLICATION

This application claims priority to and the benefit of Korean Patent Application No. 10-2023-0024592, filed on Feb. 23, 2023, in the Korean Intellectual Property Office, the disclosure of which is incorporated by reference herein in its entirety.

### BACKGROUND

### 1. Field

One or more aspects of embodiments of the present disclosure relate to an organic compound, an optoelectronic device including the same, and an electronic apparatus and electronic equipment that include the optoelectronic device.

### 2. Description of the Related Art

Optoelectronic devices are devices that convert optical energy or optical signals into electrical energy or electrical signals. Examples of an optoelectronic device include an optical or solar cell, which converts optical energy into electrical energy, an optical detector or sensor, which detects and converts optical energy into electrical signals, and/or the like.

Electronic apparatuses including optoelectronic devices and light-emitting devices have been developed. Light emitted from a light-emitting device may be reflected from an object (for example, a finger of a user) in contact (or in proximity) with an electronic apparatus, and then incident on an optoelectronic device. As the optoelectronic device detects incident light energy and converts it into electrical signals, the contact of the object with the electronic apparatus may be recognized. The optoelectronic device may be utilized as a fingerprint recognition sensor and/or the like.

### SUMMARY

One or more aspects of embodiments of the present disclosure are directed toward an organic compound having excellent or suitable deposition stability and heat resistance, an optoelectronic device having excellent or suitable external quantum efficiency, and a high-quality electronic apparatus and electronic equipment that utilize the optoelectronic device.

Additional aspects will be set forth in part in the description which follows and, in part, will be apparent from the description, or may be learned by practice of the presented embodiments of the disclosure.

According to one or more embodiments, an optoelectronic device includes
a first electrode,
a second electrode facing the first electrode,
an optical activation layer arranged between the first electrode and the second electrode, and
a first compound represented by Formula 1:

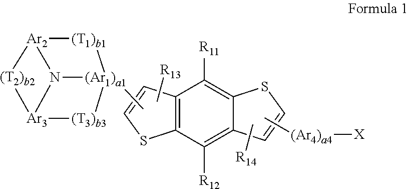

wherein, in Formula 1,
X may be an electron-acceptor group,
Ar₁ to Ar₄ may each independently be a C₃-C₆₀ carbocyclic group unsubstituted or substituted with at least one R₁₀ₐ or a C₁-C₆₀ heterocyclic group unsubstituted or substituted with at least one R₁₀ₐ,
T₁ to T₃ may each independently be selected from a single bond, *—O—*′, *—S—*′, *—C(R₂₁)(R₂₂)—*′, *—Si(R₂₁)(R₂₂)—*′, and *—N(R₂₁)—*′,
each of a1, a4, and b1 to b3 may be 0 or 1,
R₁₁ to R₁₄, R₂₁, and R₂₂ may each independently be hydrogen, deuterium, —F, —Cl, —Br, —I, a hydroxyl group, a cyano group, a nitro group, a C₁-C₆₀ alkyl group unsubstituted or substituted with at least one R₁₀ₐ, a C₂-C₆₀ alkenyl group unsubstituted or substituted with at least one R₁₀ₐ, a C₂-C₆₀ alkynyl group unsubstituted or substituted with at least one R₁₀ₐ, a C₁-C₆₀ alkoxy group unsubstituted or substituted with at least one R₁₀ₐ, a C₃-C₆₀ carbocyclic group unsubstituted or substituted with at least one R₁₀ₐ, a C₁-C₆₀ heterocyclic group unsubstituted or substituted with at least one R₁₀ₐ, a C₆-C₆₀ aryloxy group unsubstituted or substituted with at least one R₁₀ₐ, a C₆-C₆₀ arylthio group unsubstituted or substituted with at least one R₁₀ₐ, a C₇-C₆₀ arylalkyl group unsubstituted or substituted with at least one R₁₀ₐ, a C₂-C₆₀ heteroarylalkyl group unsubstituted or substituted with at least one R₁₀ₐ, —C(Q₁)(Q₂)(Q₃), —Si(Q₁)(Q₂)(Q₃), —N(Q₁)(Q₂), —B(Q₁)(Q₂), —C(═O)(Q₁), —S(═O)₂(Q₁), or —P(═O)(Q₁)(Q₂),
R₁₀ₐ may be
deuterium, —F, —Cl, —Br, —I, a hydroxyl group, a cyano group, or a nitro group,
a C₁-C₆₀ alkyl group, a C₂-C₆₀ alkenyl group, a C₂-C₆₀ alkynyl group, or a C₁-C₆₀ alkoxy group, each unsubstituted or substituted with deuterium, —F, —Cl, —Br, —I, a hydroxyl group, a cyano group, a nitro group, a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, a C₂-C₆₀ heteroarylalkyl group, —Si(Q₁₁)(Q₁₂)(Q₁₃), —N(Q₁₁)(Q₁₂), —B(Q₁₁)(Q₁₂), —C(═O)(Q₁₁), —S(═O)₂(Q₁₁), —P(═O)(Q₁₁)(Q₁₂), or any combination thereof,
a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, or a C₂-C₆₀ heteroarylalkyl group, each unsubstituted or substituted with deuterium, —F, —Cl, —Br, —I, a hydroxyl group, a cyano group, a nitro group, a C₁-C₆₀ alkyl group, a C₂-C₆₀ alkenyl group, a C₂-C₆₀ alkynyl group, a C₁-C₆₀ alkoxy group, a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, a C₂-C₆₀ heteroarylalkyl group, —Si(Q₂₁)(Q₂₂)(Q₂₃), —N(Q₂₁)(Q₂₂), —B(Q₂₁)(Q₂₂), —C(═O)(Q₂₁), —S(═O)₂(Q₂₁), —P(═O)(Q₂₁)(Q₂₂), or any combination thereof, or
—Si(Q₃₁)(Q₃₂)(Q₃₃), —N(Q₃₁)(Q₃₂), —B(Q₃₁)(Q₃₂), —C(═O)(Q₃₁), —S(═O)₂(Q₃₁), or —P(═O)(Q₃₁)(Q₃₂), and
Q₁ to Q₃, Q₁₁ to Q₁₃, Q₂₁ to Q₂₃, and Q₃₁ to Q₃₃ may each independently be
hydrogen, deuterium, —F, —Cl, —Br, —I, a hydroxyl group, a cyano group, a nitro group, a C₁-C₆₀ alkyl group, a C₂-C₆₀ alkenyl group, a C₂-C₆₀ alkynyl group, or a C₁-C₆₀ alkoxy group, or
a C₃-C₆₀ carbocyclic group, a C₁-C₆₀ heterocyclic group, a C₇-C₆₀ arylalkyl group, or a C₂-C₆₀ heteroarylalkyl group, each unsubstituted or substituted with deuterium, —F, a cyano group, a C₁-C₆₀ alkyl group, a C₁-C₆₀ alkoxy group, a phenyl group, a biphenyl group, or any combination thereof.

According to one or more embodiments, an electronic apparatus includes the optoelectronic device,
a light-emitting device configured to not overlap (e.g., is not overlapping) the optoelectronic device, and
a color filter, a color conversion layer, a touch screen layer, a polarizing layer, or any combination thereof.

According to one or more embodiments, electronic equipment includes the optoelectronic device,
wherein the electronic equipment is at least one selected from among a flat panel display, a curved display, a computer monitor, a medical monitor, a television, a billboard, an indoor or outdoor light and/or light for signal, a head-up display, a fully or partially transparent display, a flexible display, a rollable display, a foldable display, a stretchable display, a laser printer, a telephone, a portable phone, a tablet personal computer, a phablet, a personal digital assistant (PDA), a wearable device, a laptop computer, a digital camera, a camcorder, a viewfinder, a micro display, a three-dimensional (3D) display, a virtual reality or augmented reality display, a vehicle, a video wall with multiple displays tiled together, a theater or stadium screen, a phototherapy device, a signboard, a vehicle sensor, a household sensor, a solar cell, and combinations thereof.

According to one or more embodiments, provided is an organic compound represented by Formula 1.

### BRIEF DESCRIPTION OF THE DRAWINGS

The accompanying drawings are included to provide a further understanding of the above and other aspects, features, and advantages of certain embodiments of the present disclosure are incorporated in and constitute a part of this specification.

The drawings illustrate example embodiments and, together with the following description taken in conjunction with the accompanying drawings, serve to make the principles of the present disclosure more apparent. In the drawings: FIG. 1 is a schematic view of an optoelectronic device according to one or more embodiments of the present disclosure;

FIG. 2 is a schematic view of a light-emitting device included in an electronic apparatus according to one or more embodiments of the present disclosure;

FIG. 3 is a schematic view of an optoelectronic device according to one or more embodiments of the present disclosure;

FIG. 4 is a schematic view of an optoelectronic device according to one or more embodiments of the present disclosure;

FIG. 5 is a cross-sectional schematic view of an electronic apparatus according to one or more embodiments of the present disclosure;

FIG. 6 is a cross-sectional schematic view of an electronic apparatus according to one or more embodiments of the present disclosure;

FIG. 7 is a perspective view of electronic equipment including an optoelectronic device according to one or more embodiments of the present disclosure;

FIG. 8 is a schematic perspective view of the exterior of a vehicle as electronic equipment including an optoelectronic device according to one or more embodiments of the present disclosure;

FIG. 9A is a schematic view of the interior of the vehicle of FIG. 8;

FIG. 9B is a schematic view of the interior of the vehicle of FIG. 8; and

FIG. 9C is a schematic view of the interior of the vehicle of FIG. 8.

### DETAILED DESCRIPTION

Reference will now be made in more detail to embodiments, examples of which are illustrated in the accompanying drawings, wherein like reference numerals refer to like elements throughout, and duplicative descriptions thereof may not be provided. In this regard, the present embodiments may have different forms and should not be construed as being limited to the descriptions set forth herein. Accordingly, the embodiments are merely described, by referring to the drawings, to explain aspects of the present description.

As utilized herein, the term “and/or” includes any and all combinations of one or more of the associated listed items. Expressions such as “at least one of,” “one of,” and “selected from,” when preceding a list of elements, modify the entire list of elements and do not modify the individual elements of the list. For example, throughout the disclosure, the expression “at least one of a, b or c” indicates only a, only b, only c, both (e.g., simultaneously) a and b, both (e.g., simultaneously) a and c, both (e.g., simultaneously) b and c, all of a, b, and c, or variations thereof.

The terminology used herein is for the purpose of describing embodiments and is not intended to limit the embodiments described herein. Unless otherwise defined, all chemical names, technical and scientific terms, and terms defined in common dictionaries should be interpreted as having meanings consistent with the context of the related art, and should not be interpreted in an ideal or overly formal sense. It will be understood that, although the terms first, second, etc. may be used herein to describe various elements, these elements should not be limited by these terms. These terms are only used to distinguish one element from another element. Thus, a first element could be termed a second element without departing from the teachings of the present disclosure. Similarly, a second element could be termed a first element.

As used herein, singular forms such as “a,” “an,” and “the” are intended to include the plural forms as well, unless the context clearly indicates otherwise.

It will be further understood that the terms “comprise,” “comprises,” “comprising,” “has,” “have,” “having,” “include,” “includes,” and/or “including,” when used in this specification, specify the presence of stated features, steps, operations, elements, and/or components, but do not preclude the presence or addition of one or more other features, steps, operations, elements, components, and/or groups thereof.

As used herein, the terms “use,” “using,” and “used” may be considered synonymous with the terms “utilize,” “utilizing,” and “utilized,” respectively.

As used herein, the term “and/or” includes any, and all, combination(s) of one or more of the associated listed items.

The term “may” will be understood to refer to “one or more embodiments of the present disclosure,” some of which include the described element and some of which exclude that element and/or include an alternate element. Similarly, alternative language such as “or” refers to “one or more embodiments of the present disclosure,” each including a corresponding listed item.

It will be understood that when an element is referred to as being “on,” “connected to,” or “on” another element, it may be directly on, connected, or coupled to the other element or one or more intervening elements may also be present. When an element is referred to as being “directly on,” “directly connected to,” or “directly coupled to” another element, there are no intervening elements present.

Spatially relative terms, such as “beneath,” “below,” “lower,” “above,” “upper,” “bottom,” “top,” and the like, may be used herein for ease of description to describe one element or feature's relationship to another element(s) or feature(s) as illustrated in the drawings. It will be understood that the spatially relative terms are intended to encompass different orientations of the device in use or operation in addition to the orientation depicted in the drawings. For example, if the device in the drawings is turned over, elements described as “below” or “beneath” other elements or features would then be oriented “above” or “over” the other elements or features. Thus, the term “below” may encompass both an orientation of above and below. The device may be otherwise oriented (rotated 90 degrees or at other orientations), and the spatially relative descriptors used herein should be interpreted accordingly.

Unless otherwise defined, all terms (including technical and scientific terms) used herein have the same meaning as commonly understood by those of ordinary skill in the art to which the present disclosure pertains. It is also to be understood that terms defined in commonly used dictionaries should be interpreted as having meanings consistent with meanings in the context of the related art, unless expressly defined herein, and should not be interpreted in an ideal or overly formal sense.

In this context, “consisting essentially of” means that any additional components will not materially affect the chemical, physical, optical or electrical properties of the semiconductor film.

Further, in this specification, the phrase “on a plane,” or “plan view,” means viewing a target portion from the top, and the phrase “on a cross-section” means viewing a cross-section formed by vertically cutting a target portion from the side.

An aspect of the disclosure provides an optoelectronic device including:
a first electrode;
a second electrode facing the first electrode;
optical activation layer arranged between the first electrode and the second electrode; and
a first compound represented by Formula 1:

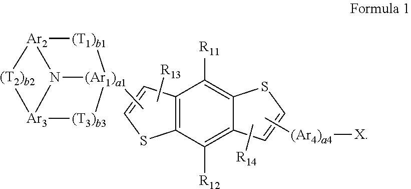

In Formula 1,
X may be an electron-acceptor group,
Ar₁ to Ar₄ may each independently be a C₃-C₆₀ carbocyclic group unsubstituted or substituted with at least one R₁₀ₐ or a C₁-C₆₀ heterocyclic group unsubstituted or substituted with at least one R₁₀ₐ,
T₁ to T₃ may each independently be selected from a single bond, *—O—*′, *—S—*′, *—C(R₂₁)(R₂₂)—*′, *—Si(R₂₁)(R₂₂)—*′, and *—N(R₂₁)—*′,
each of a1, a4, and b1 to b3 may be 0 or 1,
R₁₁ to R₁₄, R₂₁, and R₂₂ may each independently be hydrogen, deuterium, —F, —Cl, —Br, —I, a hydroxyl group, a cyano group, a nitro group, a C₁-C₆₀ alkyl group unsubstituted or substituted with at least one R₁₀ₐ, a C₂-C₆₀ alkenyl group unsubstituted or substituted with at least one R₁₀ₐ, a C₂-C₆₀ alkynyl group unsubstituted or substituted with at least one R₁₀ₐ, a C₁-C₆₀ alkoxy group unsubstituted or substituted with at least one R₁₀ₐ, a C₃-C₆₀ carbocyclic group unsubstituted or substituted with at least one R₁₀ₐ, a C₁-C₆₀ heterocyclic group unsubstituted or substituted with at least one R₁₀ₐ, a C₆-C₆₀ aryloxy group unsubstituted or substituted with at least one R₁₀ₐ, a C₆-C₆₀ arylthio group unsubstituted or substituted with at least one R₁₀ₐ, a C₇-C₆₀ arylalkyl group unsubstituted or substituted with at least one R₁₀ₐ, a C₂-C₆₀ heteroarylalkyl group unsubstituted or substituted with at least one R₁₀ₐ, —C(Q₁)(Q₂)(Q₃), —Si(Q₁)(Q₂)(Q₃), —N(Q₁)(Q₂), —B(Q₁)(Q₂), —C(═O)(Q₁), —S(═O)₂(Q₁), or —P(═O)(Q₁)(Q₂),
R₁₀ₐ may be:
deuterium, —F, —Cl, —Br, —I, a hydroxyl group, a cyano group, or a nitro group;
a C₁-C₆₀ alkyl group, a C₂-C₆₀ alkenyl group, a C₂-C₆₀ alkynyl group, or a C₁-C₆₀ alkoxy group, each unsubstituted or substituted with deuterium, —F, —Cl, —Br, —I, a hydroxyl group, a cyano group, a nitro group, a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, a C₂-C₆₀ heteroarylalkyl group, —Si(Q₁₁)(Q₁₂)(Q₁₃), —N(Q₁₁)(Q₁₂), —B(Q₁₁)(Q₁₂), —C(═O)(Q₁₁), —S(═O)₂(Q₁₁), —P(═O)(Q₁₁)(Q₁₂), or any combination thereof;
a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, or a C₂-C₆₀ heteroarylalkyl group, each unsubstituted or substituted with deuterium, —F, —Cl, —Br, —I, a hydroxyl group, a cyano group, a nitro group, a C₁-C₆₀ alkyl group, a C₂-C₆₀ alkenyl group, a C₂-C₆₀ alkynyl group, a C₁-C₆₀ alkoxy group, a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, a C₂-C₆₀ heteroarylalkyl group, —Si(Q₂₁)(Q₂₂)(Q₂₃), —N(Q₂₁)(Q₂₂), —B(Q₂₁)(Q₂₂), —C(═O)(Q₂₁), —S(═O)₂(Q₂₁), —P(═O)(Q₂₁)(Q₂₂), or any combination thereof; or
—Si(Q₃₁)(Q₃₂)(Q₃₃), —N(Q₃₁)(Q₃₂), —B(Q₃₁)(Q₃₂), —C(═O)(Q₃₁), —S(═O)₂(Q₃₁), or —P(═O)(Q₃₁)(Q₃₂), and
Q₁ to Q₃, Q₁₁ to Q₁₃, Q₂₁ to Q₂₃, and Q₃₁ to Q₃₃ may each independently be:
hydrogen, deuterium, —F, —Cl, —Br, —I, a hydroxyl group, a cyano group, a nitro group, a C₁-C₆₀ alkyl group, a C₂-C₆₀ alkenyl group, a C₂-C₆₀ alkynyl group, or a C₁-C₆₀ alkoxy group; or
a C₃-C₆₀ carbocyclic group, a C₁-C₆₀ heterocyclic group, a C₇-C₆₀ arylalkyl group, or a C₂-C₆₀ heteroarylalkyl group, each unsubstituted or substituted with deuterium, —F, a cyano group, a C₁-C₆₀ alkyl group, a C₁-C₆₀ alkoxy group, a phenyl group, a biphenyl group, or any combination thereof.

In one or more embodiments, the optoelectronic device may further include a hole transport region arranged between the first electrode and the optical activation layer and an electron transport region arranged between the optical activation layer and the second electrode. The hole transport region may include a hole injection layer, a hole transport layer, an emission auxiliary layer, an electron blocking layer, or any combination thereof. The electron transport region may include a buffer layer, a hole blocking layer, an electron control layer, an electron transport layer, an electron injection layer, or any combination thereof.

In one or more embodiments, the optoelectronic device may further include a second compound represented by one selected from among Formulae 2-1 to 2-6:

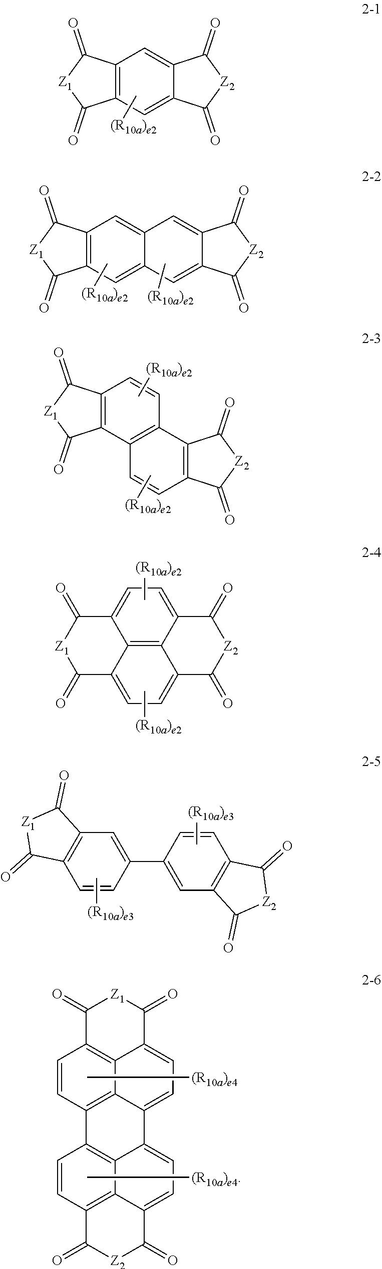

In Formulae 2-1 to 2-6,
Z₁ and Z₂ may each independently be selected from among O, S, N(R₁), C(R₁)(R₂), C(═O), C(═S), and C═C(R₁)(R₂),

R₁ and R₂ may each independently be hydrogen, deuterium, —F, —Cl, —Br, —I, a hydroxyl group, a cyano group, a nitro group, a C₁-C₆₀ alkyl group unsubstituted or substituted with at least one R₁₀ₐ, a C₂-C₆₀ alkenyl group unsubstituted or substituted with at least one R₁₀ₐ, a C₂-C₆₀ alkynyl group unsubstituted or substituted with at least one R₁₀ₐ, a C₁-C₆₀ alkoxy group unsubstituted or substituted with at least one R₁₀ₐ, a C₃-C₆₀ carbocyclic group unsubstituted or substituted with at least one R₁₀ₐ, a C₁-C₆₀ heterocyclic group unsubstituted or substituted with at least one R₁₀ₐ, a C₆-C₆₀ aryloxy group unsubstituted or substituted with at least one R₁₀ₐ, a C₆-C₆₀ arylthio group unsubstituted or substituted with at least one R₁₀ₐ, a C₇-C₆₀ arylalkyl group unsubstituted or substituted with at least one R₁₀ₐ, a C₂-C₆₀ heteroarylalkyl group unsubstituted or substituted with at least one R₁₀ₐ, —C(Q₁)(Q₂)(Q₃), —Si(Q₁)(Q₂)(Q₃), —N(Q₁)(Q₂), —B(Q₁)(Q₂), —C(═O)(Q₁), —S(═O)₂(Q₁), or —P(═O)(Q₁)(Q₂),
Q₁ to Q₃ and R₁₀ₐ may each be as defined herein,
e2 may be an integer from 0 to 2,
e3 may be an integer from 0 to 3, and
e4 may be an integer from 0 to 4.

In one or more embodiments, the optical activation layer may not include (e.g., may exclude) a (e.g., any) fullerene-based compound and/or a (e.g., any) subphthalocyanine-based compound. For example, the optical activation layer may not include (e.g., may exclude) at least one selected from among fullerene 60, fullerene 70, SubPC, and/or SubNC:

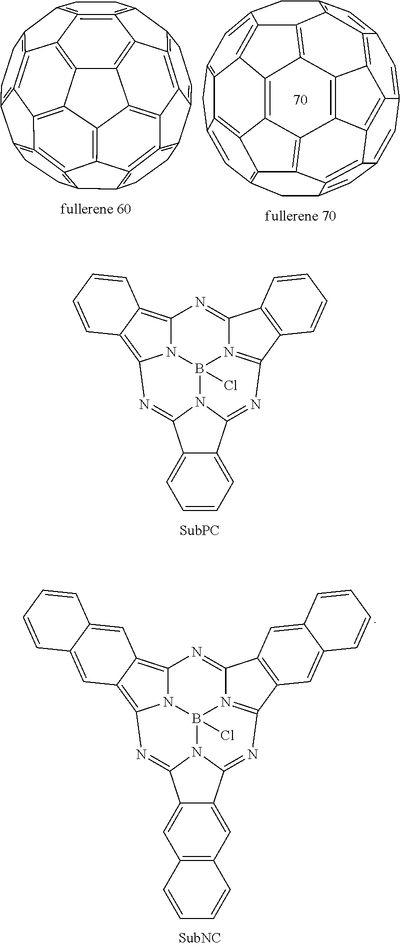

For example, the first compound may not be fullerene 60 and/or fullerene 70. The second compound may not be SubPC and/or SubNC.

In one or more embodiments, R₁ and R₂ may each independently be a C₁-C₆₀ alkyl group, a C₃-C₁₀ cycloalkyl group, a C₆-C₆₀ aryl group, or a C₁-C₆₀ heteroaryl group, each unsubstituted or substituted with deuterium, —F, —Cl, —Br, —I, a cyano group, a C₁-C₆₀ alkyl group, a C₁-C₆₀ alkoxy group, or any combination thereof.

In one or more embodiments, the second compound may be one selected from among Compounds N1 to N43:

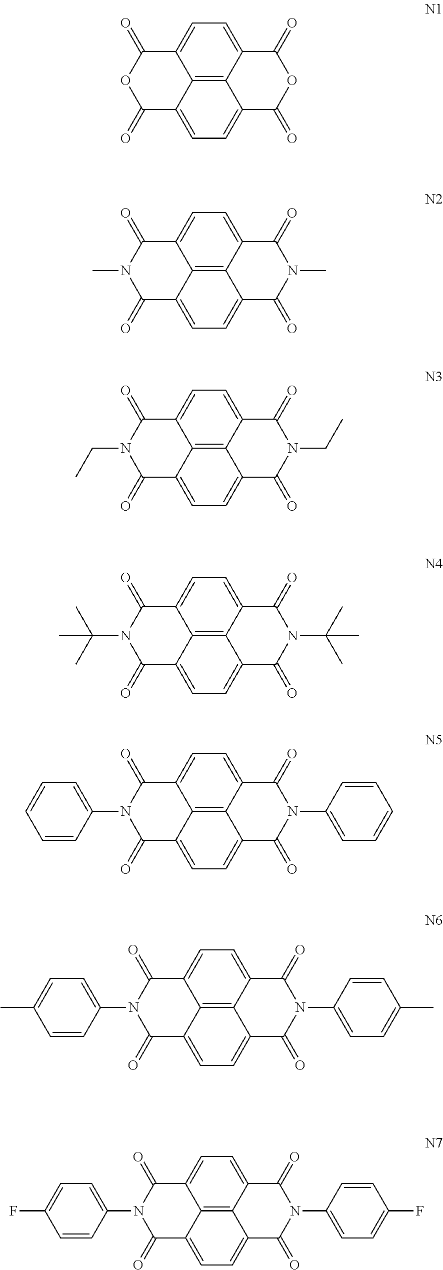

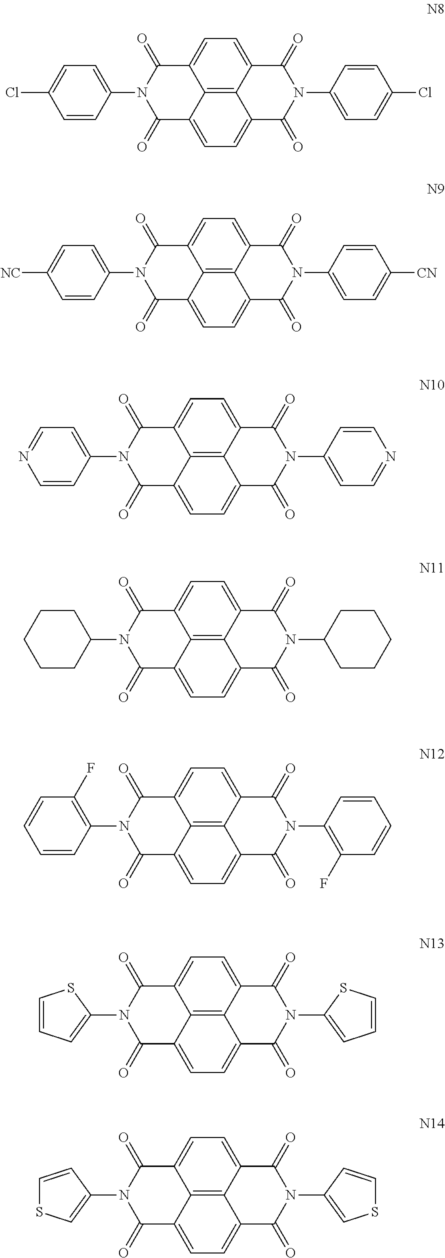

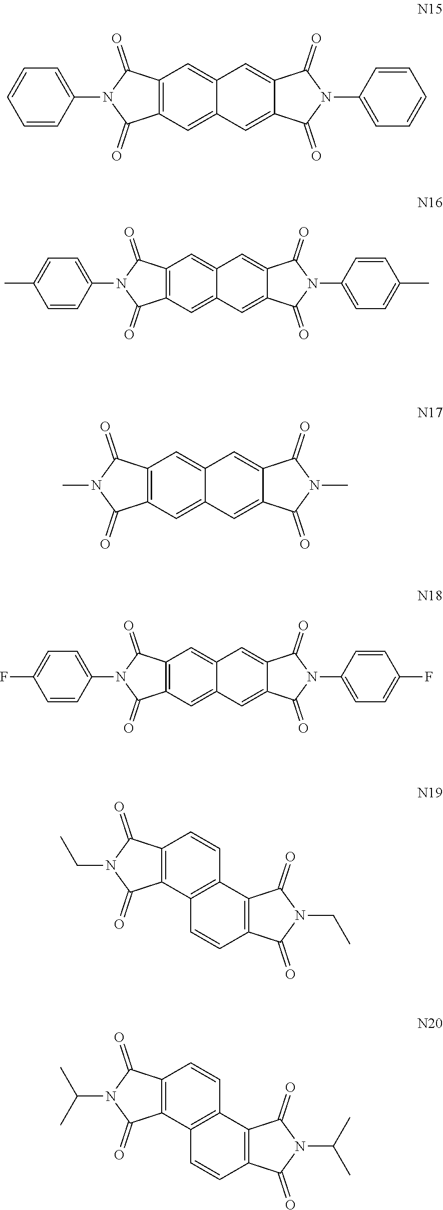

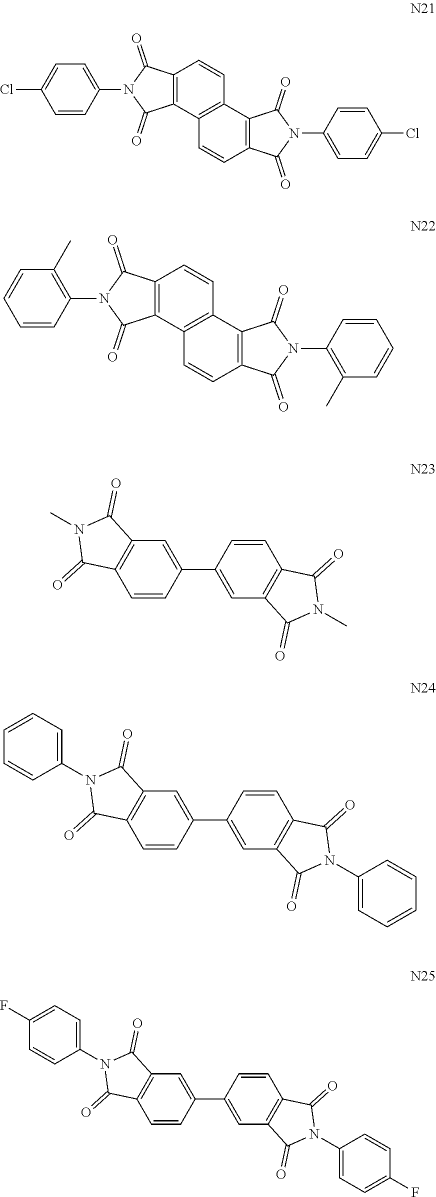

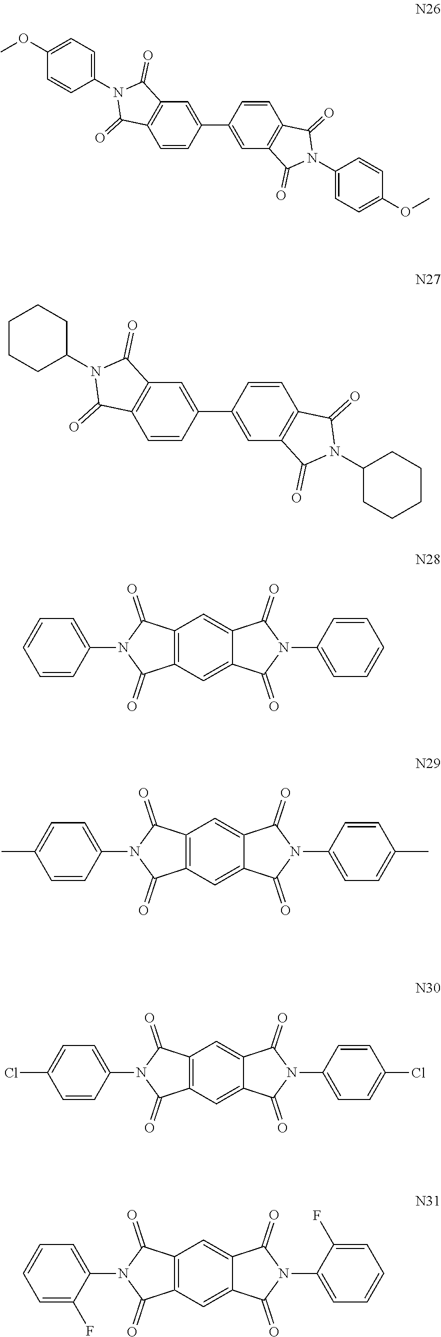

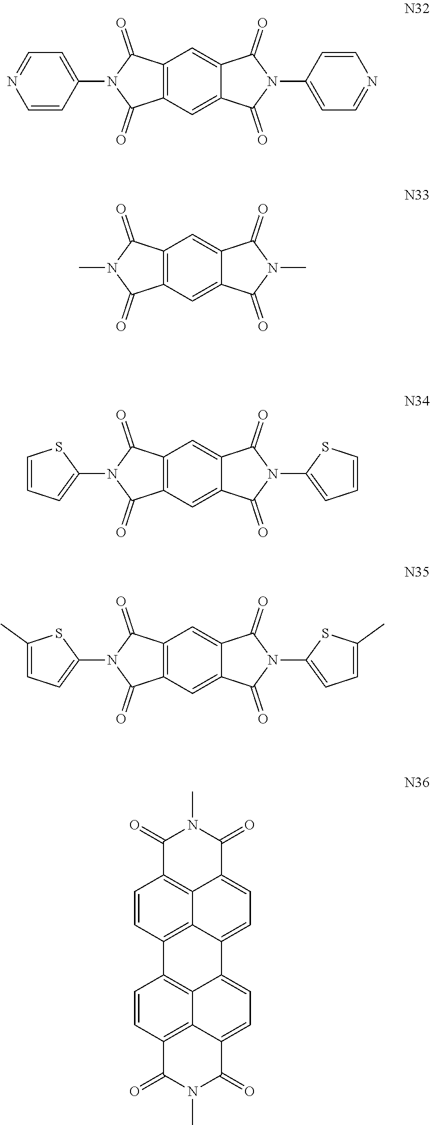

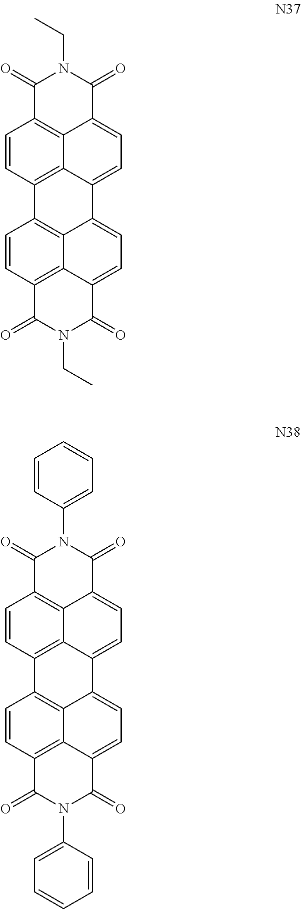 [SMILES: CCN1C(=O)c2ccc3c4ccc5c6c(ccc(c7ccc(c2c37)C1=O)c64)C(=O)N(CC)C5=O ; O=C1c2ccc3c4ccc5c6c(ccc(c7ccc(c2c37)C(=O)N1c1ccccc1)c64)C(=O)N(c1ccccc1)C5=O]

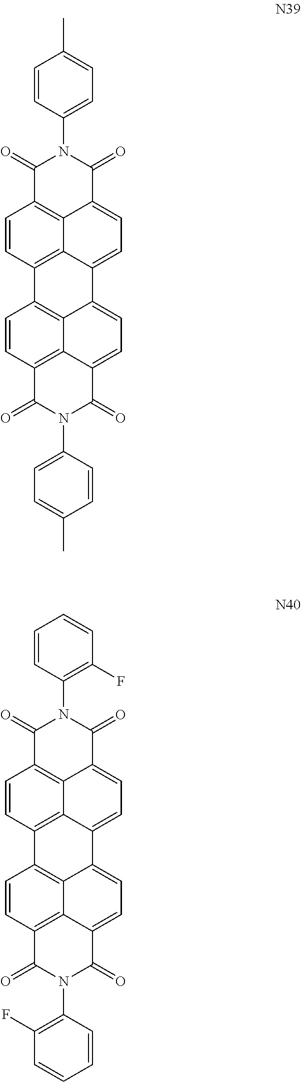 [SMILES: Cc1ccc(N2C(=O)c3ccc4c5ccc6c7c(ccc(c8ccc(c3c48)C2=O)c75)C(=O)N(c2ccc(C)cc2)C6=O)cc1 ; O=C1c2ccc3c4ccc5c6c(ccc(c7ccc(c2c37)C(=O)N1c1ccccc1F)c64)C(=O)N(c1ccccc1F)C5=O]

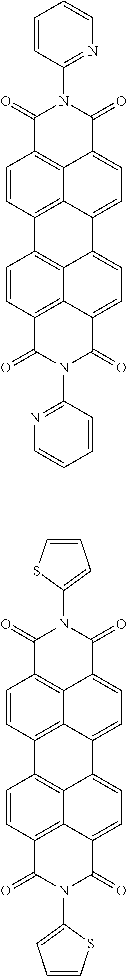 [SMILES: O=C1c2ccc3c4ccc5c6c(ccc(c7ccc(c2c37)C(=O)N1c1ccccn1)c64)C(=O)N(c1ccccn1)C5=O ; O=C1c2ccc3c4ccc5c6c(ccc(c7ccc(c2c37)C(=O)N1c1cccs1)c64)C(=O)N(c1cccs1)C5=O]

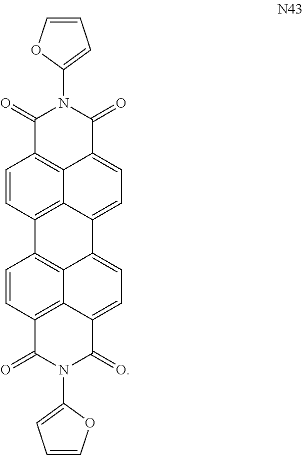 [SMILES: O=C1c2ccc3c4ccc5c6c(ccc(c7ccc(c2c37)C(=O)N1c1ccco1)c64)C(=O)N(c1ccco1)C5=O]

In one or more embodiments, the optical activation layer may include the first compound and the second compound. The optical activation layer may include a mixture of the first compound and the second compound. The optical activation layer may be a single layer. For example, the first compound and the second compound may be present in the optical activation layer in a mixed state.

In one or more embodiments, the optical activation layer may include a first layer adjacent to the first electrode and a second layer adjacent to the second electrode. For example, the first layer may be arranged between the first electrode and the second layer. The first layer may be arranged between the hole transport region and the second layer. The second layer may be arranged between the first layer and the second electrode. The second layer may be arranged between the first layer and the electron transport region.

In one or more embodiments, the first layer may include the first compound. The first layer may not include (e.g., may exclude) the second compound (e.g., free of the second compound).

In one or more embodiments, the second layer may include the second compound. The second layer may not include (e.g., may exclude) the first compound (e.g., free of the first compound).

For example, the optical activation layer may have a two-layer structure divided into the first layer including the first compound and the second layer including the second compound. For example, the first compound and the second compound may be present in the optical activation layer in a non-mixed state.

In one or more embodiments, the optical activation layer may further include a third layer arranged between the first layer and the second layer. The third layer may include the first compound and/or the second compound. The third layer may include a mixture of the first compound and the second compound. For example, the first compound and the second compound may be present in the third layer in a mixed state. For example, the optical activation layer may have a three-layer structure divided into i) the first layer including the first compound and not including (e.g., excluding) the second compound, ii) the third layer including both (e.g., simultaneously) the first compound and the second compound, and iii) the second layer including the second compound and not including (e.g., excluding) the first compound.

In one or more embodiments, the optical activation layer may be to (e.g., be configured to) absorb light having a wavelength in a range of about 400 nm to about 1,000 nm. For example, the optical activation layer may be to (e.g., be configured to) absorb red light, green light, blue light, near-infrared light, and/or any combination thereof. For example, the first compound in the optical activation layer may be to (e.g., be configured to) absorb light having a wavelength in a range of about 400 nm to about 1,000 nm.

In one or more embodiments, a maximum absorption wavelength of the optical activation layer and/or the first compound may be in a range of about 490 nm to about 750 nm. For example, the maximum absorption wavelength of the optical activation layer and/or the first compound may be in a range of about 490 nm to about 700 nm, about 490 nm to about 650 nm, about 490 nm to about 630 nm, about 520 nm to about 750 nm, about 520 nm to about 700 nm, about 520 nm to about 650 nm, about 520 nm to about 630 nm, or about 530 nm to about 630 nm. For example, the optical activation layer and/or the first compound may be to (e.g., be configured to) absorb green light and/or red light.

Another aspect of the disclosure provides an electronic apparatus including: the optoelectronic device;
a light-emitting device configured to not overlap (e.g., is not overlapping) the optoelectronic device; and
a color filter, a color conversion layer, a touch screen layer, a polarizing layer, or any combination thereof.

In one or more embodiments, the light-emitting device may include an emission layer.

Another aspect of the disclosure provides electronic equipment including the optoelectronic device,
wherein the electronic equipment may be at least one selected from among a flat panel display, a curved display, a computer monitor, a medical monitor, a television, a billboard, an indoor or outdoor light and/or light for signal, a head-up display, a fully or partially transparent display, a flexible display, a rollable display, a foldable display, a stretchable display, a laser printer, a telephone, a portable phone, a tablet personal computer, a phablet, a personal digital assistant (PDA), a wearable device, a laptop computer, a digital camera, a camcorder, a viewfinder, a micro display, a three-dimensional (3D) display, a virtual reality or augmented reality display, a vehicle, a video wall with multiple displays tiled together, a theater or stadium screen, a phototherapy device, a signboard, a vehicle sensor, a household sensor, a solar cell, and combinations thereof.

Another aspect of the disclosure provides an organic compound represented by Formula 1 (the first compound).

In one or more embodiments, the organic compound may be represented by Formula 1-1:

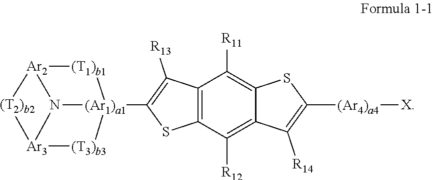

In Formula 1-1,
X may be an electron-acceptor group,
Ar₁ to Ar₄ may each independently be a C₃-C₆₀ carbocyclic group unsubstituted or substituted with at least one R₁₀ₐ or a C₁-C₆₀ heterocyclic group unsubstituted or substituted with at least one R₁₀ₐ,
T₁ to T₃ may each independently be selected from a single bond, *—O—*′, *—S—*′, *—C(R₂₁)(R₂₂)—*′, *—Si(R₂₁)(R₂₂)—*′, and *—N(R₂₁)—*′,
each of a1, a4, and b1 to b3 may be 0 or 1,
R₁₁ to R₁₄, R₂₁, and R₂₂ may each independently be hydrogen, deuterium, —F, —Cl, —Br, —I, a hydroxyl group, a cyano group, a nitro group, a C₁-C₆₀ alkyl group unsubstituted or substituted with at least one R₁₀ₐ, a C₂-C₆₀ alkenyl group unsubstituted or substituted with at least one R₁₀ₐ, a C₂-C₆₀ alkynyl group unsubstituted or substituted with at least one R₁₀ₐ, a C₁-C₆ alkoxy group unsubstituted or substituted with at least one R₁₀ₐ, a C₃-C₆₀ carbocyclic group unsubstituted or substituted with at least one R₁₀ₐ, a C₁-C₆₀ heterocyclic group unsubstituted or substituted with at least one R₁₀ₐ, a C₆-C₆₀ aryloxy group unsubstituted or substituted with at least one R₁₀ₐ, a C₆-C₆₀ arylthio group unsubstituted or substituted with at least one R₁₀ₐ, a C₇-C₆₀ arylalkyl group unsubstituted or substituted with at least one R₁₀ₐ, a C₂-C₆₀ heteroarylalkyl group unsubstituted or substituted with at least one R₁₀ₐ, —C(Q₁)(Q₂)(Q₃), —Si(Q₁)(Q₂)(Q₃), —N(Q₁)(Q₂), —B(Q₁)(Q₂), —C(═O)(Q₁), —S(═O)₂(Q₁), or —P(═O)(Q₁)(Q₂),
R₁₀ₐ may be:
deuterium, —F, —Cl, —Br, —I, a hydroxyl group, a cyano group, or a nitro group;
a C₁-C₆₀ alkyl group, a C₂-C₆₀ alkenyl group, a C₂-C₆₀ alkynyl group, or a C₁-C₆₀ alkoxy group, each unsubstituted or substituted with deuterium, —F, —Cl, —Br, —I, a hydroxyl group, a cyano group, a nitro group, a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, a C₂-C₆₀ heteroarylalkyl group, —Si(Q₁₁)(Q₁₂)(Q₁₃), —N(Q₁₁)(Q₁₂), —B(Q₁₁)(Q₁₂), —C(═O)(Q₁₁), —S(═O)₂(Q₁₁), —P(═O)(Q₁₁)(Q₁₂), or any combination thereof;
a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, or a C₂-C₆₀ heteroarylalkyl group, each unsubstituted or substituted with deuterium, —F, —Cl, —Br, —I, a hydroxyl group, a cyano group, a nitro group, a C₁-C₆₀ alkyl group, a C₂-C₆₀ alkenyl group, a C₂-C₆₀ alkynyl group, a C₁-C₆₀ alkoxy group, a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, a C₂-C₆₀ heteroarylalkyl group, —Si(Q₂₁)(Q₂₂)(Q₂₃), —N(Q₂₁)(Q₂₂), —B(Q₂₁)(Q₂₂), —C(═O)(Q₂₁), —S(═O)₂(Q₂₁), —P(═O)(Q₂₁)(Q₂₂), or any combination thereof; or
—Si(Q₃₁)(Q₃₂)(Q₃₃), —N(Q₃₁)(Q₃₂), —B(Q₃₁)(Q₃₂), —C(═O)(Q₃₁), —S(═O)₂(Q₃₁), or —P(═O)(Q₃₁)(Q₃₂), and
Q₁ to Q₃, Q₁₁ to Q₁₃, Q₂₁ to Q₂₃, and Q₃₁ to Q₃₃ may each independently be:
hydrogen, deuterium, —F, —Cl, —Br, —I, a hydroxyl group, a cyano group, a nitro group, a C₁-C₆₀ alkyl group, a C₂-C₆₀ alkenyl group, a C₂-C₆₀ alkynyl group, or a C₁-C₆₀ alkoxy group; or
a C₃-C₆₀ carbocyclic group, a C₁-C₆₀ heterocyclic group, a C₇-C₆₀ arylalkyl group, or a C₂-C₆₀ heteroarylalkyl group, each unsubstituted or substituted with deuterium, —F, a cyano group, a C₁-C₆₀ alkyl group, a C₁-C₆₀ alkoxy group, a phenyl group, a biphenyl group, or any combination thereof.

According to one or more embodiments, in Formulae 1 and 1-1,
X may be selected from among groups represented by Formulae 1-A, 1-B-1 to 1-B-5, and/or 1-C-1 to 1-C-6:

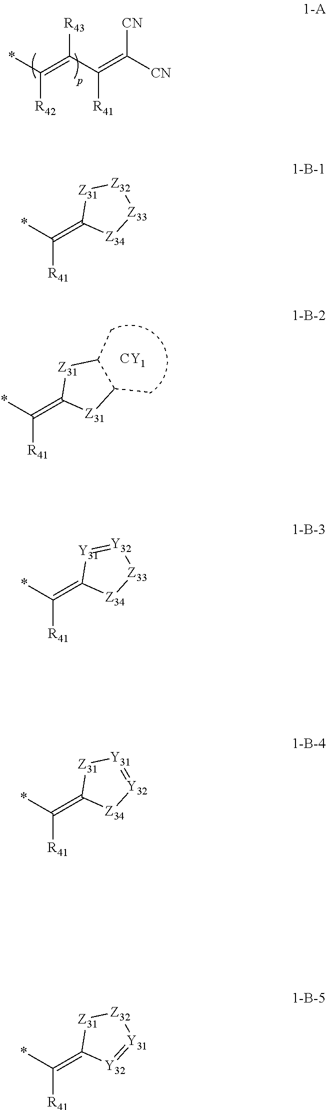

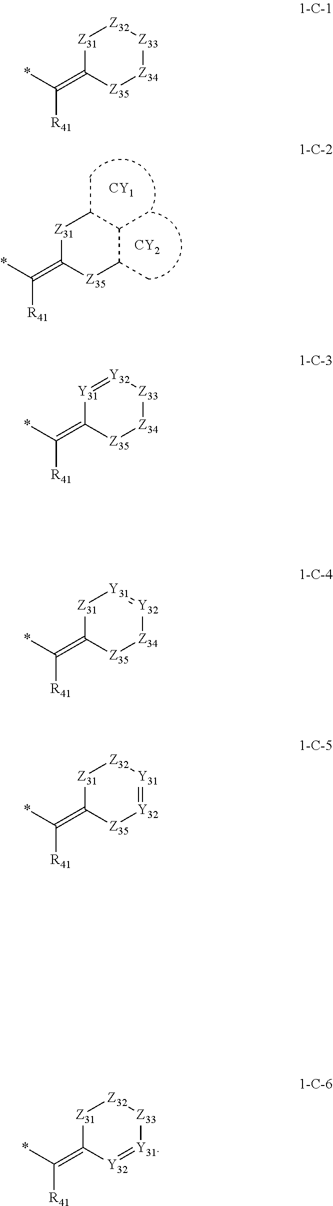

In Formulae 1-A, 1-B-1 to 1-B-5, and 1-C-1 to 1C-6,
Z₃₁ to Z₃₅ may each independently be selected from among O, S, N(R₃₁), C(R₃₁)(R₃₂), C(═O), C(═S), and C═C(R₃₁)(R₃₂),
each of Y₃₁ and Y₃₂ may be N or C(R₃₃),
R₃₁ to R₃₃ and R₄₁ to R₄₃ may each independently be hydrogen, deuterium, —F, —Cl, —Br, —I, a hydroxyl group, a cyano group, a nitro group, a C₁-C₆₀ alkyl group unsubstituted or substituted with at least one R₁₀ₐ, a C₂-C₆₀ alkenyl group unsubstituted or substituted with at least one R₁₀ₐ, a C₂-C₆₀ alkynyl group unsubstituted or substituted with at least one R₁₀ₐ, a C₁-C₆₀ alkoxy group unsubstituted or substituted with at least one R₁₀ₐ, a C₃-C₆₀ carbocyclic group unsubstituted or substituted with at least one R₁₀ₐ, a C₁-C₆₀ heterocyclic group unsubstituted or substituted with at least one R₁₀ₐ, a C₆-C₆₀ aryloxy group unsubstituted or substituted with at least one R₁₀ₐ, a C₆-C₆₀ arylthio group unsubstituted or substituted with at least one R₁₀ₐ, a C₇-C₆₀ arylalkyl group unsubstituted or substituted with at least one R₁₀ₐ, a C₂-C₆₀ heteroarylalkyl group unsubstituted or substituted with at least one R₁₀ₐ, —C(Q₁)(Q₂)(Q₃), —Si(Q₁)(Q₂)(Q₃), —N(Q₁)(Q₂), —B(Q₁)(Q₂), —C(═O)(Q₁), —S(═O)₂(Q₁), or —P(═O)(Q₁)(Q₂),
CY₁ and CY₂ may each independently be a C₃-C₆₀ carbocyclic group unsubstituted or substituted with at least one R₁₀ₐ or a C₁-C₆₀ heterocyclic group unsubstituted or substituted with at least one R₁₀ₐ,
p may be an integer from 0 to 2,
Q₁ to Q₃ and R₁₀ₐ may each be as defined herein, and
* indicates a binding site to a neighboring atom.

According to one or more embodiments, in Formulae 1 and 1-1,
Ar₁ to Ar₄ may each independently be a benzene group, a pyridine group, a pyrimidine group, a triazine group, a naphthalene group, an anthracene group, a phenanthrene group, a phenalene group, a thiophene group, a furan group, a benzothiophene group, a benzofuran group, a dibenzothiophene group, or a dibenzofuran group, each unsubstituted or substituted with at least one R₁₀ₐ.

According to one or more embodiments, in Formulae 1 and 1-1, a4 may be 0.

According to one or more embodiments, in Formulae 1 and 1-1, R₁₁ to R₁₄ may each independently be hydrogen, deuterium, —F, —Cl, —Br, —I, a cyano group, a C₁-C₆₀ alkyl group unsubstituted or substituted with at least one R₁₀ₐ, a C₁-C₆₀ alkoxy group unsubstituted or substituted with at least one R₁₀ₐ, or a C₃-C₆₀ aryl group unsubstituted or substituted with at least one R₁₀ₐ. For example, each of R₁₁ and R₁₂ may not be a C₁-C₆₀ heteroaryl group unsubstituted or substituted with at least one R₁₀ₐ. For example, each of R₁₁ and R₁₂ may not be a thiophene group.

According toAccording to one or more embodiments, in Formulae 1 and 1-1,
T₁ to T₃ may each independently be selected from among a single bond, *—O—*′, *—S—*′, and *—C(R₂₁)(R₂₂)—*′, and
R₂₁ and R₂₂ may each independently be hydrogen, deuterium, —F, —Cl, —Br, —I, a cyano group, a C₁-C₆₀ alkyl group unsubstituted or substituted with at least one R₁₀ₐ, a C₁-C₆₀ alkoxy group unsubstituted or substituted with at least one R₁₀ₐ, a C₃-C₆₀ aryl group unsubstituted or substituted with at least one R₁₀ₐ, or a C₁-C₆₀ heteroaryl group unsubstituted or substituted with at least one R₁₀ₐ.

According to one or more embodiments, in Formulae 1 and 1-1, the sum of b1 to b3 may be 0 or 1. For example, i) each of b1 to b3 may be 0, ii) each of b1 and b2 may be 0, and b3 may be 1, iii) each of b1 and b3 may be 0, and b2 may be 1, or iv) each of b2 and b3 may be 0, and b1 may be 1.

In one or more embodiments, when a1 is 0, b1 may be 0, and b3 may be 0.

According to one or more embodiments, in Formulae 1 and 1-1,
a group represented by

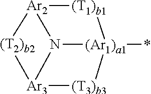

may be a group selected from among Formulae L1 to L26:

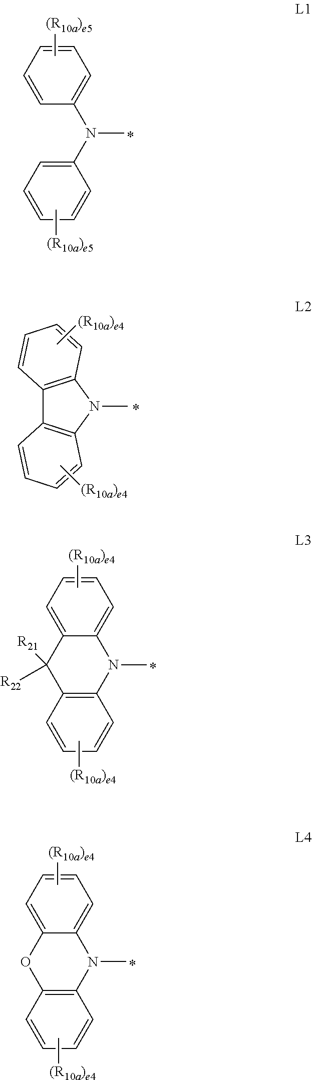

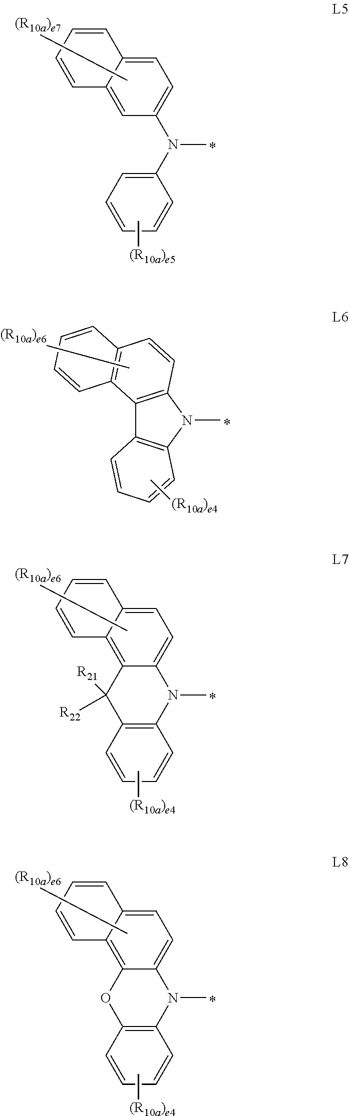

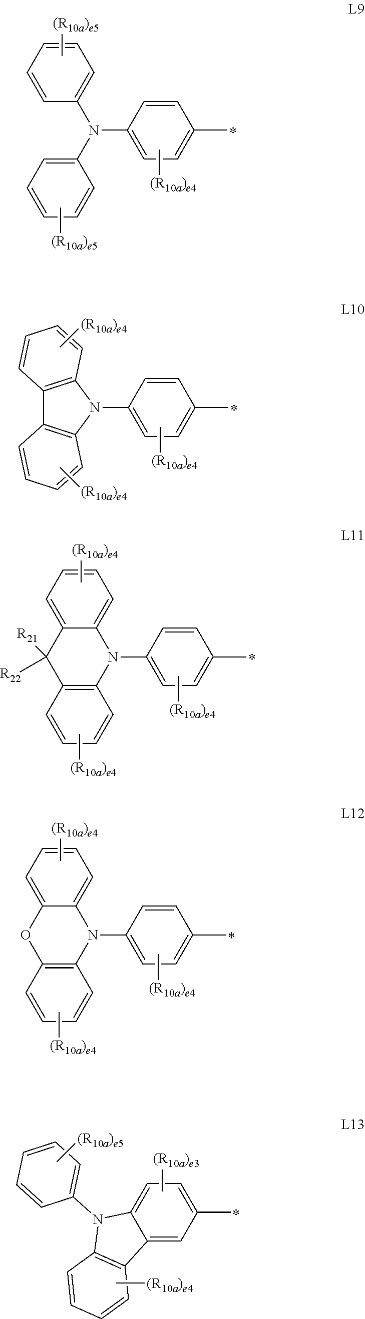

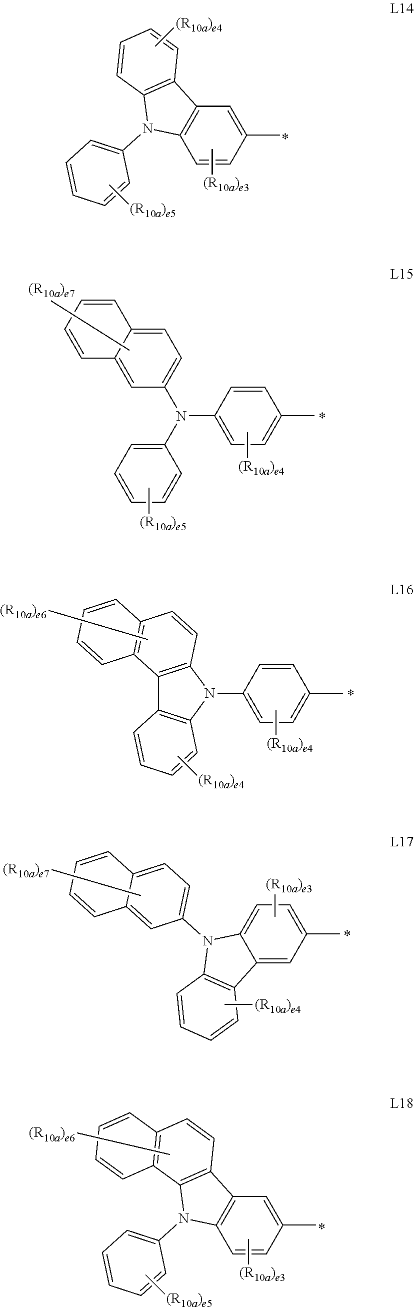

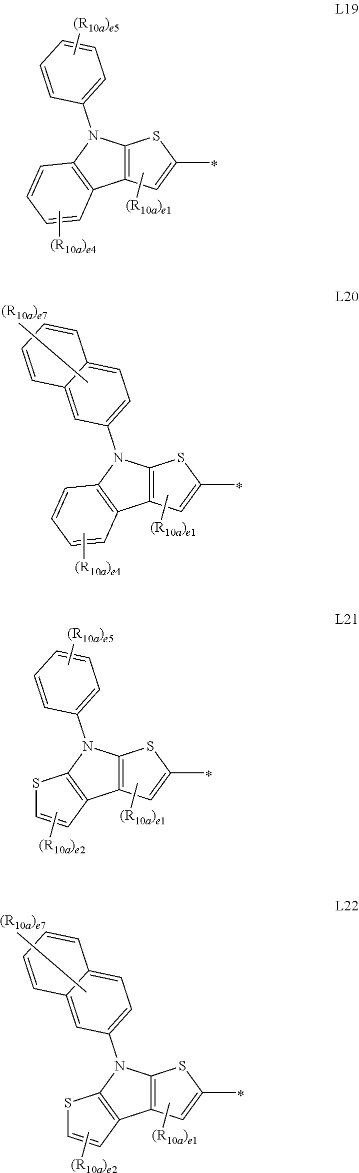

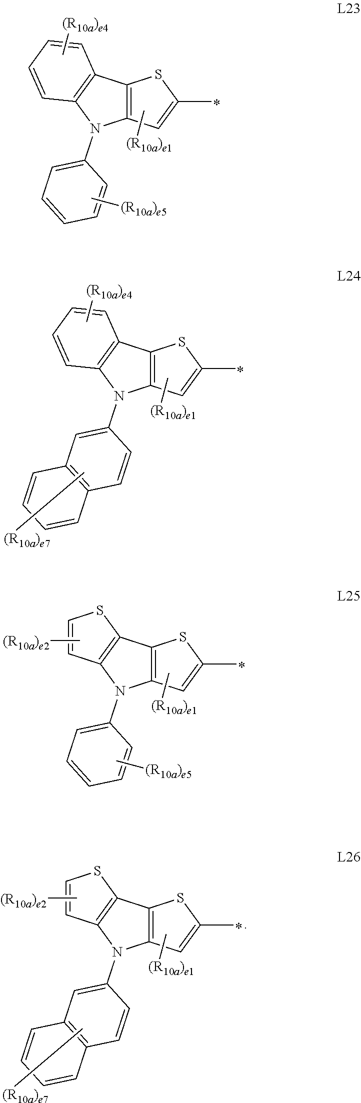

In Formulae L1 to L26,
R₁₀ₐ may be as defined herein,
e1 may be 0 or 1,
e2 may be an integer from 0 to 2,
e3 may be an integer from 0 to 3,
e4 may be an integer from 0 to 4,
e5 may be an integer from 0 to 5,
e6 may be an integer from 0 to 6,
e7 may be an integer from 0 to 7, and
* indicates a binding site to a neighboring atom.

In one or more embodiments, a highest occupied molecular orbital (HOMO) energy level of the organic compound may be in a range of about −5.8 eV to about −5.0 eV, and/or a lowest unoccupied molecular orbital (LUMO) energy level of the organic compound may be in a range of about −2.7 eV to about −3.9 eV.

In one or more embodiments, the organic compound may be one selected from among Compounds P1 to P35:

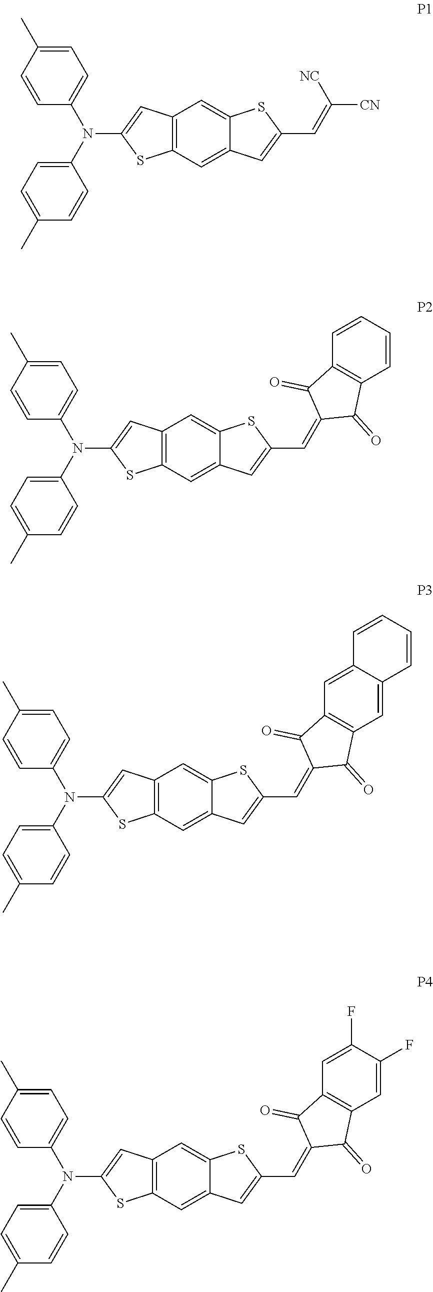 [SMILES: Cc1ccc(N(c2ccc(C)cc2)c2cc3cc4sc(C=C(C#N)C#N)cc4cc3s2)cc1 ; Cc1ccc(N(c2ccc(C)cc2)c2cc3cc4sc(C=C5C(=O)c6ccccc6C5=O)cc4cc3s2)cc1 ; Cc1ccc(N(c2ccc(C)cc2)c2cc3cc4sc(C=C5C(=O)c6cc7ccccc7cc6C5=O)cc4cc3s2)cc1 ; Cc1ccc(N(c2ccc(C)cc2)c2cc3cc4sc(C=C5C(=O)c6cc(F)c(F)cc6C5=O)cc4cc3s2)cc1]

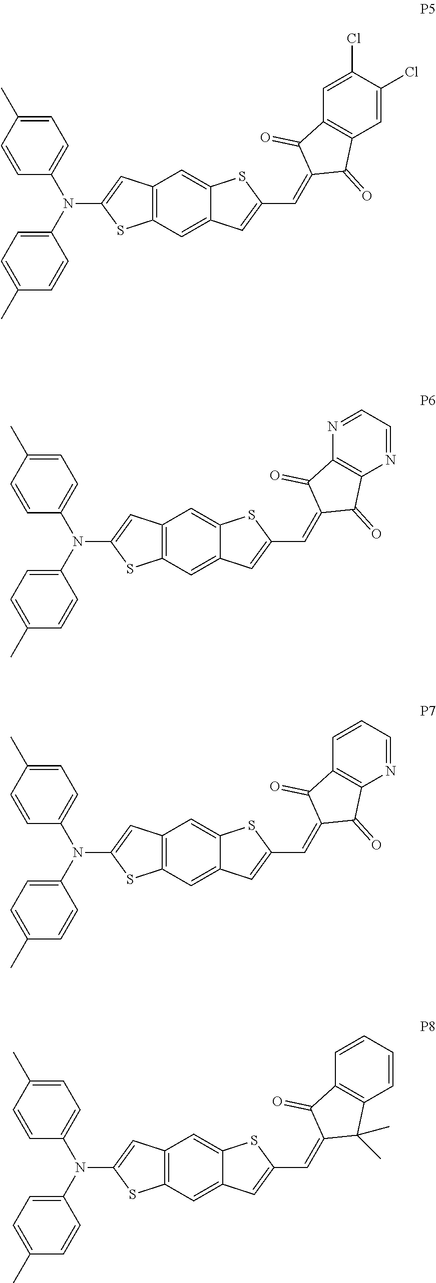 [SMILES: Cc1ccc(N(c2ccc(C)cc2)c2cc3cc4sc(C=C5C(=O)c6cc(Cl)c(Cl)cc6C5=O)cc4cc3s2)cc1 ; Cc1ccc(N(c2ccc(C)cc2)c2cc3cc4sc(C=C5C(=O)c6nccnc6C5=O)cc4cc3s2)cc1 ; Cc1ccc(N(c2ccc(C)cc2)c2cc3cc4sc(/C=C5\C(=O)c6cccnc6C5=O)cc4cc3s2)cc1 ; Cc1ccc(N(c2ccc(C)cc2)c2cc3cc4sc(/C=C5\C(=O)c6ccccc6C5(C)C)cc4cc3s2)cc1]

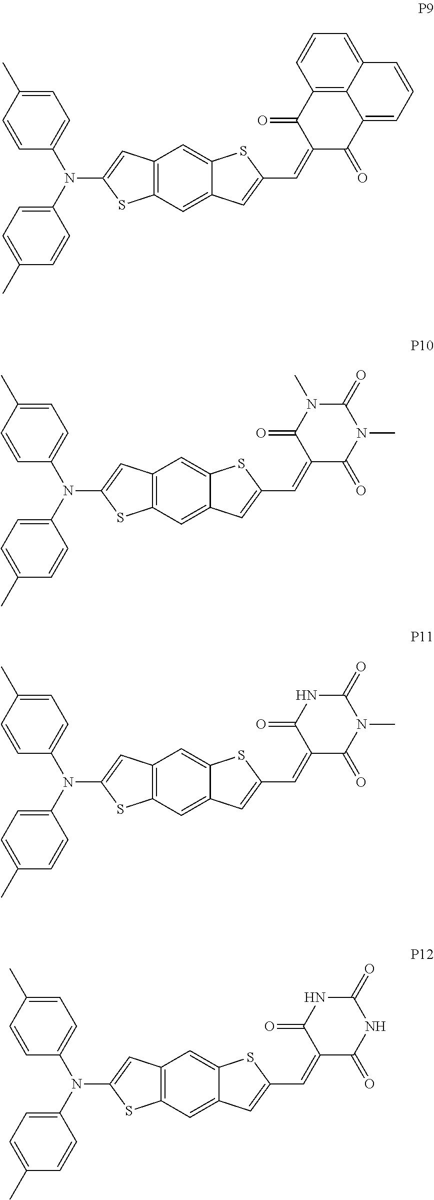 [SMILES: Cc1ccc(N(c2ccc(C)cc2)c2cc3cc4sc(C=c5c(=O)c6cccc7cccc(c5=O)c76)cc4cc3s2)cc1 ; Cc1ccc(N(c2ccc(C)cc2)c2cc3cc4sc(C=C5C(=O)N(C)C(=O)N(C)C5=O)cc4cc3s2)cc1 ; Cc1ccc(N(c2ccc(C)cc2)c2cc3cc4sc(/C=C5\C(=O)NC(=O)N(C)C5=O)cc4cc3s2)cc1 ; Cc1ccc(N(c2ccc(C)cc2)c2cc3cc4sc(C=C5C(=O)NC(=O)NC5=O)cc4cc3s2)cc1]

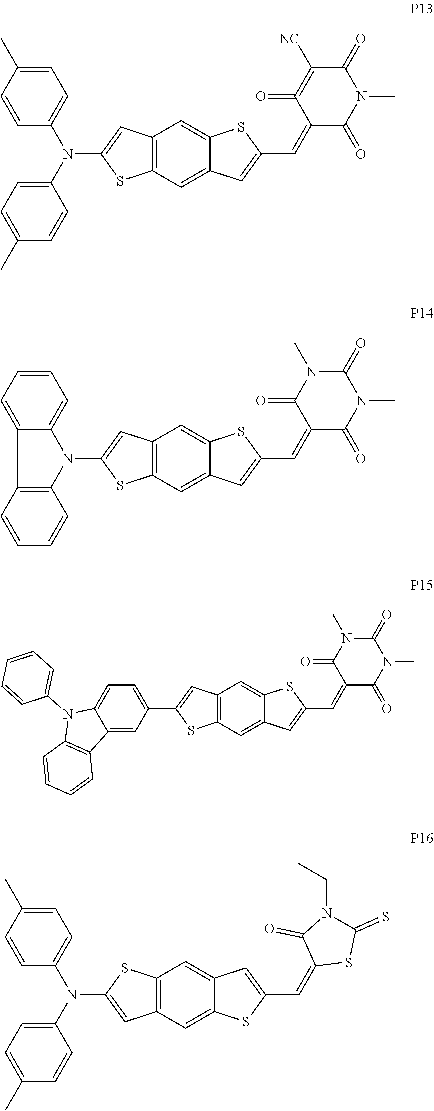

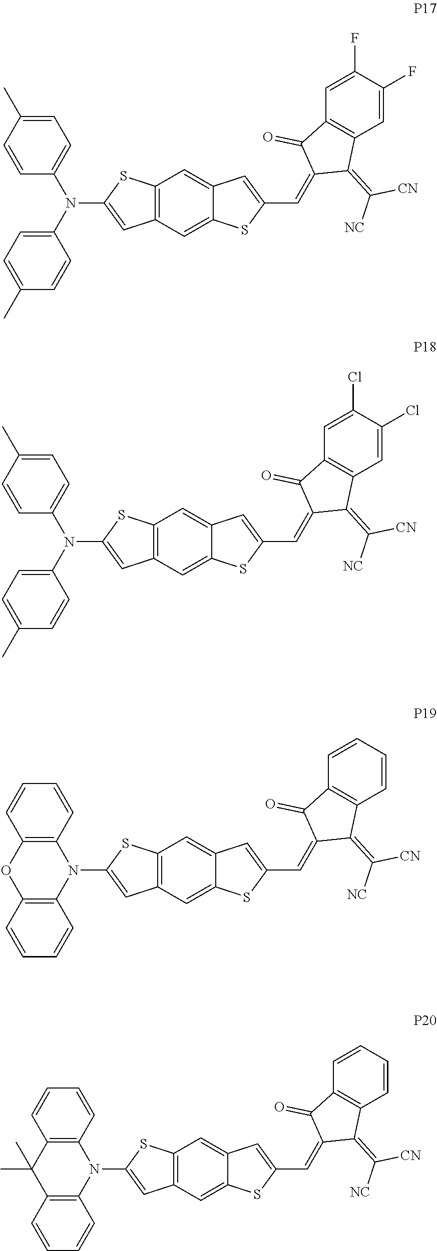 [SMILES: Cc1ccc(N(c2ccc(C)cc2)c2cc3cc4sc(/C=C5\C(=O)c6cc(F)c(F)cc6C5=C(C#N)C#N)cc4cc3s2)cc1 ; Cc1ccc(N(c2ccc(C)cc2)c2cc3cc4sc(/C=C5\C(=O)c6cc(Cl)c(Cl)cc6C5=C(C#N)C#N)cc4cc3s2)cc1 ; N#CC(C#N)=C1/C(=C/c2cc3cc4sc(N5c6ccccc6Oc6ccccc65)cc4cc3s2)C(=O)c2ccccc21 ; CC1(C)c2ccccc2N(c2cc3cc4sc(/C=C5\C(=O)c6ccccc6C5=C(C#N)C#N)cc4cc3s2)c2ccccc21]

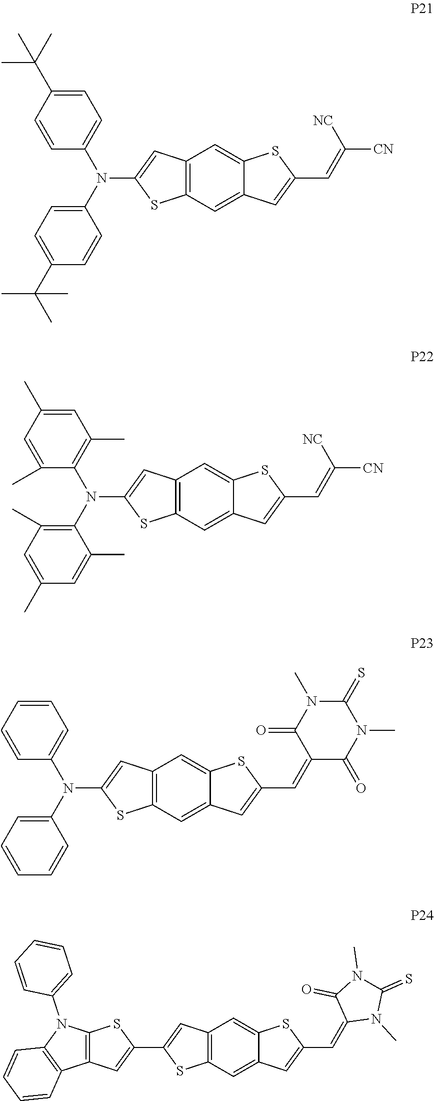 [SMILES: CC(C)(C)c1ccc(N(c2ccc(C(C)(C)C)cc2)c2cc3cc4sc(C=C(C#N)C#N)cc4cc3s2)cc1 ; Cc1cc(C)c(N(c2cc3cc4sc(C=C(C#N)C#N)cc4cc3s2)c2c(C)cc(C)cc2C)c(C)c1 ; CN1C(=O)C(=Cc2cc3cc4sc(N(c5ccccc5)c5ccccc5)cc4cc3s2)C(=O)N(C)C1=S ; CN1C(=O)/C(=C\c2cc3cc4sc(-c5cc6c7ccccc7n(-c7ccccc7)c6s5)cc4cc3s2)N(C)C1=S]

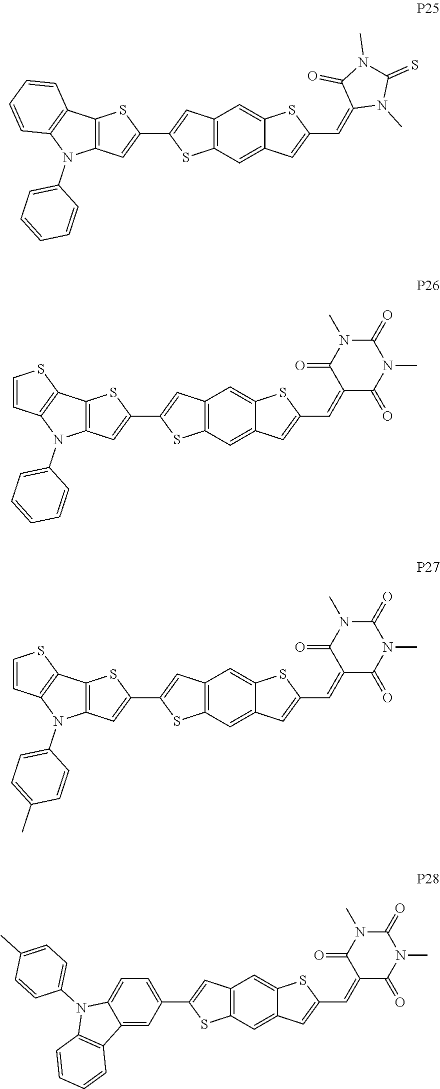

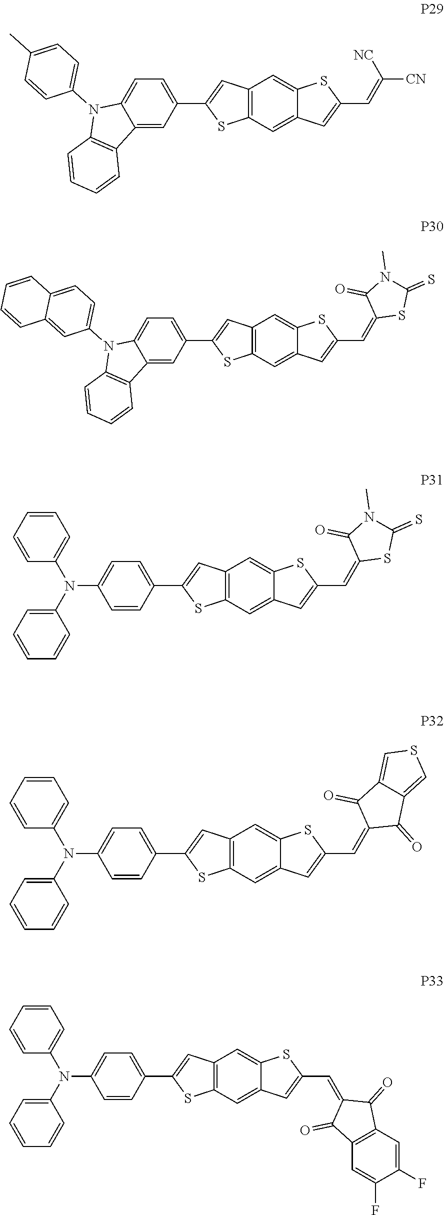 [SMILES: Cc1ccc(-n2c3ccccc3c3cc(-c4cc5cc6sc(C=C(C#N)C#N)cc6cc5s4)ccc32)cc1 ; CN1C(=O)/C(=C\c2cc3cc4sc(-c5ccc6c(c5)c5ccccc5n6-c5ccc6ccccc6c5)cc4cc3s2)SC1=S ; CN1C(=O)/C(=C\c2cc3cc4sc(-c5ccc(N(c6ccccc6)c6ccccc6)cc5)cc4cc3s2)SC1=S ; O=C1C(=Cc2cc3cc4sc(-c5ccc(N(c6ccccc6)c6ccccc6)cc5)cc4cc3s2)C(=O)c2cscc21 ; O=C1C(=Cc2cc3cc4sc(-c5ccc(N(c6ccccc6)c6ccccc6)cc5)cc4cc3s2)C(=O)c2cc(F)c(F)cc21]

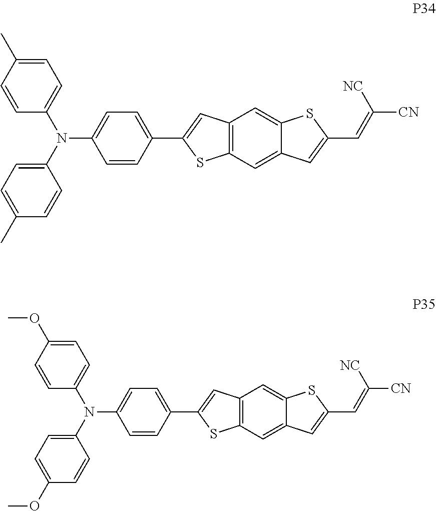 [SMILES: Cc1ccc(N(c2ccc(C)cc2)c2ccc(-c3cc4cc5sc(C=C(C#N)C#N)cc5cc4s3)cc2)cc1 ; COc1ccc(N(c2ccc(OC)cc2)c2ccc(-c3cc4cc5sc(C=C(C#N)C#N)cc5cc4s3)cc2)cc1]

For example, due to the inclusion of an electron-donor group represented by

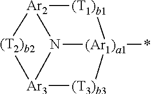

an electron-acceptor group represented by X, and/or a group represented by

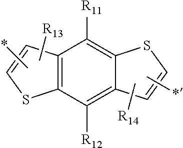

with an appropriate or suitable conjugation length between the electron-donor group and the electron-acceptor group, the organic compound represented by Formula 1 may be to (be configured to) absorb green light and/or red light, and may have excellent or suitable deposition stability and excellent or suitable heat resistance. Accordingly, the optical activation layer (for example, the first layer) formed by depositing the organic compound may have excellent or suitable purity, and thus, the optoelectronic device including the organic compound may have excellent or suitable external quantum efficiency and/or excellent or suitable dark current density.

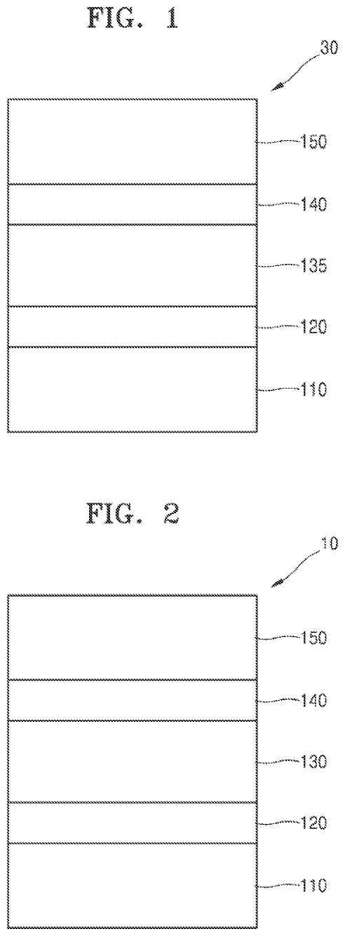

### Description of FIGS. 1 and 2

FIG. 1 is a schematic view of an optoelectronic device 30 according to one or more embodiments. The optoelectronic device 30 may include a first electrode 110, a hole transport region 120, an optical activation layer 135, an electron transport region 140, and a second electrode 150.

FIG. 2 is a schematic view of a light-emitting device 10. The light-emitting device 10 may include the first electrode 110, the hole transport region 120, an emission layer 130, the electron transport region 140, and the second electrode 150.

In one or more embodiments, each of the first electrode 110, the hole transport region 120, the electron transport region 140, and the second electrode 150 of the optoelectronic device 30 may be substantially integrated in one body with each of the first electrode 110, the hole transport region 120, the electron transport region 140, and/or the second electrode 150 of the light-emitting device 10, respectively. In one or more embodiments, each of the first electrode 110, the hole transport region 120, the electron transport region 140, and the second electrode 150 of the optoelectronic device 30 may be substantially spaced (e.g. apart) from each of the first electrode 110, the hole transport region 120, the electron transport region 140, and/or the second electrode 150 of the light-emitting device 10, respectively, but may substantially include the same materials and be formed at the same time.

Hereinafter, the structures of the optoelectronic device 30 and the light-emitting device 10 according to embodiments and a method of manufacturing the same will be described in connection with FIGS. 1 and 2.

### First Electrode 110

In FIG. 1, a substrate may be additionally arranged under the first electrode 110 or on the second electrode 150. As the substrate, a glass substrate and/or a plastic substrate may be utilized. The substrate may be a flexible substrate. For example, the substrate may include plastic having excellent or suitable heat resistance and durability, such as polyimide, polyethylene terephthalate (PET), polycarbonate, polyethylene naphthalate, polyarylate (PAR), polyetherimide, or any combination thereof.

The first electrode 110 may be formed by, for example, depositing or sputtering a material for forming the first electrode 110 on the substrate. When the first electrode 110 is an anode, a material for forming the first electrode 110 may be a high work-function material that facilitates injection of holes. The “term” high work-function material” as utilized herein refers to a substance (e.g., a metal or metal alloy) that requires a relatively high amount of energy to emit electrons from its surface.

The first electrode 110 may be a reflective electrode, a semi-transmissive electrode, or a transmissive electrode. In one or more embodiments, when the first electrode 110 is a transmissive electrode, a material for forming the first electrode 110 may include indium tin oxide (ITO), indium zinc oxide (IZO), tin oxide (SnO₂), zinc oxide (ZnO), or any combination thereof. In one or more embodiments, when the first electrode 110 is a semi-transmissive electrode or a reflective electrode, a material for forming the first electrode 110 may include magnesium (Mg), silver (Ag), aluminum (Al), aluminum-lithium (Al—Li), calcium (Ca), magnesium-indium (Mg—In), magnesium-silver (Mg—Ag), or any combination thereof.

The first electrode 110 may have a single-layer structure including (e.g., consisting of) a single layer or a multi-layer structure including multiple layers. For example, the first electrode 110 may have a three-layer structure of ITO/Ag/ITO.

### Hole Transport Region 120

The hole transport region 120 may have i) a single-layer structure including (e.g., consisting of) a single layer including (e.g., consisting of) a single material, ii) a single-layer structure including (e.g., consisting of) a single layer including multiple different materials, or iii) a multi-layer structure including multiple layers including multiple different materials.

The hole transport region 120 may include a hole injection layer, a hole transport layer, an emission auxiliary layer, an electron blocking layer, or any combination thereof.

For example, the hole transport region 120 may have a multi-layer structure including a hole injection layer/hole transport layer structure, a hole injection layer/hole transport layer/emission auxiliary layer structure, a hole injection layer/emission auxiliary layer structure, a hole transport layer/emission auxiliary layer structure, or a hole injection layer/hole transport layer/electron blocking layer structure. In one or more embodiments, constituent layers of each structure are stacked sequentially from the first electrode 110.

The hole transport region 120 may include a compound represented by Formula 201, a compound represented by Formula 202, and/or any combination thereof:

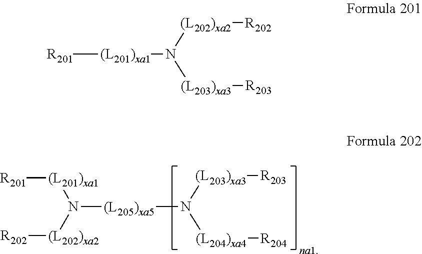

In Formulae 201 and 202,
L₂₀₁ to L₂₀₄ may each independently be a C₃-C₆₀ carbocyclic group unsubstituted or substituted with at least one R₁₀ₐ or a C₁-C₆₀ heterocyclic group unsubstituted or substituted with at least one R₁₀ₐ,
L₂₀₅ may be *—O—*′, *—S—*′, *—N(Q₂₀₁)-*′, a C₁-C₂₀ alkylene group unsubstituted or substituted with at least one R₁₀ₐ, a C₂-C₂₀ alkenylene group unsubstituted or substituted with at least one R₁₀ₐ, a C₃-C₆₀ carbocyclic group unsubstituted or substituted with at least one R₁₀ₐ, or a C₁-C₆₀ heterocyclic group unsubstituted or substituted with at least one R₁₀ₐ,
xa1 to xa4 may each independently be an integer from 0 to 5,
xa5 may be an integer from 1 to 10,
R₂₀₁ to R₂₀₄ and Q₂₀₁ may each independently be a C₃-C₆₀ carbocyclic group unsubstituted or substituted with at least one R₁₀ₐ or a C₁-C₆₀ heterocyclic group unsubstituted or substituted with at least one R₁₀ₐ,
R₂₀₁ and R₂₀₂ may optionally be linked to each other via a single bond, a C₁-C₅ alkylene group unsubstituted or substituted with at least one R₁₀ₐ, or a C₂-C₅ alkenylene group unsubstituted or substituted with at least one R₁₀ₐ, to form a C₈-C₆₀ polycyclic group (for example, a carbazole group, etc.) unsubstituted or substituted with at least one R₁₀ₐ (for example, Compound HT16, etc.),

R₂₀₃ and R₂₀₄ may optionally be linked to each other via a single bond, a C₁-C₅ alkylene group unsubstituted or substituted with at least one R₁₀ₐ, or a C₂-C₅ alkenylene group unsubstituted or substituted with at least one R₁₀ₐ, to form a C₈-C₆₀ polycyclic group unsubstituted or substituted with at least one R₁₀ₐ, and
na1 may be an integer from 1 to 4.

For example, each of Formulae 201 and 202 may include at least one selected from among groups represented by Formulae CY201 to CY217:

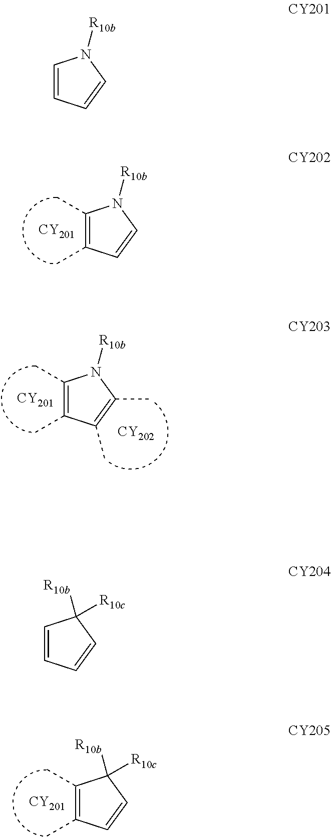

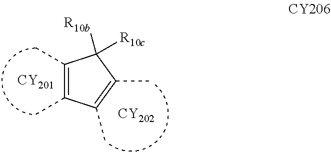

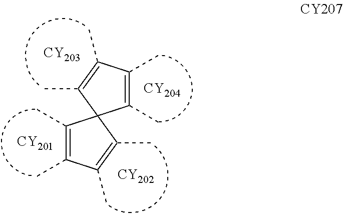

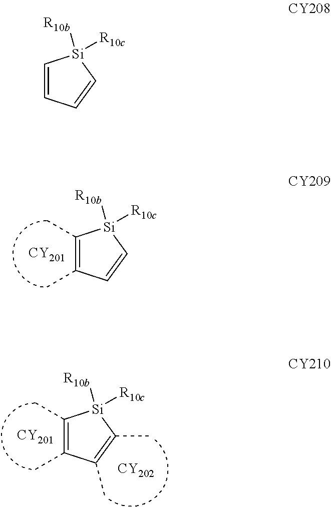

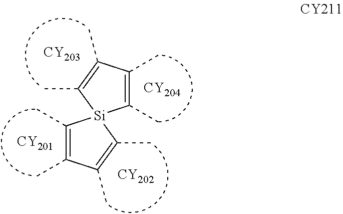

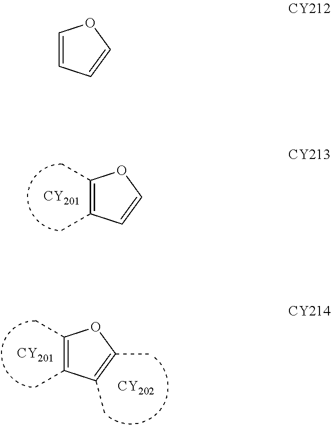

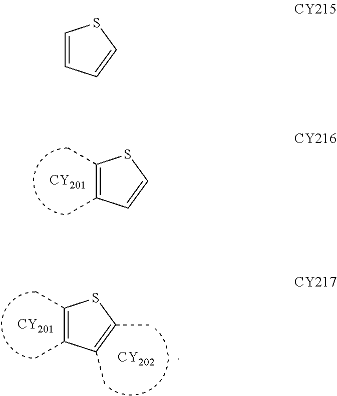

In Formulae CY201 to CY217, R_{10b} and R_{10c} may each be defined as in connection with R₁₀ₐ, ring CY₂₀₁ to ring CY₂₀₄ may each independently be a C₃-C₂₀ carbocyclic group or a C₁-C₂₀ heterocyclic group, and at least one hydrogen in Formulae CY201 to CY217 may be unsubstituted or substituted with R₁₀ₐ.

According to one or more embodiments, in Formulae CY201 to CY217, ring CY₂₀₁ to ring CY₂₀₄ may each independently be a benzene group, a naphthalene group, a phenanthrene group, or an anthracene group.

In one or more embodiments, each of Formulae 201 and 202 may include at least one selected from among the groups represented by Formulae CY201 to CY203.

In one or more embodiments, Formula 201 may include at least one selected from among the groups represented by Formulae CY201 to CY203 and at least one selected from among the groups represented by Formulae CY204 to CY217.

According to one or more embodiments, in Formula 201, xa1 may be 1, R₂₀₁ may be one selected from among the groups represented by Formulae CY201 to CY203, xa2 may be 0, and R₂₀₂ may be one selected from among the groups represented by Formulae CY204 to CY207.

In one or more embodiments, each of Formulae 201 and 202 may not include (e.g., may exclude) the (e.g., any) groups represented by Formulae CY201 to CY203.

In one or more embodiments, each of Formulae 201 and 202 may not include (e.g., may exclude) the (e.g., any) groups represented by Formulae CY201 to CY203, and may include at least one of the groups represented by Formulae CY204 to CY217.

In one or more embodiments, each of Formulae 201 and 202 may not include (e.g., may exclude) the (e.g., any) groups represented by Formulae CY201 to CY217.

For example, the hole transport region 120 may include at least one selected from among Compounds HT1 to HT46, m-MTDATA, TDATA, 2-TNATA, NPB(NPD), β-NPB, TPD, Spiro-TPD, Spiro-NPB, methylated NPB, TAPC, HMTPD, 4,4′,4″-tris(N-carbazolyl)triphenylamine (TCTA), polyaniline/dodecylbenzenesulfonic acid (PANI/DBSA), poly(3,4-ethylenedioxythiophene)/poly(4-styrenesulfonate) (PEDOT/PSS), polyaniline/camphor sulfonic acid (PANI/CSA), polyaniline/poly(4-styrenesulfonate) (PANI/PSS), and/or any combination thereof:

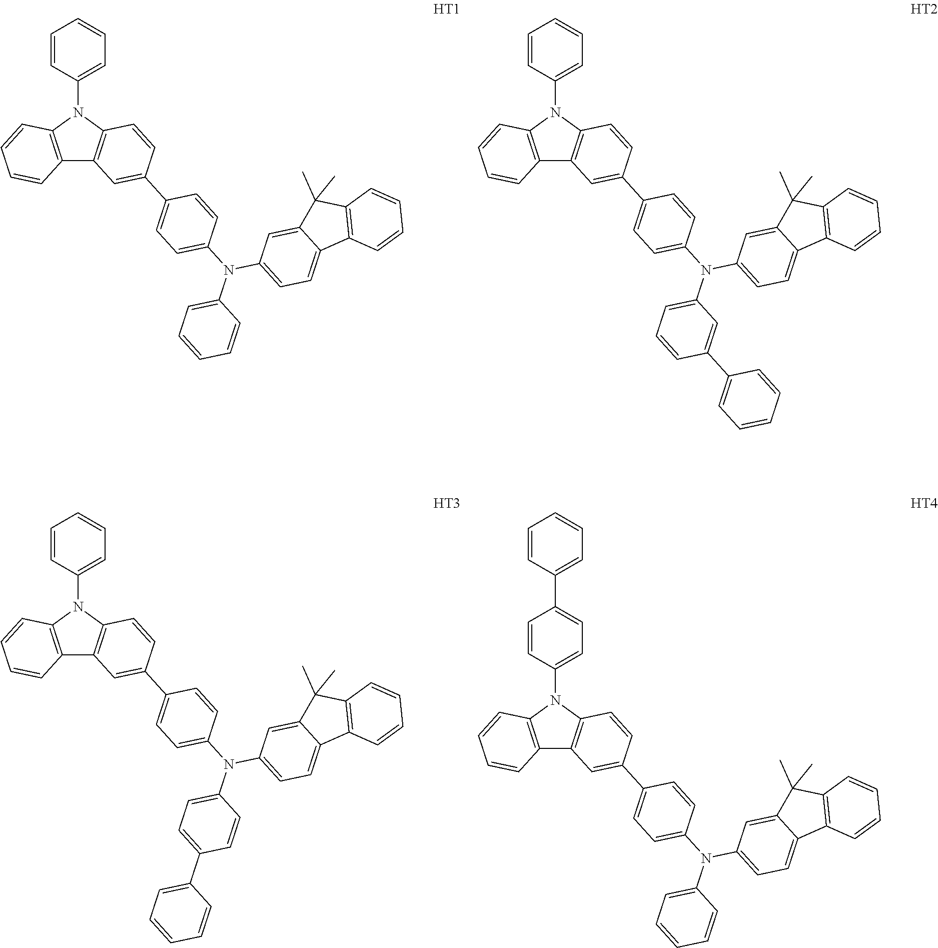 [SMILES: CC1(C)c2ccccc2-c2ccc(N(c3ccccc3)c3ccc(-c4ccc5c(c4)c4ccccc4n5-c4ccccc4)cc3)cc21 ; CC1(C)c2ccccc2-c2ccc(N(c3ccc(-c4ccc5c(c4)c4ccccc4n5-c4ccccc4)cc3)c3cccc(-c4ccccc4)c3)cc21 ; CC1(C)c2ccccc2-c2ccc(N(c3ccc(-c4ccccc4)cc3)c3ccc(-c4ccc5c(c4)c4ccccc4n5-c4ccccc4)cc3)cc21 ; CC1(C)c2ccccc2-c2ccc(N(c3ccccc3)c3ccc(-c4ccc5c(c4)c4ccccc4n5-c4ccc(-c5ccccc5)cc4)cc3)cc21]

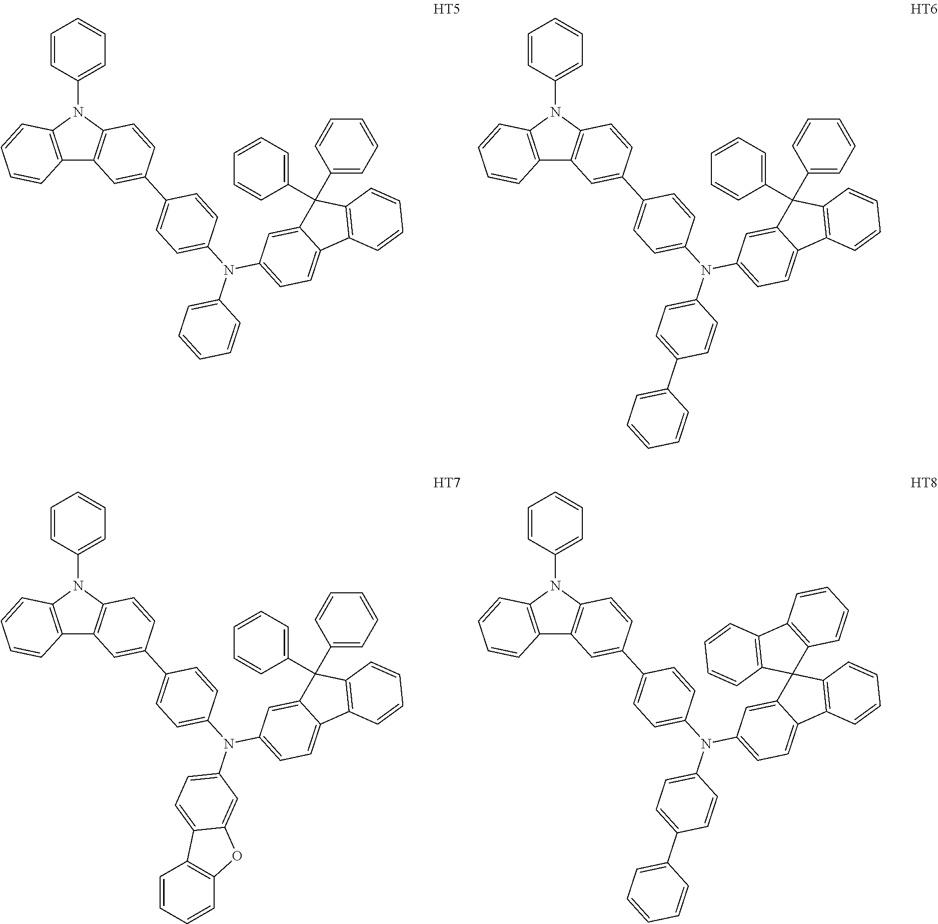 [SMILES: c1ccc(N(c2ccc(-c3ccc4c(c3)c3ccccc3n4-c3ccccc3)cc2)c2ccc3c(c2)C(c2ccccc2)(c2ccccc2)c2ccccc2-3)cc1 ; c1ccc(-c2ccc(N(c3ccc(-c4ccc5c(c4)c4ccccc4n5-c4ccccc4)cc3)c3ccc4c(c3)C(c3ccccc3)(c3ccccc3)c3ccccc3-4)cc2)cc1 ; c1ccc(-n2c3ccccc3c3cc(-c4ccc(N(c5ccc6c(c5)C(c5ccccc5)(c5ccccc5)c5ccccc5-6)c5ccc6c(c5)oc5ccccc56)cc4)ccc32)cc1 ; c1ccc(-c2ccc(N(c3ccc(-c4ccc5c(c4)c4ccccc4n5-c4ccccc4)cc3)c3ccc4c(c3)C3(c5ccccc5-c5ccccc53)c3ccccc3-4)cc2)cc1]

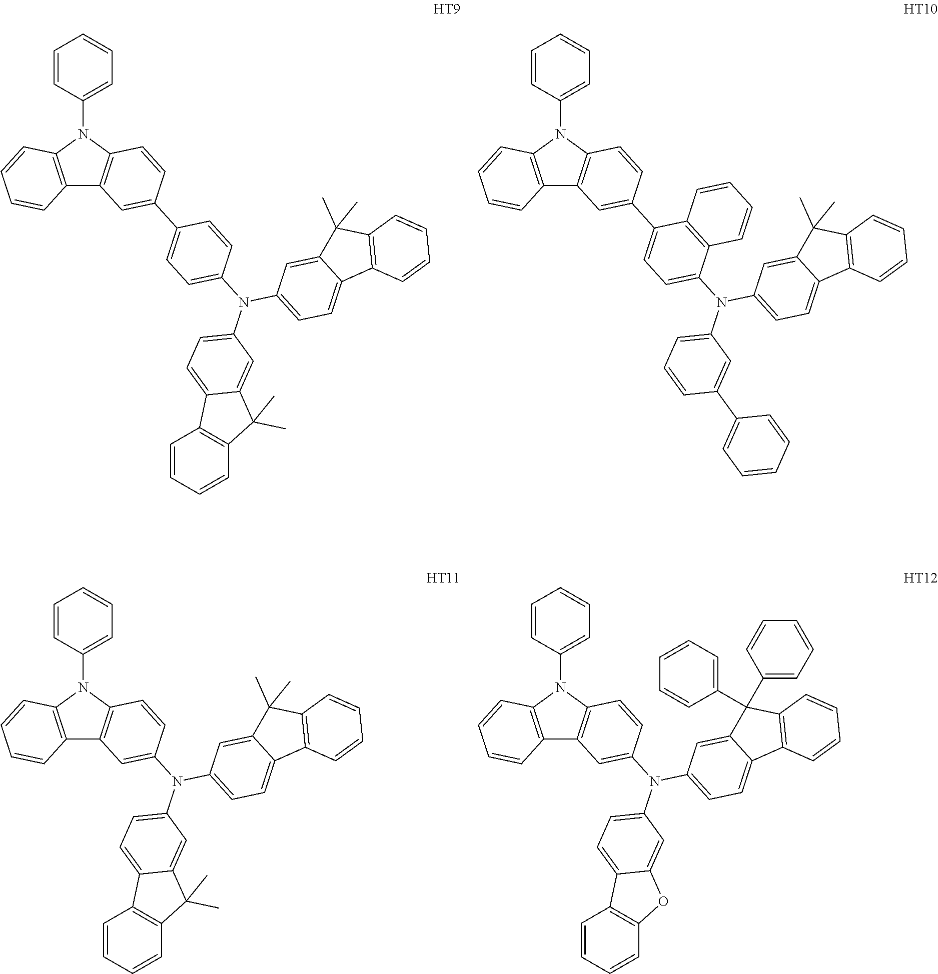 [SMILES: CC1(C)c2ccccc2-c2ccc(N(c3ccc(-c4ccc5c(c4)c4ccccc4n5-c4ccccc4)cc3)c3ccc4c(c3)C(C)(C)c3ccccc3-4)cc21 ; CC1(C)c2ccccc2-c2ccc(N(c3cccc(-c4ccccc4)c3)c3ccc(-c4ccc5c(c4)c4ccccc4n5-c4ccccc4)c4ccccc34)cc21 ; CC1(C)c2ccccc2-c2ccc(N(c3ccc4c(c3)C(C)(C)c3ccccc3-4)c3ccc4c(c3)c3ccccc3n4-c3ccccc3)cc21 ; c1ccc(-n2c3ccccc3c3cc(N(c4ccc5c(c4)C(c4ccccc4)(c4ccccc4)c4ccccc4-5)c4ccc5c(c4)oc4ccccc45)ccc32)cc1]

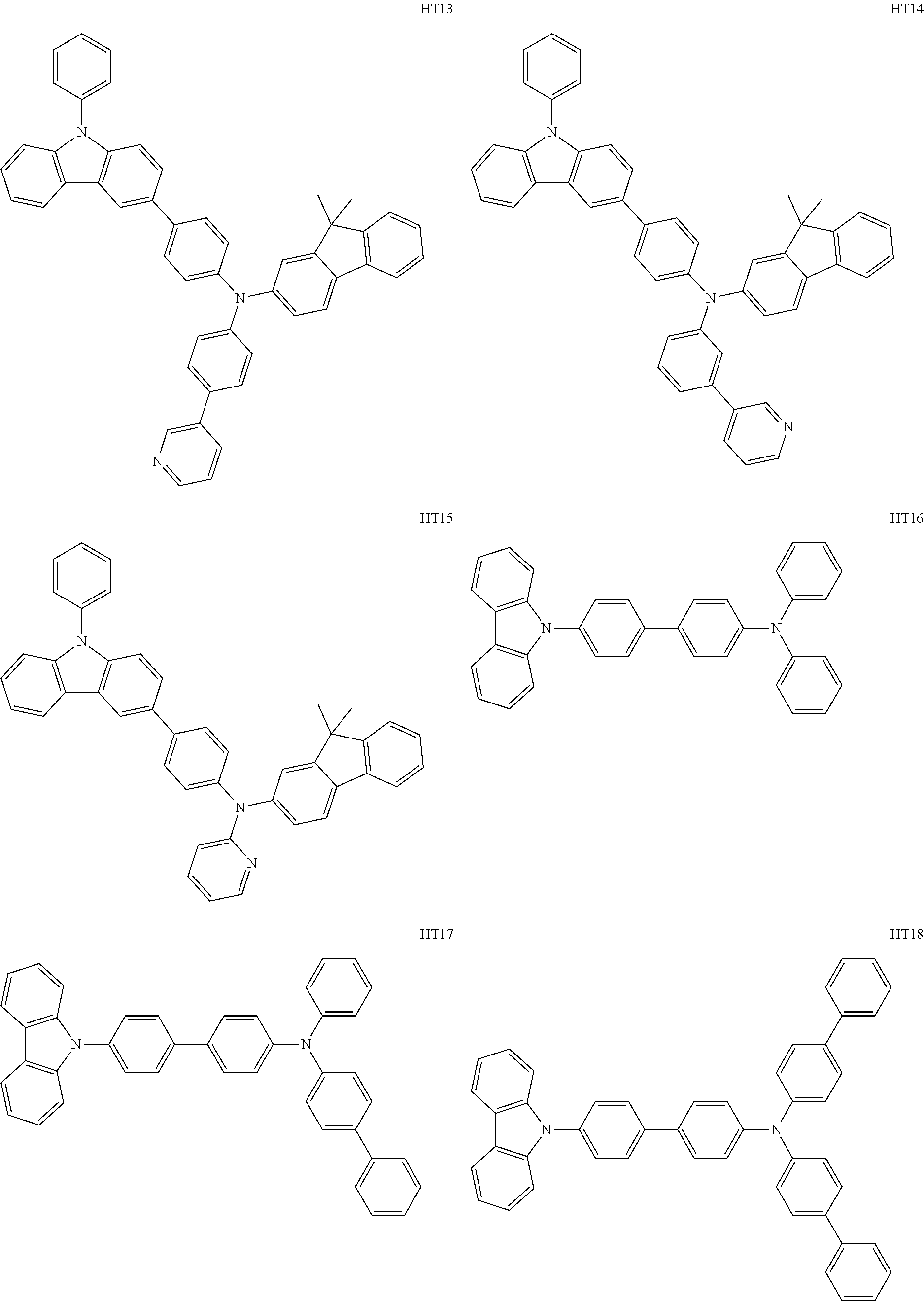 [SMILES: CC1(C)c2ccccc2-c2ccc(N(c3ccc(-c4cccnc4)cc3)c3ccc(-c4ccc5c(c4)c4ccccc4n5-c4ccccc4)cc3)cc21 ; CC1(C)c2ccccc2-c2ccc(N(c3ccc(-c4ccc5c(c4)c4ccccc4n5-c4ccccc4)cc3)c3cccc(-c4cccnc4)c3)cc21 ; CC1(C)c2ccccc2-c2ccc(N(c3ccc(-c4ccc5c(c4)c4ccccc4n5-c4ccccc4)cc3)c3ccccn3)cc21 ; c1ccc(N(c2ccccc2)c2ccc(-c3ccc(-n4c5ccccc5c5ccccc54)cc3)cc2)cc1 ; c1ccc(-c2ccc(N(c3ccccc3)c3ccc(-c4ccc(-n5c6ccccc6c6ccccc65)cc4)cc3)cc2)cc1 ; c1ccc(-c2ccc(N(c3ccc(-c4ccccc4)cc3)c3ccc(-c4ccc(-n5c6ccccc6c6ccccc65)cc4)cc3)cc2)cc1]

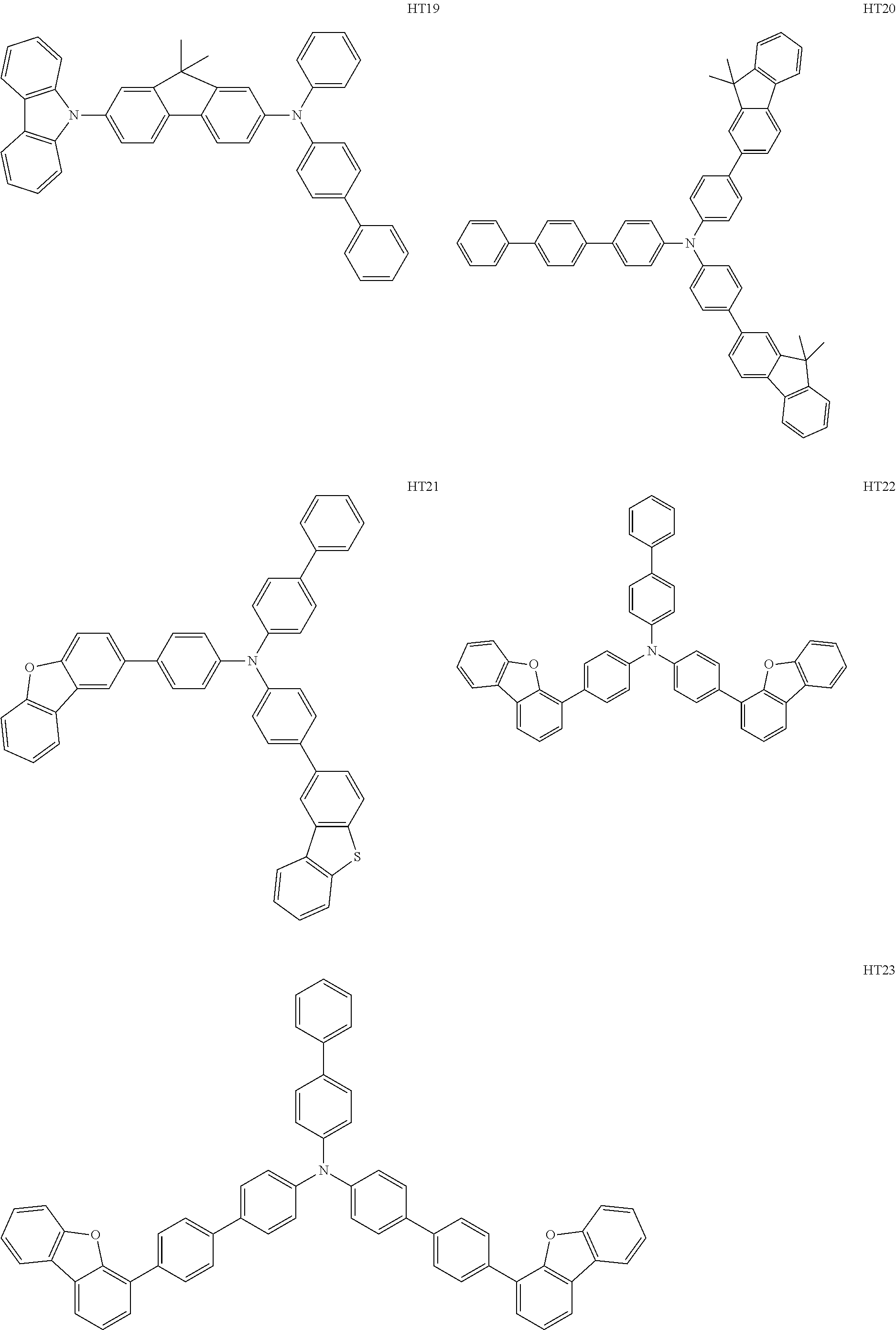 [SMILES: CC1(C)c2cc(N(c3ccccc3)c3ccc(-c4ccccc4)cc3)ccc2-c2ccc(-n3c4ccccc4c4ccccc43)cc21 ; CC1(C)c2ccccc2-c2ccc(-c3ccc(N(c4ccc(-c5ccc(-c6ccccc6)cc5)cc4)c4ccc(-c5ccc6c(c5)C(C)(C)c5ccccc5-6)cc4)cc3)cc21 ; c1ccc(-c2ccc(N(c3ccc(-c4ccc5oc6ccccc6c5c4)cc3)c3ccc(-c4ccc5sc6ccccc6c5c4)cc3)cc2)cc1 ; c1ccc(-c2ccc(N(c3ccc(-c4cccc5c4oc4ccccc45)cc3)c3ccc(-c4cccc5c4oc4ccccc45)cc3)cc2)cc1 ; c1ccc(-c2ccc(N(c3ccc(-c4ccc(-c5cccc6c5oc5ccccc56)cc4)cc3)c3ccc(-c4ccc(-c5cccc6c5oc5ccccc56)cc4)cc3)cc2)cc1]

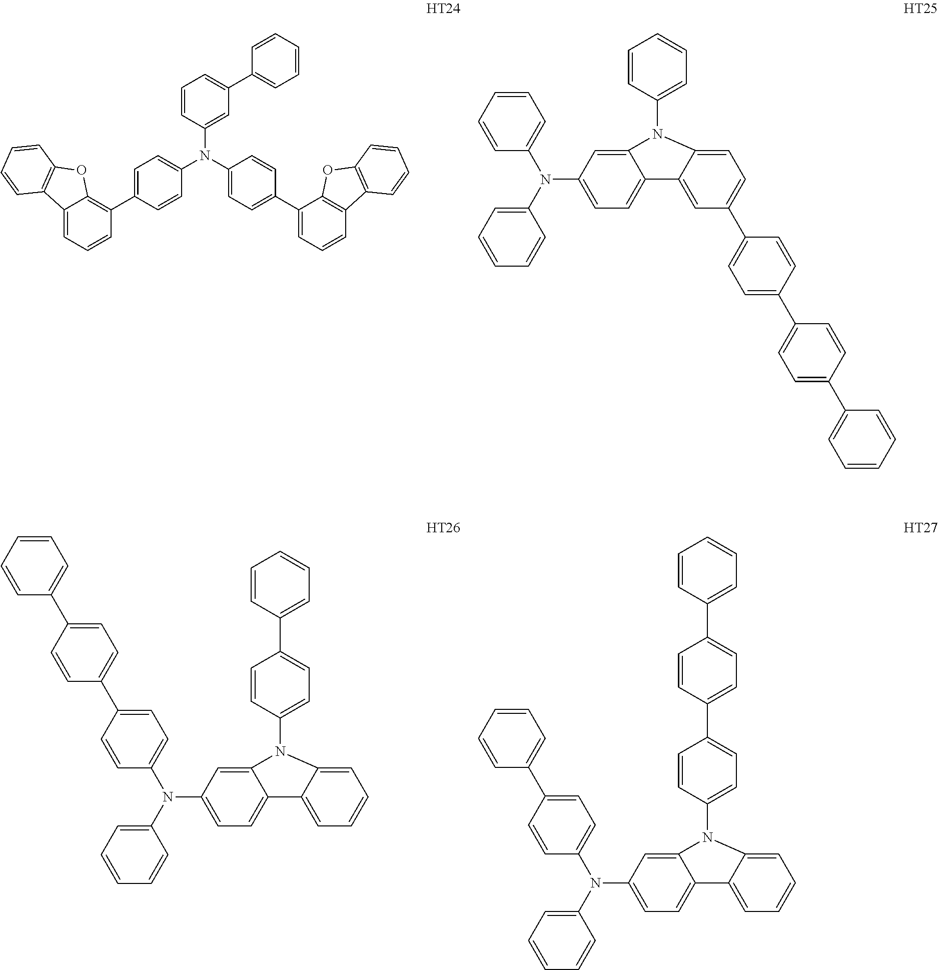 [SMILES: c1ccc(-c2cccc(N(c3ccc(-c4cccc5c4oc4ccccc45)cc3)c3ccc(-c4cccc5c4oc4ccccc45)cc3)c2)cc1 ; c1ccc(-c2ccc(-c3ccc(-c4ccc5c(c4)c4ccc(N(c6ccccc6)c6ccccc6)cc4n5-c4ccccc4)cc3)cc2)cc1 ; c1ccc(-c2ccc(-c3ccc(N(c4ccccc4)c4ccc5c6ccccc6n(-c6ccc(-c7ccccc7)cc6)c5c4)cc3)cc2)cc1 ; c1ccc(-c2ccc(-c3ccc(-n4c5ccccc5c5ccc(N(c6ccccc6)c6ccc(-c7ccccc7)cc6)cc54)cc3)cc2)cc1]

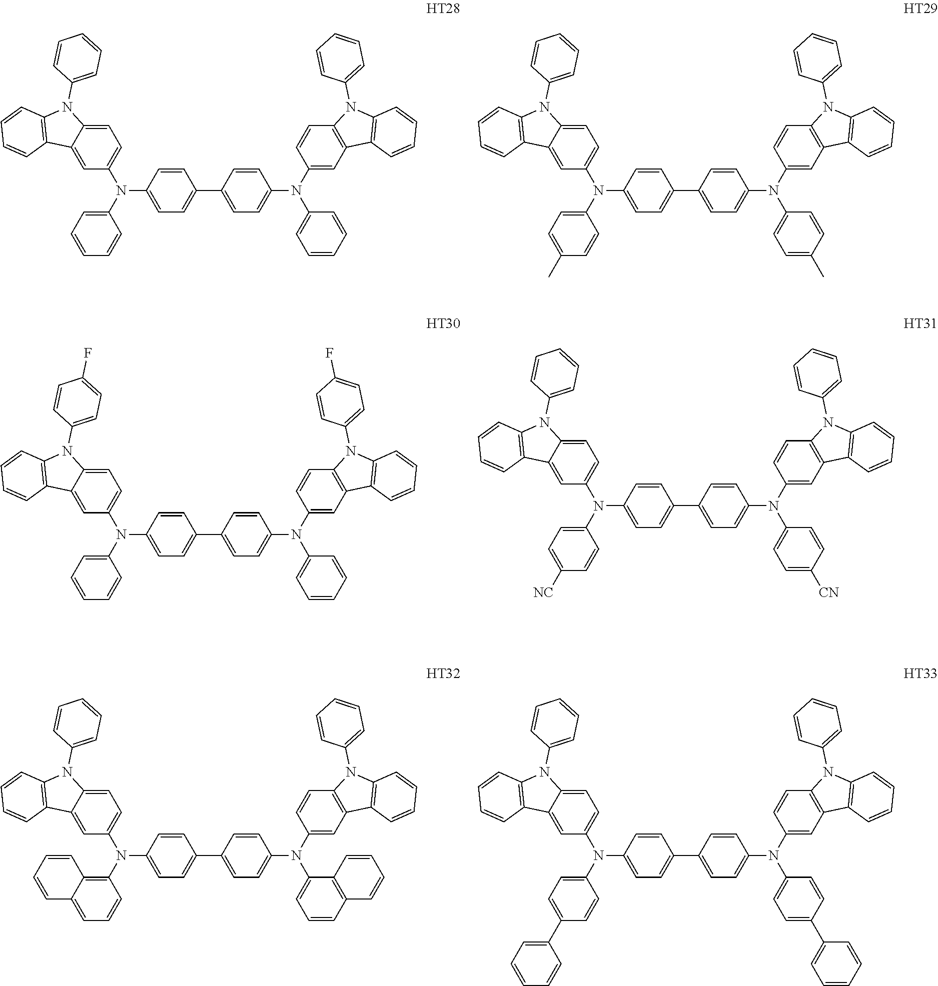

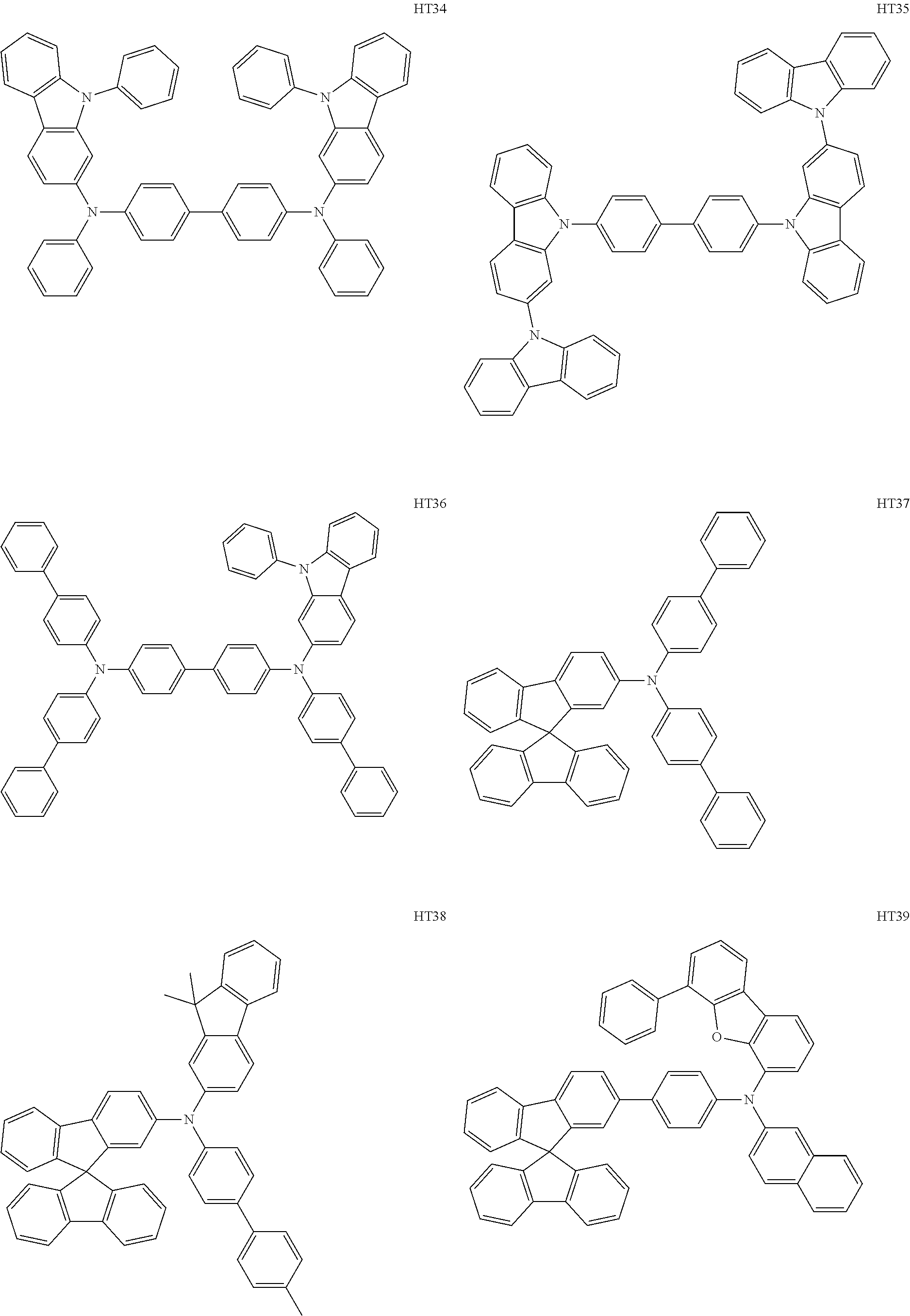

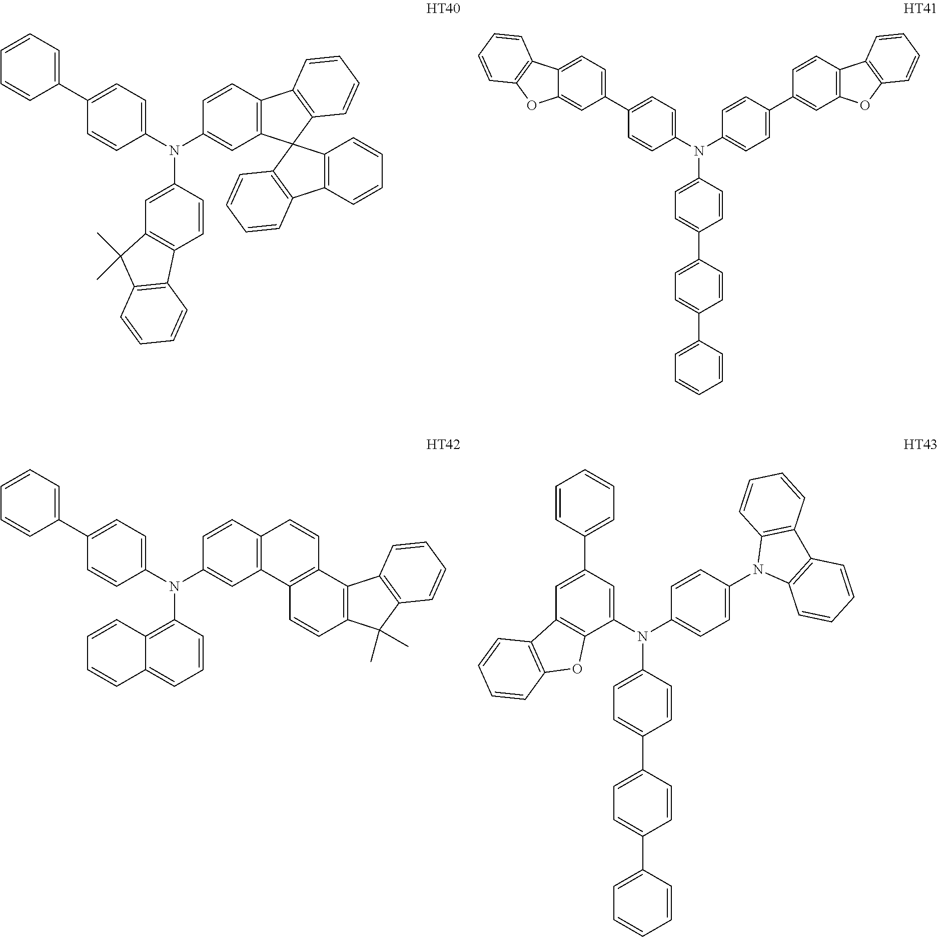 [SMILES: CC1(C)c2ccccc2-c2ccc(N(c3ccc(-c4ccccc4)cc3)c3ccc4c(c3)C3(c5ccccc5-c5ccccc53)c3ccccc3-4)cc21 ; c1ccc(-c2ccc(-c3ccc(N(c4ccc(-c5ccc6c(c5)oc5ccccc56)cc4)c4ccc(-c5ccc6c(c5)oc5ccccc56)cc4)cc3)cc2)cc1 ; CC1(C)c2ccccc2-c2c1ccc1c2ccc2ccc(N(c3ccc(-c4ccccc4)cc3)c3cccc4ccccc34)cc21 ; c1ccc(-c2ccc(-c3ccc(N(c4ccc(-n5c6ccccc6c6ccccc65)cc4)c4cc(-c5ccccc5)cc5c4oc4ccccc45)cc3)cc2)cc1]

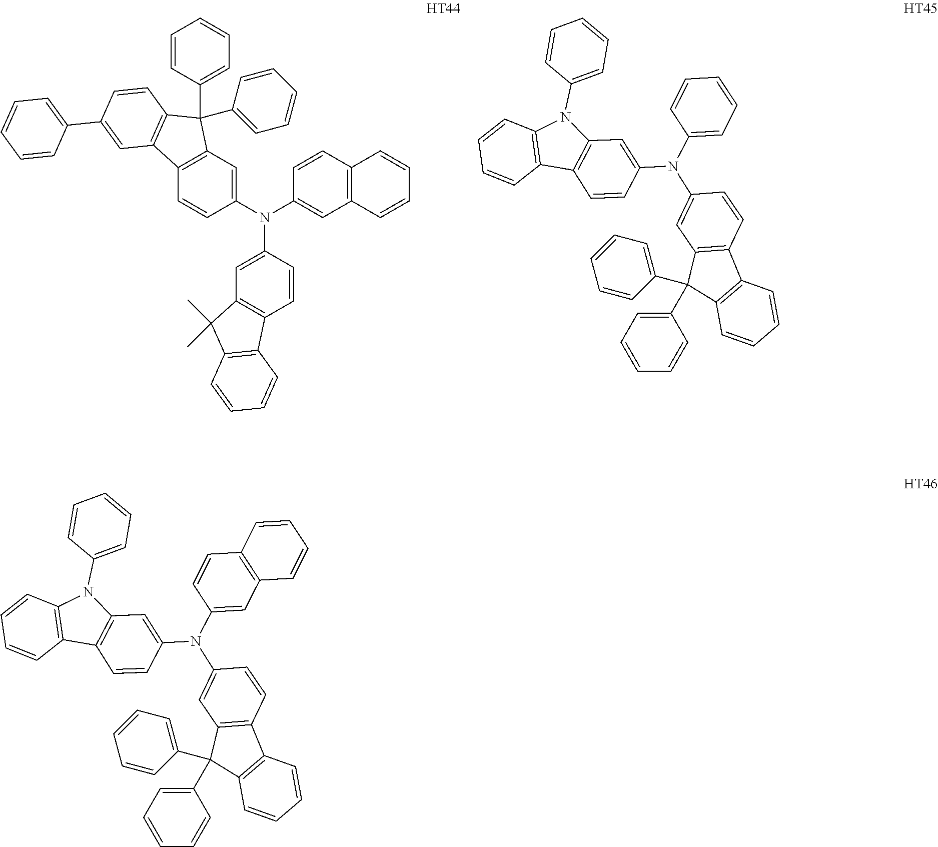 [SMILES: CC1(C)c2ccccc2-c2ccc(N(c3ccc4c(c3)C(c3ccccc3)(c3ccccc3)c3ccc(-c5ccccc5)cc3-4)c3ccc4ccccc4c3)cc21 ; c1ccc(N(c2ccc3c(c2)C(c2ccccc2)(c2ccccc2)c2ccccc2-3)c2ccc3c4ccccc4n(-c4ccccc4)c3c2)cc1 ; c1ccc(-n2c3ccccc3c3ccc(N(c4ccc5c(c4)C(c4ccccc4)(c4ccccc4)c4ccccc4-5)c4ccc5ccccc5c4)cc32)cc1]

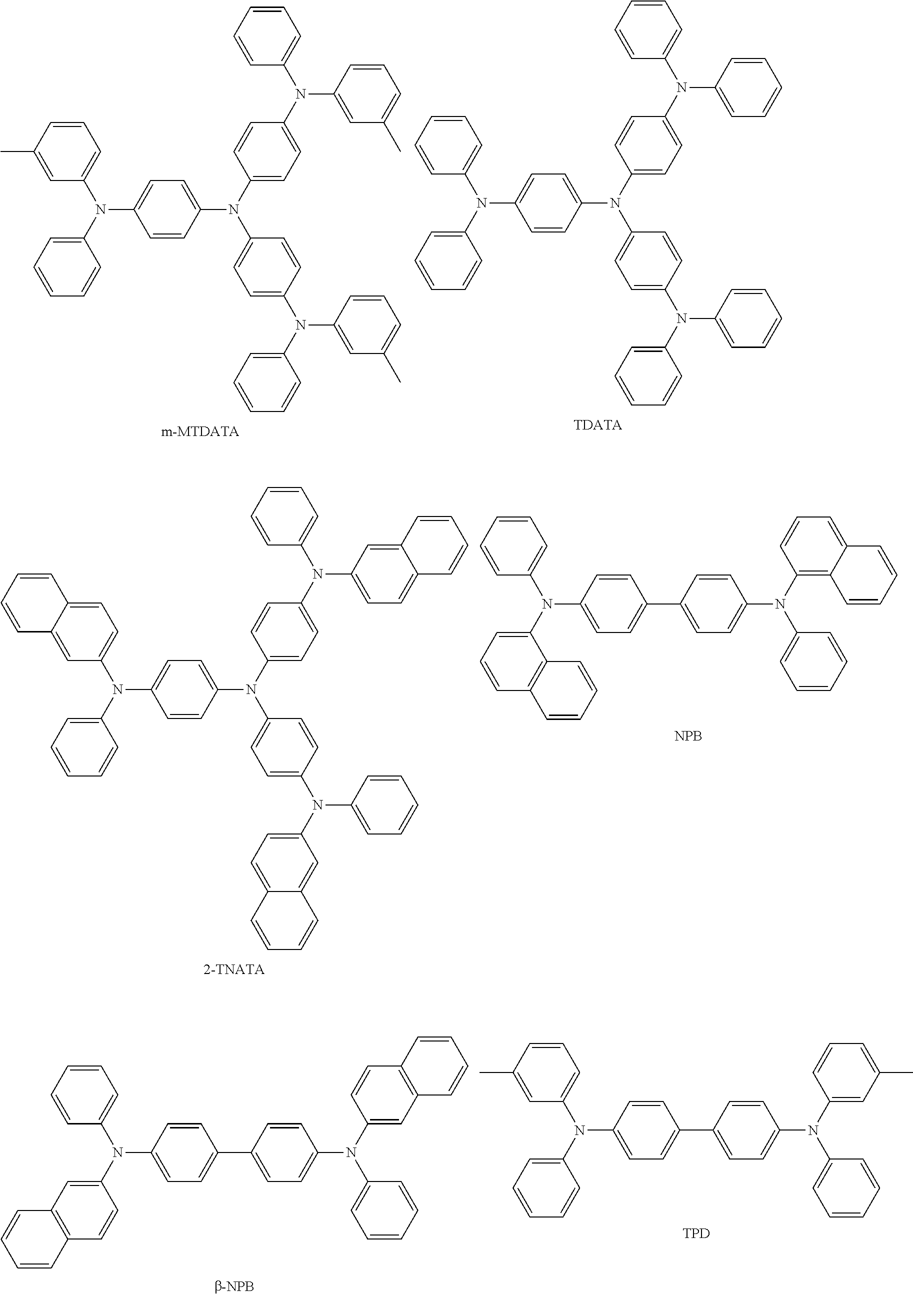

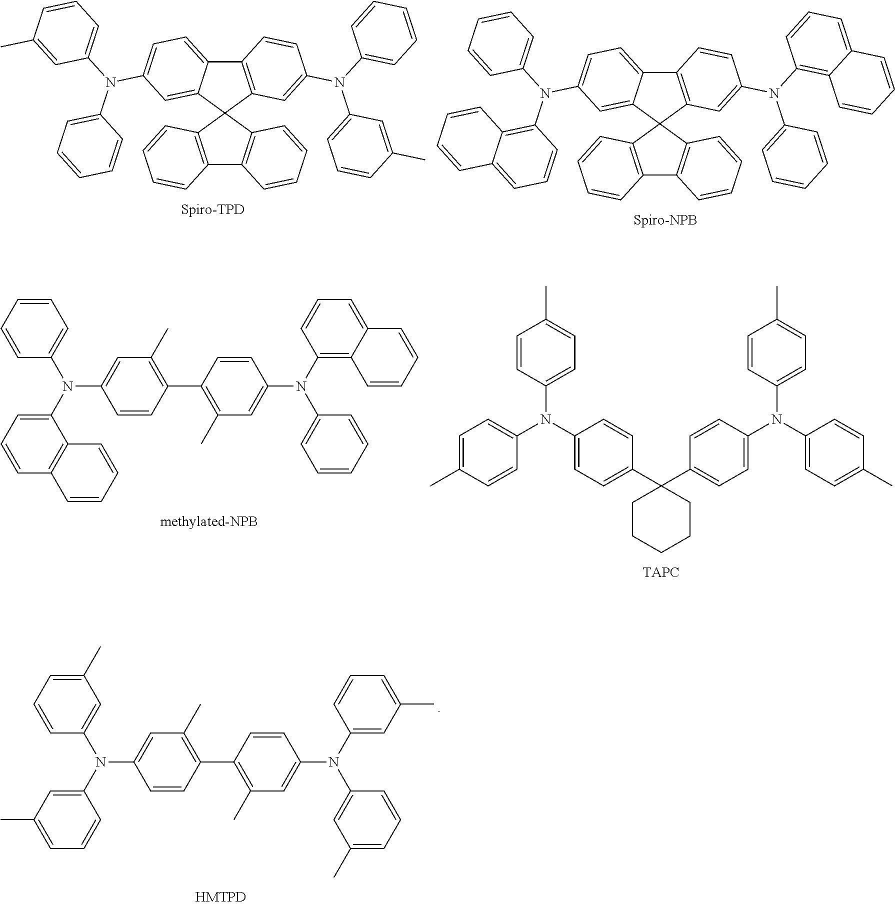

A thickness of the hole transport region 120 may be in a range of about 50 angstrom (Å) to about 10,000 Å, for example, about 100 Å to about 4,000 Å. When the hole transport region 120 includes a hole injection layer, a hole transport layer, or any combination thereof, a thickness of the hole injection layer may be in a range of about 100 Å to about 9,000 Å, for example, about 100 Å to about 1,000 Å, and a thickness of the hole transport layer may be in a range of about 50 Å to about 2,000 Å, for example, about 100 Å to about 1,500 Å. When the thicknesses of the hole transport region 120, the hole injection layer, and the hole transport layer are within the ranges described above, satisfactory hole transporting characteristics may be obtained without a substantial increase in driving voltage.

The emission auxiliary layer may increase light-emission efficiency by compensating for an optical resonance distance according to the wavelength of light emitted by the emission layer 130. In one or more embodiments, the electron blocking layer may block or reduce the leakage of electrons from the emission layer 130 to the hole transport region 120. Materials that may be included in the hole transport region 120 may be included in the emission auxiliary layer and the electron blocking layer.

p-dopant

The hole transport region 120 may further include, in addition to the materials described above, a charge-generation material for the improvement of conductive properties. The charge-generation material may be uniformly or non-uniformly dispersed in the hole transport region 120 (for example, in the form of a single layer consisting of a charge-generation material).

The charge-generation material may be, for example, a p-dopant.

For example, a LUMO energy level of the p-dopant may be less than or equal to −3.5 eV.

In one or more embodiments, the p-dopant may include a quinone derivative, a cyano group-containing compound, a compound including element EL1 and element EL2, or any combination thereof.

Examples of the quinone derivative are TCNQ, F4-TCNQ, and/or the like.

Examples of the cyano group-containing compound are HAT-CN, a compound represented by Formula 221, and/or the like:

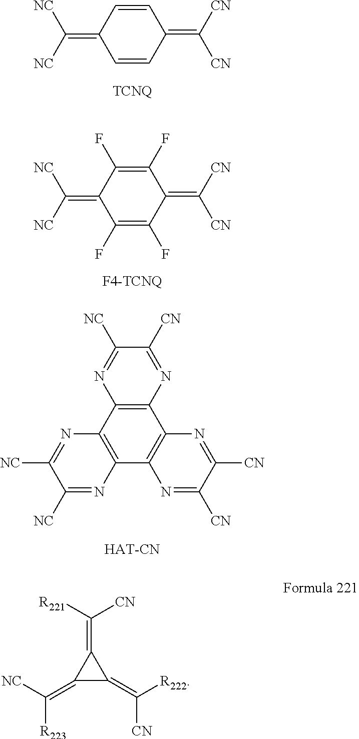

In Formula 221,
R₂₂₁ to R₂₂₃ may each independently be a C₃-C₆₀ carbocyclic group unsubstituted or substituted with at least one R₁₀ₐ or a C₁-C₆₀ heterocyclic group unsubstituted or substituted with at least one R₁₀ₐ, and
at least one selected from among R₂₂₁ to R₂₂₃ may each independently be a C₃-C₆₀ carbocyclic group or a C₁-C₆₀ heterocyclic group, each substituted with: a cyano group; —F; —Cl; —Br; —I; a C₁-C₂₀ alkyl group substituted with a cyano group, —F, —Cl, —Br, —I, or any combination thereof; or any combination thereof.

In the compound including element EL1 and element EL2, element EL1 may be metal, metalloid, or any combination thereof, and element EL2 may be non-metal, metalloid, or any combination thereof.

Examples of the metal are: an alkali metal (for example, lithium (Li), sodium (Na), potassium (K), rubidium (Rb), cesium (Cs), etc.); an alkaline earth metal (for example, beryllium (Be), magnesium (Mg), calcium (Ca), strontium (Sr), barium (Ba), etc.); a transition metal (for example, titanium (Ti), zirconium (Zr), hafnium (Hf), vanadium (V), niobium (Nb), tantalum (Ta), chromium (Cr), molybdenum (Mo), tungsten (W), manganese (Mn), technetium (Tc), rhenium (Re), iron (Fe), ruthenium (Ru), osmium (Os), cobalt (Co), rhodium (Rh), iridium (Ir), nickel (Ni), palladium (Pd), platinum (Pt), copper (Cu), silver (Ag), gold (Au), etc.); a post-transition metal (for example, zinc (Zn), indium (In), tin (Sn), etc.); a lanthanide metal (for example, lanthanum (La), cerium (Ce), praseodymium (Pr), neodymium (Nd), promethium (Pm), samarium (Sm), europium (Eu), gadolinium (Gd), terbium (Tb), dysprosium (Dy), holmium (Ho), erbium (Er), thulium (Tm), ytterbium (Yb), lutetium (Lu), etc.); and/or the like.

Examples of the metalloid are silicon (Si), antimony (Sb), tellurium (Te), and/or the like.

Examples of the non-metal are oxygen (O), halogen (for example, F, Cl, Br, I, etc.), and/or the like.

Examples of the compound including element EL1 and element EL2 are metal oxide, metal halide (for example, metal fluoride, metal chloride, metal bromide, metal iodide, etc.), metalloid halide (for example, metalloid fluoride, metalloid chloride, metalloid bromide, metalloid iodide, etc.), metal telluride, or any combination thereof.

Examples of the metal oxide are tungsten oxide (for example, WO, W₂O₃, WO₂, WO₃, W₂O₅, etc.), vanadium oxide (for example, VO, V₂O₃, VO₂, V₂O₅, etc.), molybdenum oxide (for example, MoO, Mo₂O₃, MoO₂, MoO₃, Mo₂O₅, etc.), rhenium oxide (for example, ReO₃, etc.), and/or the like.

Examples of the metal halide are alkali metal halide, alkaline earth metal halide, transition metal halide, post-transition metal halide, lanthanide metal halide, and/or the like.

Examples of the alkali metal halide are LiF, NaF, KF, RbF, CsF, LiCl, NaCl, KCl, RbCl, CsCl, LiBr, NaBr, KBr, RbBr, CsBr, LiI, NaI, KI, RbI, CsI, and/or the like.

Examples of the alkaline earth metal halide are BeF₂, MgF₂, CaF₂, SrF₂, BaF₂, BeCl₂, MgCl₂, CaCl₂), SrCl₂, BaCl₂, BeBr₂, MgBr₂, CaBr₂, SrBr₂, BaBr₂, Be₁₂, MgI₂, CaI₂, SrI₂, BaI₂, and/or the like.

Examples of the transition metal halide are titanium halide (for example, TiF₄, TiCl₄, TiBr₄, TiI₄, etc.), zirconium halide (for example, ZrF₄, ZrCl₄, ZrBr₄, ZrI₄, etc.), hafnium halide (for example, HfF₄, HfCl₄, HfBr₄, HfI₄, etc.), vanadium halide (for example, VF₃, VCI₃, VBr₃, VI₃, etc.), niobium halide (for example, NbF₃, NbCl₃, NbBr₃, NbI₃, etc.), tantalum halide (for example, TaF₃, TaCl₃, TaBr₃, TaI₃, etc.), chromium halide (for example, CrF₃, CrCl₃, CrBr₃, CrI₃, etc.), molybdenum halide (for example, MoF₃, MoCl₃, MoBr₃, MoI₃, etc.), tungsten halide (for example, WF₃, WCl₃, WBr₃, WI₃, etc.), manganese halide (for example, MnF₂, MnCl₂, MnBr₂, MnI₂, etc.), technetium halide (for example, TcF₂, TcCl₂, TcBr₂, TcI₂, etc.), rhenium halide (for example, ReF₂, ReCl₂, ReBr₂, ReI₂, etc.), iron halide (for example, FeF₂, FeCl₂, FeBr₂, FeI₂, etc.), ruthenium halide (for example, RuF₂, RuCl₂, RuBr₂, RuI₂, etc.), osmium halide (for example, OsF₂, OsCl₂, OsBr₂, OsI₂, etc.), cobalt halide (for example, CoF₂, CoCl₂, CoBr₂, CoI₂, etc.), rhodium halide (for example, RhF₂, RhCl₂, RhBr₂, RhI₂, etc.), iridium halide (for example, IrF₂, IrCl₂, IrBr₂, IrI₂, etc.), nickel halide (for example, NiF₂, NiCl₂, NiBr₂, NiI₂, etc.), palladium halide (for example, PdF₂, PdCl₂, PdBr₂, PdI₂, etc.), platinum halide (for example, PtF₂, PtCl₂, PtBr₂, PtI₂, etc.), copper halide (for example, CuF, CuCl, CuBr, CuI, etc.), silver halide (for example, AgF, AgCl, AgBr, AgI, etc.), gold halide (for example, AuF, AuCl, AuBr, AuI, etc.), and/or the like.

Examples of the post-transition metal halide are zinc halide (for example, ZnF₂, ZnCl₂, ZnBr₂, ZnI₂, etc.), indium halide (for example, InI₃, etc.), tin halide (for example, SnI₂, etc.), and/or the like.

Examples of the lanthanide metal halide are YbF, YbF₂, YbF₃, SmF₃, YbCl, YbCl₂, YbCl₃, SmCl₃, YbBr, YbBr₂, YbBr₃, SmBr₃, YbI, YbI₂, YbI₃, SmI₃, and/or the like.

Examples of the metalloid halide are antimony halide (for example, SbCl₅, etc.) and/or the like.

Examples of the metal telluride are alkali metal telluride (for example, Li₂Te, Na₂Te, K₂Te, Rb₂Te, Cs₂Te, etc.), alkaline earth metal telluride (for example, BeTe, MgTe, CaTe, SrTe, BaTe, etc.), transition metal telluride (for example, TiTe₂, ZrTe₂, HfTe₂, V₂Te₃, Nb₂Te₃, Ta₂Te₃, Cr₂Te₃, Mo₂Te₃, W₂Te₃, MnTe, TcTe, ReTe, FeTe, RuTe, OsTe, CoTe, RhTe, IrTe, NiTe, PdTe, PtTe, Cu₂Te, CuTe, Ag₂Te, AgTe, Au₂Te, etc.), post-transition metal telluride (for example, ZnTe, etc.), lanthanide metal telluride (for example, LaTe, CeTe, PrTe, NdTe, PmTe, EuTe, GdTe, TbTe, DyTe, HoTe, ErTe, TmTe, YbTe, LuTe, etc.), and/or the like.

### Emission Layer 130

The light-emitting device 10 may include the emission layer 130 on the hole transport region 120.

The emission layer 130 may further include, in addition to one or more suitable organic materials, a metal-containing compound such as an organometallic compound, an inorganic material such as a quantum dot, and/or the like.

In one or more embodiments, the emission layer 130 may include i) two or more emitting units sequentially stacked between the first electrode 110 and the second electrode 150, and ii) a charge generation layer arranged between the two or more emitting units. When the emission layer 130 includes the emitting units and the charge generation layer described above, the light-emitting device 10 may be a tandem light-emitting device.

When the light-emitting device 10 is a full-color light-emitting device, the emission layer 130 may be patterned into a red emission layer, a green emission layer, and/or a blue emission layer, according to a sub-pixel. In one or more embodiments, the emission layer 130 may have a stacked structure of two or more layers of a red emission layer, a green emission layer, and a blue emission layer, in which the two or more layers contact each other or are separated from each other, to emit white light. In one or more embodiments, the emission layer 130 may include two or more materials of a red light-emitting material, a green light-emitting material, and a blue light-emitting material, in which the two or more materials are mixed with each other in a single layer, to emit white light.

The emission layer 130 may include a host and a dopant. The dopant may include a phosphorescent dopant, a fluorescent dopant, or any combination thereof.

An amount of the dopant in the emission layer 130 may be in a range of about 0.01 part by weight to about 15 parts by weight based on 100 parts by weight of the host.

In one or more embodiments, the emission layer 130 may include a quantum dot.

In one or more embodiments, the emission layer 130 may include a delayed fluorescence material. The delayed fluorescence material may act as a host or a dopant in the emission layer 130.

A thickness of the emission layer 130 may be in a range of about 100 Å to about 1,000 Å, for example, about 200 Å to about 600 Å. When the thickness of the emission layer 130 is within the ranges described above, excellent or suitable luminescence characteristics may be obtained without a substantial increase in driving voltage.

### Host

The host may include a compound represented by Formula 301:

[Ar₃₀₁]_{xb11}-[(L₃₀₁)_{xb1}-R₃₀₁]_{xb21}. Formula 301

In Formula 301,
Ar₃₀₁ and L₃₀₁ may each independently be a C₃-C₆₀ carbocyclic group unsubstituted or substituted with at least one R₁₀ₐ or a C₁-C₆₀ heterocyclic group unsubstituted or substituted with at least one R₁₀ₐ,
xb11 may be 1, 2, or 3,
xb1 may be an integer from 0 to 5,
R₃₀₁ may be hydrogen, deuterium, —F, —Cl, —Br, —I, a hydroxyl group, a cyano group, a nitro group, a C₁-C₆₀ alkyl group unsubstituted or substituted with at least one R₁₀ₐ, a C₂-C₆₀ alkenyl group unsubstituted or substituted with at least one R₁₀ₐ, a C₂-C₆₀ alkynyl group unsubstituted or substituted with at least one R₁₀ₐ, a C₁-C₆₀ alkoxy group unsubstituted or substituted with at least one R₁₀ₐ, a C₃-C₆₀ carbocyclic group unsubstituted or substituted with at least one R₁₀ₐ, a C₁-C₆₀ heterocyclic group unsubstituted or substituted with at least one R₁₀ₐ, —Si(Q₃₀₁)(Q₃₀₂)(Q₃₀₃), —N(Q₃₀₁)(Q₃₀₂), —B(Q₃₀₁)(Q₃₀₂), —C(═O)(Q₃₀₁), —S(═O)₂(Q₃₀₁), or —P(═O)(Q₃₀₁)(Q₃₀₂),
xb21 may be an integer from 1 to 5, and
Q₃₀₁ to Q₃₀₃ may each be defined as in connection with Q₁.

For example, when xb11 in Formula 301 is 2 or more, two or more of Ar₃₀₁ may be linked to each other via a single bond.

In one or more embodiments, the host may include a compound represented by Formula 301-1, a compound represented by Formula 301-2, or any combination thereof:

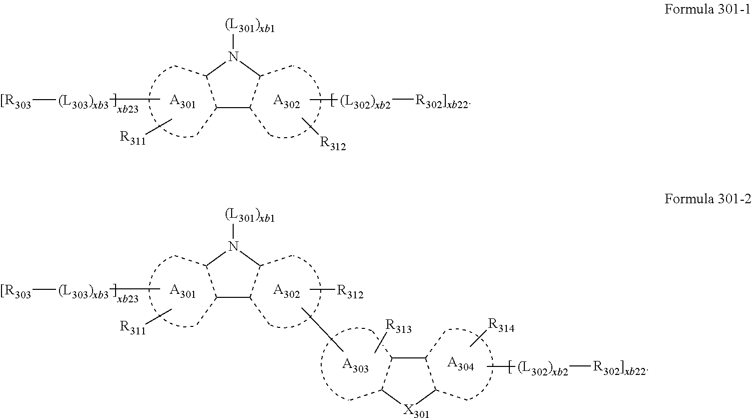

In Formulae 301-1 and 301-2,
ring A₃₀₁ to ring A₃₀₄ may each independently be a C₃-C₆₀ carbocyclic group unsubstituted or substituted with at least one R₁₀ₐ or a C₁-C₆₀ heterocyclic group unsubstituted or substituted with at least one R₁₀ₐ,
X₃₀₁ may be O, S, N[(L₃₀₄)_{xb4}-R₃₀₄], C(R₃₀₄)(R₃₀₅), or Si(R₃₀₄)(R₃₀₅),
xb22 and xb23 may each independently be 0, 1, or 2,
L₃₀₁, xb1, and R₃₀₁ may each be as defined herein,
L₃₀₂ to L₃₀₄ may each independently be defined as in connection with L₃₀₁,
xb2 to xb4 may each independently be defined as in connection with xb1, and
R₃₀₂ to R₃₀₅ and R₃₁₁ to R₃₁₄ may each be defined as in connection with R₃₀₁.

In one or more embodiments, the host may include an alkali earth metal complex, a post-transition metal complex, or any combination thereof. For example, the host may include a Be complex (for example, Compound H55), a Mg complex, a Zn complex, or any combination thereof.

In one or more embodiments, the host may include: at least one selected from among Compounds H1 to H128; 9,10-di(2-naphthyl)anthracene (ADN); 2-methyl-9,10-bis(naphthalen-2-yl)anthracene (MADN); 9,10-di-(2-naphthyl)-2-t-butyl-anthracene (TBADN); 4,4′-bis(N-carbazolyl)-1,1′-biphenyl (CBP); 1,3-di(carbazol-9-yl)benzene (mCP); 1,3,5-tri(carbazol-9-yl)benzene (TCP); and/or any combination thereof:

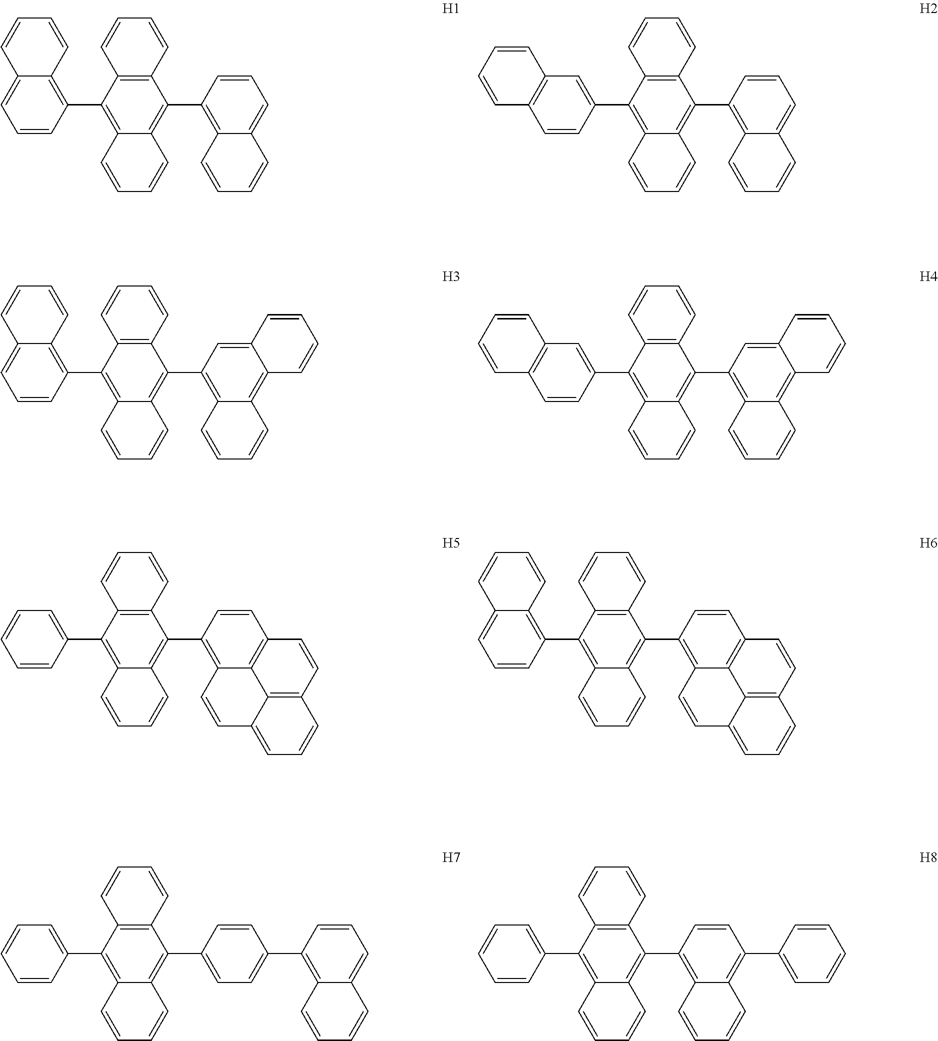

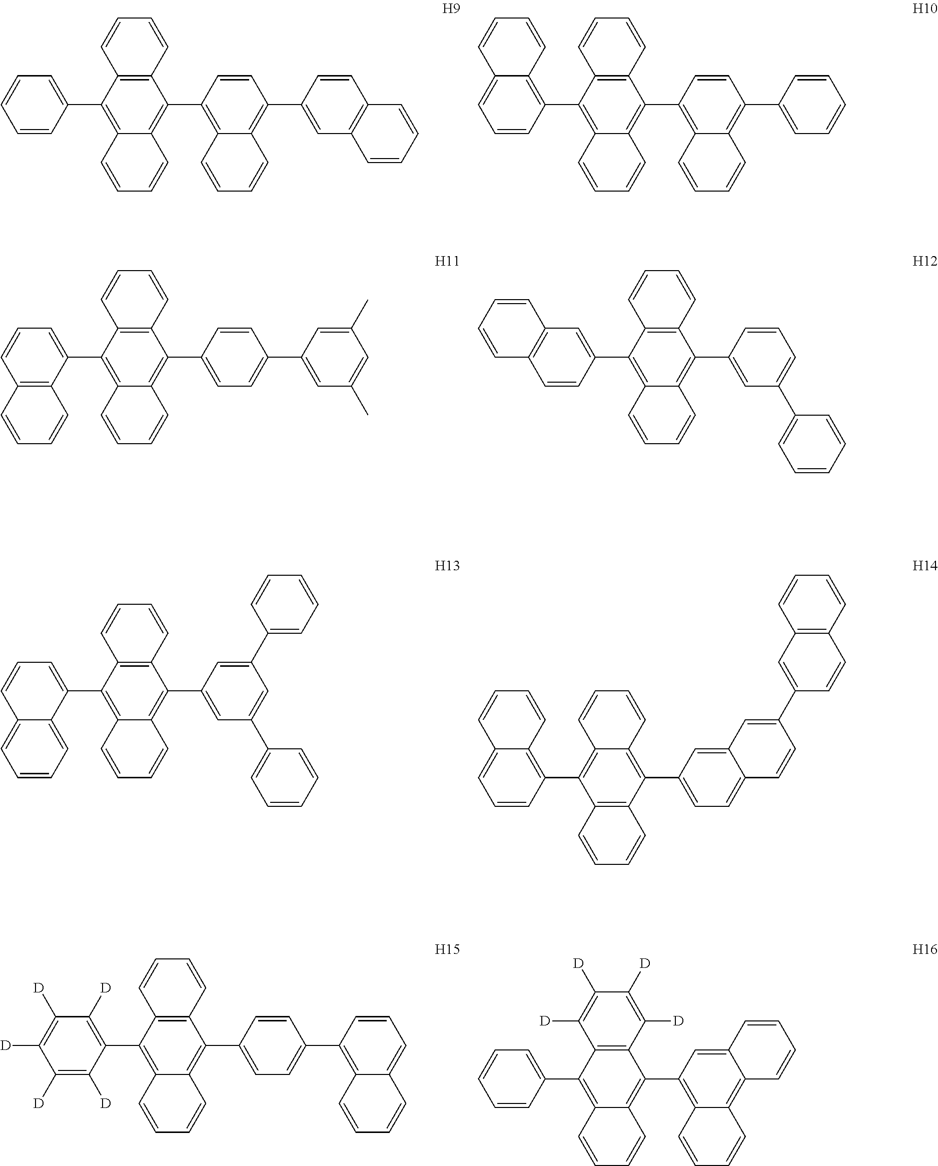

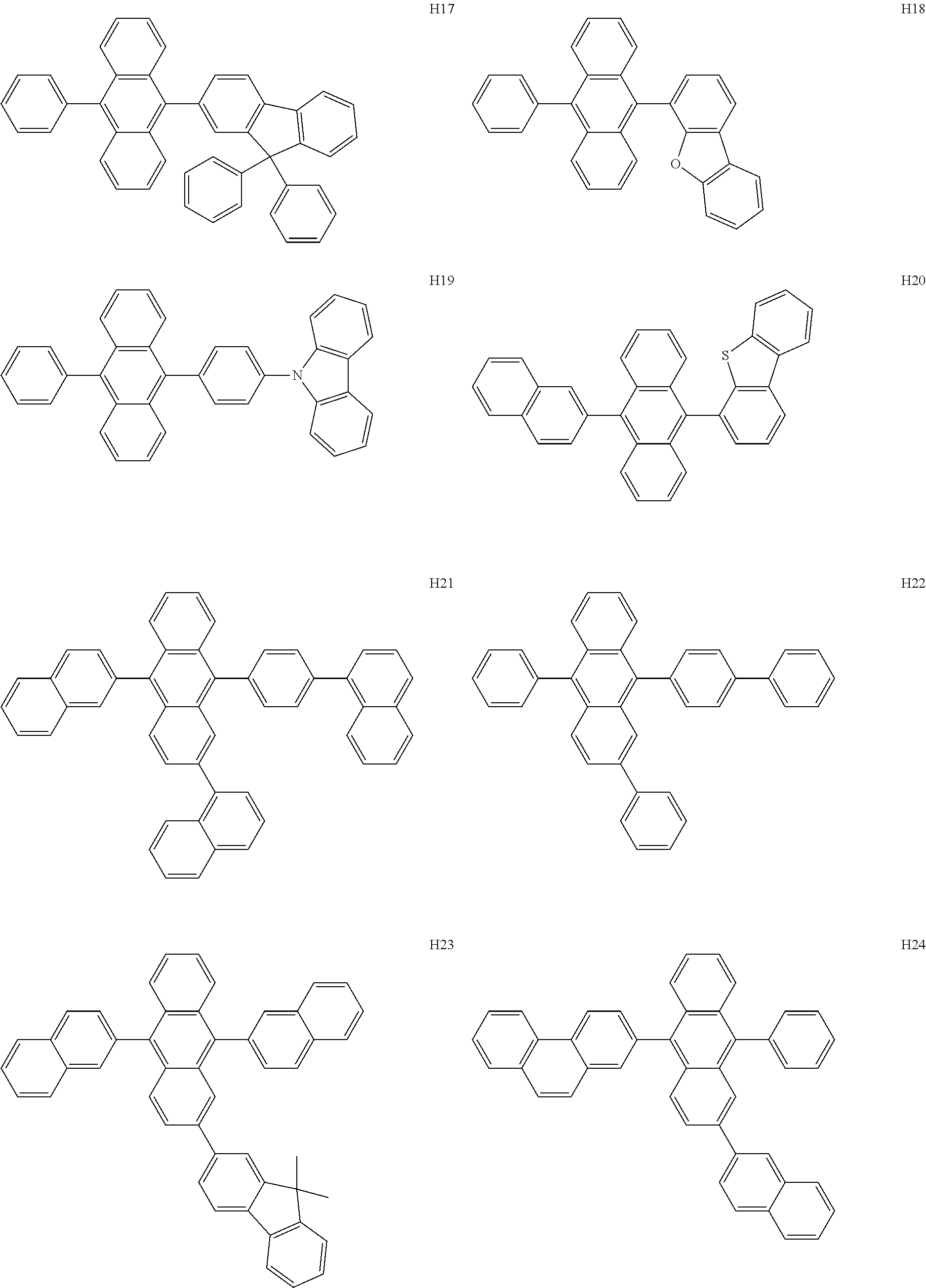

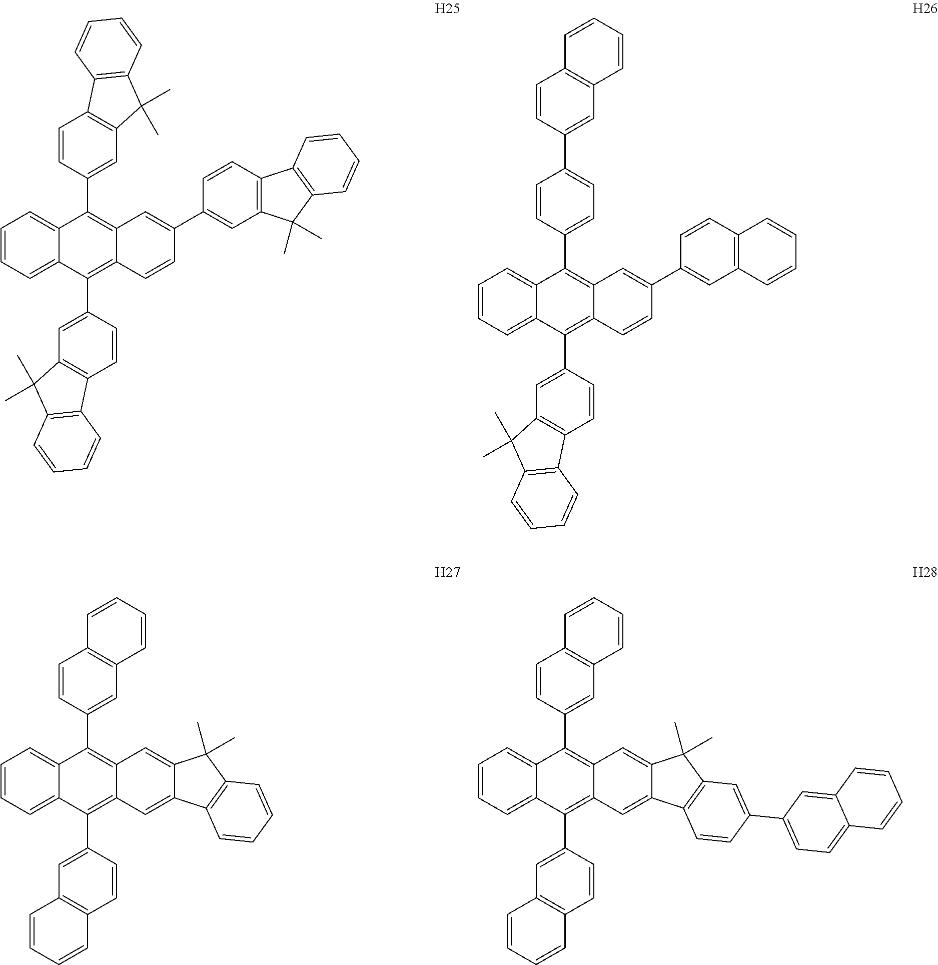

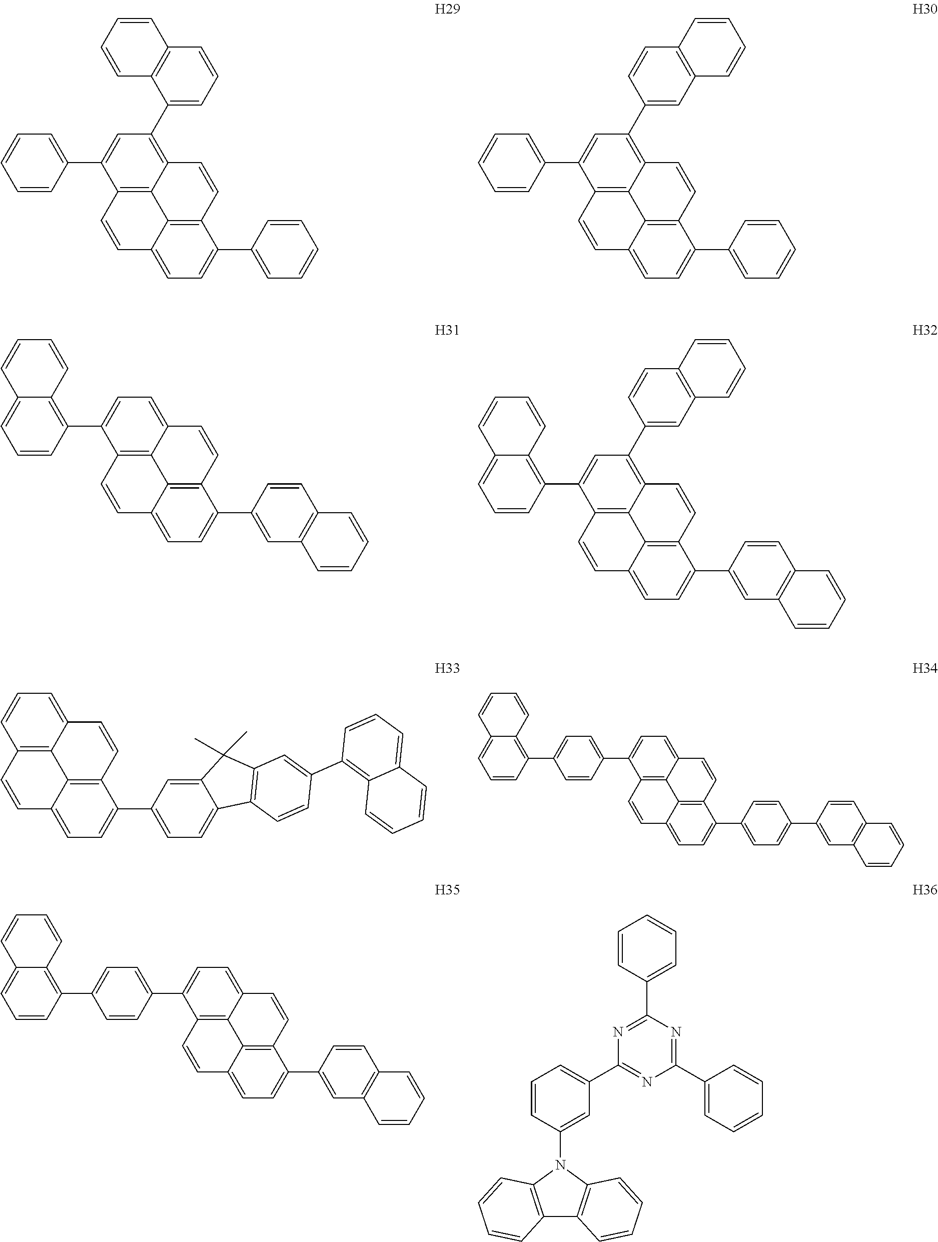

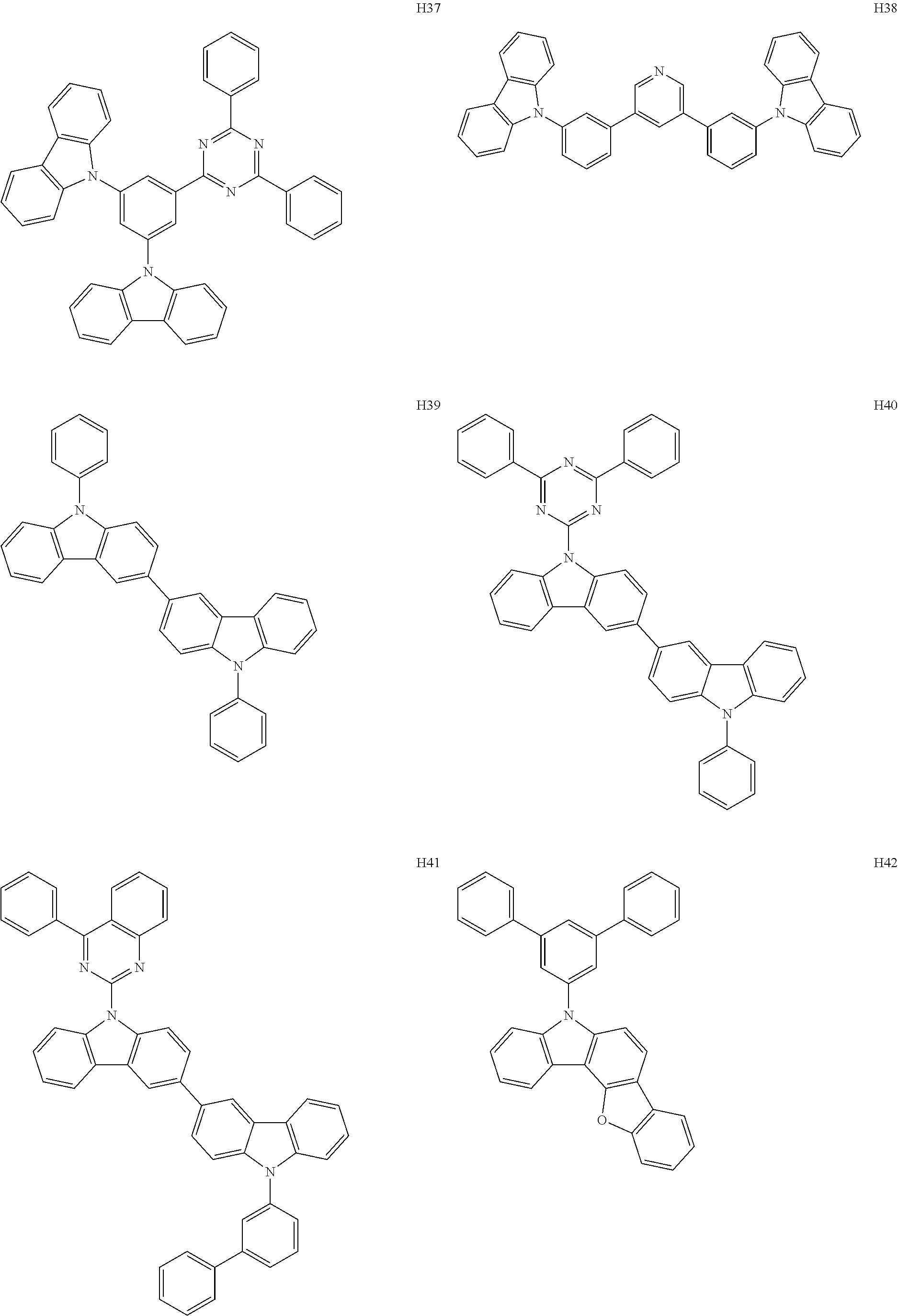 [SMILES: c1ccc(-c2nc(-c3ccccc3)nc(-c3cc(-n4c5ccccc5c5ccccc54)cc(-n4c5ccccc5c5ccccc54)c3)n2)cc1 ; c1cc(-c2cncc(-c3cccc(-n4c5ccccc5c5ccccc54)c3)c2)cc(-n2c3ccccc3c3ccccc32)c1 ; c1ccc(-n2c3ccccc3c3cc(-c4ccc5c(c4)c4ccccc4n5-c4ccccc4)ccc32)cc1 ; c1ccc(-c2nc(-c3ccccc3)nc(-n3c4ccccc4c4cc(-c5ccc6c(c5)c5ccccc5n6-c5ccccc5)ccc43)n2)cc1 ; c1ccc(-c2cccc(-n3c4ccccc4c4cc(-c5ccc6c(c5)c5ccccc5n6-c5nc(-c6ccccc6)c6ccccc6n5)ccc43)c2)cc1 ; c1ccc(-c2cc(-c3ccccc3)cc(-n3c4ccccc4c4c5oc6ccccc6c5ccc43)c2)cc1]

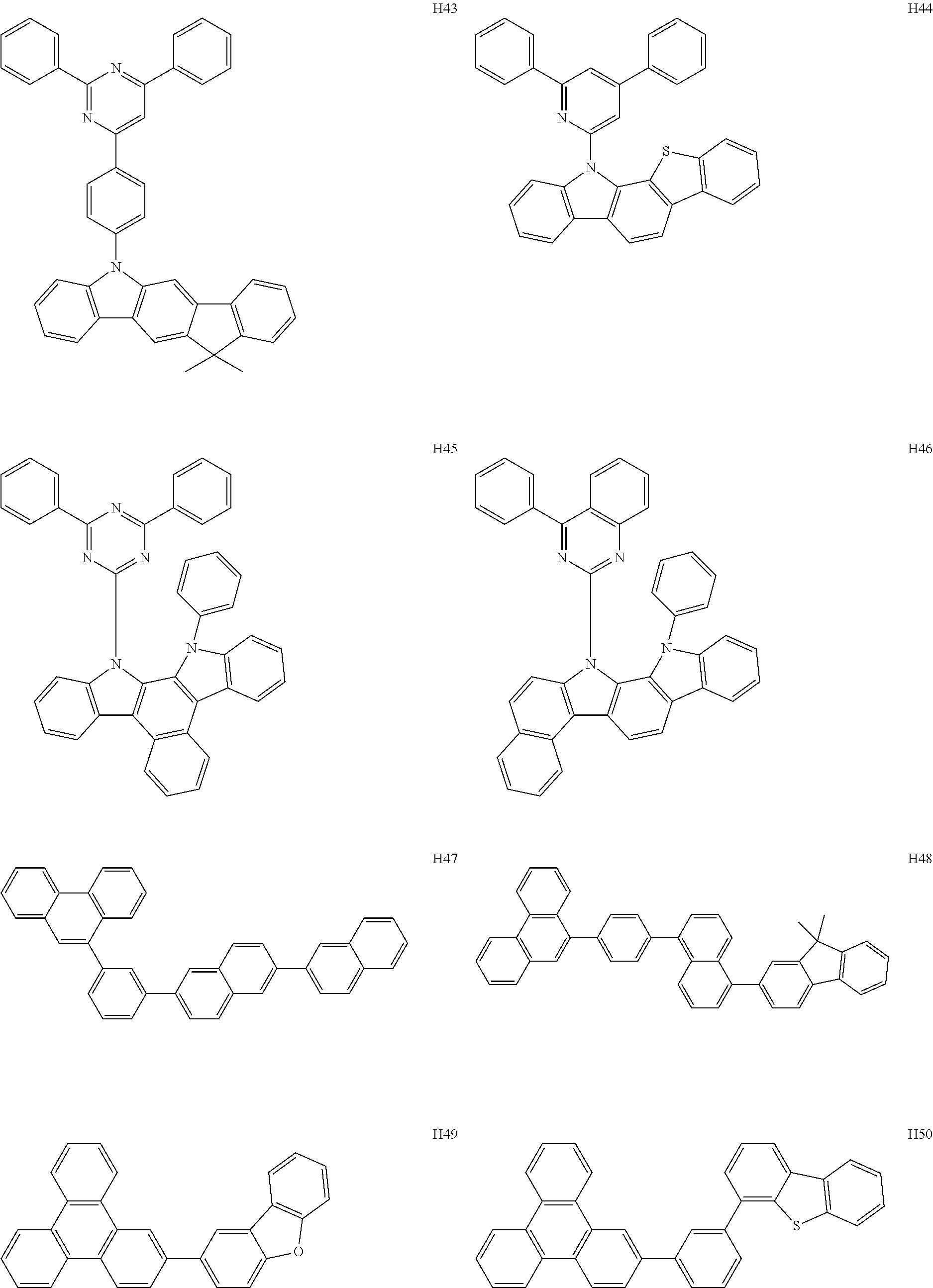

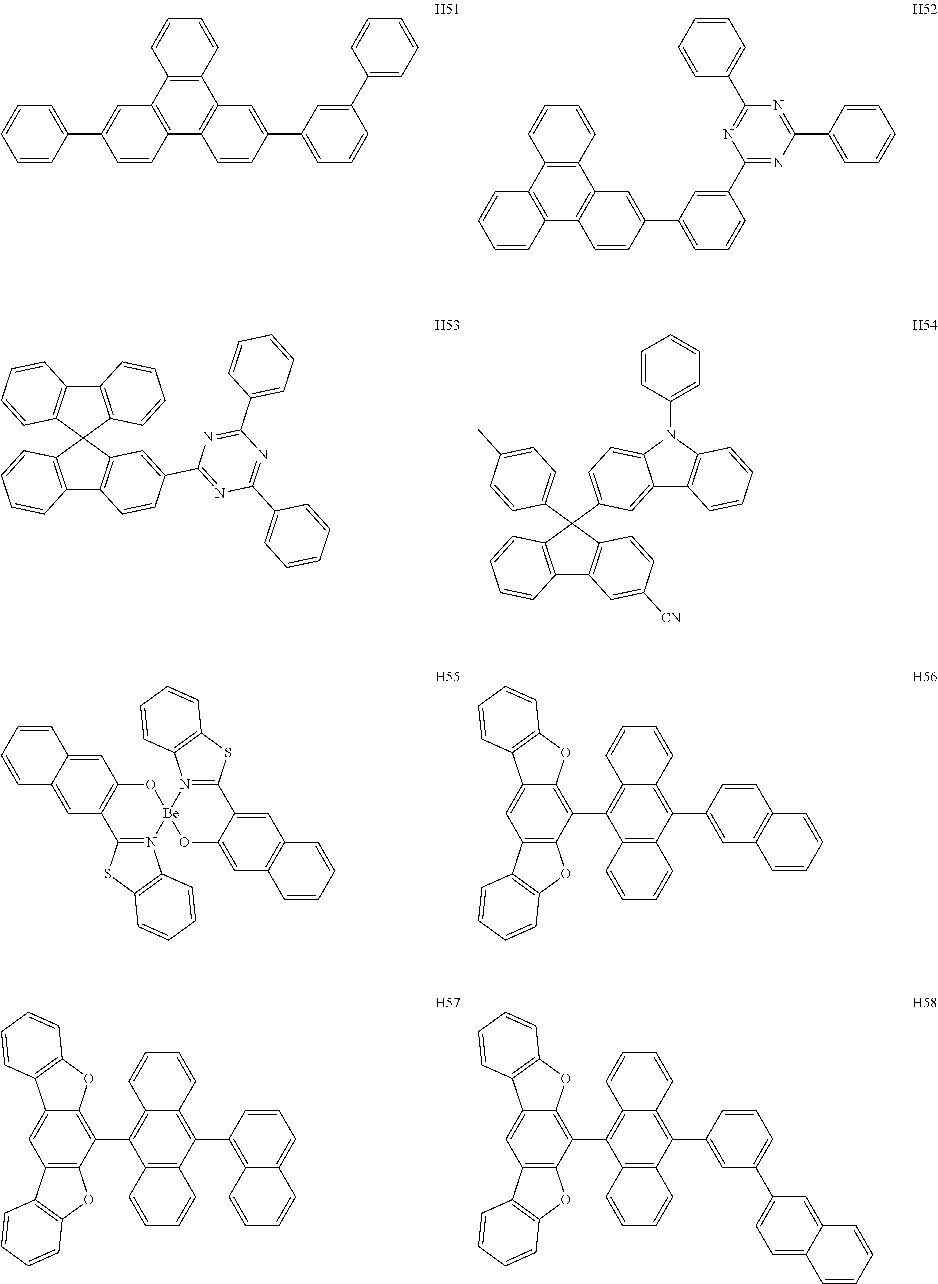

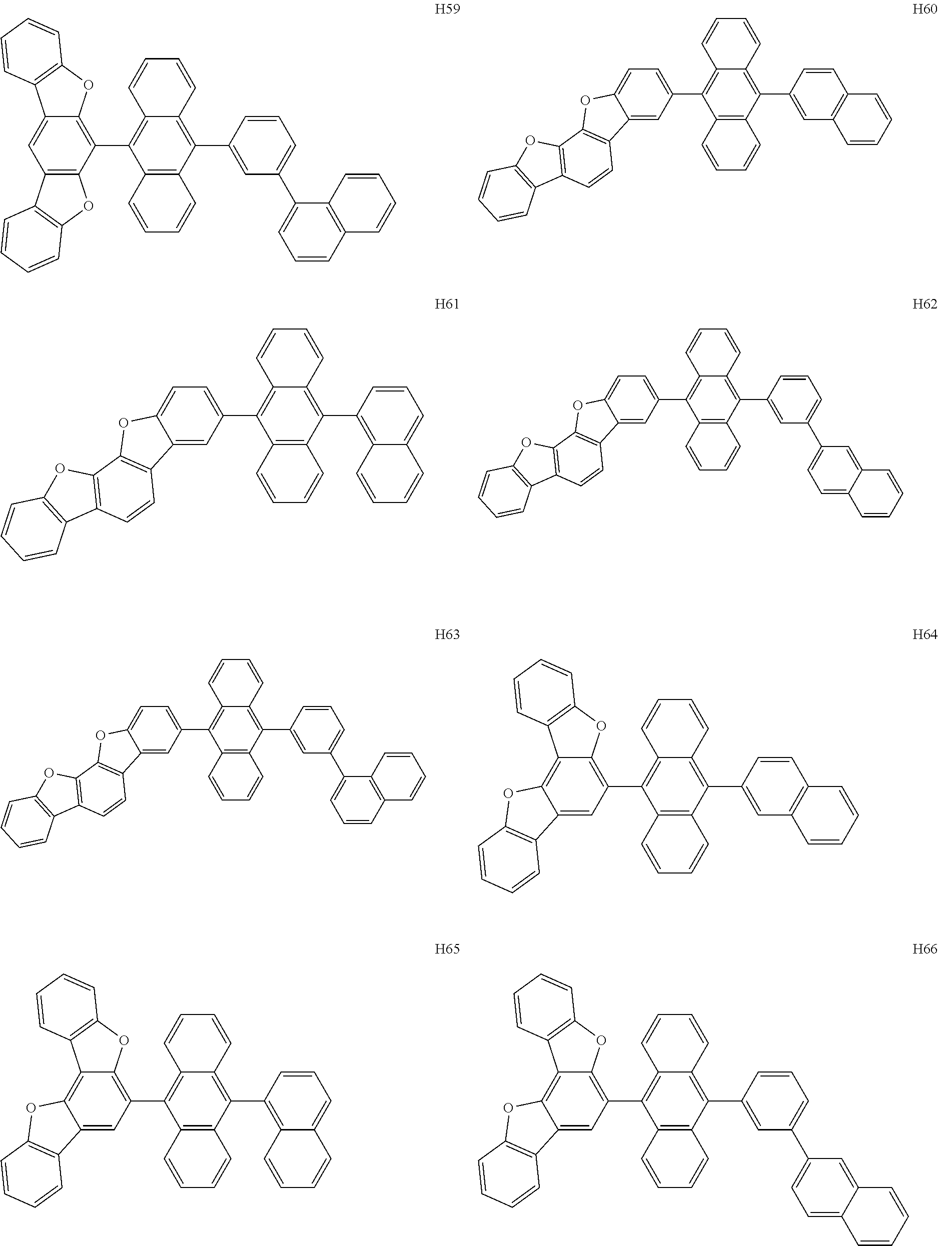

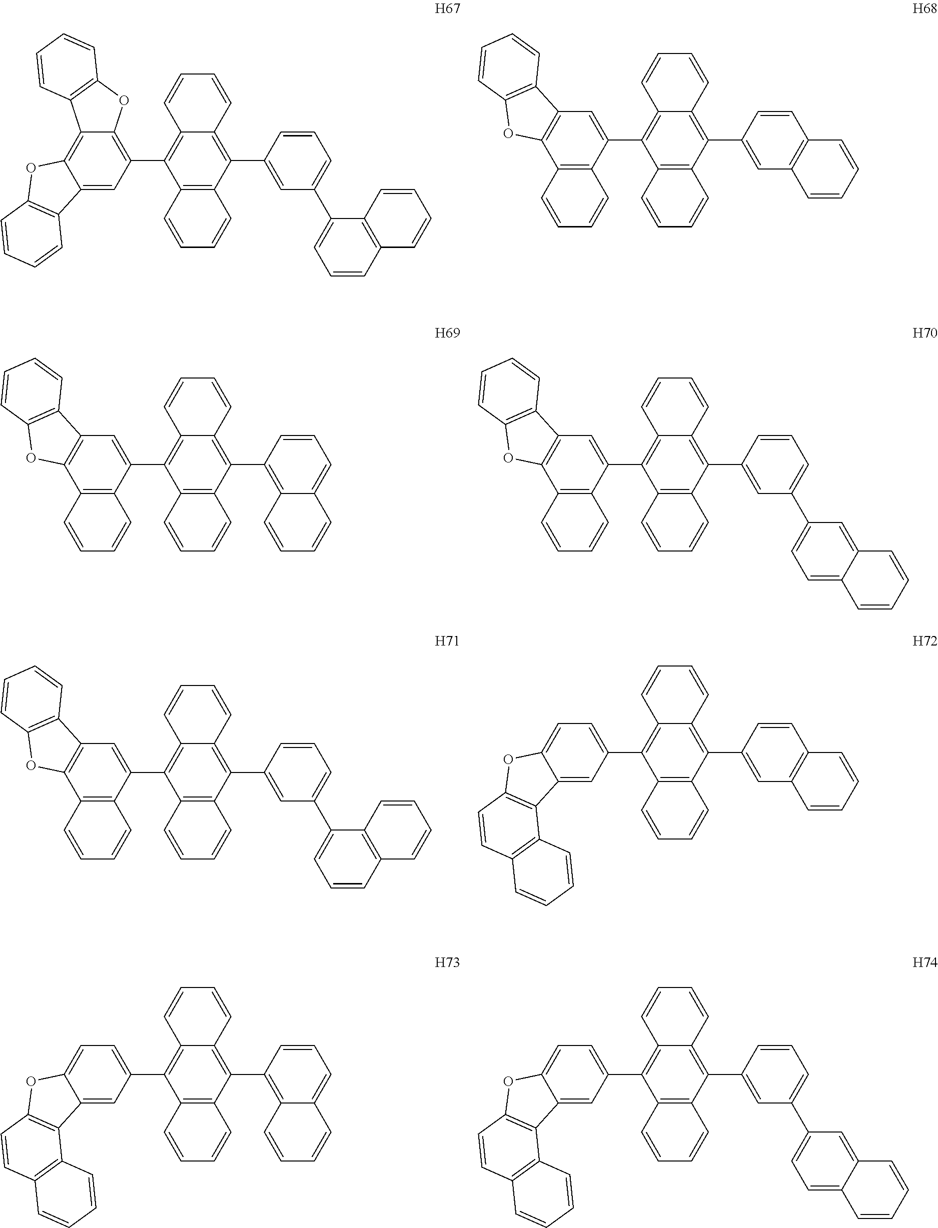

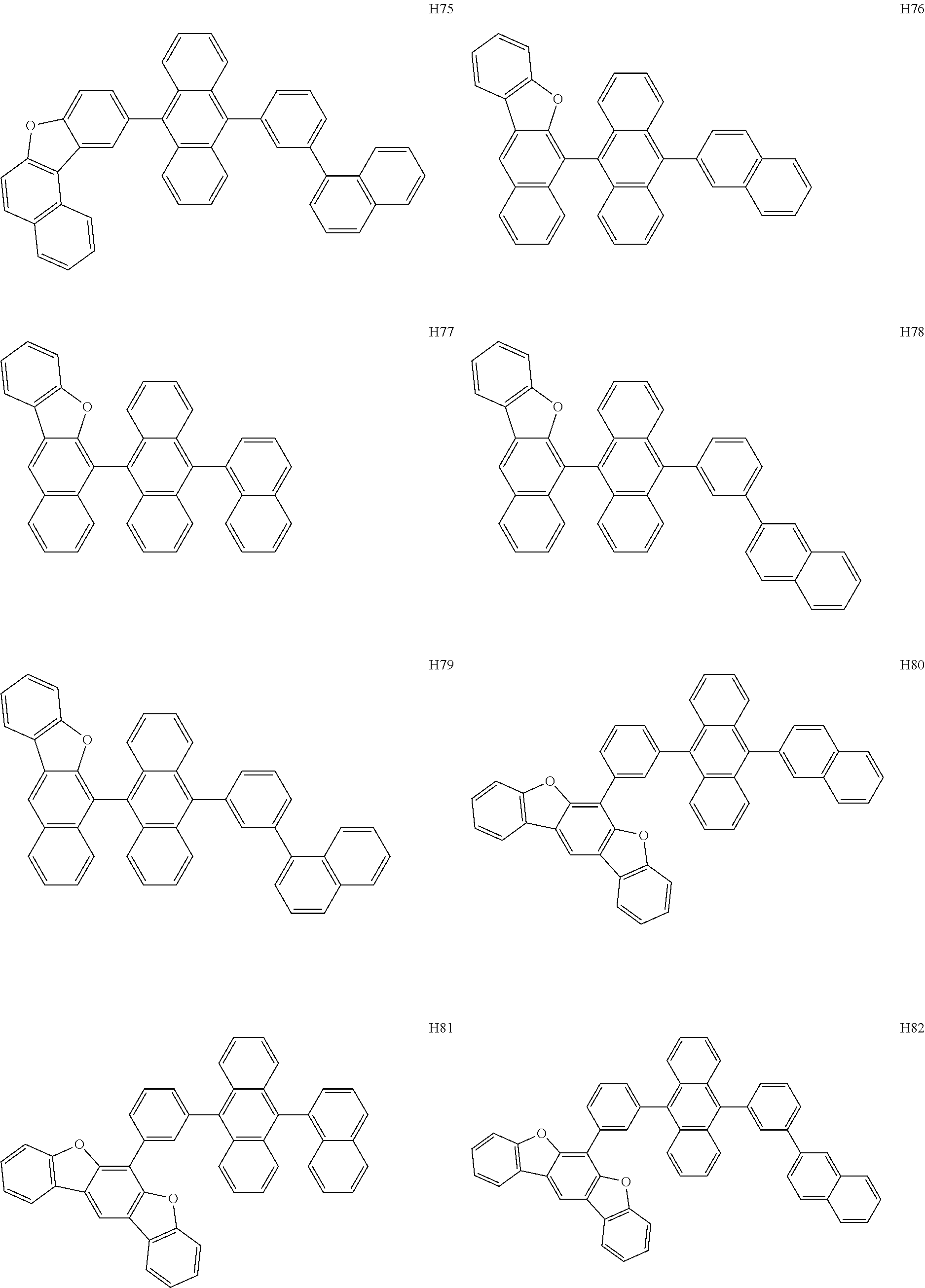

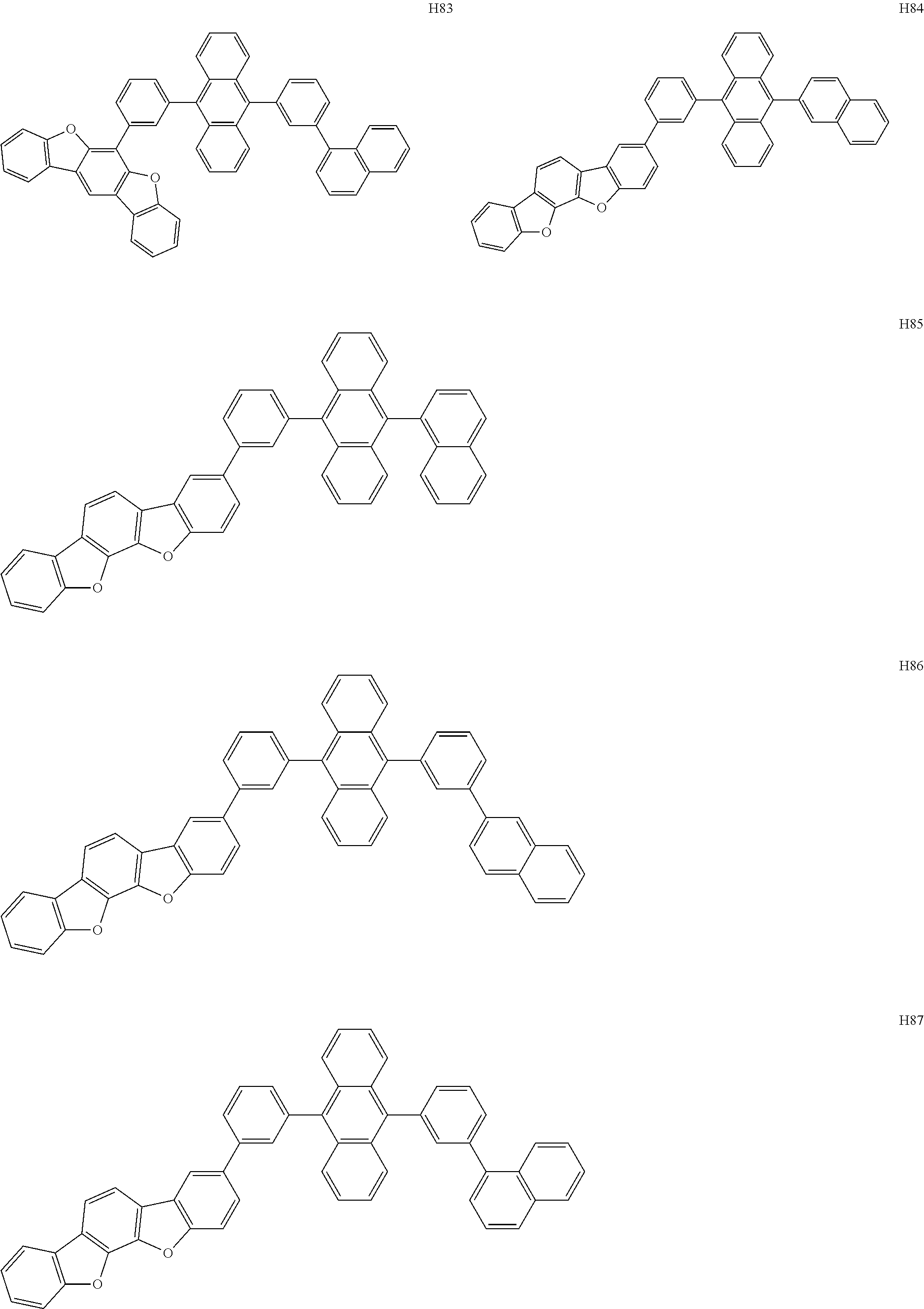 [SMILES: c1cc(-c2cccc3ccccc23)cc(-c2c3ccccc3c(-c3cccc(-c4c5oc6ccccc6c5cc5c4oc4ccccc45)c3)c3ccccc23)c1 ; c1cc(-c2ccc3oc4c(ccc5c6ccccc6oc54)c3c2)cc(-c2c3ccccc3c(-c3ccc4ccccc4c3)c3ccccc23)c1 ; c1cc(-c2ccc3oc4c(ccc5c6ccccc6oc54)c3c2)cc(-c2c3ccccc3c(-c3cccc4ccccc34)c3ccccc23)c1 ; c1cc(-c2ccc3ccccc3c2)cc(-c2c3ccccc3c(-c3cccc(-c4ccc5oc6c(ccc7c8ccccc8oc76)c5c4)c3)c3ccccc23)c1 ; c1cc(-c2ccc3oc4c(ccc5c6ccccc6oc54)c3c2)cc(-c2c3ccccc3c(-c3cccc(-c4cccc5ccccc45)c3)c3ccccc23)c1]

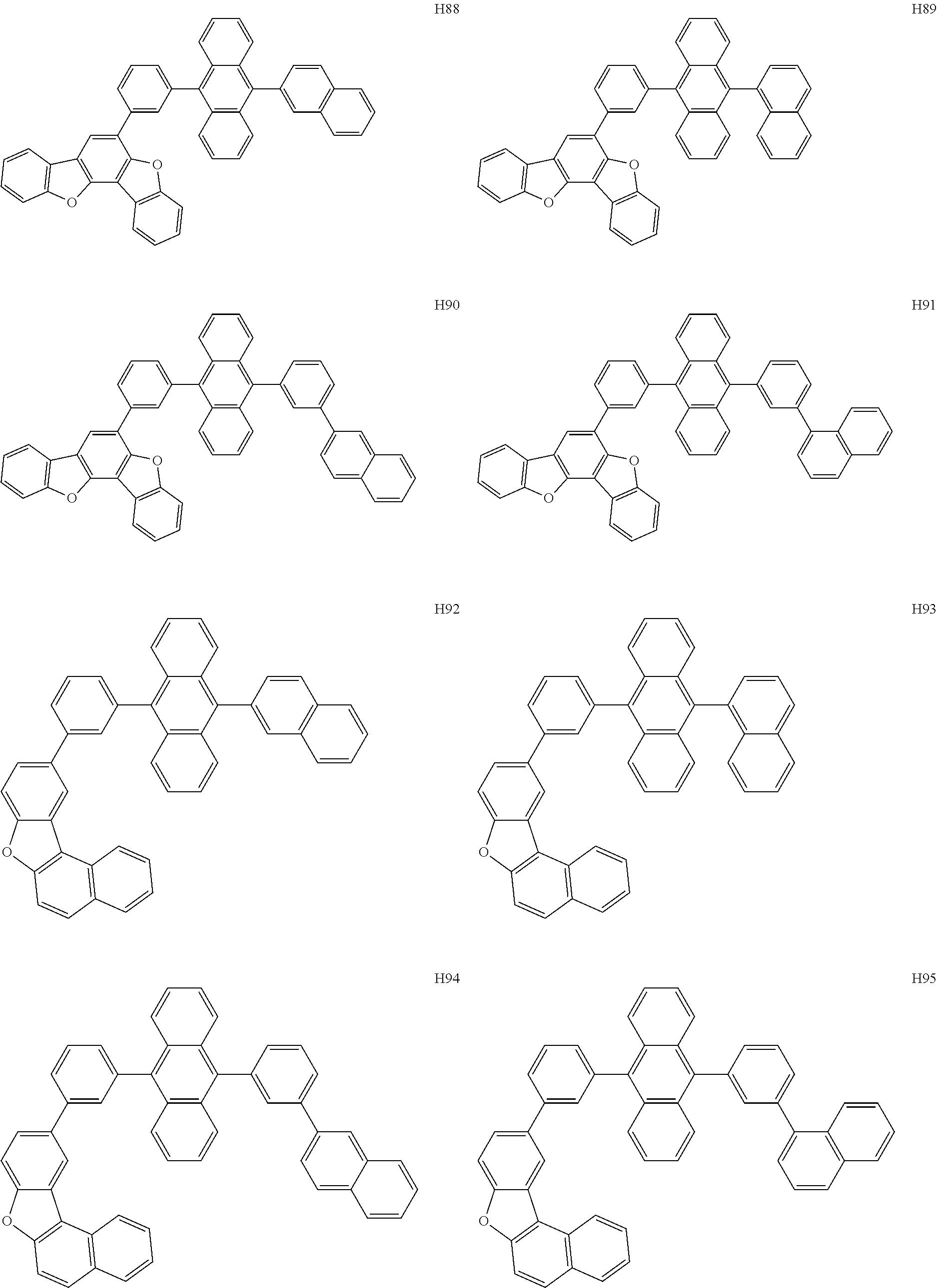

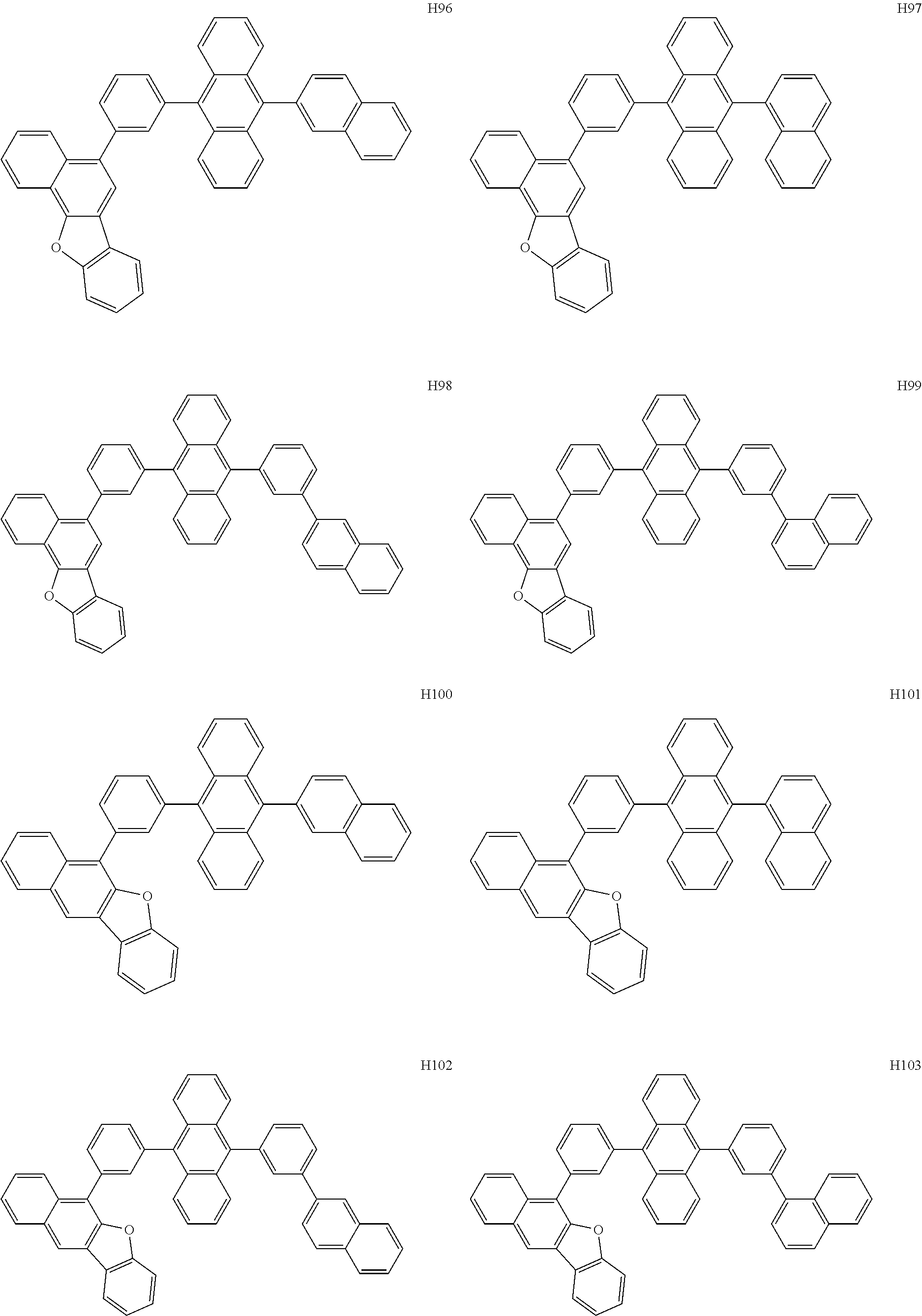

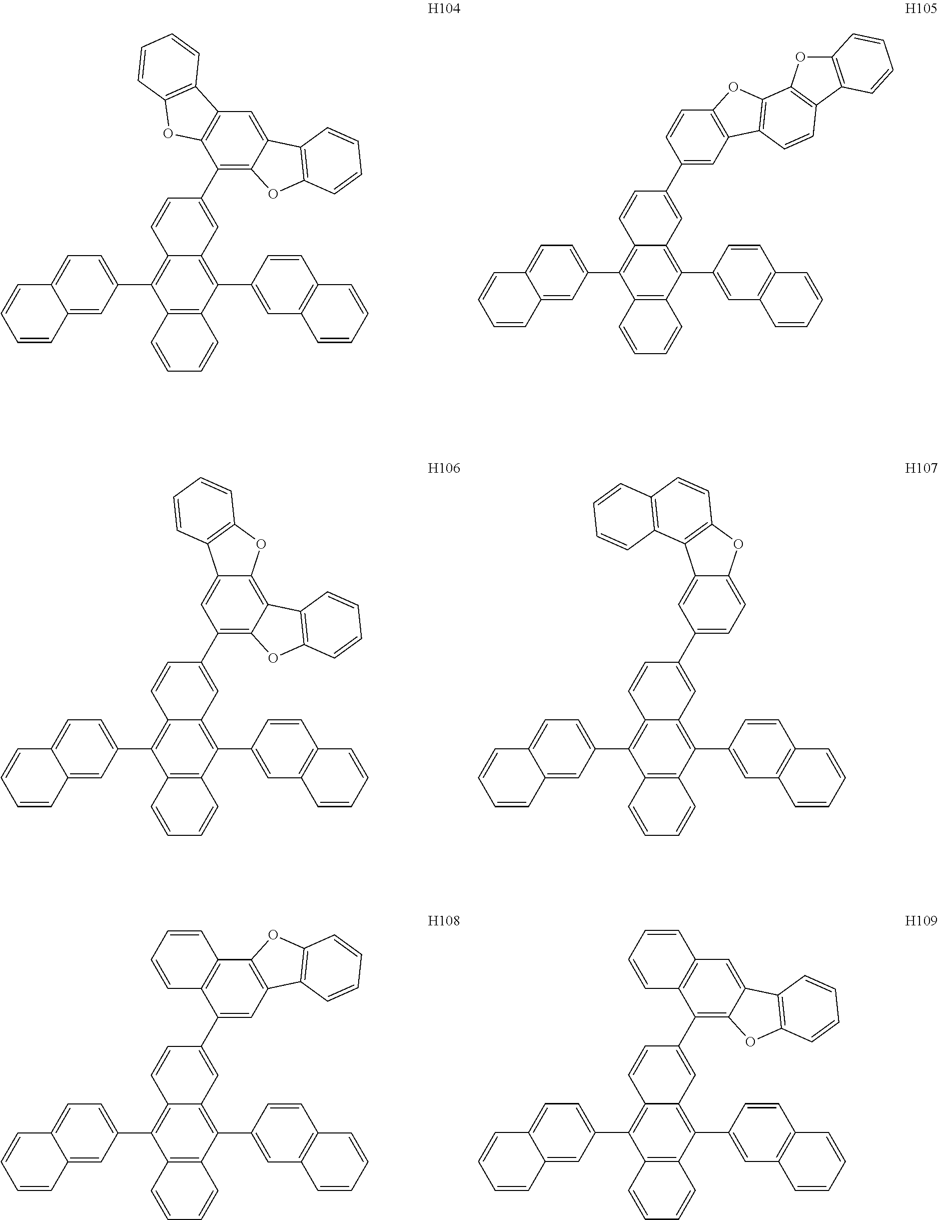

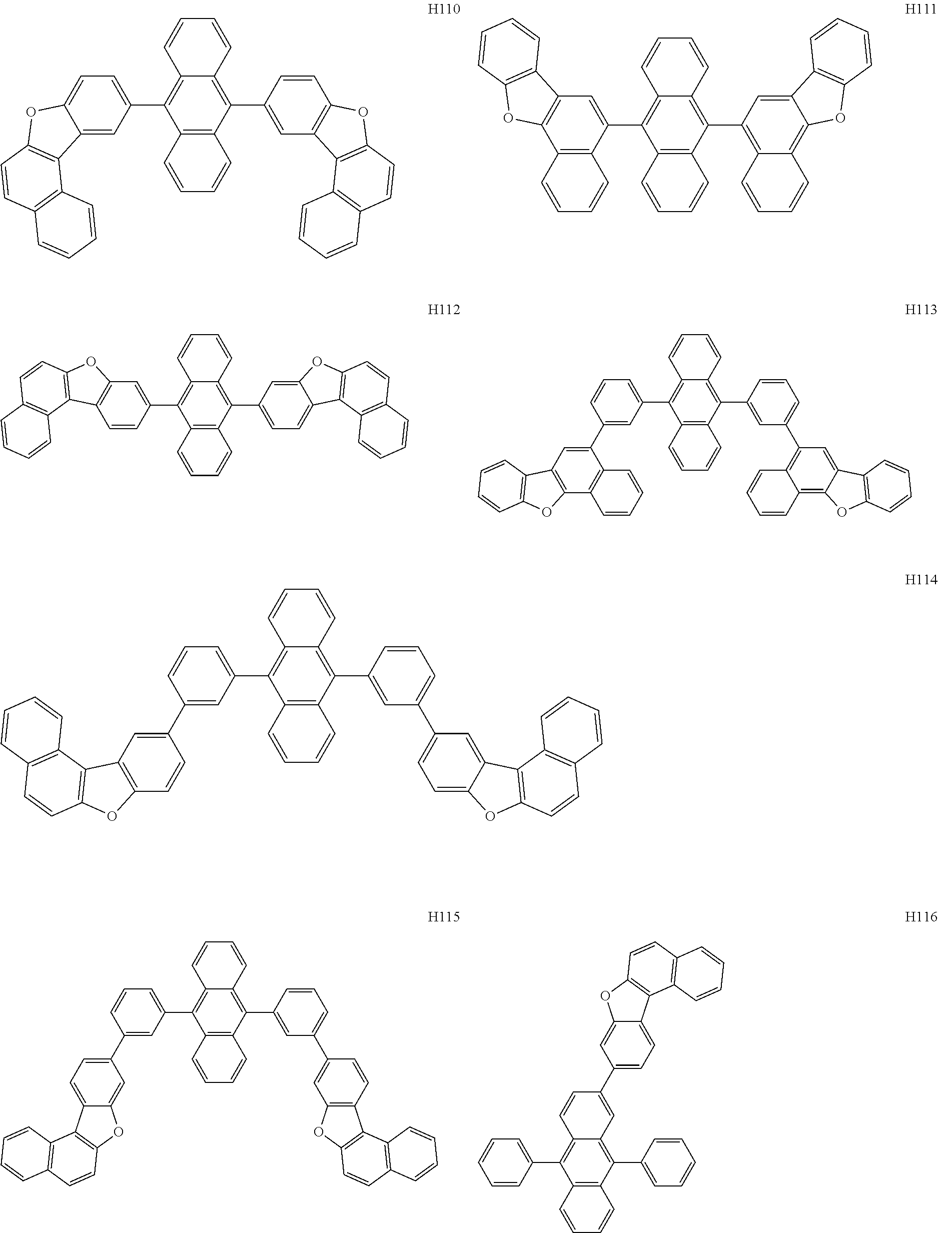

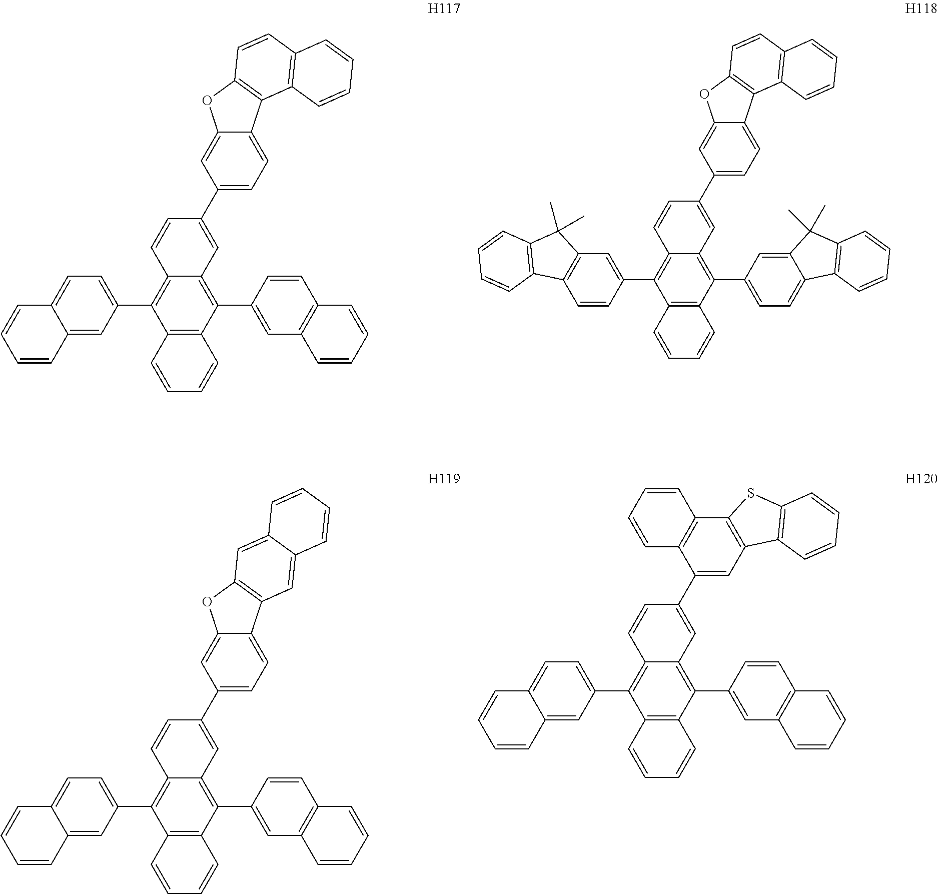 [SMILES: c1ccc2cc(-c3c4ccccc4c(-c4ccc5ccccc5c4)c4cc(-c5ccc6c(c5)oc5ccc7ccccc7c56)ccc34)ccc2c1 ; CC1(C)c2ccccc2-c2ccc(-c3c4ccccc4c(-c4ccc5c(c4)C(C)(C)c4ccccc4-5)c4cc(-c5ccc6c(c5)oc5ccc7ccccc7c56)ccc34)cc21 ; c1ccc2cc(-c3c4ccccc4c(-c4ccc5ccccc5c4)c4cc(-c5ccc6c(c5)oc5cc7ccccc7cc56)ccc34)ccc2c1 ; c1ccc2cc(-c3c4ccccc4c(-c4ccc5ccccc5c4)c4cc(-c5cc6c7ccccc7sc6c6ccccc56)ccc34)ccc2c1]

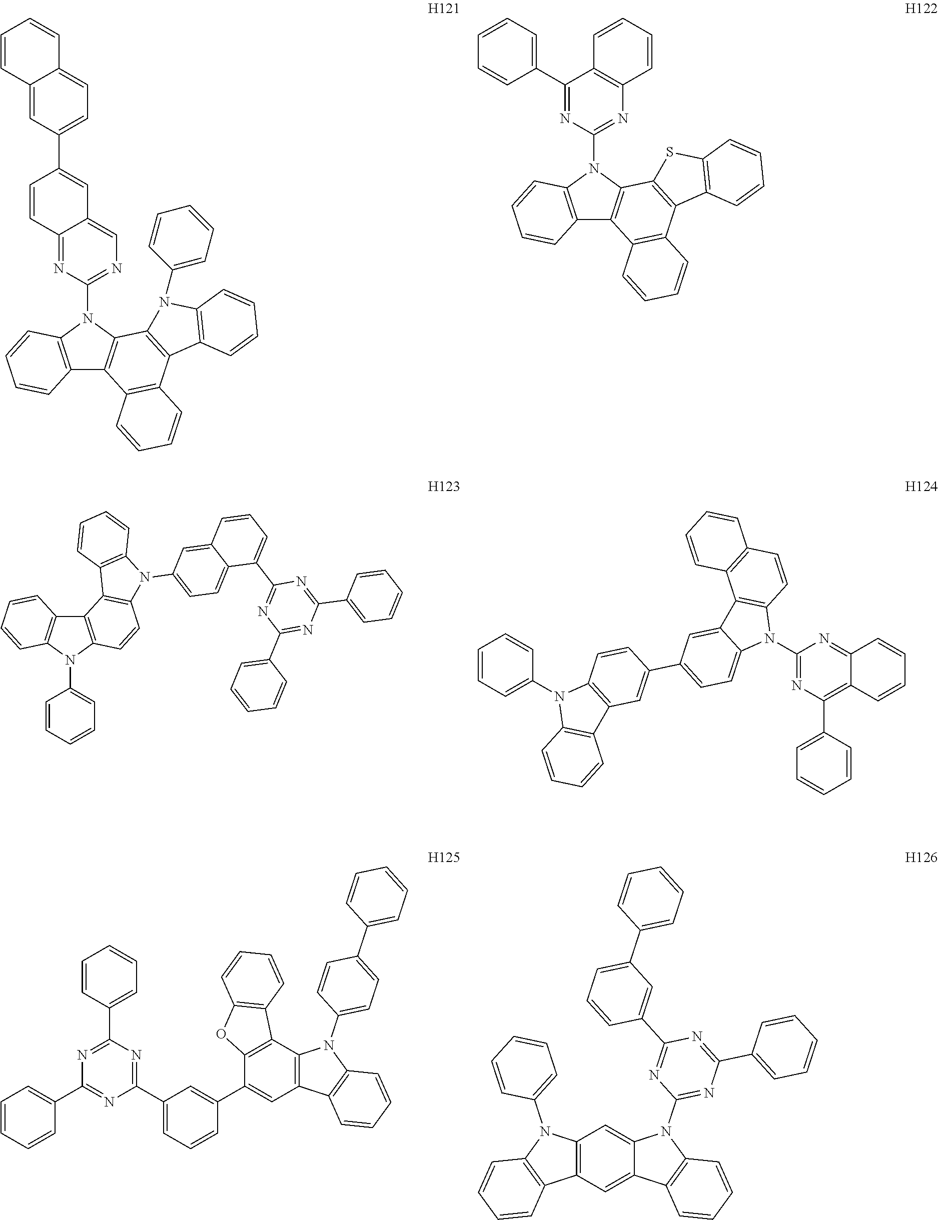

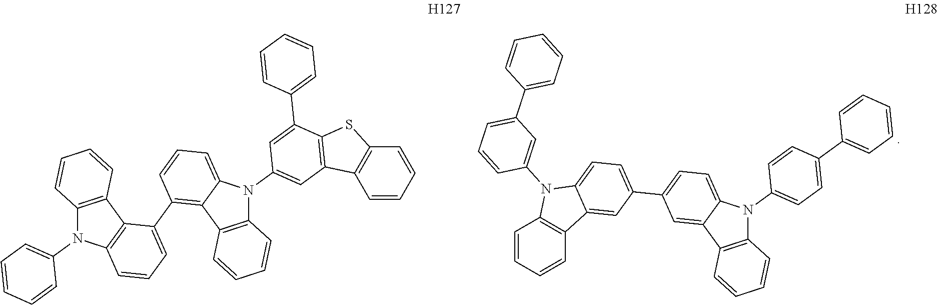 [SMILES: c1ccc(-c2cc(-n3c4ccccc4c4c(-c5cccc6c5c5ccccc5n6-c5ccccc5)cccc43)cc3c2sc2ccccc23)cc1 ; c1ccc(-c2ccc(-n3c4ccccc4c4cc(-c5ccc6c(c5)c5ccccc5n6-c5cccc(-c6ccccc6)c5)ccc43)cc2)cc1]

### Phosphorescent Dopant

The phosphorescent dopant may include at least one transition metal as a central metal.

The phosphorescent dopant may include a monodentate ligand, a bidentate ligand, a tridentate ligand, a tetradentate ligand, a pentadentate ligand, a hexadentate ligand, or any combination thereof.

The phosphorescent dopant may be electrically neutral.

For example, the phosphorescent dopant may include an organometallic compound represented by Formula 401:

M(L₄₀₁)_{xc1}(L₄₀₂)_{xc2} Formula 401

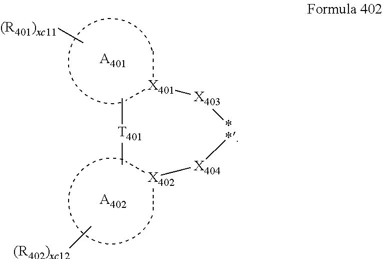

In Formulae 401 and 402,
M may be a transition metal (for example, iridium (Ir), platinum (Pt), palladium (Pd), osmium (Os), titanium (Ti), gold (Au), hafnium (Hf), europium (Eu), terbium (Tb), rhodium (Rh), rhenium (Re), or thulium (Tm)),
L₄₀₁ may be a ligand represented by Formula 402, and xc1 is 1, 2, or 3, wherein, when xc1 is 2 or more, two or more of L₄₀₁ may be identical to or different from each other,
L₄₀₂ may be an organic ligand, and xc2 may be 0, 1, 2, 3, or 4, wherein, when xc2 is 2 or more, two or more of L₄₀₂ may be identical to or different from each other,
X₄₀₁ and X₄₀₂ may each independently be N or C,
ring A₄₀₁ and ring A₄₀₂ may each independently be a C₃-C₆₀ carbocyclic group or a C₁-C₆₀ heterocyclic group,
T₄₀₁ may be a single bond, *—O—*′, *—S—*′, *—C(═O)—*′, *—N(Q₄₁₁)-*′, *—C(Q₄₁₁)(Q₄₁₂)-*′, *—C(Q₄₁₁)═C(Q₄₁₂)-*′, *—C(Q₄₁₁)=*′, or *═C═*′,
X₄₀₃ and X₄₀₄ may each independently be a chemical bond (for example, a covalent bond or a coordinate bond), O, S, N(Q₄₁₃), B(Q₄₁₃), P(Q₄₁₃), C(Q₄₁₃)(Q₄₁₄), or Si(Q₄₁₃)(Q₄₁₄),
Q₄₁₁ to Q₄₁₄ may each be defined as in connection with Q₁,
R₄₀₁ and R₄₀₂ may each independently be hydrogen, deuterium, —F, —Cl, —Br, —I, a hydroxyl group, a cyano group, a nitro group, a C₁-C₂₀ alkyl group unsubstituted or substituted with at least one R₁₀ₐ, a C₁-C₂₀ alkoxy group unsubstituted or substituted with at least one R₁₀ₐ, a C₃-C₆₀ carbocyclic group unsubstituted or substituted with at least one R₁₀ₐ, a C₁-C₆₀ heterocyclic group unsubstituted or substituted with at least one R₁₀ₐ, —Si(Q₄₀₁)(Q₄₀₂)(Q₄₀₃), —N(Q₄₀₁)(Q₄₀₂), —B(Q₄₀₁)(Q₄₀₂), —C(═O)(Q₄₀₁), —S(═O)₂(Q₄₀₁), or —P(═O)(Q₄₀₁)(Q₄₀₂),
Q₄₀₁ to Q₄₀₃ may each be defined as in connection with Q₁,
xc11 and xc12 may each independently be an integer from 0 to 10, and
* and *′ in Formula 402 each indicate a binding site to M in Formula 401.

For example, in Formula 402, i) X₄₀₁ may be nitrogen and X₄₀₂ may be carbon, or ii) each of X₄₀₁ and X₄₀₂ may be nitrogen.

In one or more embodiments, when xc1 in Formula 401 is 2 or more, two ring A₄₀₁(s) among two or more of L₄₀₁ may optionally be linked to each other via T₄₀₂, which is a linking group, and two ring A₄₀₂(s) among two or more of L₄₀₁ may optionally be linked to each other via T₄₀₃, which is a linking group (see Compounds PD1 to PD4 and PD7). T₄₀₂ and T₄₀₃ may each be defined as in connection with T₄₀₁.

In Formula 401, L₄₀₂ may be an organic ligand. For example, L₄₀₂ may include a halogen group, a diketone group (for example, an acetylacetonate group), a carboxylic acid group (for example, a picolinate group), —C(═O), an isonitrile group, a —CN group, a phosphorus group (for example, a phosphine group, a phosphite group, etc.), or any combination thereof.

The phosphorescent dopant may include, for example, at least one selected from among Compounds PD1 to PD39, and/or any combination thereof:

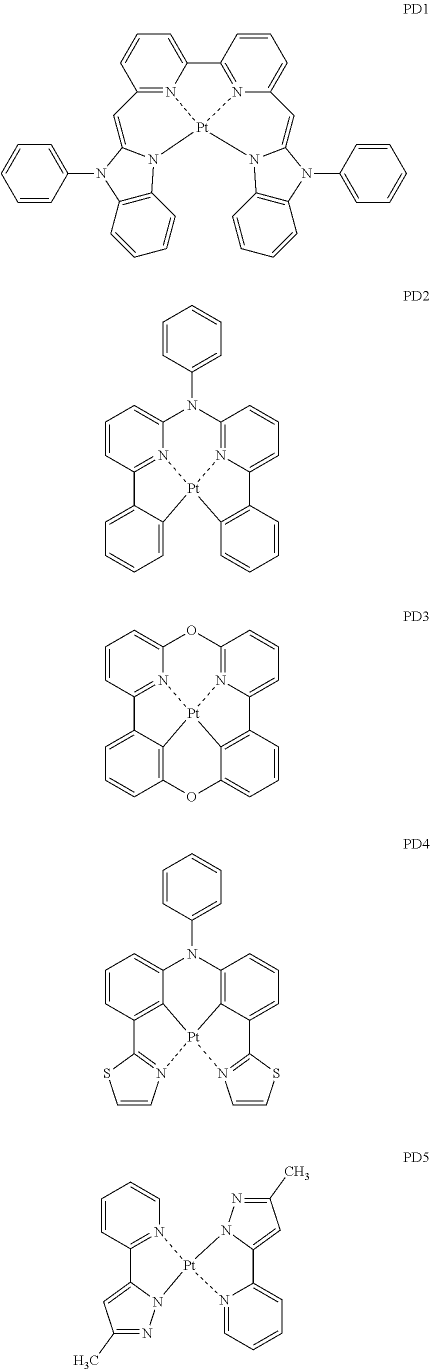

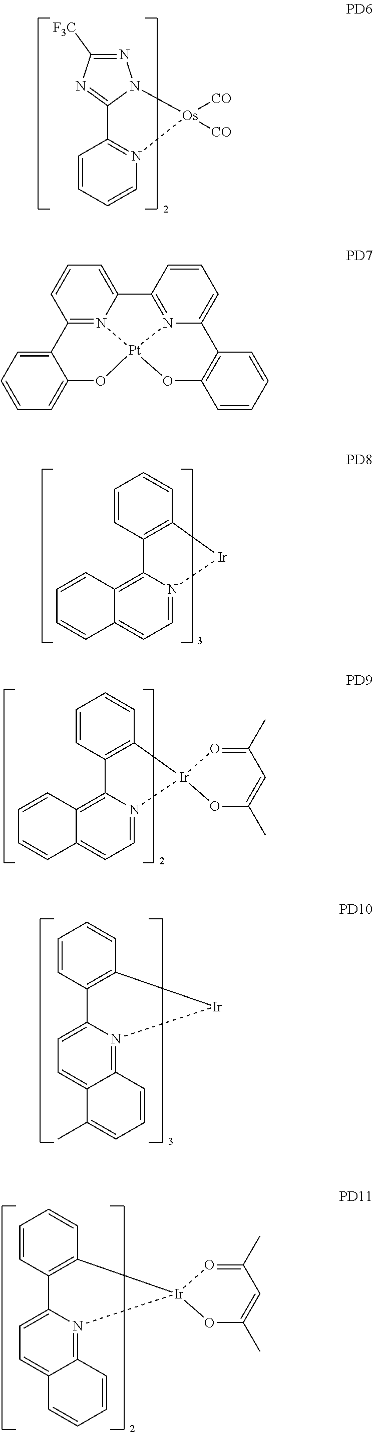

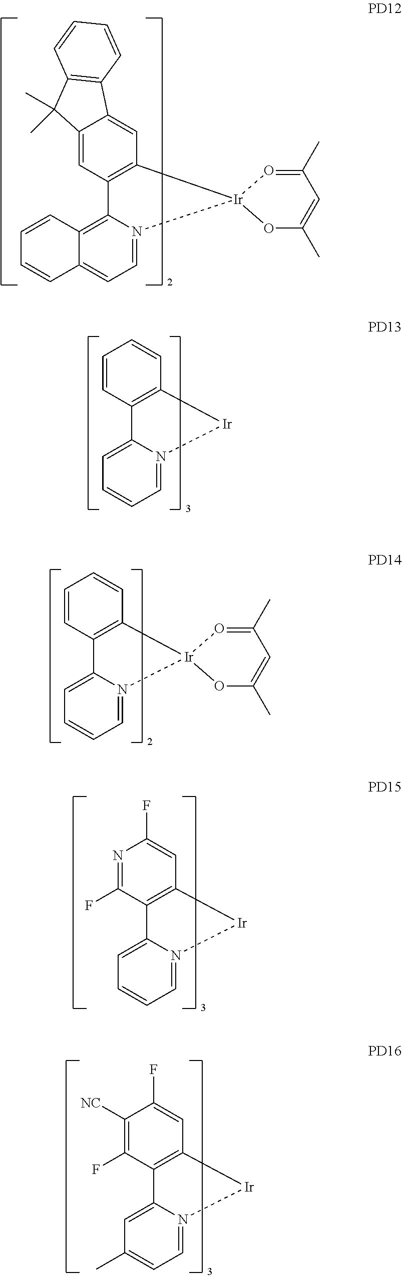

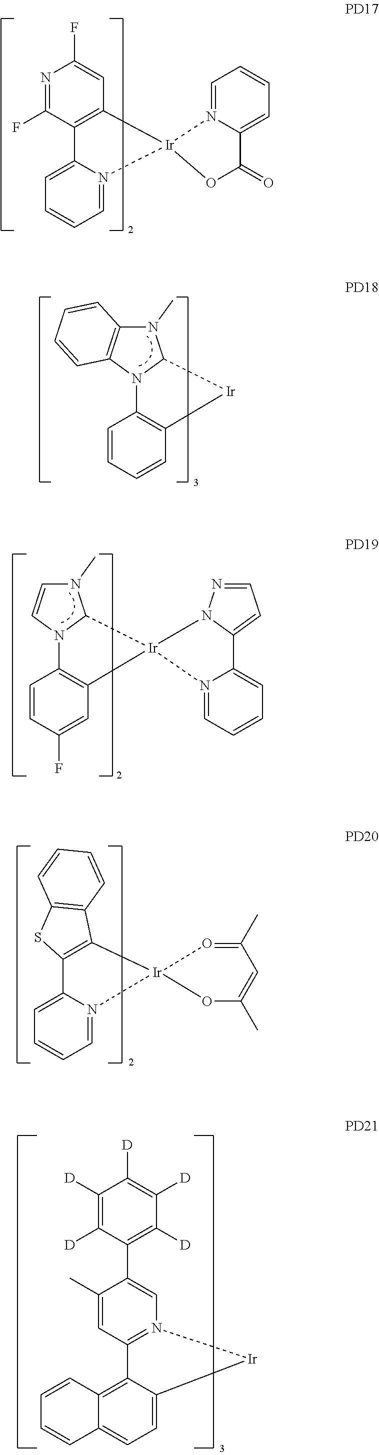

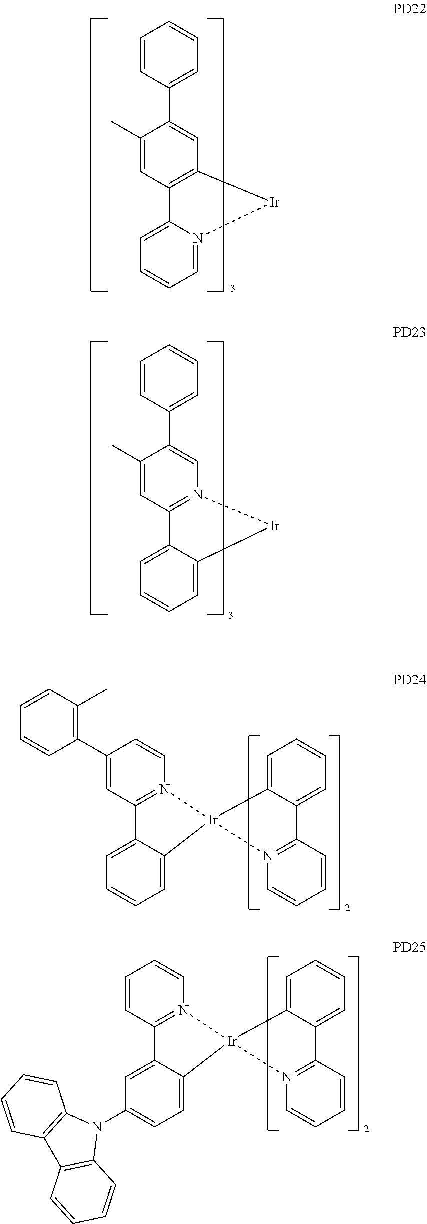

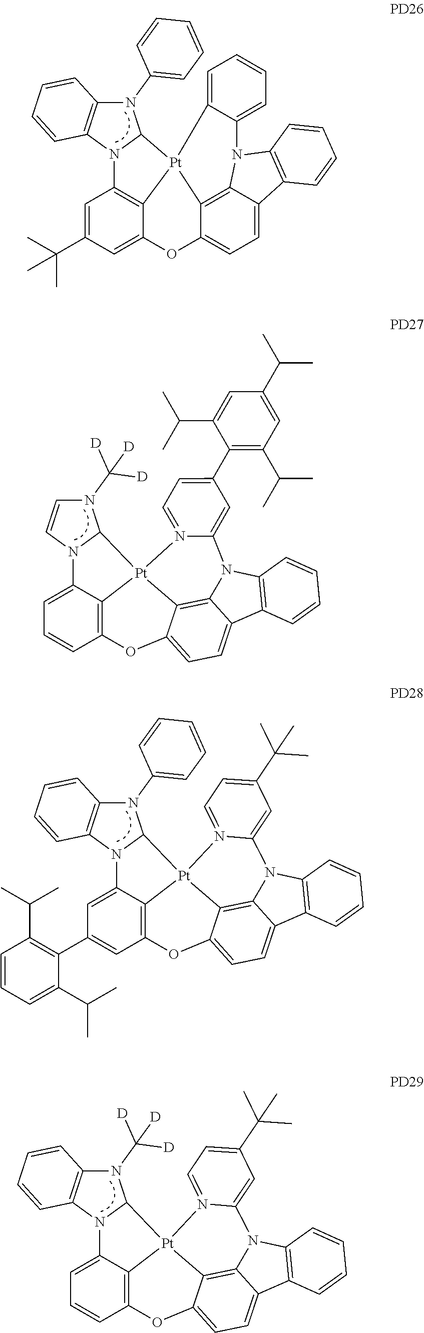

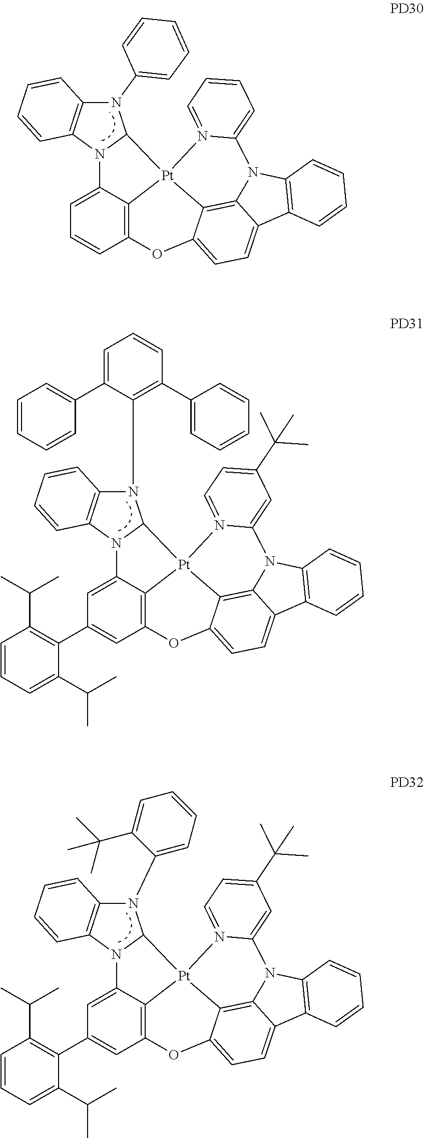

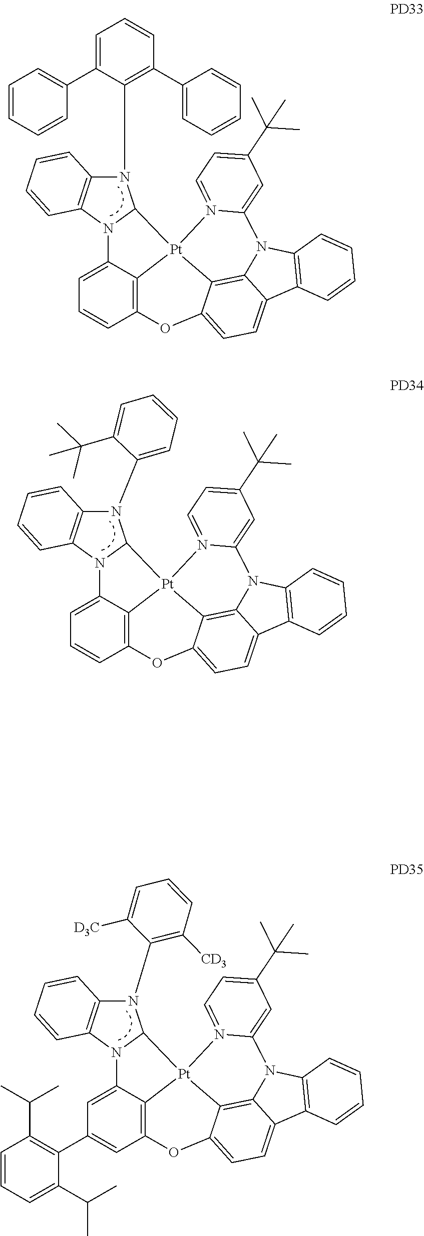

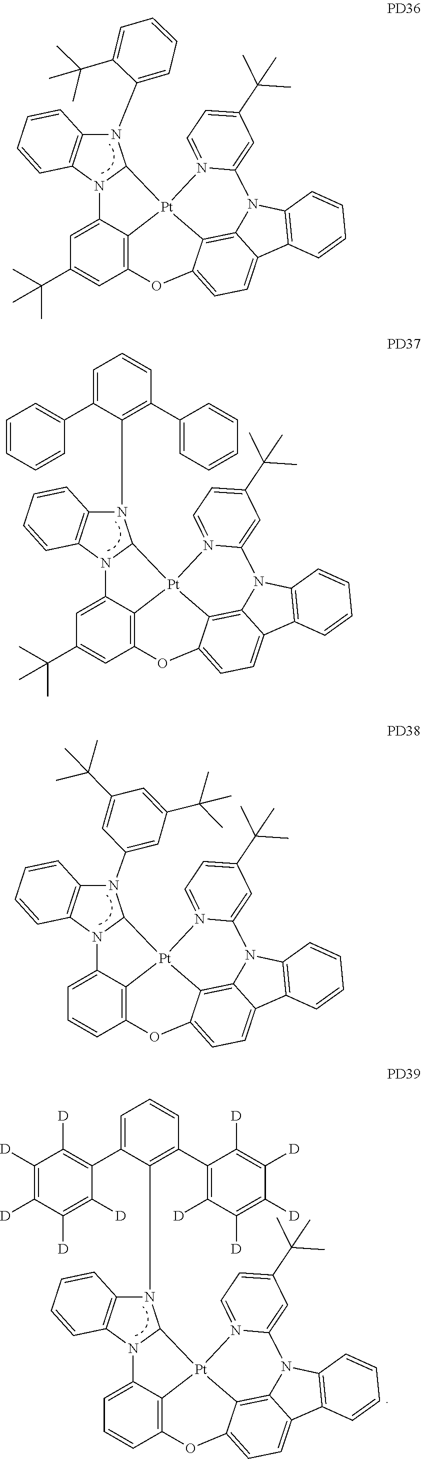

### Fluorescent Dopant

The fluorescent dopant may include an amine group-containing compound, a styryl group-containing compound, or any combination thereof.

For example, the fluorescent dopant may include a compound represented by Formula 501:

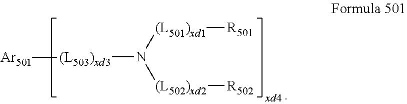

In Formula 501,
Ar₅₀₁, L₅₀₁ to L₅₀₃, R₅₀₁, and R₅₀₂ may each independently be a C₃-C₆₀ carbocyclic group unsubstituted or substituted with at least one R₁₀ₐ or a C₁-C₆₀ heterocyclic group unsubstituted or substituted with at least one R₁₀ₐ,
xd1 to xd3 may each independently be 0, 1, 2, or 3, and
xd4 may be 1, 2, 3, 4, 5, or 6.

In one or more embodiments, Ar₅₀₁ in Formula 501 may be a condensed cyclic group (for example, an anthracene group, a chrysene group, a pyrene group, etc.) in which three or more monocyclic groups are condensed together.

In one or more embodiments, xd4 in Formula 501 may be 2.

In one or more embodiments, the fluorescent dopant may include: at least one selected from among Compounds FD1 to FD37; DPVBi; DPAVBi; and/or any combination thereof:

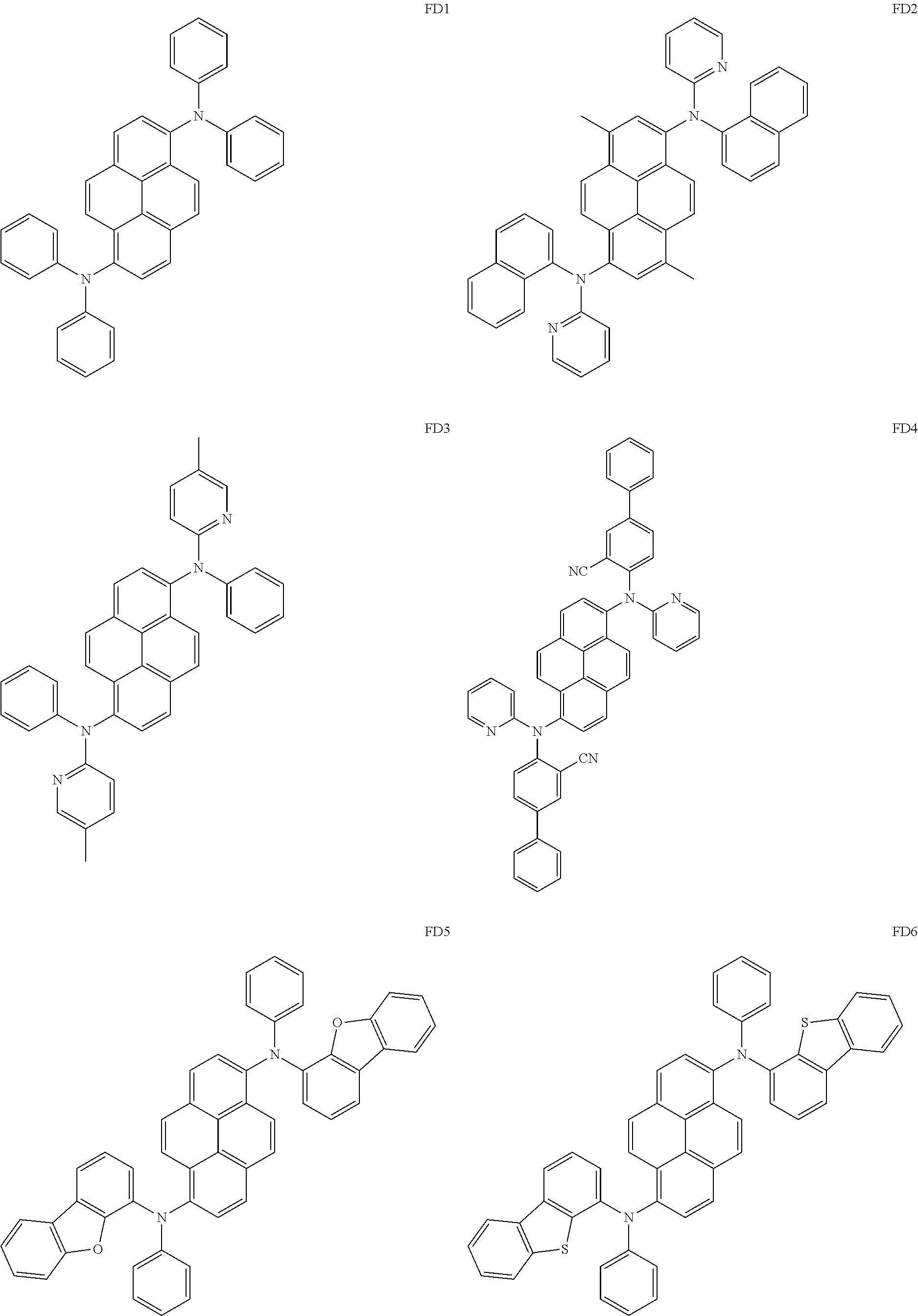

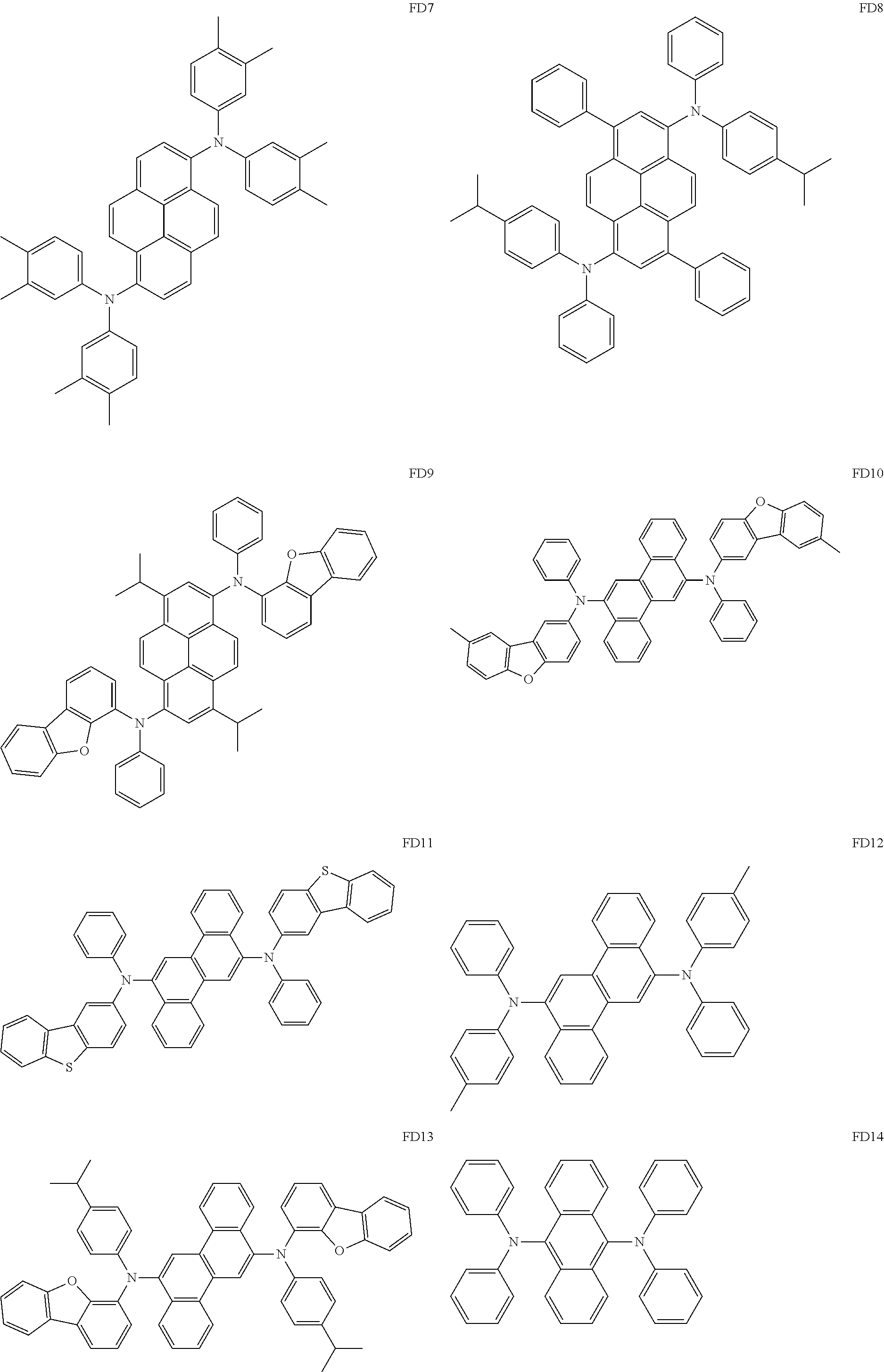

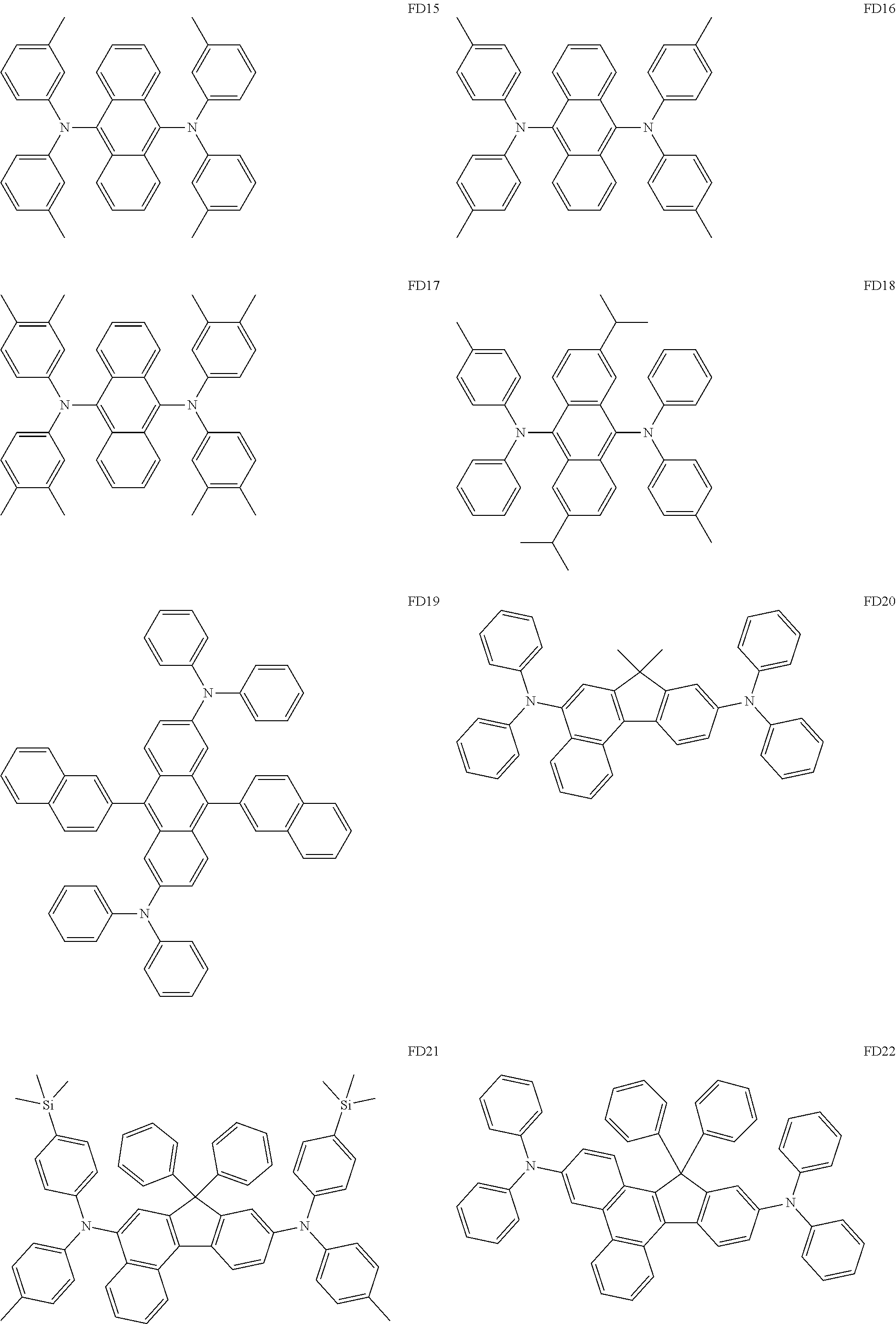

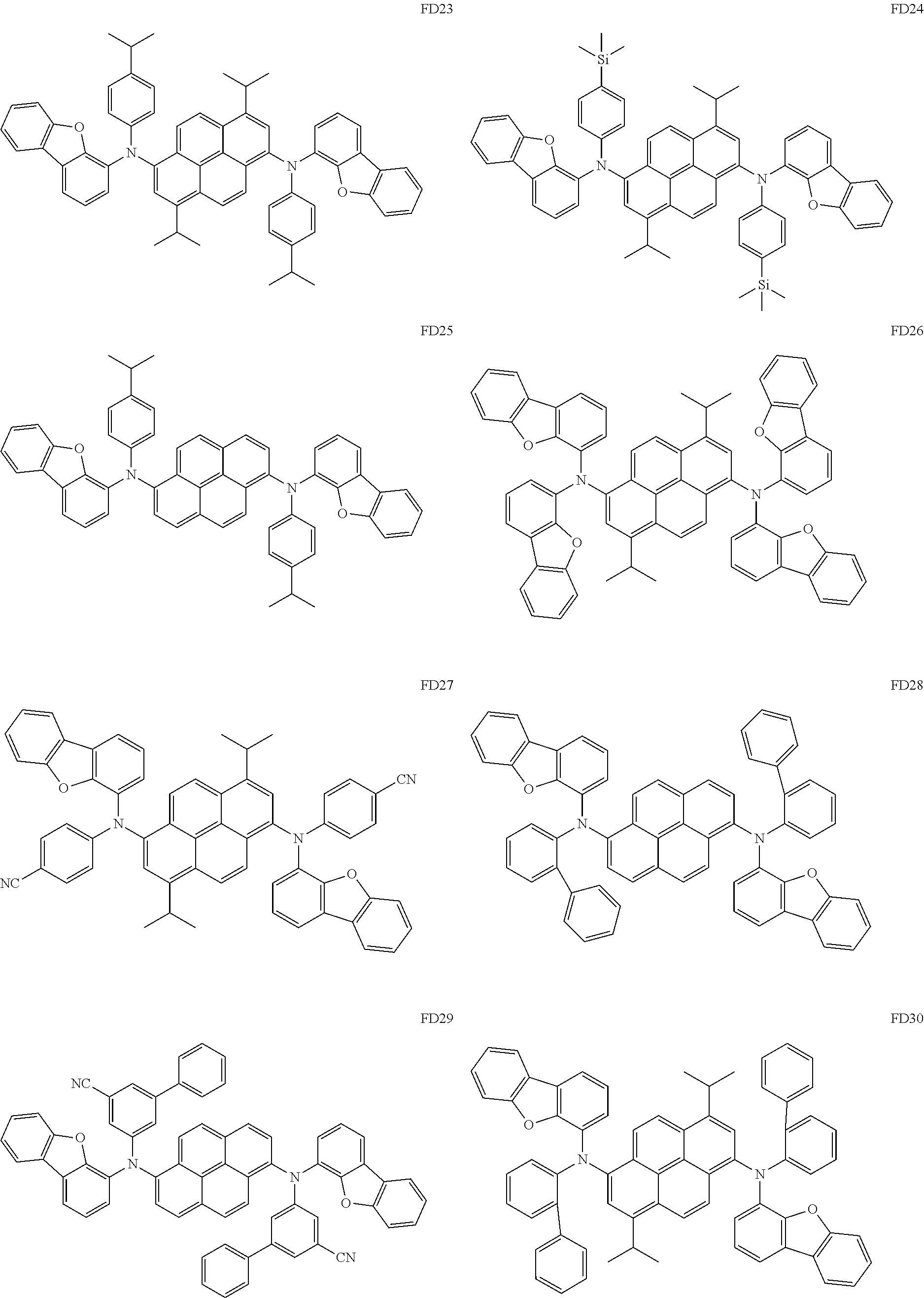

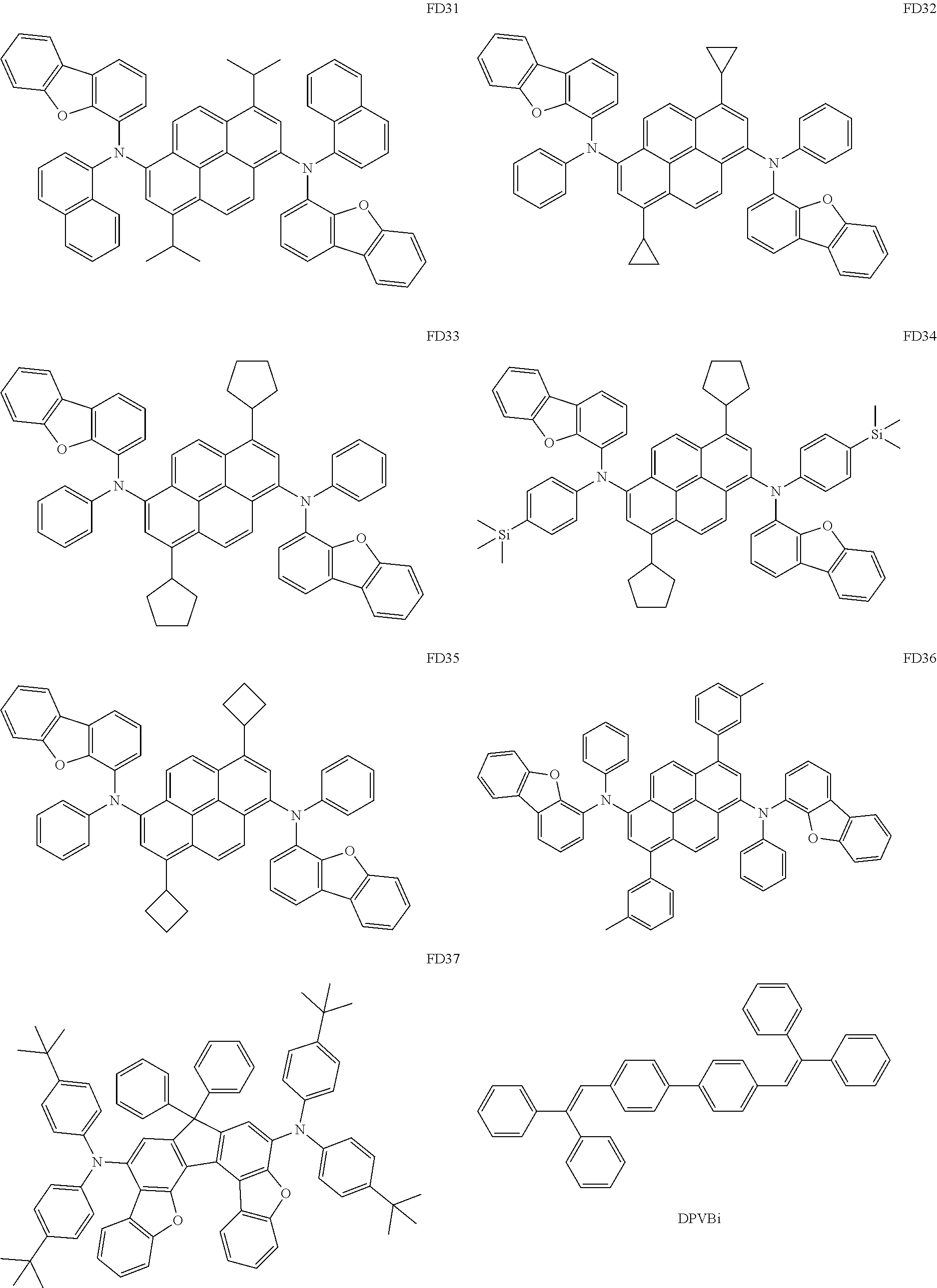

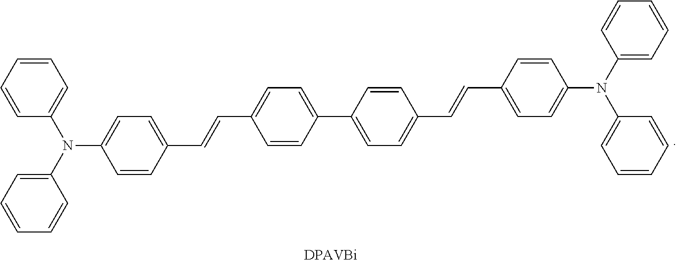 [SMILES: C(=C\c1ccc(N(c2ccccc2)c2ccccc2)cc1)/c1ccc(-c2ccc(/C=C/c3ccc(N(c4ccccc4)c4ccccc4)cc3)cc2)cc1 ; "DPAVBi"]

### Delayed Fluorescence Material

The emission layer 130 may include a delayed fluorescence material.

Herein, the delayed fluorescence material may be selected from compounds capable of emitting delayed fluorescence based on a delayed fluorescence emission mechanism.

The delayed fluorescence material included in the emission layer 130 may act as a host or a dopant depending on the type or kind of other materials included in the emission layer 130.

In one or more embodiments, a difference between a triplet energy level (eV) of the delayed fluorescence material and a singlet energy level (eV) of the delayed fluorescence material may be greater than or equal to 0 eV and less than or equal to 0.5 eV. When the difference between the triplet energy level (eV) of the delayed fluorescence material and the singlet energy level (eV) of the delayed fluorescence material is satisfied within the range above, up-conversion from the triplet state to the singlet state of the delayed fluorescence materials may effectively occur, and thus, the light-emitting device 10 may have improved luminescence efficiency.

For example, the delayed fluorescence material may include i) a material including at least one electron donor (for example, a π electron-rich C₃-C₆₀ cyclic group, such as a carbazole group, etc.) and at least one electron acceptor (for example, a sulfoxide group, a cyano group, a π electron-deficient nitrogen-containing C₁-C₆₀ cyclic group, etc.), ii) a material including a C₈-C₆₀ polycyclic group including at least two cyclic groups condensed to each other while sharing boron (B), and/or the like.

Examples of the delayed fluorescence material may include at least one of Compounds DF1 to DF14:

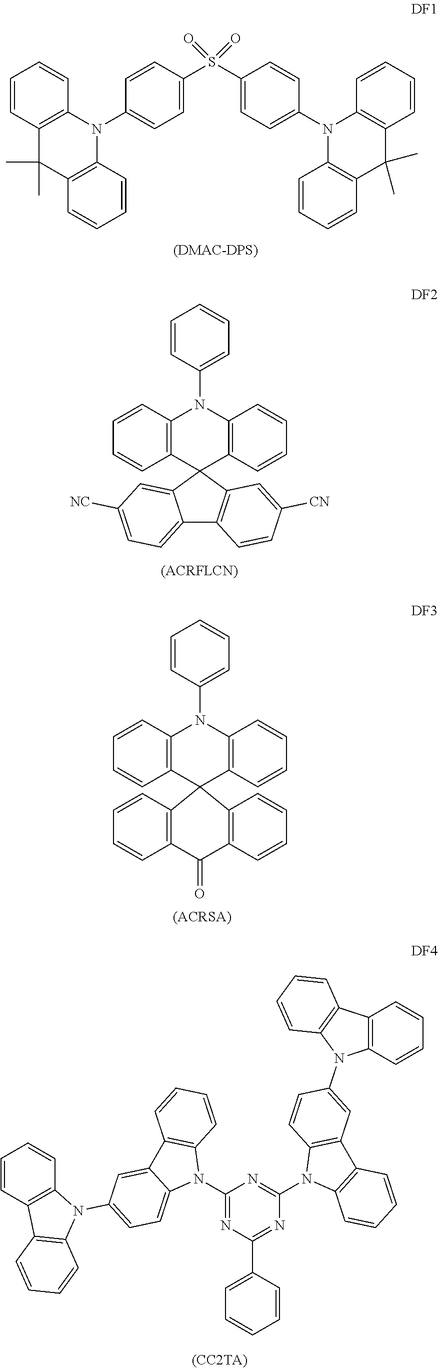

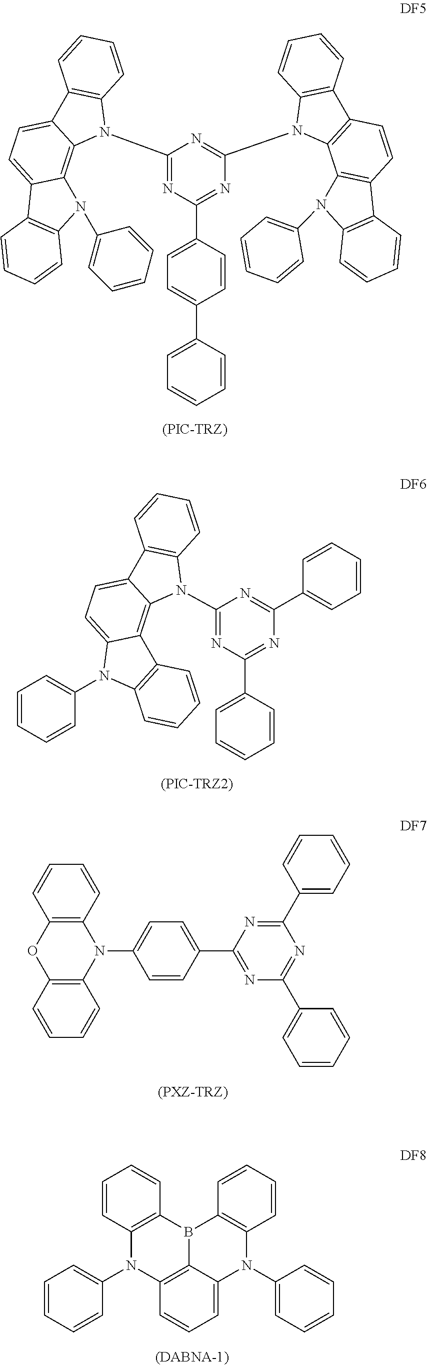

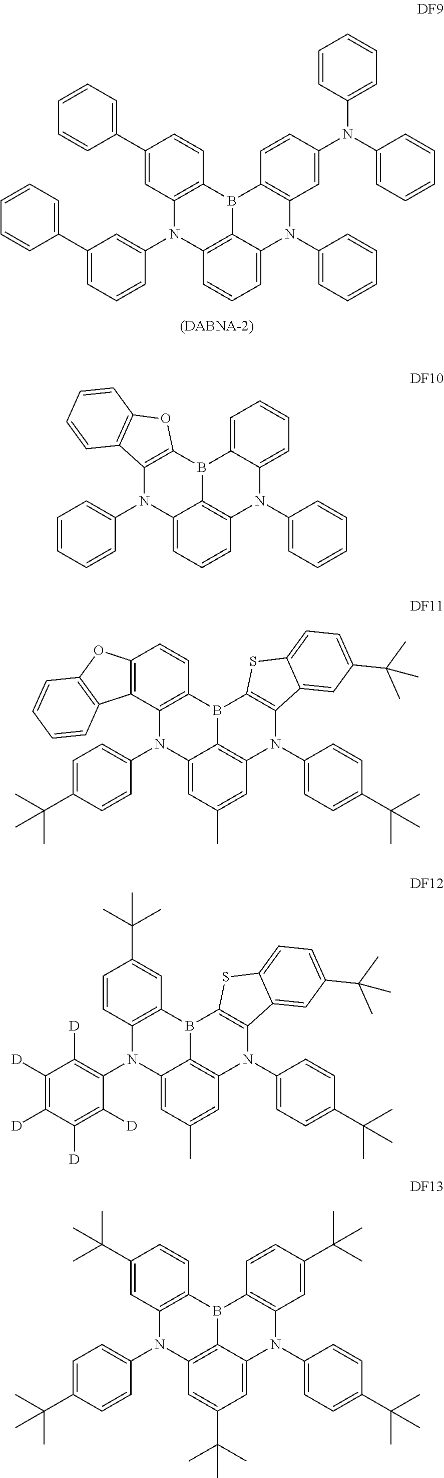

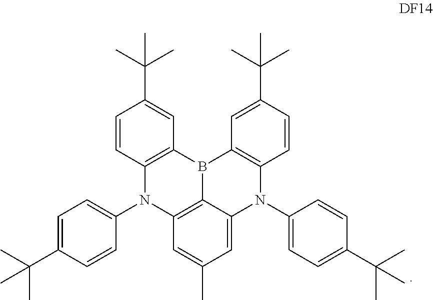 [SMILES: Cc1cc2c3c(c1)N(c1ccc(C(C)(C)C)cc1)c1ccc(C(C)(C)C)cc1B3c1cc(C(C)(C)C)ccc1N2c1ccc(C(C)(C)C)cc1]

### Quantum Dot

The emission layer 130 may include a quantum dot.

The term “quantum dot” as utilized herein refers to a crystal of a semiconductor compound, and may include any material capable of emitting light of one or more suitable emission wavelengths according to the size of the crystal.

A diameter of the quantum dot may be, for example, in a range of about 1 nanometer (nm) to about 10 nm. In the present disclosure, when quantum dots are spherical, “diameter” indicates a particle diameter or an average particle diameter, and when the dots are non-spherical, the “diameter” indicates a major axis length or an average major axis length.

The quantum dot may be synthesized by a wet chemical process, a metal organic chemical vapor deposition process, a molecular beam epitaxy process, or any process similar thereto.

The wet chemical process is a method including mixing a precursor material with an organic solvent and then growing quantum dot particle crystals. When the crystal grows, the organic solvent naturally acts as a dispersant coordinated on the surface of the quantum dot crystal and controls the growth of the crystal so that the growth of quantum dot particles can be controlled or selected through a process which costs lower, and is easier than vapor deposition methods, such as metal organic chemical vapor deposition (MOCVD) or molecular beam epitaxy (MBE).

The quantum dot may include: a Group II-VI semiconductor compound; a Group III-V semiconductor compound; a Group III-VI semiconductor compound; a Group I-III-VI semiconductor compound; a Group IV-VI semiconductor compound; a Group IV element or compound; or any combination thereof.

Examples of the Group II-VI semiconductor compound are: a binary compound, such as CdS, CdSe, CdTe, ZnS, ZnSe, ZnTe, ZnO, HgS, HgSe, HgTe, MgSe, MgS, and/or the like; a ternary compound, such as CdSeS, CdSeTe, CdSTe, ZnSeS, ZnSeTe, ZnSTe, HgSeS, HgSeTe, HgSTe, CdZnS, CdZnSe, CdZnTe, CdHgS, CdHgSe, CdHgTe, HgZnS, HgZnSe, HgZnTe, MgZnSe, MgZnS, and/or the like; a quaternary compound, such as CdZnSeS, CdZnSeTe, CdZnSTe, CdHgSeS, CdHgSeTe, CdHgSTe, HgZnSeS, HgZnSeTe, HgZnSTe, and/or the like; or any combination thereof.

Examples of the Group III-V semiconductor compound are: a binary compound, such as GaN, GaP, GaAs, GaSb, AlN, AlP, AlAs, AlSb, InN, InP, InAs, InSb, and/or the like; a ternary compound, such as GaNP, GaNAs, GaNSb, GaPAs, GaPSb, AlNP, AlNAs, AlNSb, AlPAs, AlPSb, InGaP, InNP, InAlP, InNAs, InNSb, InPAs, InPSb, and/or the like; a quaternary compound, such as GaAlNP, GaAlNAs, GaAlNSb, GaAlPAs, GaAlPSb, GaInNP, GaInNAs, GalnNSb, GaInPAs, GalnPSb, InAlNP, InAlNAs, InAlNSb, InAlPAs, InAlPSb, and/or the like; or any combination thereof. In one or more embodiments, the Group III-V semiconductor compound may further include a Group II element. Examples of the Group III-V semiconductor compound further including the Group II element are InZnP, InGaZnP, InAlZnP, and/or the like.

Examples of the Group III-VI semiconductor compound are: a binary compound, such as GaS, GaSe, Ga₂Se₃, GaTe, InS, InSe, In₂S₃, In₂Se₃, InTe, and/or the like; a ternary compound, such as InGaS₃, InGaSe₃, and/or the like; or any combination thereof.

Examples of the Group I-III-VI semiconductor compound are: a ternary compound, such as AgInS, AgInS₂, CuInS, CuInS₂, CuGaO₂, AgGaO₂, AgAlO₂, and/or the like; a quaternary compound, such as AgInGaS, AgInGaS₂, and/or the like; or any combination thereof.

Examples of the Group IV-VI semiconductor compound are: a binary compound, such as SnS, SnSe, SnTe, PbS, PbSe, PbTe, and/or the like; a ternary compound, such as SnSeS, SnSeTe, SnSTe, PbSeS, PbSeTe, PbSTe, SnPbS, SnPbSe, SnPbTe, and/or the like; a quaternary compound, such as SnPbSSe, SnPbSeTe, SnPbSTe, and/or the like; or any combination thereof.

Examples of the Group IV element or compound are: a single element, such as Si, Ge, and/or the like; a binary compound, such as SiC, SiGe, and/or the like; or any combination thereof.

Each element included in a multi-element compound, such as the binary compound, the ternary compound, and the quaternary compound, may be present at a substantially uniform concentration or non-substantially uniform concentration in a particle.

In one or more embodiments, the quantum dot may have a single structure in which the concentration of each element in the quantum dot is substantially uniform, or may have a core-shell dual structure. For example, a material included in the core and a material included in the shell may be different from each other.

The shell of the quantum dot may act as a protective layer which prevents chemical denaturation of the core to maintain semiconductor characteristics, and/or as a charging layer which imparts electrophoretic characteristics to the quantum dot. The shell may be a single layer or a multi-layer. The interface between the core and the shell may have a concentration gradient in which the concentration of an element existing in the shell decreases toward the center of the core.

Examples of the shell of the quantum dot are an oxide of metal, metalloid, or non-metal, a semiconductor compound, or a combination thereof. Examples of the oxide of metal, metalloid, or non-metal are: a binary compound, such as SiO₂, Al₂O₃, TiO₂, ZnO, MnO, Mn₂O₃, Mn₃O₄, CuO, FeO, Fe₂O₃, Fe₃O₄, CoO, Co₃O₄, NiO, and/or the like; a ternary compound, such as MgAl₂O₄, CoFe₂O₄, NiFe₂O₄, CoMn₂O₄, and/or the like; or any combination thereof. Examples of the semiconductor compound are: as described herein, a Group II-VI semiconductor compound; a Group III-V semiconductor compound; a Group III-VI semiconductor compound; a Group I-III-VI semiconductor compound; a Group IV-VI semiconductor compound; or any combination thereof. Examples of the semiconductor compound are CdS, CdSe, CdTe, ZnS, ZnSe, ZnTe, ZnSeS, ZnTeS, GaAs, GaP, GaSb, HgS, HgSe, HgTe, InAs, InP, InGaP, InSb, AlAs, AlP, AlSb, or any combination thereof.

The quantum dot may have a full width at half maximum (FWHM) of the emission wavelength spectrum of less than or equal to about 45 nm, less than or equal to about 40 nm, or for example, less than or equal to about 30 nm. When the FWHM of the quantum dot is within these ranges, the quantum dot may have enhanced or improved color purity or enhanced or improved color reproducibility. In some embodiments, because light emitted through the quantum dot is emitted in all directions, the wide viewing angle may be improved, (e.g., the viewing angle of the light-emitting device may be increased, enhanced, and/or improved).

In some embodiments, the quantum dot(s) may be in the form of spherical, pyramidal, multi-arm, or cubic nanoparticles, nanotubes, nanowires, nanofibers, and/or nanoplate particles.

Because the energy band gap may be adjusted by controlling the size of the quantum dot, light having one or more suitable wavelength bands may be obtained from the quantum dot emission layer. Accordingly, by utilizing quantum dots of different sizes, a light-emitting device that emits light of one or more suitable wavelengths may be implemented. In one or more embodiments, the size of the quantum dot may be selected to emit red light, green light, and/or blue light. In some embodiments, the size of the quantum dot may be configured to emit white light by combination of light of one or more suitable colors.

### Optical Activation Layer 135

The optoelectronic device 30 may include the optical activation layer 135 on the hole transport region 120. The optical activation layer 135 may be arranged between the hole transport region 120 and the electron transport region 140. In one or more embodiments, the optical activation layer 135 may be arranged between the hole transport layer included in the hole transport region 120 and a buffer layer included in the electron transport region 140. In one or more embodiments, the optical activation layer 135 may be arranged between the emission auxiliary layer included in the hole transport region 120 and the buffer layer included in the electron transport region 140.

The optical activation layer 135 may include the first compound and the second compound. The first compound may be referred to as an electron-donor compound or a p-type or kind compound, and the second compound may be referred to as an electron-acceptor compound or an n-type or kind compound.

The first compound and the second compound may be mixed and included in the optical activation layer 135. For example, the optical activation layer 135 may be a single layer including the first compound and the second compound.

The optical activation layer 135 may be to (e.g., be configured to) absorb incident light to generate excitons. The excitons may generate holes and electrons. The holes generated by the optical activation layer 135 may move to the first electrode 110 through the hole transport region 120. The electrons generated by the optical activation layer 135 may move to the second electrode 150 through the electron transport region 140.

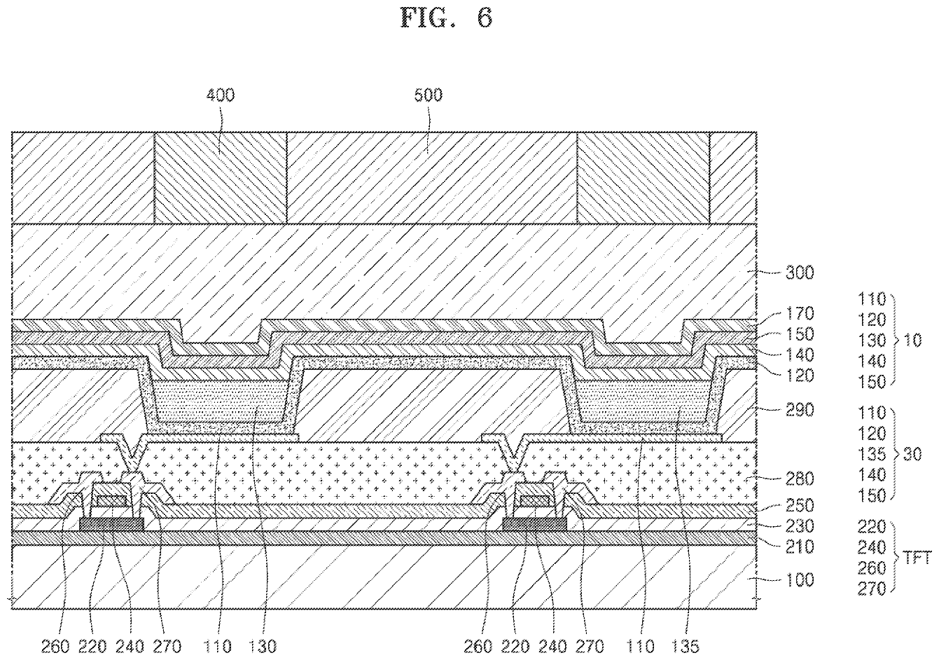

For example, the optical activation layer 135 may be to (e.g., be configured to) absorb light to generate an electrical signal. In one or more embodiments, the first compound included in the optical activation layer 135 may serve as a donor for supplying electrons, and the second compound included in the optical activation layer 135 may serve as an acceptor for receiving electrons. Accordingly, the optoelectronic device 30 including the optical activation layer 135 may serve as an optical sensor. For example, the optoelectronic device 30 may serve as a fingerprint recognition sensor, which is described with reference to FIG. 5.

### Electron Transport Region 140

The electron transport region 140 may have i) a single-layer structure including (e.g., consisting of) a single layer including (e.g., consisting of) a single material, ii) a single-layer structure including (e.g., consisting of) a single layer including multiple different materials, or iii) a multi-layer structure including multiple layers including multiple different materials.

The electron transport region 140 may include a buffer layer, a hole blocking layer, an electron control layer, an electron transport layer, an electron injection layer, or any combination thereof.

For example, the electron transport region 140 may have an electron transport layer/electron injection layer structure, a hole blocking layer/electron transport layer/electron injection layer structure, an electron control layer/electron transport layer/electron injection layer structure, or a buffer layer/electron transport layer/electron injection layer structure, wherein constituent layers of each structure are sequentially stacked from the emission layer 130.

The electron transport region 140 (for example, the buffer layer, the hole blocking layer, the electron control layer, or the electron transport layer in the electron transport region 140) may include a metal-free compound including at least one π electron-deficient nitrogen-containing C₁-C₆₀ cyclic group.

For example, the electron transport region 140 may include a compound represented by Formula 601:

[Ar₆₀₁]ₓₑ₁₁[(L₆₀₁)ₓₑ₁-R₆₀₁]ₓₑ₂₁. Formula 601

In Formula 601,
Ar₆₀₁ and L₆₀₁ may each independently be a C₃-C₆₀ carbocyclic group unsubstituted or substituted with at least one R₁₀ₐ or a C₁-C₆₀ heterocyclic group unsubstituted or substituted with at least one R₁₀ₐ,
xe11 may be 1, 2, or 3,
xe1 may be 0, 1, 2, 3, 4, or 5,
R₆₀₁ may be a C₃-C₆₀ carbocyclic group unsubstituted or substituted with at least one R₁₀ₐ, a C₁-C₆₀ heterocyclic group unsubstituted or substituted with at least one R₁₀ₐ, —Si(Q₆₀₁)(Q₆₀₂)(Q₆₀₃), —C(═O)(Q₆₀₁), —S(═O)₂(Q₆₀₁), or —P(═O)(Q₆₀₁)(Q₆₀₂),
Q₆₀₁ to Q₆₀₃ may each be defined as in connection with Q₁,
xe21 may be 1, 2, 3, 4, or 5, and
at least one selected from among Ar₆₀₁, L₆₀₁, and R₆₀₁ may each independently be a π electron-deficient nitrogen-containing C₁-C₆₀ cyclic group unsubstituted or substituted with at least one R₁₀ₐ.

In one or more embodiments, when xe11 in Formula 601 is 2 or more, two or more of Ar₆₀₁ may be linked to each other via a single bond.

In one or more embodiments, Ar₆₀₁ in Formula 601 may be an anthracene group unsubstituted or substituted with at least one R₁₀ₐ.

In one or more embodiments, the electron transport region 140 may include a compound represented by Formula 601-1:

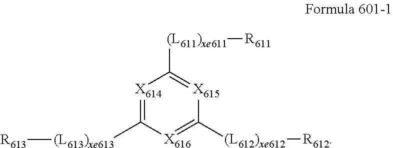

In Formula 601-1,
X₆₁₄ may be N or C(R₆₁₄), X₆₁₅ may be N or C(R₆₁₅), X₆₁₆ may be N or C(R₆₁₆), and at least one selected from among X₆₁₄ to X₆₁₆ may be N,
L611 to L₆₁₃ may each be defined as in connection with L₆₀₁,
xe611 to xe613 may each be defined as in connection with xe1,
R₆₁₁ to R₆₁₃ may each be defined as in connection with R₆₀₁, and
R₆₁₄ to R₆₁₆ may each independently be hydrogen, deuterium, —F, —Cl, —Br, —I, a hydroxyl group, a cyano group, a nitro group, a C₁-C₂₀ alkyl group, a C₁-C₂₀ alkoxy group, a C₃-C₆₀ carbocyclic group unsubstituted or substituted with at least one R₁₀ₐ, or a C₁-C₆₀ heterocyclic group unsubstituted or substituted with at least one R₁₀ₐ.

For example, xe1 and xe611 to xe613 in Formulae 601 and 601-1 may each independently be 0, 1, or 2.

The electron transport region 140 may include: at least one selected from among Compounds ET1 to ET45; 2,9-dimethyl-4,7-diphenyl-1,10-phenanthroline (BCP); 4,7-diphenyl-1,10-phenanthroline (Bphen); Alq₃; BAlq; TAZ; NTAZ; and/or any combination thereof:

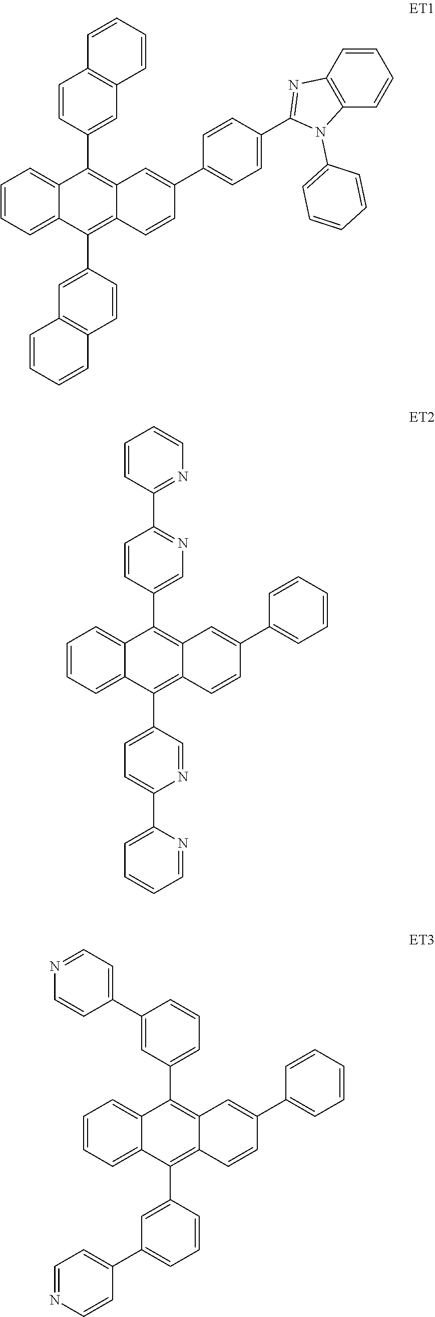 [SMILES: c1ccc(-n2c(-c3ccc(-c4ccc5c(-c6ccc7ccccc7c6)c6ccccc6c(-c6ccc7ccccc7c6)c5c4)cc3)nc3ccccc32)cc1 ; c1ccc(-c2ccc3c(-c4ccc(-c5ccccn5)nc4)c4ccccc4c(-c4ccc(-c5ccccn5)nc4)c3c2)cc1 ; c1ccc(-c2ccc3c(-c4cccc(-c5ccncc5)c4)c4ccccc4c(-c4cccc(-c5ccncc5)c4)c3c2)cc1]

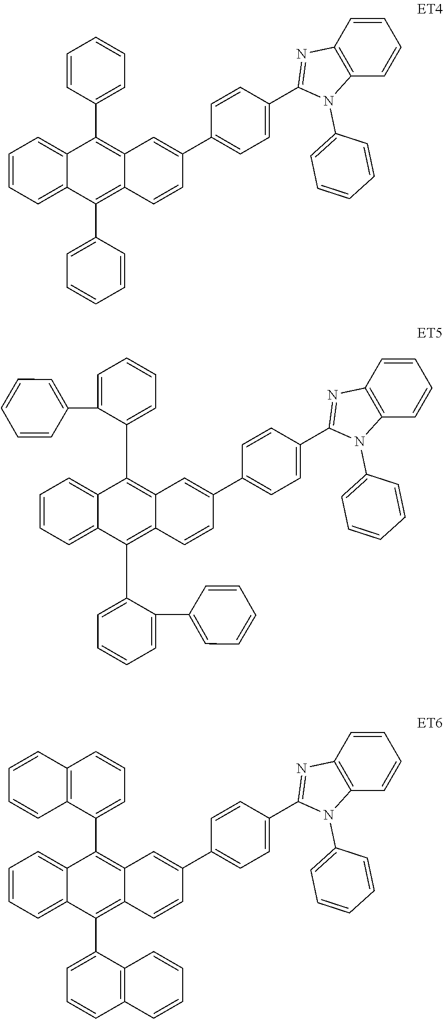 [SMILES: c1ccc(-c2c3ccccc3c(-c3ccccc3)c3cc(-c4ccc(-c5nc6ccccc6n5-c5ccccc5)cc4)ccc23)cc1 ; c1ccc(-c2ccccc2-c2c3ccccc3c(-c3ccccc3-c3ccccc3)c3cc(-c4ccc(-c5nc6ccccc6n5-c5ccccc5)cc4)ccc23)cc1 ; c1ccc(-n2c(-c3ccc(-c4ccc5c(-c6cccc7ccccc67)c6ccccc6c(-c6cccc7ccccc67)c5c4)cc3)nc3ccccc32)cc1]

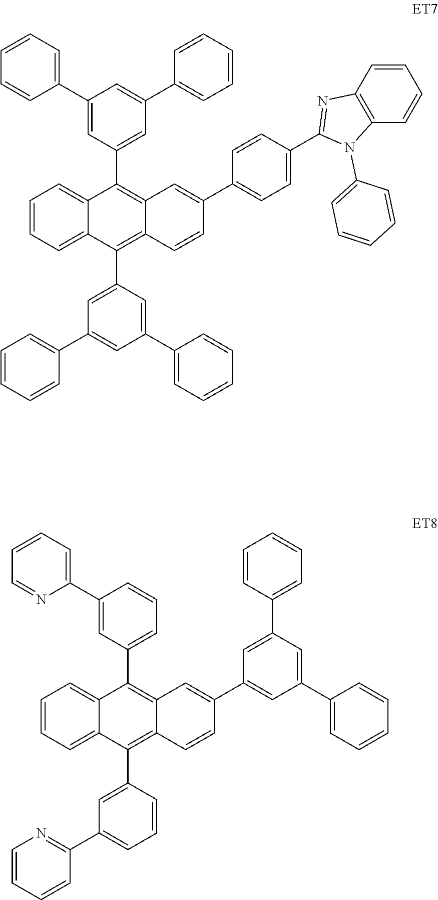 [SMILES: c1ccc(-c2cc(-c3ccccc3)cc(-c3c4ccccc4c(-c4cc(-c5ccccc5)cc(-c5ccccc5)c4)c4cc(-c5ccc(-c6nc7ccccc7n6-c6ccccc6)cc5)ccc34)c2)cc1 ; c1ccc(-c2cc(-c3ccccc3)cc(-c3ccc4c(-c5cccc(-c6ccccn6)c5)c5ccccc5c(-c5cccc(-c6ccccn6)c5)c4c3)c2)cc1]

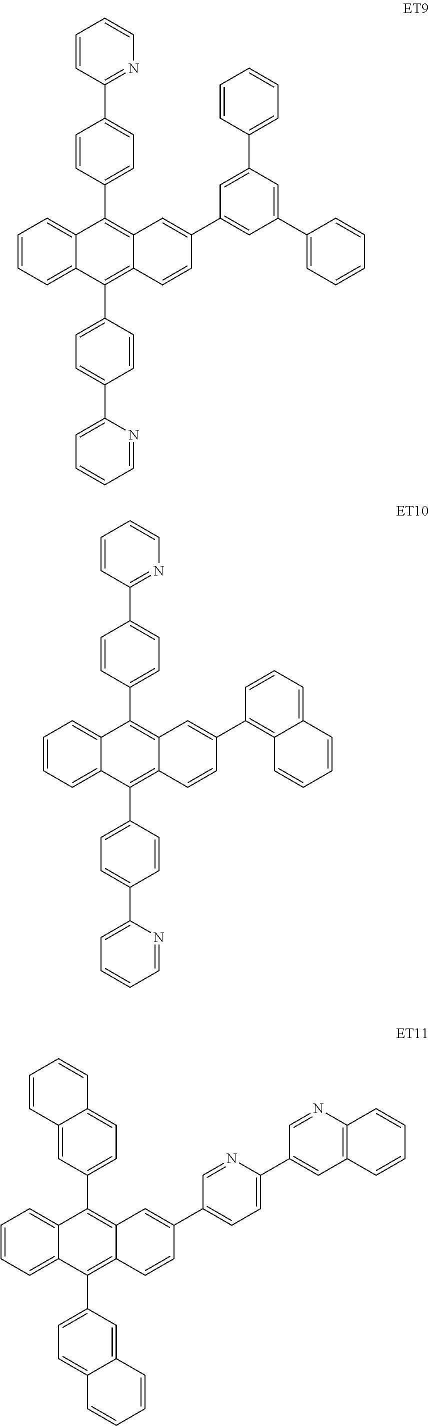

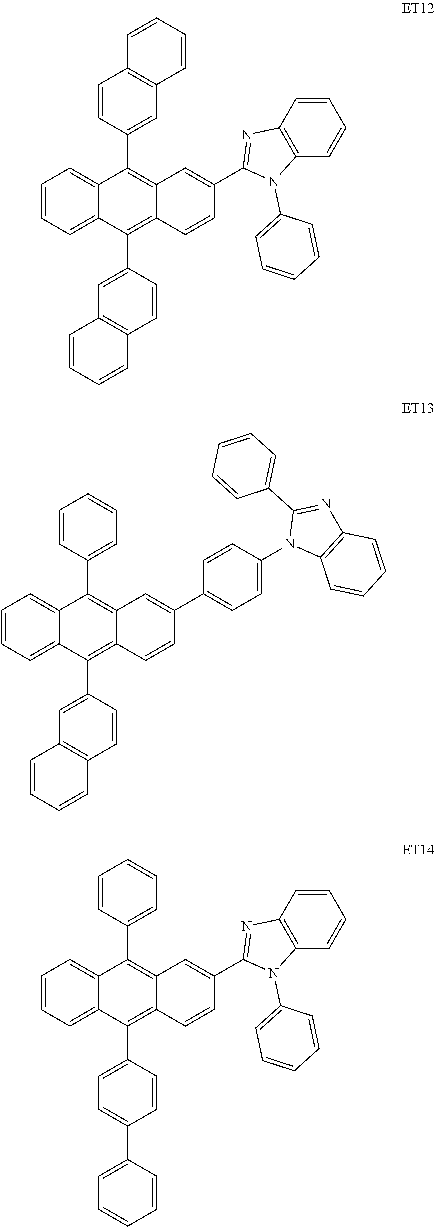 [SMILES: c1ccc(-n2c(-c3ccc4c(-c5ccc6ccccc6c5)c5ccccc5c(-c5ccc6ccccc6c5)c4c3)nc3ccccc32)cc1 ; c1ccc(-c2c3ccccc3c(-c3ccc4ccccc4c3)c3ccc(-c4ccc(-n5c(-c6ccccc6)nc6ccccc65)cc4)cc23)cc1 ; c1ccc(-c2ccc(-c3c4ccccc4c(-c4ccccc4)c4cc(-c5nc6ccccc6n5-c5ccccc5)ccc34)cc2)cc1]

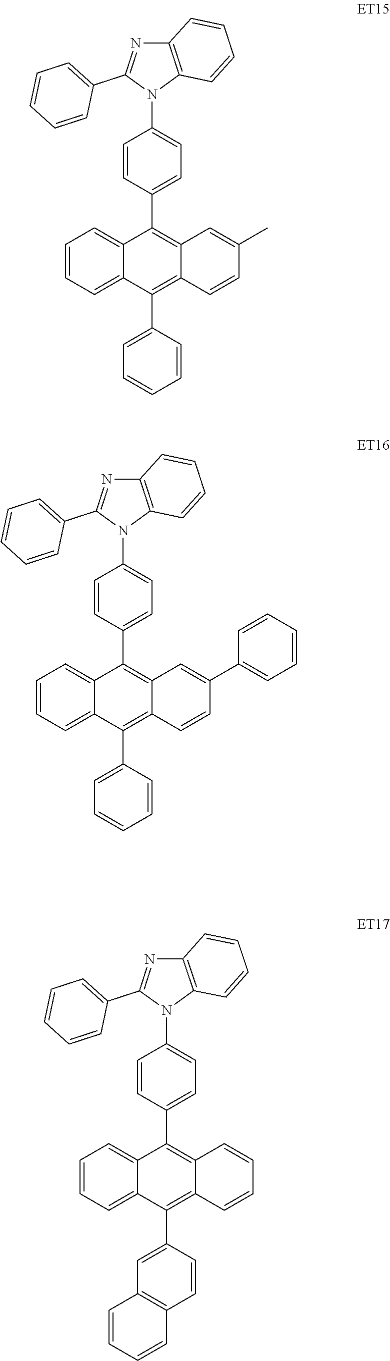 [SMILES: Cc1ccc2c(-c3ccccc3)c3ccccc3c(-c3ccc(-n4c(-c5ccccc5)nc5ccccc54)cc3)c2c1 ; c1ccc(-c2ccc3c(-c4ccccc4)c4ccccc4c(-c4ccc(-n5c(-c6ccccc6)nc6ccccc65)cc4)c3c2)cc1 ; c1ccc(-c2nc3ccccc3n2-c2ccc(-c3c4ccccc4c(-c4ccc5ccccc5c4)c4ccccc34)cc2)cc1]

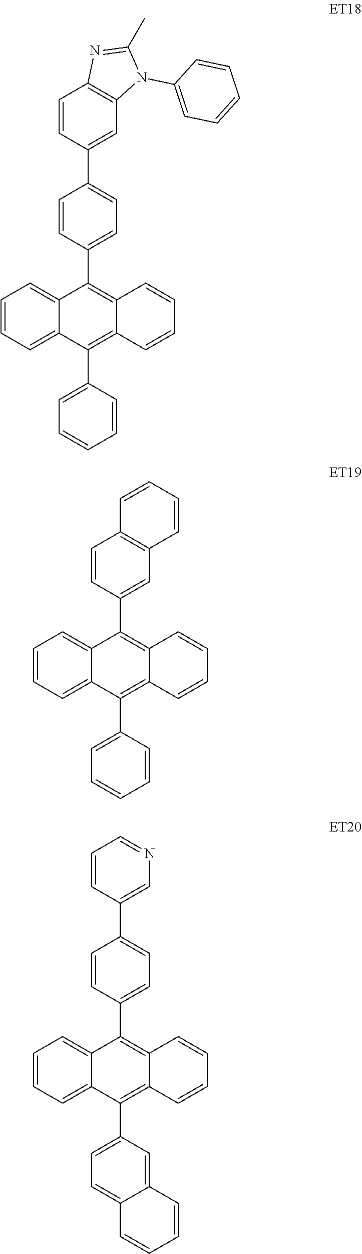

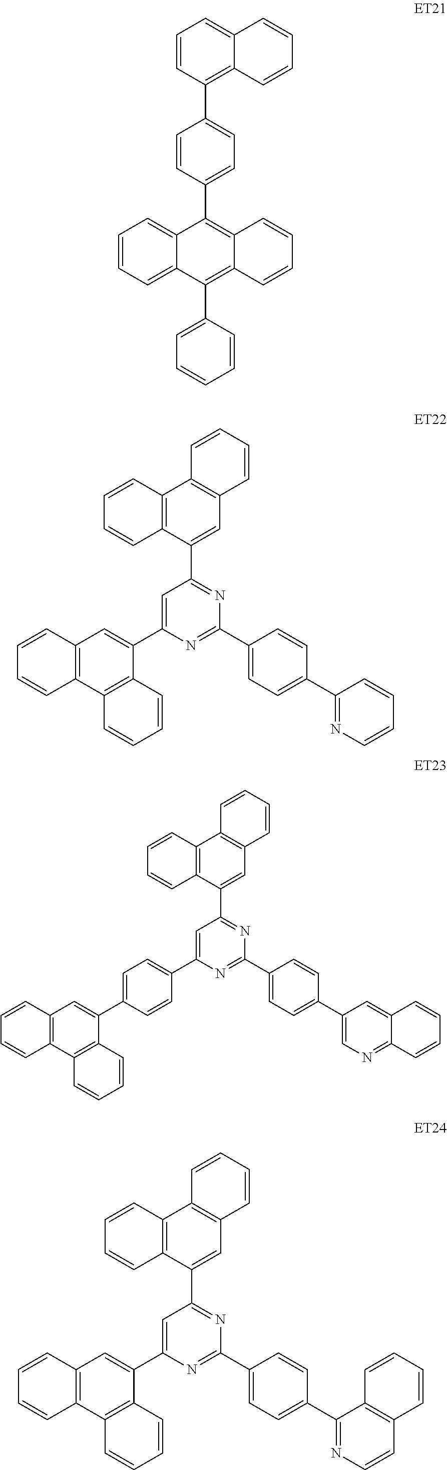

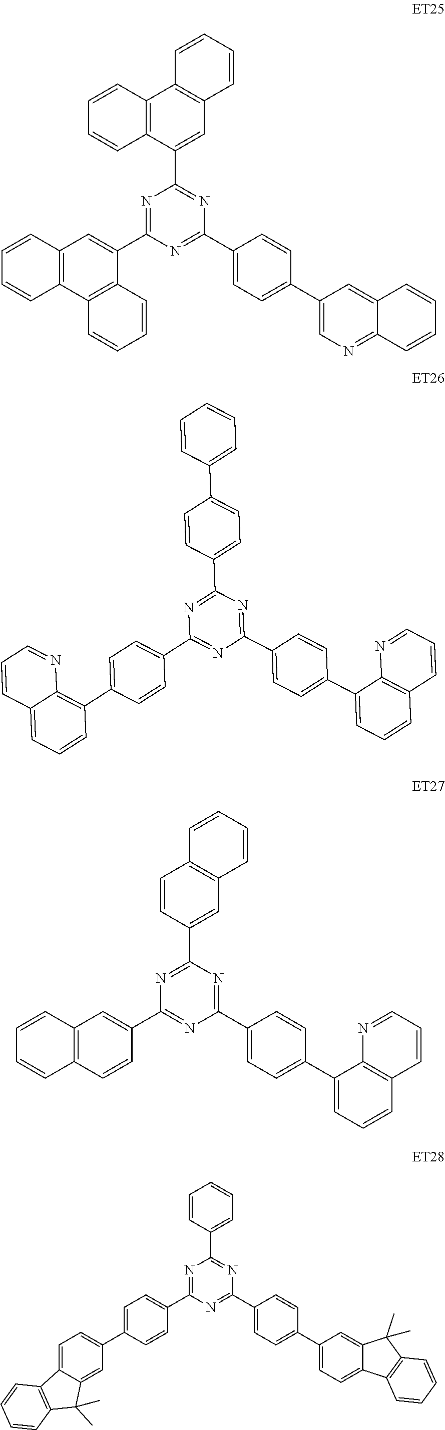 [SMILES: c1ccc2ncc(-c3ccc(-c4nc(-c5cc6ccccc6c6ccccc56)nc(-c5cc6ccccc6c6ccccc56)n4)cc3)cc2c1 ; c1ccc(-c2ccc(-c3nc(-c4ccc(-c5cccc6cccnc56)cc4)nc(-c4ccc(-c5cccc6cccnc56)cc4)n3)cc2)cc1 ; c1ccc2cc(-c3nc(-c4ccc(-c5cccc6cccnc56)cc4)nc(-c4ccc5ccccc5c4)n3)ccc2c1 ; CC1(C)c2ccccc2-c2ccc(-c3ccc(-c4nc(-c5ccccc5)nc(-c5ccc(-c6ccc7c(c6)C(C)(C)c6ccccc6-7)cc5)n4)cc3)cc21]

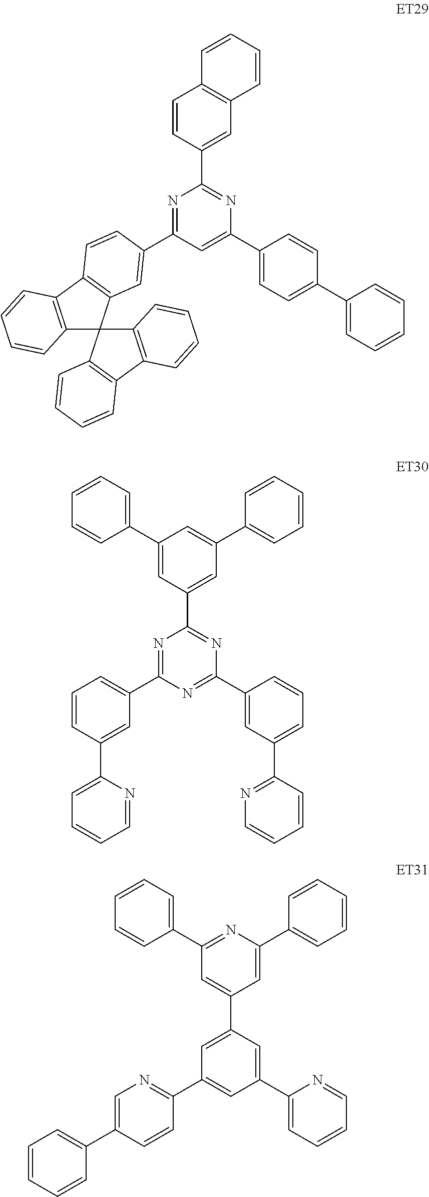 [SMILES: c1ccc(-c2ccc(-c3cc(-c4ccc5c(c4)C4(c6ccccc6-c6ccccc64)c4ccccc4-5)nc(-c4ccc5ccccc5c4)n3)cc2)cc1 ; c1ccc(-c2cc(-c3ccccc3)cc(-c3nc(-c4cccc(-c5ccccn5)c4)nc(-c4cccc(-c5ccccn5)c4)n3)c2)cc1 ; c1ccc(-c2ccc(-c3cc(-c4cc(-c5ccccc5)nc(-c5ccccc5)c4)cc(-c4ccccn4)c3)nc2)cc1]

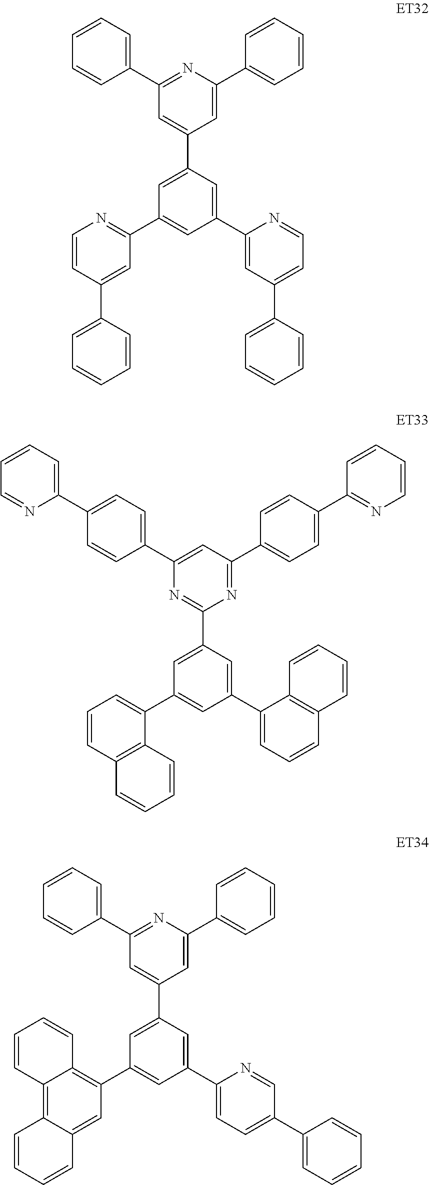 [SMILES: c1ccc(-c2ccnc(-c3cc(-c4cc(-c5ccccc5)nc(-c5ccccc5)c4)cc(-c4cc(-c5ccccc5)ccn4)c3)c2)cc1 ; c1ccc(-c2ccc(-c3cc(-c4ccc(-c5ccccn5)cc4)nc(-c4cc(-c5cccc6ccccc56)cc(-c5cccc6ccccc56)c4)n3)cc2)nc1 ; c1ccc(-c2ccc(-c3cc(-c4cc(-c5ccccc5)nc(-c5ccccc5)c4)cc(-c4cc5ccccc5c5ccccc45)c3)nc2)cc1]

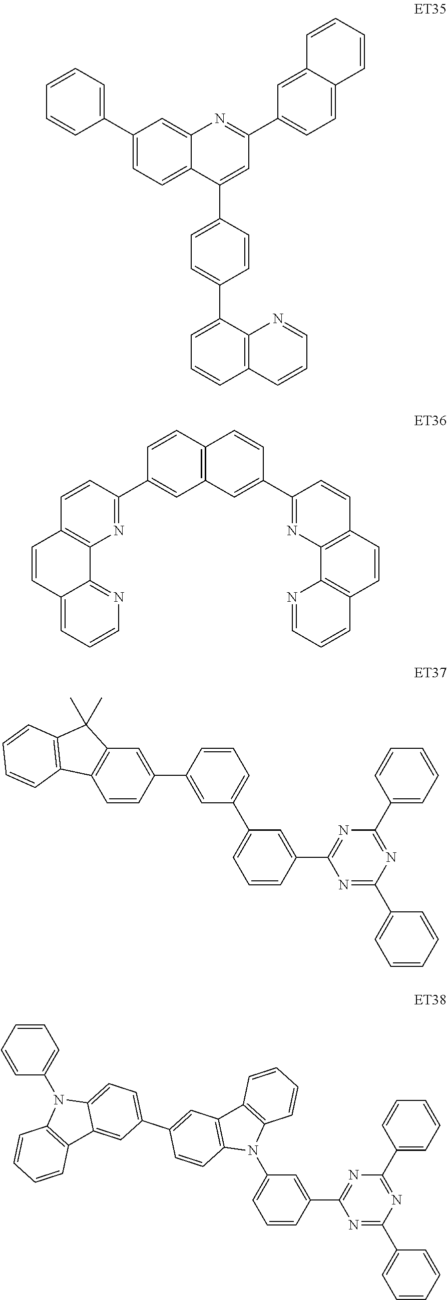 [SMILES: c1ccc(-c2ccc3c(-c4ccc(-c5cccc6cccnc56)cc4)cc(-c4ccc5ccccc5c4)nc3c2)cc1 ; c1cnc2c(c1)ccc1ccc(-c3ccc4ccc(-c5ccc6ccc7cccnc7c6n5)cc4c3)nc12 ; CC1(C)c2ccccc2-c2ccc(-c3cccc(-c4cccc(-c5nc(-c6ccccc6)nc(-c6ccccc6)n5)c4)c3)cc21 ; c1ccc(-c2nc(-c3ccccc3)nc(-c3cccc(-n4c5ccccc5c5cc(-c6ccc7c(c6)c6ccccc6n7-c6ccccc6)ccc54)c3)n2)cc1]

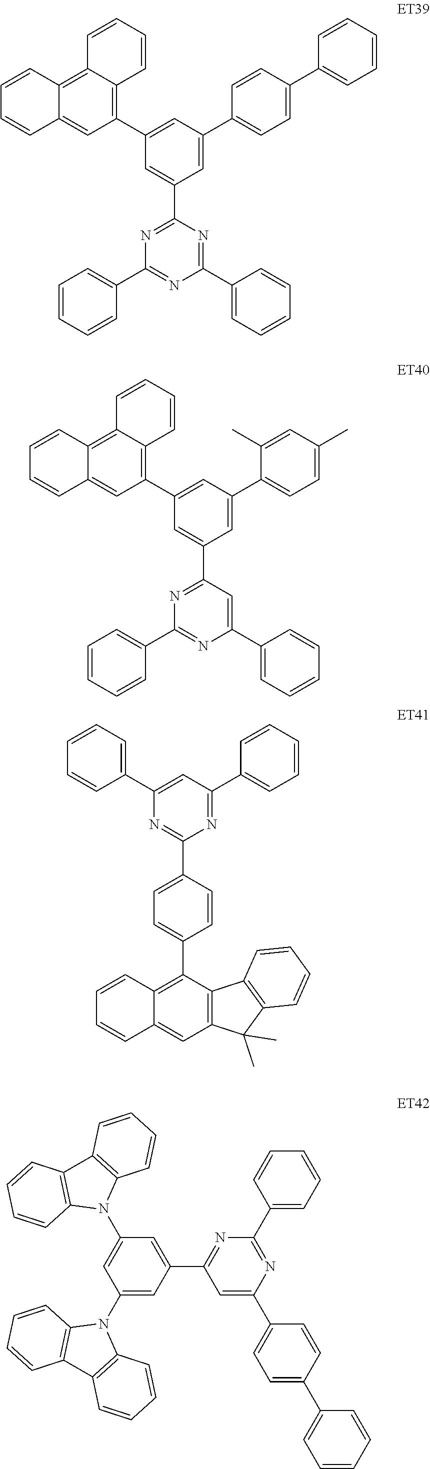

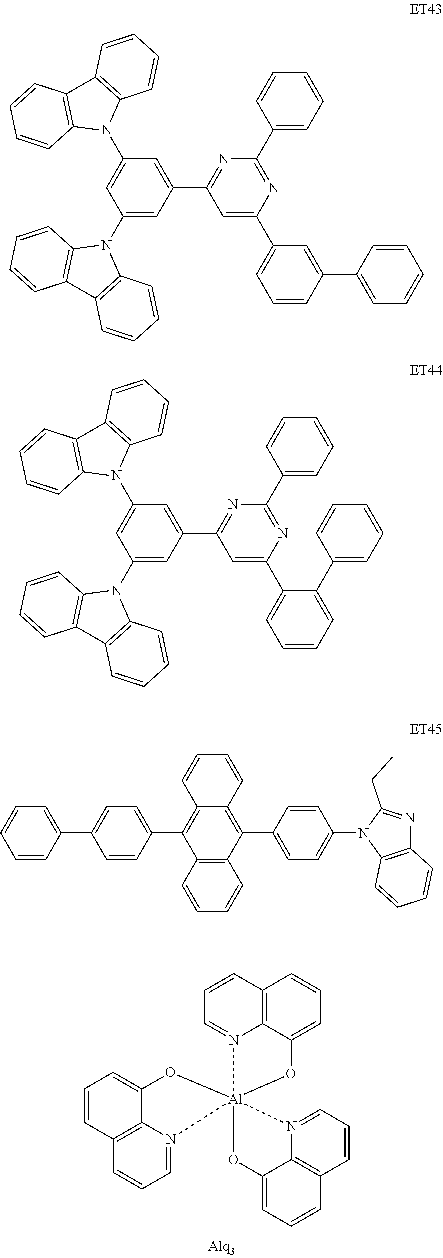

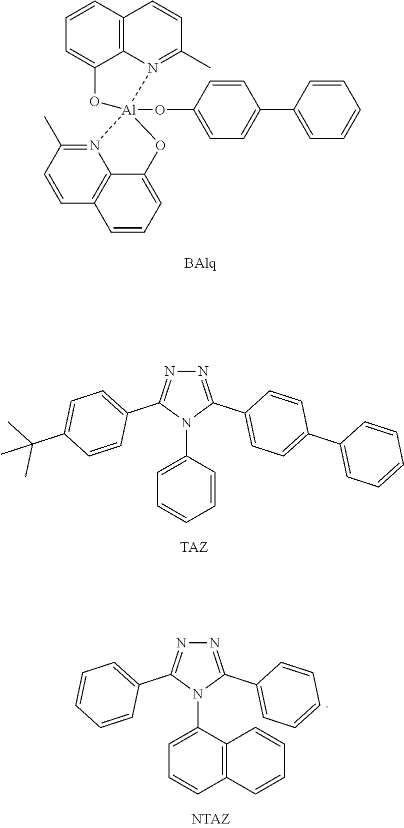

A thickness of the electron transport region 140 may be in a range of about 100 Å to about 5,000 Å, for example, about 160 Å to about 4,000 Å. When the electron transport region 140 includes a buffer layer, a hole blocking layer, an electron control layer, an electron transport layer, or any combination thereof, a thickness of the buffer layer, the hole blocking layer, or the electron control layer may be in a range of about 20 Å to about 1,000 Å, for example, about 30 Å to about 300 Å, and a thickness of the electron transport layer may be in a range of about 100 Å to about 1,000 Å, for example, about 150 Å to about 500 Å. When the thicknesses of the buffer layer, the hole blocking layer, the electron control layer, the electron transport layer, and/or the electron transport region 140 are within the ranges described above, satisfactory electron transporting characteristics may be obtained without a substantial increase in driving voltage.

The electron transport region 140 (for example, the electron transport layer in the electron transport region 140) may further include, in addition to the materials described above, a metal-containing material.

The metal-containing material may include an alkali metal complex, an alkaline earth metal complex, or any combination thereof. The metal ion of an alkali metal complex may be a Li ion, a Na ion, a K ion, a Rb ion, or a Cs ion, and the metal ion of an alkaline earth metal complex may be a Be ion, a Mg ion, a Ca ion, a Sr ion, or a Ba ion. A ligand coordinated with the metal ion of the alkali metal complex or the alkaline earth-metal complex may include a hydroxyquinoline, a hydroxyisoquinoline, a hydroxybenzoquinoline, a hydroxyacridine, a hydroxyphenanthridine, a hydroxyphenyloxazole, a hydroxyphenylthiazole, a hydroxyphenyloxadiazole, a hydroxyphenylthiadiazole, a hydroxyphenylpyridine, a hydroxyphenylbenzimidazole, a hydroxyphenylbenzothiazole, a bipyridine, a phenanthroline, a cyclopentadiene, or any combination thereof.

For example, the metal-containing material may include a Li complex. The Li complex may include, for example, Compound ET-D1 (LiQ) or ET-D2:

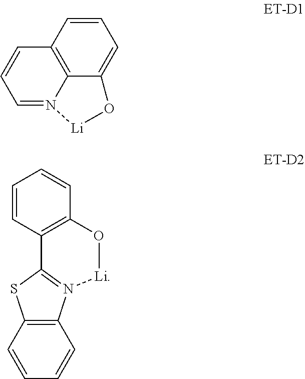

The electron transport region 140 may include an electron injection layer that facilitates the injection of electrons from the second electrode 150. The electron injection layer may directly contact the second electrode 150, but embodiments are not limited thereto.

The electron injection layer may have i) a single-layer structure including (e.g., consisting of) a single layer including (e.g., consisting of) a single material, ii) a single-layer structure including (e.g., consisting of) a single layer including multiple different materials, or iii) a multi-layer structure including multiple layers including multiple different materials.

The electron injection layer may include an alkali metal, an alkaline earth metal, a rare earth metal, an alkali metal-containing compound, an alkaline earth metal-containing compound, a rare earth metal-containing compound, an alkali metal complex, an alkaline earth metal complex, a rare earth metal complex, or any combination thereof.

The alkali metal may include Li, Na, K, Rb, Cs, or any combination thereof. The alkaline earth metal may include Mg, Ca, Sr, Ba, or any combination thereof. The rare earth metal may include Sc, Y, Ce, Tb, Yb, Gd, or any combination thereof.

The alkali metal-containing compound, the alkaline earth metal-containing compound, and the rare earth metal-containing compound may be oxides, halides (for example, fluorides, chlorides, bromides, iodides, etc.), or tellurides of the alkali metal, the alkaline earth metal, and the rare earth metal, or any combination thereof.

The alkali metal-containing compound may include: an alkali metal oxide, such as Li₂O, Cs₂O, K₂O, and/or the like; alkali metal halides, such as LiF, NaF, CsF, KF, LiI, NaI, CsI, KI, and/or the like; or any combination thereof. The alkaline earth metal-containing compound may include an alkaline earth metal compound, such as BaO, SrO, CaO, BaₓSr₁₋ₓO (wherein x is a real number satisfying 0<x<1), BaₓCa₁₋ₓO (wherein x is a real number satisfying 0<x<1), and/or the like. The rare earth metal-containing compound may include YbF₃, ScF₃, Sc₂O₃, Y₂O₃, Ce₂O₃, GdF₃, TbF₃, YbI₃, ScI₃, TbI₃, or any combination thereof. In one or more embodiments, the rare earth metal-containing compound may include lanthanide metal telluride. Examples of the lanthanide metal telluride are LaTe, CeTe, PrTe, NdTe, PmTe, SmTe, EuTe, GdTe, TbTe, DyTe, HoTe, ErTe, TmTe, YbTe, LuTe, La₂Te₃, Ce₂Te₃, Pr₂Te₃, Nd₂Te₃, Pm₂Te₃, Sm₂Te₃, Eu₂Te₃, Gd₂Te₃, Tb₂Te₃, Dy₂Te₃, Ho₂Te₃, Er₂Te₃, Tm₂Te₃, Yb₂Te₃, Lu₂Te₃, and/or the like.

The alkali metal complex, the alkaline earth-metal complex, and the rare earth metal complex may include i) at least one selected from among ions of the alkali metal, the alkaline earth metal, and the rare earth metal and ii) a ligand bonded to the metal ion, for example, hydroxyquinoline, hydroxyisoquinoline, hydroxybenzoquinoline, hydroxyacridine, hydroxyphenanthridine, hydroxyphenyloxazole, hydroxyphenylthiazole, hydroxyphenyloxadiazole, hydroxyphenylthiadiazole, hydroxyphenylpyridine, hydroxyphenyl benzimidazole, hydroxyphenylbenzothiazole, bipyridine, phenanthroline, cyclopentadiene, or any combination thereof.

In one or more embodiments, the electron injection layer may include (e.g., consist of) an alkali metal, an alkaline earth metal, a rare earth metal, an alkali metal-containing compound, an alkaline earth metal-containing compound, a rare earth metal-containing compound, an alkali metal complex, an alkaline earth metal complex, a rare earth metal complex, or any combination thereof, as described above. In one or more embodiments, the electron injection layer may further include an organic material (for example, a compound represented by Formula 601).

In one or more embodiments, the electron injection layer may include (e.g., consist of) i) an alkali metal-containing compound (for example, alkali metal halide), ii) a) an alkali metal-containing compound (for example, alkali metal halide); and b) an alkali metal, an alkaline earth metal, a rare earth metal, or any combination thereof. For example, the electron injection layer may be a KI:Yb co-deposited layer, an RbI:Yb co-deposited layer, a LiF:Yb co-deposited layer, and/or the like.

When the electron injection layer further includes an organic material, an alkali metal, an alkaline earth metal, a rare earth metal, an alkali metal-containing compound, an alkaline earth metal-containing compound, a rare earth metal-containing compound, an alkali metal complex, an alkaline earth-metal complex, a rare earth metal complex, or any combination thereof may be uniformly or non-uniformly dispersed in a matrix including the organic material.

A thickness of the electron injection layer may be in a range of about 1 Å to about 100 Å, and, for example, about 3 Å to about 90 Å. When the thickness of the electron injection layer is within the ranges described above, satisfactory electron injection characteristics may be obtained without a substantial increase in driving voltage.

### Second Electrode 150

The second electrode 150 may be arranged on the electron transport region 140. The second electrode 150 may be a cathode, which is an electron injection electrode, and as a material for forming the second electrode 150, a metal, an alloy, an electrically conductive compound, or any combination thereof, each having a low work-function, may be utilized. The term “low work-function material” as utilized herein refers to a substance (e.g., a metal or metal alloy) that requires a relatively small, or low, amount of energy to emit electrons from its surface.

The second electrode 150 may include lithium (Li), silver (Ag), magnesium (Mg), aluminum (Al), aluminum-lithium (Al—Li), calcium (Ca), magnesium-indium (Mg—In), magnesium-silver (Mg—Ag), ytterbium (Yb), silver-ytterbium (Ag—Yb), ITO, IZO, or any combination thereof. The second electrode 150 may be a transmissive electrode, a semi-transmissive electrode, or a reflective electrode.

The second electrode 150 may have a single-layer structure or a multi-layer structure including multiple layers.

### Capping Layer

A first capping layer may be arranged outside (e.g., on) the first electrode 110, and/or a second capping layer may be arranged outside (e.g., on) the second electrode 150. For example, the light-emitting device 10 may have a structure in which the first capping layer, the first electrode 110, the emission layer 130, and the second electrode 150 are sequentially stacked in the stated order, a structure in which the first electrode 110, the emission layer 130, the second electrode 150, and the second capping layer are sequentially stacked in the stated order, or a structure in which the first capping layer, the first electrode 110, the emission layer 130, the second electrode 150, and the second capping layer are sequentially stacked in the stated order.

Light generated in the emission layer 130 of the light-emitting device 10 may be extracted toward the outside through the first electrode 110 which is a semi-transmissive electrode or a transmissive electrode, and the first capping layer. Light generated in the emission layer 130 of the light-emitting device 10 may be extracted toward the outside through the second electrode 150 which is a semi-transmissive electrode or a transmissive electrode, and the second capping layer.

The first capping layer and the second capping layer may increase external emission efficiency according to the principle of constructive interference. Accordingly, the light extraction efficiency of the light-emitting device 10 may be increased, so that the luminescence efficiency of the light-emitting device 10 may be enhanced or improved.

Each of the first capping layer and the second capping layer may include a material having a refractive index of greater or equal to about 1.6 (at about 520 nm to about 630 nm).

The first capping layer and the second capping layer may each independently be an organic capping layer including an organic material, an inorganic capping layer including an inorganic material, or an organic-inorganic composite capping layer including an organic material and an inorganic material.

At least one selected from among the first capping layer or the second capping layer may each independently include a carbocyclic compound, a heterocyclic compound, an amine group-containing compound, a porphine derivative, a phthalocyanine derivative, a naphthalocyanine derivative, an alkali metal complex, an alkaline earth metal complex, or any combination thereof. The carbocyclic compound, the heterocyclic compound, and the amine group-containing compound may optionally be substituted with a substituent including O, N, S, Se, Si, F, Cl, Br, I, or any combination thereof. In one or more embodiments, at least one selected from among the first capping layer or the second capping layer may each independently include an amine group-containing compound.

In one or more embodiments, at least one selected from among the first capping layer and/or the second capping layer may each independently include a compound represented by Formula 201, a compound represented by Formula 202, or any combination thereof.

In one or more embodiments, at least one selected from among the first capping layer and/or the second capping layer may each independently include: one selected from among Compounds HT28 to HT33; Compounds CP1 to CP6; β-NPB; and/or any combination thereof:

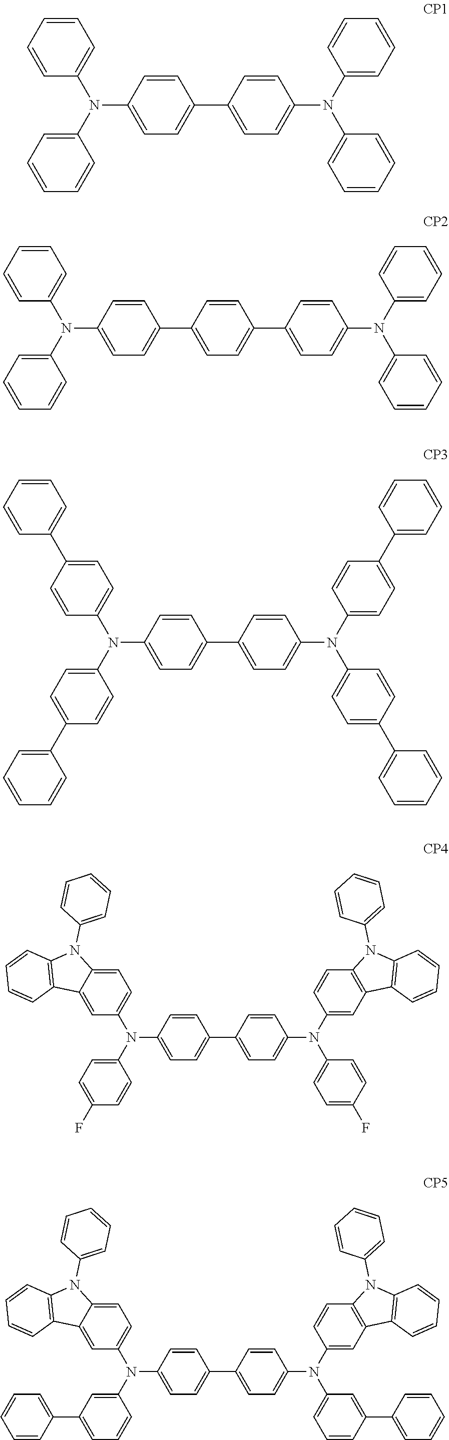 [SMILES: c1ccc(N(c2ccccc2)c2ccc(-c3ccc(N(c4ccccc4)c4ccccc4)cc3)cc2)cc1 ; c1ccc(N(c2ccccc2)c2ccc(-c3ccc(-c4ccc(N(c5ccccc5)c5ccccc5)cc4)cc3)cc2)cc1 ; c1ccc(-c2ccc(N(c3ccc(-c4ccccc4)cc3)c3ccc(-c4ccc(N(c5ccc(-c6ccccc6)cc5)c5ccc(-c6ccccc6)cc5)cc4)cc3)cc2)cc1 ; Fc1ccc(N(c2ccc(-c3ccc(N(c4ccc(F)cc4)c4ccc5c(c4)c4ccccc4n5-c4ccccc4)cc3)cc2)c2ccc3c(c2)c2ccccc2n3-c2ccccc2)cc1 ; c1ccc(-c2cccc(N(c3ccc(-c4ccc(N(c5cccc(-c6ccccc6)c5)c5ccc6c(c5)c5ccccc5n6-c5ccccc5)cc4)cc3)c3ccc4c(c3)c3ccccc3n4-c3ccccc3)c2)cc1]

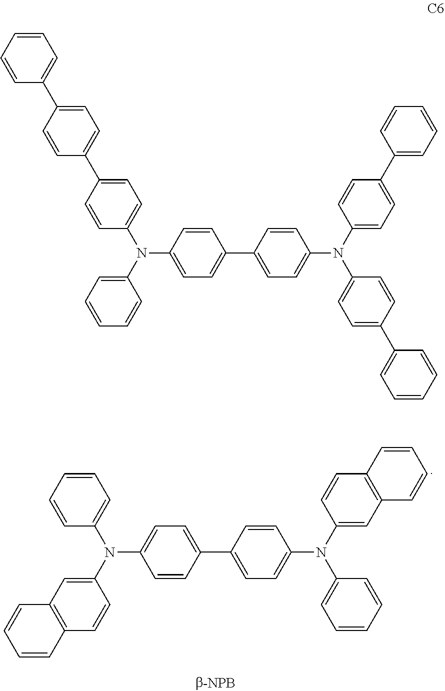 [SMILES: c1ccc(-c2ccc(-c3ccc(N(c4ccccc4)c4ccc(-c5ccc(N(c6ccc(-c7ccccc7)cc6)c6ccc(-c7ccccc7)cc6)cc5)cc4)cc3)cc2)cc1 ; c1ccc(N(c2ccc(-c3ccc(N(c4ccccc4)c4ccc5ccccc5c4)cc3)cc2)c2ccc3ccccc3c2)cc1]

### Film

In one or more embodiments, the electronic apparatus may include a film. The film may be, for example, an optical member (or a light control refers to) (for example, a color filter, a color conversion member, a capping layer, a light extraction efficiency enhancement layer, a selective light absorbing layer, a polarizing layer, a quantum dot-containing layer, etc.), a light blocking member (for example, a light reflective layer, a light absorbing layer, etc.), a protective member (for example, an insulating layer, a dielectric layer, etc.), and/or the like.

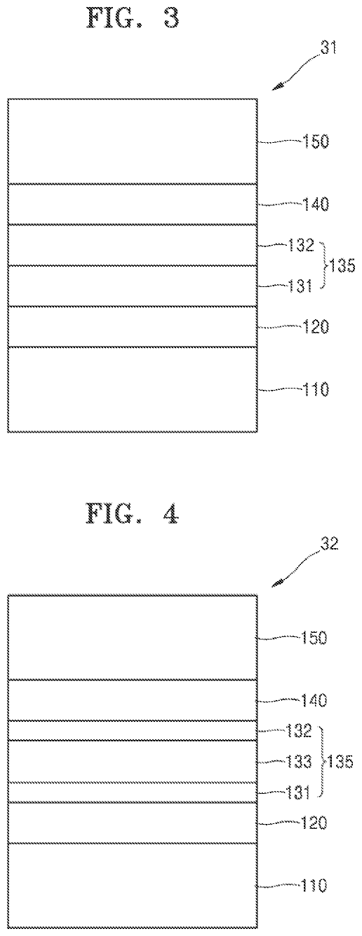

### Description of FIG. 3

FIG. 3 is a schematic view of an optoelectronic device 31 according to another embodiment.

The optoelectronic device 31 shown in FIG. 3 may be the same as and/or similar to the optoelectronic device 30 shown in FIG. 1, except for the optical activation layer 135, and thus, descriptions of other components will not be provided.

The optoelectronic device 31 may include the optical activation layer 135 arranged between the hole transport region 120 and the electron transport region 140. In one or more embodiments, the optical activation layer 135 may be arranged between the hole transport layer included in the hole transport region 120 and the buffer layer included in the electron transport region 140. In one or more embodiments, the optical activation layer 135 may be arranged between the emission auxiliary layer included in the hole transport region 120 and the buffer layer included in the electron transport region 140.

The optical activation layer 135 may include a first layer 131 adjacent to the hole transport region 120 and a second layer 132 adjacent to the electron transport region 140. For example, the first layer 131 may contact (e.g., directly contact) the second layer 132.

In one or more embodiments, the first layer 131 may contact (e.g., directly contact) the hole transport layer included in the hole transport region 120. In one or more embodiments, the first layer 131 may contact (e.g., directly contact) the emission auxiliary layer arranged on the hole transport layer.

In one or more embodiments, the second layer 132 may contact (e.g., directly contact) the buffer layer included in the electron transport region 140.

The first layer 131 may include the first compound. The first layer 131 may include (e.g., consist of) the first compound. For example, the first layer 131 may not include (e.g., may exclude) the second compound (e.g., be substantially free or free of the second compound). The first layer 131 may be referred to as a p-type or kind optical activation layer or a donor layer.

The second layer 132 may include the second compound. The second layer 132 may include (e.g., consist of) the second compound. For example, the second layer 132 may not include (e.g., may exclude) the first compound (e.g., be substantially free or free of the first compound). The second layer 132 may be referred to as an n-type or kind optical activation layer or an acceptor layer.

For example, the optical activation layer 135 may have a two-layer structure divided into the first layer 131 including the first compound and the second layer 132 including the second compound.

The optical activation layer 135 may be to (e.g., be configured to) absorb incident light to generate excitons. The excitons may generate holes and electrons. The holes generated by the optical activation layer 135 may move to the first electrode 110 through the hole transport region 120. The electrons generated by the optical activation layer 135 may move to the second electrode 150 through the electron transport region 140.

For example, the optical activation layer 135 may be to (e.g., be configured to) absorb light to generate an electrical signal. In detail, the first compound included in the first layer 131 may serve as a donor for supplying electrons, and the second compound included in the second layer 132 may serve as an acceptor for receiving electrons. Accordingly, the optoelectronic device 31 including the optical activation layer 135 may serve as an optical sensor. For example, the optoelectronic device 31 may serve as a fingerprint recognition sensor, which is described with reference to FIG. 5.

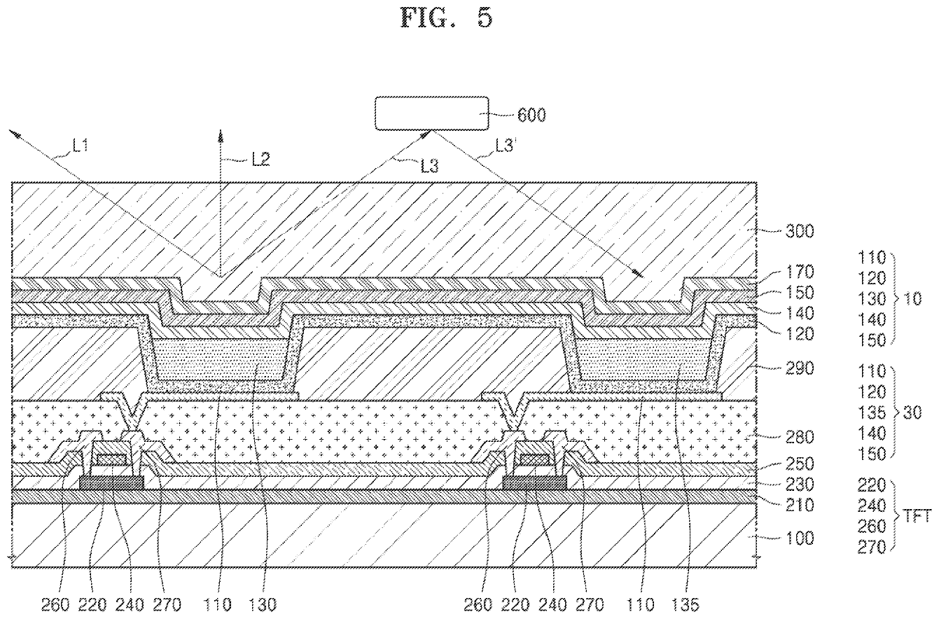

### Description of FIG. 4

FIG. 4 is a schematic view of an optoelectronic device 32 according to another embodiment.

The optoelectronic device 32 shown in FIG. 4 may be the same as and/or similar to the optoelectronic device 31 shown in FIG. 3, except for the optical activation layer 135, and thus, descriptions of other components will not be provided.

The optoelectronic device 32 may include the optical activation layer 135 arranged between the hole transport region 120 and the electron transport region 140. In one or more embodiments, the optical activation layer 135 may be arranged between the hole transport layer included in the hole transport region 120 and the buffer layer included in the electron transport region 140. In one or more embodiments, the optical activation layer 135 may be arranged between the emission auxiliary layer included in the hole transport region 120 and the buffer layer included in the electron transport region 140.

The optical activation layer 135 may include the first layer 131 adjacent to the hole transport region 120, the second layer 132 adjacent to the electron transport region 140, and a third layer 133 arranged between the first layer 131 and the second layer 132. For example, the third layer 133 may contact (e.g., directly contact) the first layer 131 and/or the second layer 132.

In one or more embodiments, the first layer 131 may contact (e.g., directly contact) the hole transport layer included in the hole transport region 120. In one or more embodiments, the first layer 131 may contact (e.g., directly contact) the emission auxiliary layer arranged on the hole transport layer.

In one or more embodiments, the second layer 132 may contact (e.g., directly contact) the buffer layer included in the electron transport region 140.

The first layer 131 may include the first compound (e.g., as its major or primary component). The first layer 131 may include (e.g., consist of) the first compound. For example, the first layer 131 may not include (e.g., may exclude) the second compound. The first layer 131 may be referred to as a p-type or kind optical activation layer or a donor layer.

The second layer 132 may include the second compound (e.g., as its major or primary component). The second layer 132 may include (e.g., consist of) the second compound. For example, the second layer 132 may not include (e.g., may exclude) the first compound. The second layer 132 may be referred to as an n-type or kind optical activation layer or an acceptor layer.

The third layer 133 may include the first compound and the second compound (e.g., as its two major components). For example, the first compound and the second compound may be mixed and included in the third layer 133. The third layer 133 may be referred to as a mixing (i.e., mixed) layer.

For example, the optical activation layer 135 may have a three-layer structure divided into the first layer 131 including the first compound, the third layer 133 including both (e.g., simultaneously) the first compound and the second compound, and the second layer 132 including the second compound.

The optical activation layer 135 may be to (e.g., be configured to) absorb incident light to generate excitons. The excitons may generate holes and electrons. The holes generated by the optical activation layer 135 may move to the first electrode 110 through the hole transport region 120. The electrons generated by the optical activation layer 135 may move to the second electrode 150 through the electron transport region 140.

For example, the optical activation layer 135 may be to (e.g., be configured to) absorb light to generate an electrical signal. In detail, the first compound included in each of the first layer 131 and the third layer 133 may serve as a donor for supplying electrons, and the second compound included in each of the second layer 132 and the third layer 133 may serve as an acceptor for receiving electrons. Accordingly, the optoelectronic device 32 including the optical activation layer 135 may serve as an optical sensor. For example, the optoelectronic device 32 may serve as a fingerprint recognition sensor, which is described with reference to FIG. 5.

### Electronic Apparatus

The light-emitting device 10 and the optoelectronic device 30, 31, or 32 may be included in one or more suitable electronic apparatuses. For example, the electronic apparatus may be a display apparatus, a light-emitting apparatus, an authentication apparatus, and/or the like.

The electronic apparatus (for example, a light-emitting apparatus) may further include, in addition to the light-emitting device 10 and the optoelectronic device 30, 31, or 32, i) a color filter, ii) a color conversion layer, or iii) a color filter and a color conversion layer. The color filter and/or the color conversion layer may be arranged in at least one traveling direction of light emitted from the light-emitting device 10. For example, the light emitted from the light-emitting device 10 may be blue light or white light. Details of the light-emitting device 10 may be as described herein. In one or more embodiments, the color conversion layer may include a quantum dot. The quantum dot may be, for example, the quantum dot as described herein.

The electronic apparatus may include a first substrate. The first substrate may include a plurality of subpixel areas, the color filter may include a plurality of color filter areas respectively corresponding to the subpixel areas, and the color conversion layer may include a plurality of color conversion areas respectively corresponding to the subpixel areas.

A pixel-defining film may be arranged among the subpixel areas to define each of the subpixel areas.

The color filter may further include a plurality of color filter areas and light-shielding patterns arranged among the color filter areas, and the color conversion layer may further include a plurality of color conversion areas and light-shielding patterns arranged among the color conversion areas.

The plurality of color filter areas (or the plurality of color conversion areas) may include a first area emitting first color light, a second area emitting second color light, and/or a third area emitting third color light, wherein the first color light, the second color light, and/or the third color light may have different maximum emission wavelengths. For example, the first color light may be red light, the second color light may be green light, and the third color light may be blue light. For example, the plurality of color filter areas (or the plurality of color conversion areas) may include quantum dots. In one or more embodiments, the first area may include a red quantum dot, the second area may include a green quantum dot, and the third area may be quantum dot free, (e.g., not include (e.g., may exclude) a (e.g., any) quantum dot). Details of the quantum dot may be as described herein. The first area, the second area, and/or the third area may each further include a scatter.

For example, the light-emitting device 10 may be to (e.g., be configured to) emit first light, the first area may be to (e.g., be configured to)absorb the first light to emit first-first color light, the second area may be to (e.g., be configured to)absorb the first light to emit second-first color light, and the third area may be to (e.g., be configured to) absorb the first light to emit third-first color light. In this regard, the first-first color light, the second-first color light, and the third-first color light may have different maximum emission wavelengths. In detail, the first light may be blue light, the first-first color light may be red light, the second-first color light may be green light, and the third-first color light may be blue light.

The electronic apparatus may further include a thin-film transistor, in addition to the optoelectronic device 30, 31, or 32 and the light-emitting device 10 described above. The thin-film transistor may include a source electrode, a drain electrode, and an activation layer, wherein any one of the source electrode and the drain electrode may be electrically connected to any one of the first electrode 110 and the second electrode 150 of the light-emitting device 10.

The thin-film transistor may further include a gate electrode, a gate insulating film, and/or the like.

The activation layer may include crystalline silicon, amorphous silicon, an organic semiconductor, an oxide semiconductor, and/or the like.

The electronic apparatus may further include a sealing portion for sealing the optoelectronic device 30, 31, or 32 and the light-emitting device 10. The sealing portion may be arranged between the color filter and/or the color conversion layer and the light-emitting device 10. The sealing portion allows light from the light-emitting device 10 to be extracted to the outside, and concurrently (e.g., simultaneously) prevents ambient air and moisture from penetrating into the optoelectronic device 30, 31, or 32 and the light-emitting device 10. The sealing portion may be a sealing substrate including a transparent glass substrate or a plastic substrate. The sealing portion may be a thin-film encapsulation layer including at least one layer of an organic layer and/or an inorganic layer. When the sealing portion is a thin-film encapsulation layer, the electronic apparatus may be flexible.

One or more functional layers may be additionally arranged on the sealing portion, in addition to the color filter and/or the color conversion layer, according to the utilize of the electronic apparatus. Examples of the functional layers may include a touch screen layer, a polarizing layer, and/or the like. The touch screen layer may be a pressure-sensitive touch screen layer, a capacitive touch screen layer, or an infrared touch screen layer. The authentication apparatus may be, for example, a biometric authentication apparatus that authenticates an individual by utilizing biometric information of a living body (for example, fingertips, pupils, etc.).

The authentication apparatus may further include a biometric information collector, in addition to the optoelectronic device 30, 31, or 32 and the light-emitting device 10 described herein.

The electronic apparatus may be applied to one or more suitable displays, light sources, lighting, personal computers (for example, a mobile personal computer), mobile phones, digital cameras, electronic organizers, electronic dictionaries, electronic game machines, medical instruments (for example, electronic thermometers, sphygmomanometers, blood glucose meters, pulse measurement devices, pulse wave measurement devices, electrocardiogram displays, ultrasonic diagnostic devices, or endoscope displays), fish finders, one or more suitable measuring instruments, meters (for example, meters for a vehicle, an aircraft, and a vessel), projectors, sensors (for example, vehicle sensors or household sensors), solar cells, and/or the like.

### Electronic Equipment

The optoelectronic device 30, 31, or 32 may be included in one or more suitable electronic equipment.

For example, the electronic equipment including the optoelectronic device 30, 31, or 32 may be at least one selected from among a flat panel display, a curved display, a computer monitor, a medical monitor, a television, a billboard, an indoor or outdoor light and/or light for signal, a head-up display, a fully or partially transparent display, a flexible display, a rollable display, a foldable display, a stretchable display, a laser printer, a telephone, a portable phone, a tablet personal computer, a phablet, a personal digital assistant (PDA), a wearable device, a laptop computer, a digital camera, a camcorder, a viewfinder, a micro display, a three-dimensional (3D) display, a virtual reality or augmented reality display, a vehicle, a video wall with multiple displays tiled together, a theater or stadium screen, a phototherapy device, a signboard, a vehicle sensor, a household sensor, a solar cell, and combinations thereof.

Because the optoelectronic device 30, 31, or 32 has excellent or suitable optoelectronic characteristics, the electronic equipment including the optoelectronic device 30, 31, or 32 may have the function of an optical sensor, such as a fingerprint recognition sensor.

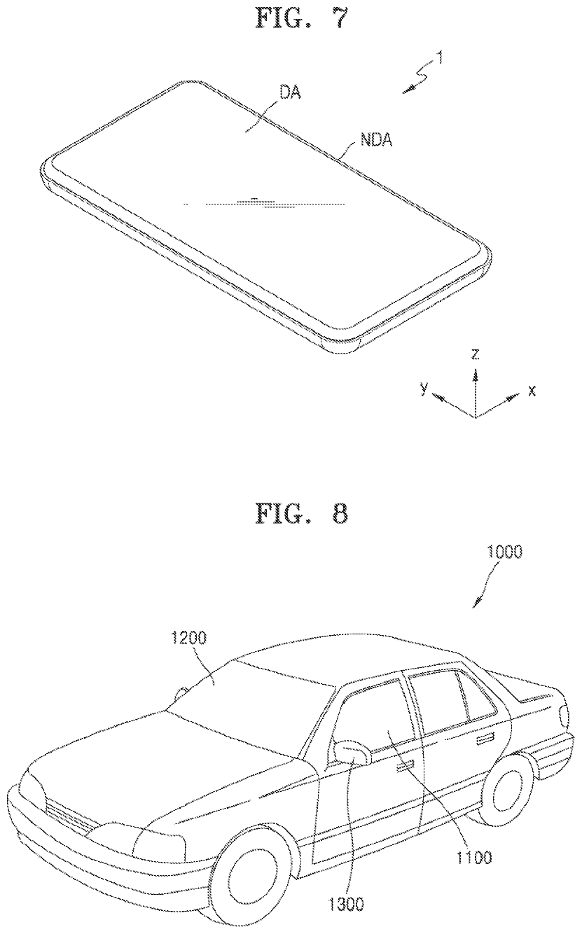

### Description of FIGS. 5 and 6

FIG. 5 is a cross-sectional schematic view of an electronic apparatus according to one or more embodiments.

The electronic apparatus of FIG. 5 includes a substrate 100, a thin-film transistor TFT, the light-emitting device 10, the optoelectronic device 30, and an encapsulation portion 300. The optoelectronic device 30 of FIG. 5 may be the optoelectronic device 30 described with reference to FIG. 1, but embodiments are not limited thereto. For example, the optoelectronic device 30 of FIG. 5 may be the optoelectronic device 31 of FIG. 3 or the optoelectronic device 32 of FIG. 4.

The substrate 100 may be a flexible substrate, a glass substrate, or a metal substrate. A buffer layer 210 may be arranged on the substrate 100. The buffer layer 210 may prevent or reduce penetration of impurities through the substrate 100 and may provide a flat surface on the substrate 100.

The thin-film transistor TFT may be arranged on the buffer layer 210. The thin-film transistor TFT may include an activation layer 220, a gate electrode 240, a source electrode 260, and a drain electrode 270.

The activation layer 220 may include an inorganic semiconductor such as silicon or polysilicon, an organic semiconductor, or an oxide semiconductor, and may include a source region, a drain region, and a channel region.

A gate insulating film 230 for insulating the activation layer 220 from the gate electrode 240 may be arranged on the activation layer 220, and the gate electrode 240 may be arranged on the gate insulating film 230.

An interlayer insulating film 250 may be arranged on the gate electrode 240. The interlayer insulating film 250 may be arranged between the gate electrode 240 and the source electrode 260 and between the gate electrode 240 and the drain electrode 270, to insulate from one another.

The source electrode 260 and the drain electrode 270 may be arranged on the interlayer insulating film 250. The interlayer insulating film 250 and the gate insulating film 230 may be formed to expose the source region and the drain region of the activation layer 220, and the source electrode 260 and the drain electrode 270 may be arranged in contact with the exposed portions of the source region and the drain region of the activation layer 220.

The light-emitting device 10 and the optoelectronic device 30 may be arranged on the thin-film transistor TFT.

The thin-film transistor TFT electrically connected to the light-emitting device 10 may transmit an electrical signal for driving the light-emitting device 10. The thin-film transistor TFT electrically connected to the optoelectronic device 30 may transmit an electrical signal generated by the optoelectronic device 30. The thin-film transistor TFT is covered and protected by a passivation layer 280. The passivation layer 280 may include an inorganic insulating film, an organic insulating film, or any combination thereof. The light-emitting device 10 and the optoelectronic device 30 are provided on the passivation layer 280.

The light-emitting device 10 includes the first electrode 110, the hole transport region 120, the emission layer 130, the electron transport region 140, and the second electrode 150. The optoelectronic device 30 includes the first electrode 110, the hole transport region 120, the optical activation layer 135, the electron transport region 140, and the second electrode 150. The first electrode 110 may be arranged on the passivation layer 280. The passivation layer 280 may be arranged to expose portions of the source electrode 260 and the drain electrode 270, not fully covering the source electrode 260 and the drain electrode 270, and the first electrode 110 may be arranged to be connected to the exposed portions of the source electrode 260 and the drain electrode 270.

A pixel defining layer 290 including an insulating material may be arranged on the first electrode 110. The pixel defining layer 290 may expose a portion of the first electrode 110. The pixel defining layer 290 may be a polyimide or polyacrylic organic film.

The hole transport region 120 may be arranged on the pixel defining layer 290. The hole transport region 120 included in the light-emitting device 10 and the hole transport region 120 included in the optoelectronic device 30 may be integrally formed as a single body. The hole transport region 120 included in the light-emitting device 10 and the hole transport region 120 included in the optoelectronic device 30 may be arranged on the pixel defining layer 290, be connected to each other, include substantially the same materials, and be formed substantially at the same time.

Each of the emission layer 130 and the optical activation layer 135 may be arranged on the hole transport region 120. Each of the emission layer 130 and the optical activation layer 135 may overlap the portion of the first electrode 110 which is exposed by the pixel defining layer 290.

The electron transport region 140 may be arranged on the emission layer 130 and the optical activation layer 135. The electron transport region 140 included in the light-emitting device 10 and the electron transport region 140 included in the optoelectronic device 30 may be integrally formed as a single body. The electron transport region 140 included in the light-emitting device 10 and the electron transport region 140 included in the optoelectronic device 30 may be arranged on the pixel defining layer 290, be connected to each other, include substantially the same materials, and be formed substantially at the same time.

The second electrode 150 may be arranged on the electron transport region 140. The second electrode 150 included in the light-emitting device 10 and the second electrode 150 included in the optoelectronic device 30 may be integrally formed as a single body. The second electrode 150 included in the light-emitting device 10 and the second electrode 150 included in the optoelectronic device 30 may be arranged on the pixel defining layer 290, be connected to each other, include substantially the same materials, and be formed substantially at the same time.

A capping layer 170 may be additionally formed on the second electrode 150. The capping layer 170 may be formed to cover the second electrode 150.

The encapsulation portion 300 may be arranged on the capping layer 170. The encapsulation portion 300 may be arranged on the light-emitting device 10 and the optoelectronic device 30 to protect the light-emitting device 10 and the optoelectronic device 30 from moisture and/or oxygen. The encapsulation portion 300 may include: an inorganic film including silicon nitride (SiNx), silicon oxide (SiOx), indium tin oxide (ITO), indium zinc oxide (IZO), or any combination thereof; an organic film including polyethylene terephthalate, polyethylene naphthalate, polycarbonate, polyimide, polyethylene sulfonate, polyoxymethylene, polyarylate, hexamethyldisiloxane, an acrylic resin (for example, polymethyl methacrylate, polyacrylic acid, etc.), an epoxy-based resin (for example, aliphatic glycidyl ether (AGE), etc.), or any combination thereof; or any combination of the inorganic film(s) and the organic film(s).

The light-emitting device 10 may emit lights L1, L2, and L3. For example, the lights L1, L2, and L3 may each be red light, green light, blue light, or near-infrared light.

The light L3 among the lights L1, L2, and L3 that have been emitted may be incident on an object 600 outside the electronic apparatus. For example, the object 600 may be a finger of a user of the electronic apparatus. A light L3′ reflected from the object 600 may be incident on the optoelectronic device 30.

The optical activation layer 135 may absorb the light L3′ incident on the optoelectronic device 30 to generate excitons. The excitons may generate holes and electrons. For example, the optical activation layer 135 may be to absorb light to generate an electrical signal. In detail, the first compound included in the optical activation layer 135 may serve as a donor for supplying electrons, and the second compound included in the optical activation layer 135 may serve as an acceptor for receiving electrons. For example, the optoelectronic device 30 may detect and convert energy of the light L3′ into an electrical signal. Accordingly, the optoelectronic device 30 may recognize the object 600 that has contacted (or approached) the electronic apparatus. Accordingly, the optoelectronic device 30 including the optical activation layer 135 may serve as an optical sensor (for example, a fingerprint recognition sensor).

FIG. 6 is a cross-sectional schematic view of an electronic apparatus according to another embodiment.

The electronic apparatus of FIG. 6 is the same as the electronic apparatus of FIG. 5, except that a light-shielding pattern 500 and a functional region 400 are additionally arranged on the encapsulation portion 300. The functional region 400 may be i) a color filter area, ii) a color conversion area, or iii) a combination of the color filter area and the color conversion area. In one or more embodiments, the light-emitting device 10 included in the electronic apparatus of FIG. 6 may be a tandem light-emitting device.

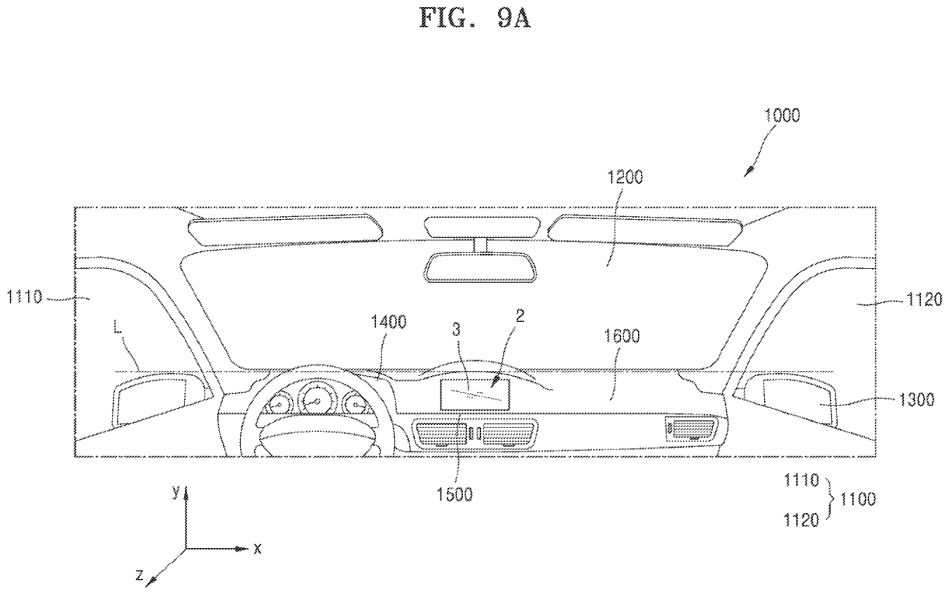

### Description of FIG. 7

FIG. 7 is a schematic perspective view of electronic equipment 1 including an optoelectronic device according to one or more embodiments. The electronic equipment 1 may be, as an apparatus that displays a moving image or still image, portable electronic equipment, such as a mobile phone, a smart phone, a tablet personal computer (PC), a mobile communication terminal, an electronic notebook, an electronic book, a portable multimedia player (PMP), a navigation, or a ultra mobile PC (UMPC), as well as one or more suitable products, such as a television, a laptop, a monitor, a billboard, or an Internet of things (IoT). The electronic equipment 1 may be such a product above or a part thereof. In some embodiments, the electronic equipment 1 may be a wearable device, such as a smart watch, a watch phone, a glasses-type or kind display, or a head mounted display (HMD), or a part of the wearable device. However, embodiments are not limited thereto. For example, the electronic equipment 1 may include a dashboard of a vehicle, a center fascia of a vehicle, a center information display arranged on a dashboard of a vehicle, a room mirror display replacing a side mirror of a vehicle, an entertainment display for the rear seat of a vehicle or a display arranged on the back of the front seat, or a head up display (HUD) installed in the front of a vehicle or projected on a front window glass, a computer generated hologram augmented reality head up display (CGH AR HUD). FIG. 7 illustrates a case in which the electronic equipment 1 is a smart phone for convenience of explanation.

The electronic equipment 1 may include a display area DA and a non-display area NDA outside the display area DA. A display apparatus may implement an image through an array of a plurality of pixels that are two-dimensionally arranged in the display area DA.

The non-display area NDA is an area that does not display an image, and may entirely surround the display area DA. On the non-display area NDA, a driver for providing electrical signals and/or power to display devices arranged on the display area DA may be arranged. On the non-display area NDA, a pad, which is an area to which an electronic device or a printing circuit board may be electrically connected, may be arranged.

In the electronic equipment 1, a length in the x-axis direction and a length in the y-axis direction may be different from each other. In one or more embodiments, as shown in FIG. 7, the length in the x-axis direction may be shorter than the length in the y-axis direction. In one or more embodiments, the length in the x-axis direction may be the same as the length in the y-axis direction. In one or more embodiments, the length in the x-axis direction may be longer than the length in the y-axis direction.

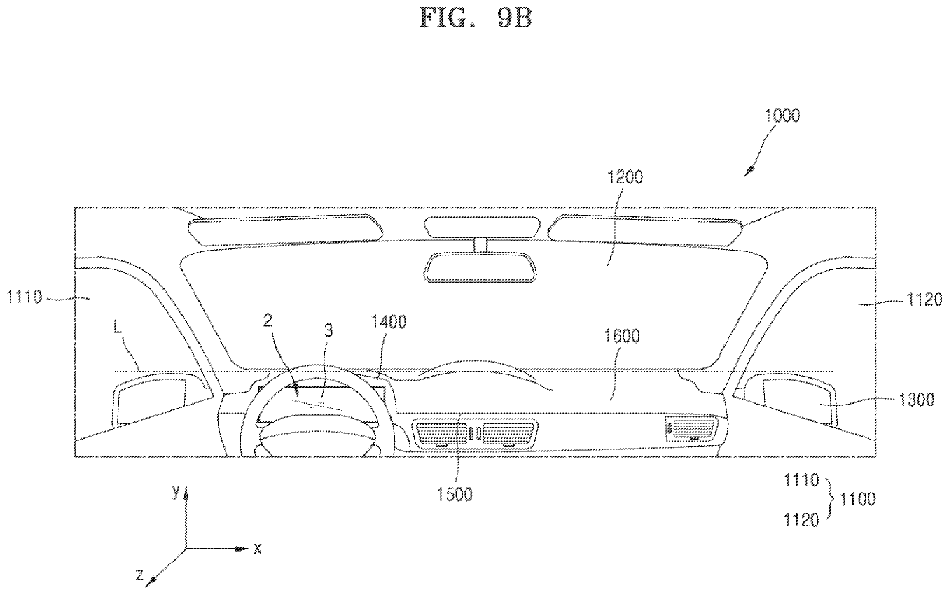

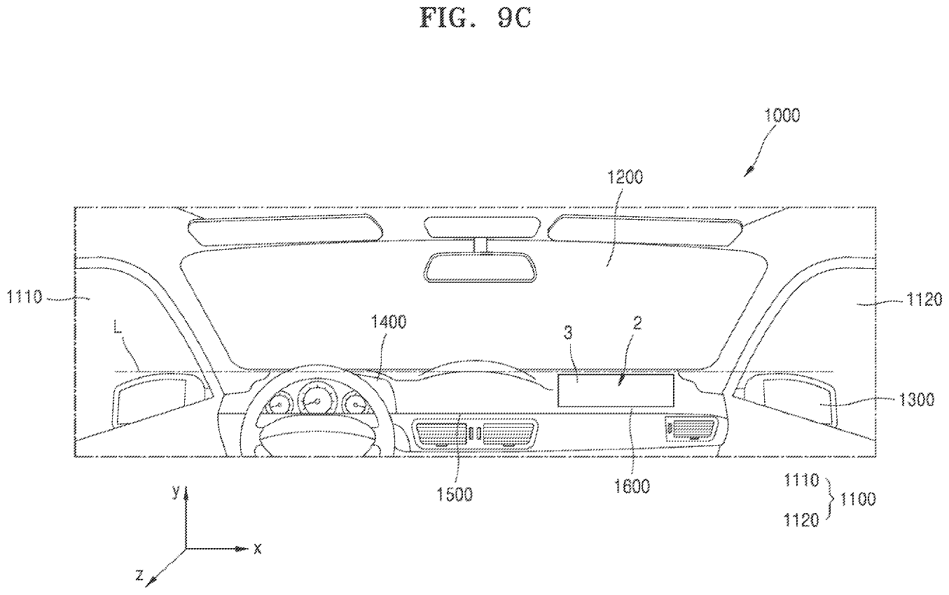

### Description of FIGS. 8 and 9A to 9C

FIG. 8 is a schematic perspective view of the exterior of a vehicle 1000 as electronic equipment including an optoelectronic device according to one or more embodiments. FIGS. 9A to 9C are each a schematic view of the interior of the vehicle 1000 according to one or more embodiments.

Referring to FIGS. 8, 9A, 9B, and 9C, the vehicle 1000 may refer to one or more suitable apparatus(es) for moving a subject to be transported, such as a human, an object, or an animal, from a departure point to a destination point. The vehicle 1000 may include a vehicle (e.g., automobile) traveling on a road or track, a vessel moving over the sea or river, an airplane flying in the sky utilizing the action of air, and/or the like.

The vehicle 1000 may travel on a road or a track. The vehicle 1000 may move in a set, or predetermined, or certain direction according to rotation of at least one wheel. For example, the vehicle 1000 may include a three-wheeled or four-wheeled vehicle, a construction machine, a two-wheeled vehicle, a prime mover apparatus, a bicycle, and a train running on a track.

The vehicle 1000 may include a body having an interior and an exterior, and a chassis in which mechanical apparatuses necessary for driving are installed as other parts except for the body. The exterior of the body may include a front panel, a bonnet, a roof panel, a rear panel, a trunk, a pillar provided at a boundary between doors, and/or the like. The chassis of the vehicle 1000 may include a power generating apparatus, a power transmitting apparatus, a driving apparatus, a steering apparatus, a braking apparatus, a suspension apparatus, a transmission apparatus, a fuel apparatus, front and rear wheels, left and right wheels, and/or the like.

The vehicle 1000 may include a side window glass 1100, a front window glass 1200, a side mirror 1300, a cluster 1400, a center fascia 1500, a passenger seat dashboard 1600, and a display apparatus 2.

The side window glass 1100 and the front window glass 1200 may be partitioned by a pillar arranged between the side window glass 1100 and the front window glass 1200.

The side window glass 1100 may be installed on the side of the vehicle 1000. In one or more embodiments, the side window glass 1100 may be installed on a door of the vehicle 1000. A plurality of side window glasses 1100 may be provided and may face each other. In one or more embodiments, the side window glass 1100 may include a first side window glass 1110 and a second side window glass 1120. In one or more embodiments, the first side window glass 1110 may be arranged adjacent to the cluster 1400. The second side window glass 1120 may be arranged adjacent to the passenger seat dashboard 1600.

In one or more embodiments, the side window glasses 1100 may be spaced apart from each other in the x-direction or the −x-direction. For example, the first side window glass 1110 and the second side window glass 1120 may be spaced apart from each other in the x-direction or the −x-direction. In other words, an imaginary straight line L connecting the side window glasses 1100 to each other may extend in the x-direction or the −x-direction. For example, the imaginary straight line L connecting the first side window glass 1110 and the second side window glass 1120 to each other may extend in the x direction or the −x direction.

The front window glass 1200 may be installed in the front of the vehicle 1000. The front window glass 1200 may be arranged between the side window glasses 1100 facing each other.

The side mirror 1300 may provide a view of the rear of the vehicle 1000. The side mirror 1300 may be installed on the exterior of the body. In one or more embodiments, a plurality of side mirrors 1300 may be provided. Any one of the plurality of side mirrors 1300 may be arranged outside the first side window glass 1110. The other one of the plurality of side mirrors 1300 may be arranged outside the second side window glass 1120.

The cluster 1400 may be arranged in front of the steering wheel. The cluster 1400 may include a tachometer, a speedometer, a coolant thermometer, a fuel gauge, a turn indicator, a high beam indicator, a warning lamp, a seat belt warning lamp, an odometer, a hodometer, an automatic shift selector indicator lamp, a door open warning lamp, an engine oil warning lamp, and/or a low fuel warning lamp.

The center fascia 1500 may include a control panel on which a plurality of buttons for adjusting an audio apparatus, an air conditioning apparatus, and a heater of a seat are arranged. The center fascia 1500 may be arranged on one side of the cluster 1400.

The passenger seat dashboard 1600 may be spaced apart from the cluster 1400 with the center fascia 1500 arranged therebetween. In one or more embodiments, the cluster 1400 may be arranged to correspond to a driver seat, and the passenger seat dashboard 1600 may be arranged to correspond to a passenger seat. In one or more embodiments, the cluster 1400 may be adjacent to the first side window glass 1110, and the passenger seat dashboard 1600 may be adjacent to the second side window glass 1120.

In one or more embodiments, the display apparatus 2 may include a display panel 3, and the display panel 3 may display an image. The display apparatus 2 may be arranged inside the vehicle 1000. In one or more embodiments, the display apparatus 2 may be arranged between the side window glasses 1100 facing each other. The display apparatus 2 may be arranged on at least one of the cluster 1400, the center fascia 1500, and the passenger seat dashboard 1600.

The display apparatus 2 may include an organic light-emitting display apparatus, an inorganic light-emitting display apparatus, a quantum dot display apparatus, and/or the like. Hereinafter, as the display apparatus 2 according to one or more embodiments of the disclosure, an organic light-emitting display apparatus including the light-emitting device according to the disclosure will be described as an example, but one or more suitable types (kinds) of display apparatuses as described above may be utilized in embodiments of the disclosure.

Referring to FIG. 9A, the display apparatus 2 may be arranged on the center fascia 1500. In one or more embodiments, the display apparatus 2 may display navigation information. In one or more embodiments, the display apparatus 2 may display audio, video, or information regarding vehicle settings.

Referring to FIG. 9B, the display apparatus 2 may be arranged on the cluster 1400. When the display apparatus 2 is arranged on the cluster 1400, the cluster 1400 may display driving information and/or the like through the display apparatus 2. For example, the cluster 1400 may be implemented digitally. The digital cluster 1400 may display vehicle information and driving information as images. For example, a needle and a gauge of a tachometer and one or more suitable warning light icons may be displayed by a digital signal.

Referring to FIG. 9C, the display apparatus 2 may be arranged on the passenger seat dashboard 1600. The display apparatus 2 may be embedded in the passenger seat dashboard 1600 or arranged on the passenger seat dashboard 1600.

In one or more embodiments, the display apparatus 2 arranged on the passenger seat dashboard 1600 may display an image related to information displayed on the cluster 1400 and/or information displayed on the center fascia 1500. In one or more embodiments, the display apparatus 2 arranged on the passenger seat dashboard 1600 may display information different from information displayed on the cluster 1400 and/or information displayed on the center fascia 1500.

### Manufacturing Method

Respective layers included in the hole transport region, the emission layer, the optical activation layer, and/or respective layers included in the electron transport region may be formed in a suitable region by utilizing one or more suitable methods selected from vacuum deposition, spin coating, casting, Langmuir-Blodgett (LB) deposition, ink-jet printing, laser-printing, laser-induced thermal imaging, and/or the like.

When respective layers included in the hole transport region, the emission layer, the optical activation layer, and/or respective layers included in the electron transport region are formed by vacuum deposition, the deposition may be performed at a deposition temperature of about 100° C. to about 500° C., a vacuum degree of about 10⁻⁸ torr to about 10⁻³ torr, and a deposition speed of about 0.01 Å/sec to about 100 Å/sec, depending on a material to be included in a layer to be formed and the structure of a layer to be formed.

### Definition of Terms

The term “C₃-C₆₀ carbocyclic group” as utilized herein refers to a cyclic group consisting of carbon only as a ring-forming atom and having 3 to 60 carbon atoms. The term “C₁-C₆₀ heterocyclic group” as utilized herein refers to a cyclic group that has 1 to 60 carbon atoms and further has, in addition to carbon, a heteroatom as a ring-forming atom. The C₃-C₆₀ carbocyclic group and the C₁-C₆₀ heterocyclic group may each be a monocyclic group consisting of one ring or a polycyclic group in which two or more rings are condensed with each other. For example, the C₁-C₆₀ heterocyclic group may have 3 to 61 ring-forming atoms.

The “cyclic group” as utilized herein may include both (e.g., simultaneously) the C₃-C₆₀ carbocyclic group and the C₁-C₆₀ heterocyclic group.

The term “Tr electron-rich C₃-C₆₀ cyclic group” as utilized herein refers to a cyclic group that has 3 to 60 carbon atoms and does not include *—N=*′ as a ring-forming moiety.

The term “Tr electron-deficient nitrogen-containing C₁-C₆₀ cyclic group” as utilized herein refers to a heterocyclic group that has 1 to 60 carbon atoms and includes *—N=*′ as a ring-forming moiety.

For example,
the C₃-C₆₀ carbocyclic group may be i) Group T1 or ii) a condensed cyclic group in which two or more of Group T1 are condensed with each other (for example, a cyclopentadiene group, an adamantane group, a norbornane group, a benzene group, a pentalene group, a naphthalene group, an azulene group, an indacene group, an acenaphthylene group, a phenalene group, a phenanthrene group, an anthracene group, a fluoranthene group, a triphenylene group, a pyrene group, a chrysene group, a perylene group, a pentaphene group, a heptalene group, a naphthacene group, a picene group, a hexacene group, a pentacene group, a rubicene group, a coronene group, an ovalene group, an indene group, a fluorene group, a spiro-bifluorene group, a benzofluorene group, an indenophenanthrene group, or an indenoanthracene group),
the C₁-C₆₀ heterocyclic group may be i) Group T2, ii) a condensed cyclic group in which two or more of Group T2 are condensed with each other, or iii) a condensed cyclic group in which at least one Group T2 and at least one Group T2 are condensed with each other (for example, a pyrrole group, a thiophene group, a furan group, an indole group, a benzoindole group, a naphthoindole group, an isoindole group, a benzoisoindole group, a naphthoisoindole group, a benzosilole group, a benzothiophene group, a benzofuran group, a carbazole group, a dibenzosilole group, a dibenzothiophene group, a dibenzofuran group, an indenocarbazole group, an indolocarbazole group, a benzofurocarbazole group, a benzothienocarbazole group, a benzosilolocarbazole group, a benzoindolocarbazole group, a benzocarbazole group, a benzonaphthofuran group, a benzonaphthothiophene group, a benzonaphthosilole group, a benzofurodibenzofuran group, a benzofurodibenzothiophene group, a benzothienodibenzothiophene group, a pyrazole group, an imidazole group, a triazole group, an oxazole group, an isoxazole group, an oxadiazole group, a thiazole group, an isothiazole group, a thiadiazole group, a benzopyrazole group, a benzimidazole group, a benzoxazole group, a benzoisoxazole group, a benzothiazole group, a benzoisothiazole group, a pyridine group, a pyrimidine group, a pyrazine group, a pyridazine group, a triazine group, a quinoline group, an isoquinoline group, a benzoquinoline group, a benzoisoquinoline group, a quinoxaline group, a benzoquinoxaline group, a quinazoline group, a benzoquinazoline group, a phenanthroline group, a cinnoline group, a phthalazine group, a naphthyridine group, an imidazopyridine group, an imidazopyrimidine group, an imidazotriazine group, an imidazopyrazine group, an imidazopyridazine group, an azacarbazole group, an azafluorene group, an azadibenzosilole group, an azadibenzothiophene group, an azadibenzofuran group, etc.),
the π electron-rich C₃-C₆₀ cyclic group may be i) Group T1, ii) a condensed cyclic group in which two or more of Group T1 are condensed with each other, iii) Group T3, iv) a condensed cyclic group in which two or more of Group T3 are condensed with each other, or v) a condensed cyclic group in which at least one Group T3 and at least one Group T1 are condensed with each other (for example, the C₃-C₆₀ carbocyclic group, a 1H-pyrrole group, a silole group, a borole group, a 2H-pyrrole group, a 3H-pyrrole group, a thiophene group, a furan group, an indole group, a benzoindole group, a naphthoindole group, an isoindole group, a benzoisoindole group, a naphthoisoindole group, a benzosilole group, a benzothiophene group, a benzofuran group, a carbazole group, a dibenzosilole group, a dibenzothiophene group, a dibenzofuran group, an indenocarbazole group, an indolocarbazole group, a benzofurocarbazole group, a benzothienocarbazole group, a benzosilolocarbazole group, a benzoindolocarbazole group, a benzocarbazole group, a benzonaphthofuran group, a benzonaphthothiophene group, a benzonaphthosilole group, a benzofurodibenzofuran group, a benzofurodibenzothiophene group, a benzothienodibenzothiophene group, etc.),
the π electron-deficient nitrogen-containing C₁-C₆₀ cyclic group may be i) Group T4, ii) a condensed cyclic group in which two or more of Group T4 are condensed with each other, iii) a condensed cyclic group in which at least one Group T4 and at least one Group T1 are condensed with each other, iv) a condensed cyclic group in which at least one Group T4 and at least one Group T3 are condensed with each other, or v) a condensed cyclic group in which at least one Group T4, at least one Group T1, and at least one Group T3 are condensed with one another (for example, a pyrazole group, an imidazole group, a triazole group, an oxazole group, an isoxazole group, an oxadiazole group, a thiazole group, an isothiazole group, a thiadiazole group, a benzopyrazole group, a benzimidazole group, a benzoxazole group, a benzoisoxazole group, a benzothiazole group, a benzoisothiazole group, a pyridine group, a pyrimidine group, a pyrazine group, a pyridazine group, a triazine group, a quinoline group, an isoquinoline group, a benzoquinoline group, a benzoisoquinoline group, a quinoxaline group, a benzoquinoxaline group, a quinazoline group, a benzoquinazoline group, a phenanthroline group, a cinnoline group, a phthalazine group, a naphthyridine group, an imidazopyridine group, an imidazopyrimidine group, an imidazotriazine group, an imidazopyrazine group, an imidazopyridazine group, an azacarbazole group, an azafluorene group, an azadibenzosilole group, an azadibenzothiophene group, an azadibenzofuran group, etc.).

Group T1 may be a cyclopropane group, a cyclobutane group, a cyclopentane group, a cyclohexane group, a cycloheptane group, a cyclooctane group, a cyclobutene group, a cyclopentene group, a cyclopentadiene group, a cyclohexene group, a cyclohexadiene group, a cycloheptene group, an adamantane group, a norbornane (or bicyclo[2.2.1]heptane) group, a norbornene group, a bicyclo[1.1.1]pentane group, a bicyclo[2.1.1]hexane group, a bicyclo[2.2.2]octane group, or a benzene group.

Group T2 may be a furan group, a thiophene group, a 1H-pyrrole group, a silole group, a borole group, a 2H-pyrrole group, a 3H-pyrrole group, an imidazole group, a pyrazole group, a triazole group, a tetrazole group, an oxazole group, an isoxazole group, an oxadiazole group, a thiazole group, an isothiazole group, a thiadiazole group, an azasilole group, an azaborole group, a pyridine group, a pyrimidine group, a pyrazine group, a pyridazine group, a triazine group, a tetrazine group, a pyrrolidine group, an imidazolidine group, a dihydropyrrole group, a piperidine group, a tetrahydropyridine group, a dihydropyridine group, a hexahydropyrimidine group, a tetrahydropyrimidine group, a dihydropyrimidine group, a piperazine group, a tetrahydropyrazine group, a dihydropyrazine group, a tetrahydropyridazine group, or a dihydropyridazine group.

Group T3 may be a furan group, a thiophene group, a 1H-pyrrole group, a silole group, or a borole group.

Group T4 may be a 2H-pyrrole group, a 3H-pyrrole group, an imidazole group, a pyrazole group, a triazole group, a tetrazole group, an oxazole group, an isoxazole group, an oxadiazole group, a thiazole group, an isothiazole group, a thiadiazole group, an azasilole group, an azaborole group, a pyridine group, a pyrimidine group, a pyrazine group, a pyridazine group, a triazine group, or a tetrazine group.

The terms “the cyclic group, the C₃-C₆₀ carbocyclic group, the C₁-C₆₀ heterocyclic group, the π electron-rich C₃-C₆₀ cyclic group, or the π electron-deficient nitrogen-containing C₁-C₆₀ cyclic group” as utilized herein refer to a group condensed to any cyclic group, a monovalent group, or a polyvalent group (for example, a divalent group, a trivalent group, a tetravalent group, etc.) according to the structure of a formula for which the corresponding term is utilized.

For example, the “benzene group” may be a benzo group, a phenyl group, a phenylene group, and/or the like, which may be easily understood by one of ordinary skill in the art according to the structure of a formula including the “benzene group.”

Depending on context, a divalent group may refer or be a polyvalent group (e.g., trivalent, tetravalent, etc., and not just divalent) per, e.g., the structure of a formula in connection with which of the terms are utilized.

Examples of the monovalent C₃-C₆₀ carbocyclic group and monovalent C₁-C₆₀ heterocyclic group are a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₁-C₆₀ heteroaryl group, a monovalent non-aromatic condensed polycyclic group, and a monovalent non-aromatic condensed heteropolycyclic group.

Examples of the divalent C₃-C₆₀ carbocyclic group and the monovalent C₁-C₆₀ heterocyclic group are a C₃-C₁₀ cycloalkylene group, a C₁-C₁₀ heterocycloalkylene group, a C₃-C₁₀ cycloalkenylene group, a C₁-C₁₀ heterocycloalkenylene group, a C₆-C₆₀ arylene group, a C₁-C₆₀ heteroarylene group, a divalent non-aromatic condensed polycyclic group, and a substituted or unsubstituted divalent non-aromatic condensed heteropolycyclic group.

The term “C₁-C₆₀ alkyl group” as utilized herein refers to a linear or branched aliphatic hydrocarbon monovalent group that has 1 to 60 carbon atoms, and examples thereof are a methyl group, an ethyl group, an n-propyl group, an isopropyl group, an n-butyl group, a sec-butyl group, an isobutyl group, a tert-butyl group, an n-pentyl group, a tert-pentyl group, a neopentyl group, an isopentyl group, a sec-pentyl group, a 3-pentyl group, a sec-isopentyl group, an n-hexyl group, an isohexyl group, a sec-hexyl group, a tert-hexyl group, an n-heptyl group, an isoheptyl group, a sec-heptyl group, a tert-heptyl group, an n-octyl group, an isooctyl group, a sec-octyl group, a tert-octyl group, an n-nonyl group, an isononyl group, a sec-nonyl group, a tert-nonyl group, an n-decyl group, an isodecyl group, a sec-decyl group, a tert-decyl group, and/or the like.

The term “C₁-C₆₀ alkylene group” as utilized herein refers to a divalent group having the same structure as the C₁-C₆₀ alkyl group.

The term “C₂-C₆₀ alkenyl group” as utilized herein refers to a monovalent hydrocarbon group having at least one carbon-carbon double bond in the middle or at the terminus of the C₂-C₆₀ alkyl group, and examples thereof are an ethenyl group, a propenyl group, a butenyl group, and/or the like.

The term “C₂-C₆₀ alkenylene group” as utilized herein refers to a divalent group having the same structure as the C₂-C₆₀ alkenyl group.

The term “C₂-C₆₀ alkynyl group” as utilized herein refers to a monovalent hydrocarbon group having at least one carbon-carbon triple bond in the middle or at the terminus of the C₂-C₆₀ alkyl group, and examples thereof are an ethynyl group, a propynyl group, and/or the like.

The term “C₂-C₆₀ alkynylene group” as utilized herein refers to a divalent group having the same structure as the C₂-C₆₀ alkynyl group.

The term “C₁-C₆₀ alkoxy group” as utilized herein refers to a monovalent group represented by —OA₁₀₁ (wherein A₁₀₁ is the C₁-C₆₀ alkyl group), and examples thereof are a methoxy group, an ethoxy group, an isopropyloxy group, and/or the like.

The term “C₃-C₁₀ cycloalkyl group” as utilized herein refers to a monovalent saturated hydrocarbon cyclic group having 3 to 10 carbon atoms, and examples thereof are a cyclopropyl group, a cyclobutyl group, a cyclopentyl group, a cyclohexyl group, a cycloheptyl group, a cyclooctyl group, an adamantanyl group, a norbornanyl group (or bicyclo[2.2.1]heptyl group), a bicyclo[1.1.1]pentyl group, a bicyclo[2.1.1]hexyl group, a bicyclo[2.2.2]octyl group, and/or the like.

The term “C₃-C₁₀ cycloalkylene group” as utilized herein refers to a divalent group having the same structure as the C₃-C₁₀ cycloalkyl group.

The term “C₁-C₁₀ heterocycloalkyl group” as utilized herein refers to a monovalent cyclic group of 1 to 10 carbon atoms, further including, in addition to carbon atoms, at least one heteroatom, as ring-forming atoms, and examples thereof are a 1,2,3,4-oxatriazolidinyl group, a tetrahydrofuranyl group, a tetrahydrothiophenyl group, and/or the like.

The term “C₁-C₁₀ heterocycloalkylene group” as utilized herein refers to a divalent group having the same structure as the C₁-C₁₀ heterocycloalkyl group.

The term “C₃-C₁₀ cycloalkenyl group” as utilized herein refers to a monovalent cyclic group that has 3 to 10 carbon atoms and at least one carbon-carbon double bond in the ring thereof and no aromaticity, and examples thereof are a cyclopentenyl group, a cyclohexenyl group, a cycloheptenyl group, and/or the like.

The term “C₃-C₁₀ cycloalkenylene group” as utilized herein refers to a divalent group having the same structure as the C₃-C₁₀ cycloalkenyl group.

The term “C₁-C₁₀ heterocycloalkenyl group” as utilized herein refers to a monovalent cyclic group of 1 to 10 carbon atoms, further including, in addition to carbon atoms, at least one heteroatom, as ring-forming atoms, and having at least one carbon-carbon double bond in the cyclic structure thereof. Examples of the C₁-C₁₀ heterocycloalkenyl group are a 4,5-dihydro-1,2,3,4-oxatriazolyl group, a 2,3-dihydrofuranyl group, a 2,3-dihydrothiophenyl group, and/or the like.

The term “C₁-C₁₀ heterocycloalkenylene group” as utilized herein refers to a divalent group having the same structure as the C₁-C₁₀ heterocycloalkenyl group.

The term “C₆-C₆₀ aryl group” as utilized herein refers to a monovalent group having a carbocyclic aromatic system of 6 to 60 carbon atoms.

The term “C₆-C₆₀ arylene group” as utilized herein refers to a divalent group having a carbocyclic aromatic system of 6 to 60 carbon atoms.

Examples of the C₆-C₆₀ aryl group are a phenyl group, a pentalenyl group, a naphthyl group, an azulenyl group, an indacenyl group, an acenaphthyl group, a phenalenyl group, a phenanthrenyl group, an anthracenyl group, a fluoranthenyl group, a triphenylenyl group, a pyrenyl group, a chrysenyl group, a perylenyl group, a pentaphenyl group, a heptalenyl group, a naphthacenyl group, a picenyl group, a hexacenyl group, a pentacenyl group, a rubicenyl group, a coronenyl group, an ovalenyl group, and/or the like.

When the C₆-C₆₀ aryl group and the C₆-C₆₀ arylene group each include two or more rings, the rings may be condensed with each other.

The term “C₁-C₆₀ heteroaryl group” as utilized herein refers to a monovalent group having a heterocyclic aromatic system of 1 to 60 carbon atoms, further including, in addition to carbon atoms, at least one heteroatom, as ring-forming atoms.

The term “C₁-C₆₀ heteroarylene group” as utilized herein refers to a divalent group having a heterocyclic aromatic system of 1 to 60 carbon atoms, further including, in addition to carbon atoms, at least one heteroatom, as ring-forming atoms.

Examples of the C₁-C₆₀ heteroaryl group are a pyridinyl group, a pyrimidinyl group, a pyrazinyl group, a pyridazinyl group, a triazinyl group, a quinolinyl group, a benzoquinolinyl group, an isoquinolinyl group, a benzoisoquinolinyl group, a quinoxalinyl group, a benzoquinoxalinyl group, a quinazolinyl group, a benzoquinazolinyl group, a cinnolinyl group, a phenanthrolinyl group, a phthalazinyl group, a naphthyridinyl group, and/or the like.

When the C₁-C₆₀ heteroaryl group and the C₁-C₆₀ heteroarylene group each include two or more rings, the rings may be condensed with each other.

The term “monovalent non-aromatic condensed polycyclic group” as utilized herein refers to a monovalent group (for example, having 8 to 60 carbon atoms) having two or more rings condensed to each other, only carbon atoms as ring-forming atoms, and no aromaticity in its entire molecular structure. Examples of the monovalent non-aromatic condensed polycyclic group are an indenyl group, a fluorenyl group, a spiro-bifluorenyl group, a benzofluorenyl group, an indenophenanthrenyl group, an indeno anthracenyl group, and/or the like.

The term “divalent non-aromatic condensed polycyclic group” as utilized herein refers to a divalent group having the same structure as the monovalent non-aromatic condensed polycyclic group.

The term “monovalent non-aromatic condensed heteropolycyclic group” as utilized herein refers to a monovalent group (for example, having 1 to 60 carbon atoms) having two or more rings condensed to each other, further including, in addition to carbon atoms, at least one heteroatom, as ring-forming atoms, and having non-aromaticity in its entire molecular structure. Examples of the monovalent non-aromatic condensed heteropolycyclic group are a pyrrolyl group, a thiophenyl group, a furanyl group, an indolyl group, a benzoindolyl group, a naphthoindolyl group, an isoindolyl group, a benzoisoindolyl group, a naphthoisoindolyl group, a benzosilolyl group, a benzothiophenyl group, a benzofuranyl group, a carbazolyl group, a dibenzosilolyl group, a dibenzothiophenyl group, a dibenzofuranyl group, an azacarbazolyl group, an azafluorenyl group, an azadibenzosilolyl group, an azadibenzothiophenyl group, an azadibenzofuranyl group, a pyrazolyl group, an imidazolyl group, a triazolyl group, a tetrazolyl group, an oxazolyl group, an isoxazolyl group, a thiazolyl group, an isothiazolyl group, an oxadiazolyl group, a thiadiazolyl group, a benzopyrazolyl group, a benzimidazolyl group, a benzoxazolyl group, a benzothiazolyl group, a benzoxadiazolyl group, a benzothiadiazolyl group, an imidazopyridinyl group, an imidazopyrimidinyl group, an imidazotriazinyl group, an imidazopyrazinyl group, an imidazopyridazinyl group, an indenocarbazolyl group, an indolocarbazolyl group, a benzofurocarbazolyl group, a benzothienocarbazolyl group, a benzosilolocarbazolyl group, a benzoindolocarbazolyl group, a benzocarbazolyl group, a benzonaphthofuranyl group, a benzonaphthothiophenyl group, a benzonaphthosilolyl group, a benzofurodibenzofuranyl group, a benzofurodibenzothiophenyl group, a benzothienodibenzothiophenyl group, and/or the like.

The term “divalent non-aromatic condensed heteropolycyclic group” as utilized herein refers to a divalent group having the same structure as the monovalent non-aromatic condensed heteropolycyclic group.

The term “C₆-C₆₀ aryloxy group” as utilized herein refers to —OA₁₀₂ (wherein A₁₀₂ is the C₆-C₆₀ aryl group).

The term “C₆-C₆₀ arylthio group” as utilized herein refers to —SA₁₀₃ (wherein A₁₀₃ is the C₆-C₆₀ aryl group).

The term “C₇-C₆₀ arylalkyl group” as utilized herein refers to -A₁₀₄A₁₀₅ (wherein A₁₀₄ is a C₁-C₅₄ alkylene group, and A₁₀₅ is a C₆-C₅₉ aryl group).

The term “C₂-C₆₀ heteroarylalkyl group” as utilized herein refers to -A₁₀₆A₁₀₇ (wherein A₁₀₆ is a C₁-C₅₉ alkylene group, and A₁₀₇ is a C₁-C₅₉ heteroaryl group).

The term “R₁₀ₐ” as utilized herein may be:
deuterium, —F, —Cl, —Br, —I, a hydroxyl group, a cyano group, or a nitro group;
a C₁-C₆₀ alkyl group, a C₂-C₆₀ alkenyl group, a C₂-C₆₀ alkynyl group, or a C₁-C₆₀ alkoxy group, each unsubstituted or substituted with deuterium, —F, —Cl, —Br, —I, a hydroxyl group, a cyano group, a nitro group, a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, a C₂-C₆₀ heteroarylalkyl group, —Si(Q₁₁)(Q₁₂)(Q₁₃), —N(Q₁₁)(Q₁₂), —B(Q₁₁)(Q₁₂), —C(═O)(Q₁₁), —S(═O)₂(Q₁₁), —P(═O)(Q₁₁)(Q₁₂), or any combination thereof;
a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, or a C₂-C₆₀ heteroarylalkyl group, each unsubstituted or substituted with deuterium, —F, —Cl, —Br, —I, a hydroxyl group, a cyano group, a nitro group, a C₁-C₆₀ alkyl group, a C₂-C₆₀ alkenyl group, a C₂-C₆₀ alkynyl group, a C₁-C₆₀ alkoxy group, a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, a C₂-C₆₀ heteroarylalkyl group, —Si(Q₂₁)(Q₂₂)(Q₂₃), —N(Q₂₁)(Q₂₂), —B(Q₂₁)(Q₂₂), —C(═O)(Q₂₁), —S(═O)₂(Q₂₁), —P(═O)(Q₂₁)(Q₂₂), or any combination thereof; or
—Si(Q₃₁)(Q₃₂)(Q₃₃), —N(Q₃₁)(Q₃₂), —B(Q₃₁)(Q₃₂), —C(═O)(Q₃₁), —S(═O)₂(Q₃₁), or —P(═O)(Q₃₁)(Q₃₂).
Q₁ to Q₃, Q₁₁ to Q₁₃, Q₂₁ to Q₂₃, and Q₃₁ to Q₃₃ utilized herein may each independently be: hydrogen; deuterium; —F; —Cl; —Br; —I; a hydroxyl group; a cyano group; a nitro group; a C₁-C₆₀ alkyl group; a C₂-C₆₀ alkenyl group; a C₂-C₆₀ alkynyl group; a C₁-C₆₀ alkoxy group; a C₃-C₆₀ carbocyclic group or a C₁-C₆₀ heterocyclic group, each unsubstituted or substituted with deuterium, —F, a cyano group, a C₁-C₆₀ alkyl group, a C₁-C₆₀ alkoxy group, a phenyl group, a biphenyl group, or any combination thereof; a C₇-C₆₀ arylalkyl group; or a C₂-C₆₀ heteroarylalkyl group.

The term “heteroatom” as utilized herein refers to any atom other than a carbon atom. Examples of the heteroatom are O, S, N, P, Si, B, Ge, Se, and any combination thereof.

The term “third-row transition metal” as utilized herein includes hafnium (Hf), tantalum (Ta), tungsten (W), rhenium (Re), osmium (Os), iridium (Ir), platinum (Pt), gold (Au), and/or the like.

“Ph” as utilized herein refers to a phenyl group, “Me” as utilized herein refers to a methyl group, “Et” as utilized herein refers to an ethyl group, “tert-Bu” or “Bu^{t}” as utilized herein refers to a tert-butyl group, and “OMe” as utilized herein refers to a methoxy group.

The term “biphenyl group” as utilized herein refers to “a phenyl group substituted with a phenyl group.” In other words, the “biphenyl group” may be a substituted phenyl group having a C₆-C₆₀ aryl group as a substituent.

The term “terphenyl group” as utilized herein refers to “a phenyl group substituted with a biphenyl group.” In other words, the “terphenyl group” may be a substituted phenyl group having, as a substituent, a C₆-C₆₀ aryl group substituted with a C₆-C₆₀ aryl group.

* and *′ as utilized herein, unless defined otherwise, each refer to a binding site to a neighboring atom in a corresponding formula or moiety.

The x-axis, y-axis, and z-axis as utilized herein are not limited to three axes in an orthogonal coordinate system, and may be interpreted in a broad sense including these axes. For example, the x-axis, y-axis, and z-axis may refer to those orthogonal to each other, or may refer to those in different directions that are not orthogonal to each other.

Hereinafter, compounds according to embodiments and light-emitting devices according to embodiments will be described in more detail with reference to Synthesis Examples and Examples. The wording “B was utilized instead of A” utilized in describing Synthesis Examples refers to that an identical molar equivalent of B was utilized in place of A.

### EXAMPLES

### Synthesis Example 1-1: Synthesis of Compound P2

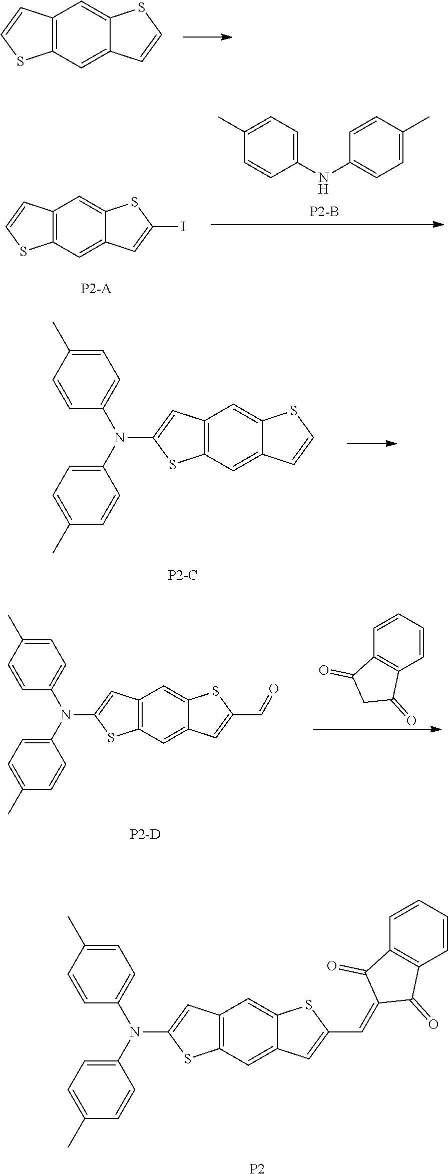

### Synthesis of Intermediate P2-A

The quantity 1.76 g (9.25 mmol) of benzo[1,2-5:4,5-b′]dithiophene was dissolved in 50 mL of dehydrated tetrahydrofuran. 3.4 mL (9.25 mmol) of 2.76 M n-butyl lithium (n-BuLi) hexane solution was added dropwise thereto at −78° C. for 5 minutes, and the mixture was stirred at room temperature for 30 minutes. After the temperature was lowered back to −78° C., 2.5 g (10 mmol) of iodine was added thereto, the mixture was stirred for 30 minutes, and the temperature was raised to room temperature. After water was added thereto to terminate the reaction, an extraction process was performed thereon three times by utilizing ethyl acetate, and the combined organic layers thus extracted was dried by adding anhydrous magnesium sulfate thereto. The product thus obtained was subjected to separation and purification through silica gel column chromatography (a volume ratio of hexane:dichloromethane was 1:1), so as to obtain 2.07 g (yield: 71%) of Intermediate P2-A. The compound thus produced was identified through MS/FAB.

C₁₀H₅IS₂: calc. 316.17, found 316.40.

### Synthesis of Intermediate P2-C

The quantity 6.32 g (20.0 mmol) of Intermediate P2-A, 3.95 g (20.0 mmol) of Intermediate P2-B, 0.37 g (0.4 mmol) of Pd₂(dba)₃, 0.08 g (0.4 mmol) of P(t-Bu)₃, and 5.76 g (60.0 mmol) of t-BuOK were dissolved in 90 mL of toluene, and the mixture was stirred at 120° C. for 24 hours. After the reaction solution was cooled to room temperature, an extraction process was performed thereon by utilizing 50 mL of water and three charges of 50 mL of diethyl ether. The combined organic layer thus collected was dried utilizing magnesium sulfate, and the residue obtained by evaporating the solvent therefrom was subjected to separation and purification through silica gel chromatography, so as to obtain 4.24 g (yield: 55%) of Intermediate P2-C. The compound thus produced was identified through MS/FAB.

C₂₄H₁₉NS₂: calc. 385.54, found 385.64.

### Synthesis of Intermediate P2-D

The quantity 1.92 (5 mmol) of Intermediate P2-C was dissolved in 50 mL of dehydrated tetrahydrofuran. 4 mL (16.0 mmol) of 2.76 M n-butyl lithium (n-BuLi) hexane solution was added dropwise thereto at −78° C. for 5 minutes, and the mixture was stirred at room temperature for 30 minutes. After the temperature was lowered back to −78° C., 0.7 g (10 mmol) of dehydrated N,N′-dimethylformamide was added thereto, the mixture was stirred for 30 minutes, and the temperature was raised to room temperature. After water was added thereto to terminate the reaction, an extraction process was performed thereon three times by utilizing ethyl acetate, and the combined organic layer thus extracted was dried by adding anhydrous magnesium sulfate thereto. The product thus obtained was subjected to separation and purification through silica gel column chromatography (a volume ratio of hexane:dichloromethane was 1:1), so as to obtain 1.07 g (yield: 71%) of Intermediate P2-D. The compound thus produced was identified through MS/FAB.

C₂₅H₁₉NOS₂: calc. 413.55, found 413.56.

### Synthesis of Compound P2

After 1.45 g (3.5 mmol) of Intermediate P2-D was dissolved in 20 mL of ethanol, 1.08 g (7.14 mmol) of 1,3-indandione was added thereto, and the mixture was stirred at 50° C. for 2 hours and then concentrated under reduced pressure. After a recrystallization process was performed thereon by utilizing chloroform and ethanol, the organic layer thus collected was dried utilizing magnesium sulfate, and the residue obtained by evaporating the solvent therefrom was subjected to separation and purification through silica gel chromatography, so as to obtain 1.29 g (yield: 68%) of Compound P2. The compound thus produced was identified through ¹H NMR (CDCl₃, 400 MHz) and MS/FAB.

δ=8.46 (s, 1H), 8.00-7.71 (m, 7H), 7.13-7.16 (m, 8H), 6.34 (s, 1H), 2.62 (s, 6H)

C₃₄H₂₃NO₂S₂: calc. 514.68, found 541.62.

### Synthesis Example 1-2 (Synthesis of Compound P5)

Compound P5 was synthesized in substantially the same manner as in the synthesis of Compound P2, except that 5,6-dichloro-1H-indene-1,3(2H)-dione was utilized instead of 1,3-indandione in the synthesis of Compound P2 of Synthesis Example 1-1. The compound thus produced was identified through ¹H NMR (CDCl₃, 400 MHz) and MS/FAB.

δ=8.45 (s, 1H), 8.00-7.78 (m, 5H), 7.16-7.13 (m, 8H), 6.34 (s, 1H), 2.62 (s, 6H)

C₃₄H₂₁C₁₂NO₂S₂: calc. 610.57, found 610.60.

### Synthesis Example 1-3 (Synthesis of Compound P10)

Compound P10 was synthesized in substantially the same manner as in the synthesis of Compound P2, except that 1,3-dimethyl-2-barbituric acid was utilized instead of 1,3-indandione in the synthesis of Compound P2 of Synthesis Example 1-1. The compound thus produced was identified through ¹H NMR (CDCl₃, 400 MHz) and MS/FAB.

δ=8.01-8.00 (m, 2H), 7.80-7.78 (m, 2H), 7.16-7.13 (m, 8H), 6.34 (s, 1H), 3.22 (s, 6H), 2.32 (s, 6H)

C₃₁H₂₅C₁₂N₃O₃S₂: calc. 551.68, found 551.71.

### Synthesis Example 1-4 (Synthesis of Compound P13)

Compound P13 was synthesized in substantially the same manner as in the synthesis of Compound P2, except that 1,2,5,6-tetrahydro-1,4-dimethyl-2,6-dioxo-3-pyridinecarbonitrile was utilized instead of 1,3-indandione in the synthesis of Compound P2 of Synthesis Example 1-1. The compound thus produced was identified through ¹H NMR (CDCl₃, 400 MHz) and MS/FAB.

δ=8.00 (s, 1H), 7.80-7.78 (m, 2H), 7.50 (s, 1H), 7.16-7.13 (m, 8H), 6.34 (s, 1H), 3.23 (s, 3H), 3.32 (s, 6H), 2.32 (s, 3H)

C₃₃H₂₅N₃O₂S₂: calc. 559.70, found 559.71.

### Synthesis Example 1-5 (Synthesis of Compound P14)

Compound P14 was synthesized in substantially the same manner as in the synthesis of Compound P2, except that, in the synthesis of Compound P2 of Synthesis Example 1-1, 9H-carbazole was utilized instead of Intermediate P2-B, and 1,3-dimethyl-2-barbituric acid was utilized instead of 1,3-indandione. The compound thus produced was identified through ¹H NMR (CDCl₃, 400 MHz) and MS/FAB.

δ=8.55-8.53 (m, 1H), 8.19-8.17 (m, 1H), 8.01-7.94 (m, 5H), 7.59-7.50 (m, 3H), 7.35-7.33 (m, 1H), 7.20-7.16 (m, 2H), 3.22 (s, 6H)

C₂₉H₁₉N₃O₃S₂: calc. 521.61, found 521.67.

### Synthesis Example 1-6 (Synthesis of Compound P15)

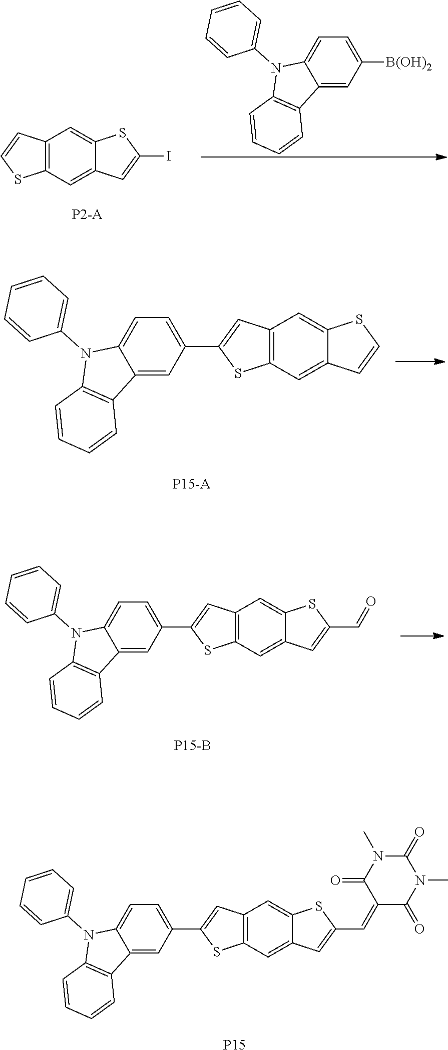

### Synthesis of Intermediate P15-A

The quantity 2.05 g (7.14 mmol) of (9-phenyl-9H-carbazol-3-yl)boronic acid, 2.26 g (7.14 mmol) of Intermediate P2-A, 116 g (1.00 mmol) of tetrakis(triphenylphosphine)palladium (Pd(PPh₃)₄), and 2.48 g (17.92 mmol) of K₂CO₃ were dissolved in 120 mL of a mixed solution of THF/H₂O (volume ratio of 2:1), and the mixture was stirred at 70° C. for 5 hours. After the reaction solution was cooled to room temperature, 60 mL of water was added thereto, and an extraction process was performed thereon three times by utilizing 80 mL of ethyl ether. The combined organic layer thus collected was dried utilizing magnesium sulfate, and the residue obtained by evaporating the solvent therefrom was subjected to separation and purification through silica gel chromatography, so as to obtain 2.28 g (yield: 74%) of Intermediate P15-A. The compound thus produced was identified through ¹H NMR (CDCl₃, 400 MHz) and MS/FAB.

C₂₈H₁₇NS: calc. 431.57, found 431.62.

### Synthesis of Intermediate P15-B

Intermediate P15-B was synthesized in substantially the same manner as in the synthesis of Intermediate P2-D, except that Intermediate P15-A was utilized instead of Intermediate P2-C in the synthesis of Intermediate P2-D of Synthesis Example 1-1. The compound thus produced was identified through ¹H NMR (CDCl₃, 400 MHz) and MS/FAB.

C₂₉H₁₇NOS₂: calc. 459.58, found 459.62.

### Synthesis of Compound P15

Compound P15 was synthesized in substantially the same manner as in the synthesis of Compound P2, except that, in the synthesis of Compound P2 of Synthesis Example 1-1, Intermediate P15-B was utilized instead of Intermediate P2-D, and 1,3-dimethyl-2-barbituric acid was utilized instead of 1,3-indandione. The compound thus produced was identified through ¹H NMR (CDCl₃, 400 MHz) and MS/FAB.

δ=8.30-8.27 (m, 1H), 8.19-8.13 (m, 2H), 8.01-7.99 (m, 3H), 7.91-7.89 (m, 2H), 7.35-7.33 (m, 1H), 7.20-7.16 (m, 2H), 3.22 (s, 6H)

C₃₅H₂₃N₃O₃S₂: calc. 597.71, found 597.76.

### Synthesis Example 1-7 (Synthesis of Compound P16)

Compound P16 was synthesized in substantially the same manner as in the synthesis of Compound P10, except that 3-ethylrhodanine was utilized instead of 1,3-dimethyl-2-barbituric acid in the synthesis of Compound P10 of Synthesis Example 1-3. The compound thus produced was identified through ¹H NMR (CDCl₃, 400 MHz) and MS/FAB.

δ=8.00 (s, 1H), 7.95 (s, 1H), 7.86 (s, 1H), 7.78 (s, 1H), 7.15-7.13 (m, 8H), 6.34 (s, 1H), 3.34 (s, 3H), 2.32 (s, 6H)

C₂₉H₂₂N₂OS₄: calc. 542.75, found 542.78.

### Synthesis Example 1-8 (Synthesis of Compound P19)

Compound P19 was synthesized in substantially the same manner as in the synthesis of Compound P2, except that, in the synthesis of Compound P2 of Synthesis Example 1-1,10H-phenoxazine was utilized instead of Intermediate P2-B, and 2-(3-oxo-2,3-dihydro-1H-inden-1-ylidene)malononitrile was utilized instead of 1,3-indandione. The compound thus produced was identified through ¹H NMR (CDCl₃, 400 MHz) and MS/FAB.

δ=8.30 (s, 1H), 7.86-7.72 (m, 3H), 7.36-7.32 (m, 3H), 7.21-7.14 (m, 3H), 7.01-6.96 (m, 6H), 6.34 (s, 1H)

C₃₅H₁₇N₃O₂S₂: calc. 575.66, found 575.68.

### Synthesis Example 1-9 (Synthesis of Compound P20)

Compound P20 was synthesized in substantially the same manner as in the synthesis of Compound P2, except that, in the synthesis of Compound P2 of Synthesis Example 1-1,9,9-dimethyl-9,10-dihydroacridine was utilized instead of Intermediate P2-B, and 2-(3-oxo-2,3-dihydro-1H-inden-1-ylidene)malononitrile was utilized instead of 1,3-indandione. The compound thus produced was identified through ¹H NMR (CDCl₃, 400 MHz) and MS/FAB.

δ=8.00 (s, 1H), 7.86 (s, 1H), 7.76-7.72 (m, 2H), 7.36-7.32 (m, 3H), 7.21-7.14 (m, 7H), 6.95-6.93 (m, 2H), 6.34 (s, 1H), 1.69 (s, 6H)

C₃₈H₂₃N₃OS₂: calc. 601.74, found 601.84.

### Synthesis Example 1-10 (Synthesis of Compound P22)

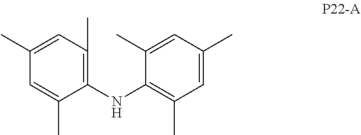

Compound P22 was synthesized in substantially the same manner as in the synthesis of Compound P2, except that, in the synthesis of Compound P2 of Synthesis Example 1-1, Intermediate P22-A was utilized instead of Intermediate P2-B, and malononitrile was utilized instead of 1,3-indandione. The compound thus produced was identified through ¹H NMR (CDCl₃, 400 MHz) and MS/FAB.

δ=7.93 (s, 1H), 7.86 (s, 1H), 7.76 (s, 1H), 7.30 (s, 1H), 6.77-6.75 (m, 4H), 6.34 (s, 1H), 2.26 (s, 6H), 2.12 (s, 12H)

C₃₂H₂₇N₃S₂: calc. 517.71, found 517.73.

### Synthesis Example 1-11 (Synthesis of Compound P26)

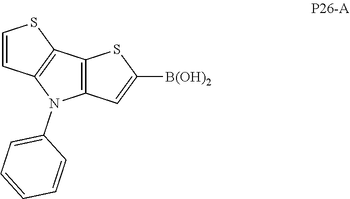 [SMILES: OB(O)c1cc2c(s1)c1sccc1n2-c1ccccc1]

Compound P26 was synthesized in substantially the same manner as in the synthesis of Compound P15, except that Intermediate P26-A was utilized instead of (9-phenyl-9H-carbazol-3-yl)boronic acid in the synthesis of Compound P15 of Synthesis Example 1-6. The compound thus produced was identified through ¹H NMR (CDCl₃, 400 MHz) and MS/FAB.

δ=8.01-8.00 (m, 2H), 7.86 (s, 1H), 7.78 (s, 1H), 7.62-7.50 (m, 6H), 7.31-7.30 (m, 1H), 7.17-7.12 (m, 2H), 3.22 (s, 6H)

C₃₁H₁₈N₃O₃S₄: calc 609.75, found 609.81.

### Synthesis Example 1-12 (Synthesis of Compound P30)

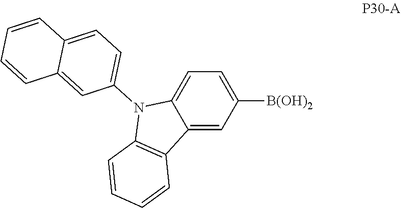 [SMILES: OB(O)c1ccc2c(c1)c1ccccc1n2-c1ccc2ccccc2c1]

Compound P30 was synthesized in substantially the same manner as in the synthesis of Compound P15, except that Intermediate P30-A was utilized instead of (9-phenyl-9H-carbazol-3-yl)boronic acid in the synthesis of Compound P15 of Synthesis Example 1-6. The compound thus produced was identified through ¹H NMR (CDCl₃, 400 MHz) and MS/FAB.

δ=8.30-7.58 (m, 12H), 7.59-7.50 (m, 5H), 7.36-7.34 (m, 1H), 7.20-7.18 (m, 1H), 3.34 (s, 3H)

C₃₇H₂₂N₂OS₄: calc 638.84, found 638.91.

### Synthesis Example 1-13 (Synthesis of Compound P34)

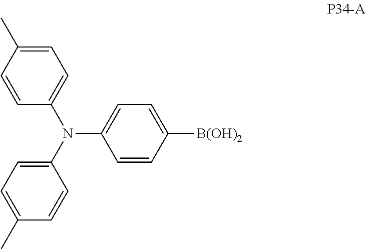 [SMILES: Cc1ccc(N(c2ccc(C)cc2)c2ccc(B(O)O)cc2)cc1]

Compound P34 was synthesized in substantially the same manner as in the synthesis of Compound P15, except that, in the synthesis of Compound P15 of Synthesis Example 1-6, Intermediate P34-A was utilized instead of (9-phenyl-9H-carbazol-3-yl)boronic acid, and malononitrile was utilized instead of 1,3-dimethyl-2-barbituric acid. The compound thus produced was identified through ¹H NMR (CDCl₃, 400 MHz) and MS/FAB.

δ=7.99-7.91 (m, 3H), 7.59 (s, 1H), 7.37-7.30 (m, 3H), 7.15-7.13 (m, 8H), 6.99-6.95 (m, 2H), 2.32 (s, 6H)

C₃₄H₂₃N₃S₂: calc 537.70, found 537.91.

### Synthesis Example 2-1 (Synthesis of Compound N1)

1,4,5,8-Naphthalenetetracarboxylic dianhydride was subjected to sublimation purification. The compound thus produced was identified through ¹H NMR (CDCl₃, 400 MHz) and MS/FAB.

δ=9.05 (s, 4H)

C₁₄H₄O₆: calc 268.18, found 268.21.

### Synthesis Example 2-2 (Synthesis of Compound N5)

A mixture of 1,4,5,8,-naphthalenetetracarboxylic dianhydride (1 eq.) and aniline (2.2 eq.) was dissolved in a dimethylformamide (DMF) solvent, placed in a two-necked, round-bottomed flask, and stirred at 180° C. for 24 hours. After the temperature was lowered to room temperature, methanol was added thereto to precipitate a product, which was then filtered to obtain a powdery material. The material was washed several times with methanol and purified by recrystallization utilizing ethyl acetate and dimethyl sulfoxide (DMSO). The product thus obtained was placed in an oven and dried in vacuum at 80° C. for 24 hours, so as to obtain Compound N5. The yield was greater than or equal to 50%.

δ=8.62 (s, 4H), 7.58-7.57 (m, 6H), 7.43-7.40 (m, 4H)

C₂₆H₁₄N₂O₄: calc 418.41, found 418.52.

### Synthesis Example 2-3 (Synthesis of Compound N6)

Compound N6 was synthesized in substantially the same manner as in the synthesis of Compound N5, except that 4-methylaniline was utilized instead of aniline in the synthesis of Compound N5 of Synthesis Example 2-2. The compound thus produced was identified through ¹H NMR (CDCl₃, 400 MHz) and MS/FAB.

δ=8.62-8.60 (m, 4H), 7.27-7.25 (m, 8H), 2.32 (s, 6H)

C₂₈H₁₈N₂O₄: calc 446.46, found 446.52.

### Synthesis Example 2-4 (Synthesis of Compound N7)

Compound N7 was synthesized in substantially the same manner as in the synthesis of Compound N5, except that 4-fluorolaniline was utilized instead of aniline in the synthesis of Compound N5 of Synthesis Example 2-2. The compound thus produced was identified through ¹H NMR (CDCl₃, 400 MHz) and MS/FAB.

δ=8.62-8.60 (m, 4H), 7.56-7.54 (m, 4H), 7.40-7.38 (m, 4H)

C₂₆H₁₂F₂N₂O₄: calc 454.39, found 454.41.

### Synthesis Example 2-5 (Synthesis of Compound N8)

Compound N8 was synthesized in substantially the same manner as in the synthesis of Compound N5, except that 4-chlorolaniline was utilized instead of aniline in the synthesis of Compound N5 of Synthesis Example 2-2. The compound thus produced was identified through ¹H NMR (CDCl₃, 400 MHz) and MS/FAB.

δ=8.85 (s, 4H), 7.63 (s, 4H), 7.60 (s, 4H).

C₂₆H₁₂C₁₂N₂O₄: calc 487.29, found 487.61.

### Example 1

As an anode, a glass substrate (product of Corning Inc.) with a 15 ohms per square centimeter (Q/cm²) (1,200 angstrom (Å)) ITO formed thereon was cut to a size of 50 mm×50 mm×0.7 mm, sonicated by utilizing isopropyl alcohol and pure water each for 5 minutes, cleaned by irradiation of ultraviolet rays and exposure of ozone thereto for 30 minutes, and then mounted on a vacuum deposition apparatus.

The material 2-TNATA was vacuum-deposited on the anode to form a hole injection layer having a thickness of 600 Å. 4,4′-bis[N-(1-naphthyl)-N-phenylamino]biphenyl (hereinafter, referred to as NPB) was vacuum-deposited on the hole injection layer to form a hole transport layer having a thickness of 300 Å. An emission auxiliary layer having a thickness of 300 Å was formed on the hole transport layer.

On the emission auxiliary layer, Compound P2 was deposited to form a first layer, and Compound N6 was deposited to form a second layer, so as to form an optical activation layer having a total thickness of 500 Å.

The material Alq₃ was vacuum-deposited on the optical activation layer to form an electron transport layer having a thickness of 300 Å. LiF was vacuum-deposited on the electron transport layer to form an electron injection layer having a thickness of 10 Å. Al was vacuum-deposited on the electron injection layer to form a cathode having a thickness of 3,000 Å, thereby completing the manufacture of an optoelectronic device.

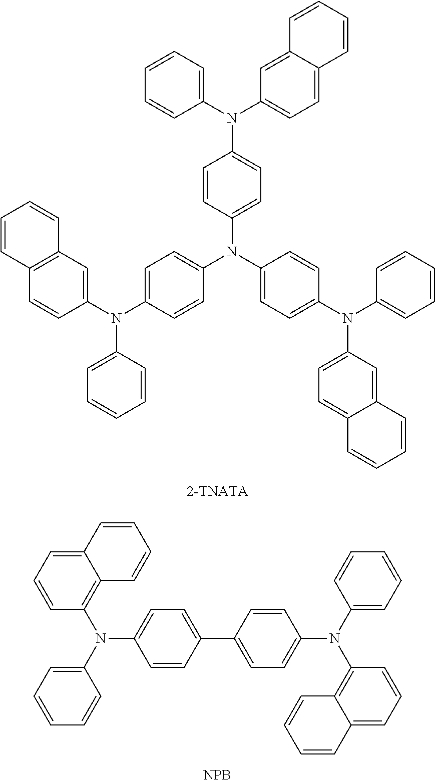 [SMILES: c1ccc(N(c2ccc(N(c3ccc(N(c4ccccc4)c4ccc5ccccc5c4)cc3)c3ccc(N(c4ccccc4)c4ccc5ccccc5c4)cc3)cc2)c2ccc3ccccc3c2)cc1 ; c1ccc(N(c2ccc(-c3ccc(N(c4ccccc4)c4cccc5ccccc45)cc3)cc2)c2cccc3ccccc23)cc1 ; "NPB"]

### Examples 2 to 13 and Comparative Examples 1 to 6

Optoelectronic devices were manufactured in substantially the same manner as in Example 1, except that compounds shown in Table 1 were utilized in forming the optical activation layer.

### Evaluation Example 1

To evaluate the characteristics of the optoelectronic device manufactured in each of Examples 1 to 13 and Comparative Examples 1 to 6, the external quantum efficiency (EQE) and dark current density (J_{dark}) thereof were measured, and the results are shown in Table 1.

Light (530 nm to 630 nm) was irradiated to the optoelectronic device by utilizing a xenon lamp apparatus. By utilizing a current meter (Keithley, Tektronix, USA), the maximum absorption wavelength during light irradiation was measured, and the converted current was measured. The external quantum efficiency (FOE) was calculated by utilizing the irradiated light and the measured current.

A voltage (−3 V) was applied to the anode by utilizing an IVL-25CH apparatus. The current flowing when voltage was applied was measured by utilizing a current meter (Keithley, Tektronix, USA). The dark current density (J_{dark}) was calculated by utilizing the measured current.

TABLE 1

| | p-type compound | n-type compound | EQE (%) | J_{dark} (mA/cm²) | λₘₐₓ (nm) |
|---|---|---|---|---|---|
| Example 1 | P2 | N6 | 35 | 3.2 × 10⁻⁶ | 530 |
| Example 2 | P5 | N6 | 40 | 4.1 × 10⁻⁶ | 620 |
| Example 3 | P10 | N8 | 41 | 2.3 × 10⁻⁶ | 530 |
| Example 4 | P13 | N5 | 38 | 2.8 × 10⁻⁶ | 525 |
| Example 5 | P14 | N8 | 35 | 2.5 × 10⁻⁶ | 520 |
| Example 6 | P15 | N8 | 38 | 2.2 × 10⁻⁶ | 521 |
| Example 7 | P16 | N5 | 35 | 4.8 × 10⁻⁶ | 522 |
| Example 8 | P19 | N5 | 31 | 5.8 × 10⁻⁶ | 625 |
| Example 9 | P20 | N8 | 32 | 3.2 × 10⁻⁶ | 630 |
| Example 10 | P22 | N8 | 38 | 3.9 × 10⁻⁶ | 528 |
| Example 11 | P26 | N1 | 32 | 3.1 × 10⁻⁶ | 530 |
| Example 12 | P30 | N1 | 36 | 4.3 × 10⁻⁶ | 535 |
| Example 13 | P34 | N7 | 39 | 2.9 × 10⁻⁶ | 530 |
| Comparative | SubPC | Fullerene 60 | 15 | 2.0 × 10⁻⁶ | 530 |
| Example 1 | | | | | |
| Comparative | SubNC | Fullerene 60 | 10 | 2.0 × 10⁻⁶ | 650 |
| Example 2 | | | | | |
| Comparative | CE1 | Fullerene 60 | 6 | 2.8 × 10⁻⁶ | 521 |
| Example 3 | | | | | |
| Comparative | CE2 | Fullerene 60 | 9 | 3.2 × 10⁻⁶ | 503 |
| Example 4 | | | | | |
| Comparative | CE3 | Fullerene 60 | 8 | 5.5 × 10⁻⁶ | 505 |
| Example 5 | | | | | |
| Comparative | CE4 | Fullerene 60 | 11 | 4.3 × 10⁻⁶ | 538 |
| Example 6 | | | | | |

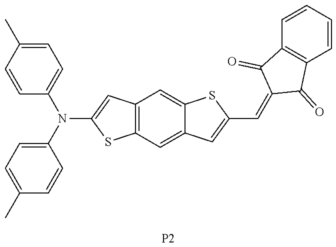 [SMILES: Cc1ccc(N(c2ccc(C)cc2)c2cc3cc4sc(C=C5C(=O)c6ccccc6C5=O)cc4cc3s2)cc1 ; "P2"]

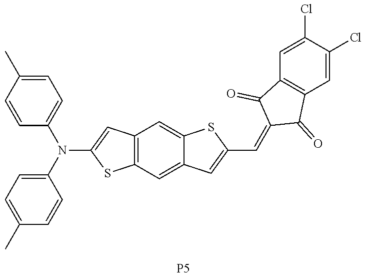 [SMILES: Cc1ccc(N(c2ccc(C)cc2)c2cc3cc4sc(C=C5C(=O)c6cc(Cl)c(Cl)cc6C5=O)cc4cc3s2)cc1 ; "P5"]

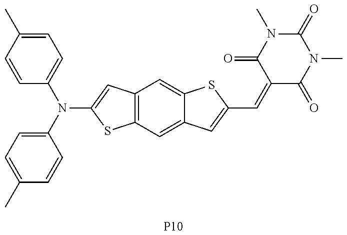 [SMILES: Cc1ccc(N(c2ccc(C)cc2)c2cc3cc4sc(C=C5C(=O)N(C)C(=O)N(C)C5=O)cc4cc3s2)cc1 ; "P10"]

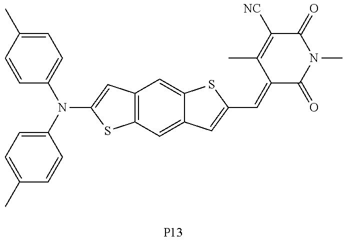

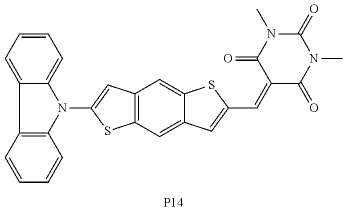

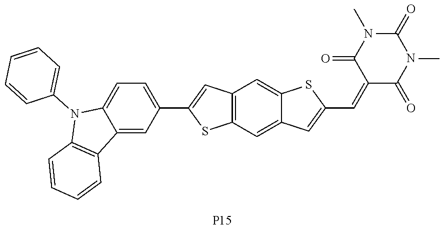

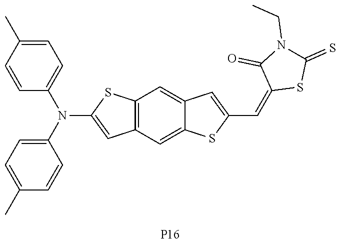

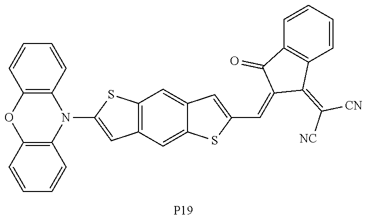

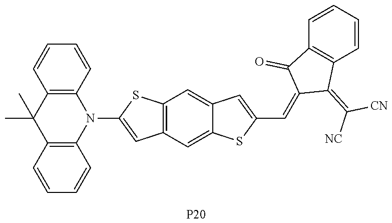

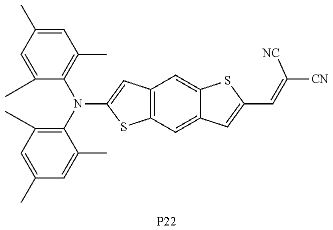

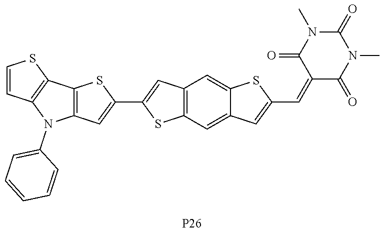

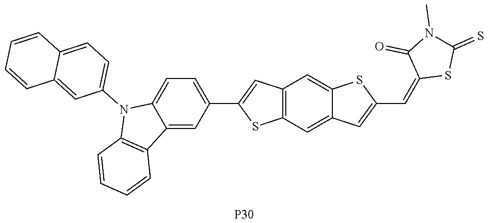

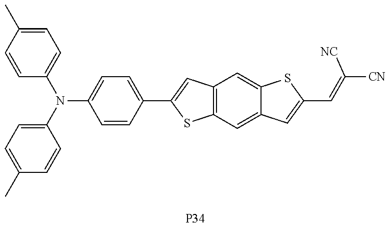

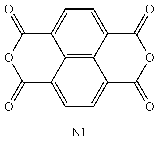

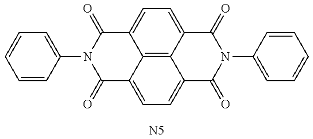

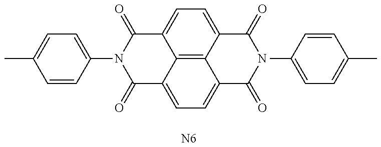

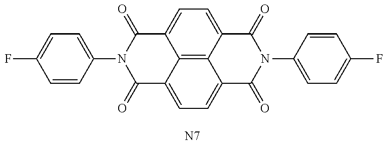

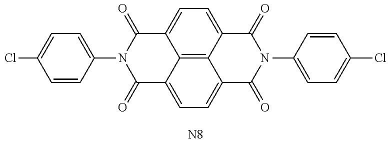

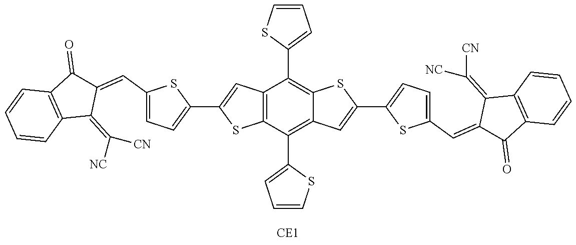 [SMILES: N#CC(C#N)=C1/C(=C\c2ccc(-c3cc4c(-c5cccs5)c5sc(-c6ccc(/C=C7/C(=O)c8ccccc8C7=C(C#N)C#N)s6)cc5c(-c5cccs5)c4s3)s2)C(=O)c2ccccc21]

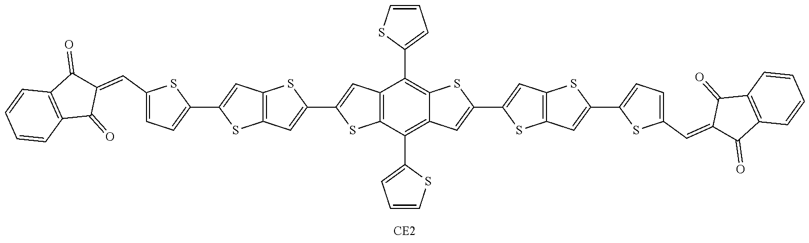 [SMILES: O=C1C(=Cc2ccc(-c3cc4sc(-c5cc6c(-c7cccs7)c7sc(-c8cc9sc(-c%10ccc(C=C%11C(=O)c%12ccccc%12C%11=O)s%10)cc9s8)cc7c(-c7cccs7)c6s5)cc4s3)s2)C(=O)c2ccccc21]

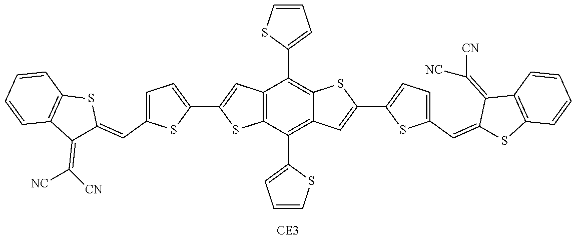 [SMILES: N#CC(C#N)=c1/c(=C/c2ccc(-c3cc4c(-c5cccs5)c5sc(-c6ccc(/C=c7/sc8ccccc8c7=C(C#N)C#N)s6)cc5c(-c5cccs5)c4s3)s2)sc2ccccc12]

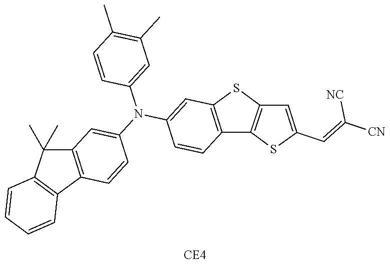 [SMILES: Cc1ccc(N(c2ccc3c(c2)C(C)(C)c2ccccc2-3)c2ccc3c(c2)sc2cc(C=C(C#N)C#N)sc23)cc1C]

From Table 1, it was confirmed that the optoelectronic devices according to Examples 1 to 13 had a maximum absorption wavelength at a wavelength corresponding to green light or red light, and had high external quantum efficiency (EQE) and/or low dark current density (J_{dark}) as compared with the optoelectronic devices according to Comparative Examples 1 to 6. Accordingly, it was confirmed that the optoelectronic devices according to Examples 1 to 13 had high optoelectronic characteristics and low noise as compared with the optoelectronic devices according to Comparative Examples 1 to 6.

Due to the inclusion of an electron-donor group, an electron-acceptor group, and a core group with an appropriate or suitable conjugation length between the electron-donor group and the electron-acceptor group, the organic compound represented by Formula 1 may be to absorb green light and/or red light, and may have excellent or suitable deposition stability and excellent or suitable heat resistance. Accordingly, an optoelectronic device utilizing the organic compound may have improved maximum quantum efficiency.

The optoelectronic device and/or any other relevant devices or components that include optoelectronic device according to embodiments of the present disclosure described herein may be implemented utilizing any suitable hardware, firmware (e.g. an application-specific integrated circuit), software, or a combination of software, firmware, and hardware. For example, the various components of the [device] may be formed on one integrated circuit (IC) chip or on separate IC chips. Further, the various components of the device may be implemented on a flexible printed circuit film, a tape carrier package (TCP), a printed circuit board (PCB), or formed on one substrate. Further, the various components of the device may be a process or thread, running on one or more processors, in one or more computing devices, executing computer program instructions and interacting with other system components for performing the various functionalities described herein. The computer program instructions are stored in a memory which may be implemented in a computing device using a standard memory device, such as, for example, a random access memory (RAM). The computer program instructions may also be stored in other non-transitory computer readable media such as, for example, a CD-ROM, flash drive, or the like. Also, a person of skill in the art should recognize that the functionality of various computing devices may be combined or integrated into a single computing device, or the functionality of a particular computing device may be distributed across one or more other computing devices without departing from the scope of the exemplary embodiments of the present disclosure.

It should be understood that embodiments described herein should be considered in a descriptive sense only and not for purposes of limitation. Descriptions of features or aspects within each embodiment should typically be considered as available for other similar features or aspects in other embodiments. While one or more embodiments have been described with reference to the drawings, it will be understood by those of ordinary skill in the art that one or more suitable changes in form and details may be made therein without departing from the spirit and scope as defined by the following claims and equivalents thereof.

What is claimed is:

1. An optoelectronic device comprising:
a first electrode;
a second electrode facing the first electrode;
an optical activation layer arranged between the first electrode and the second electrode; and
a first compound represented by Formula 1: 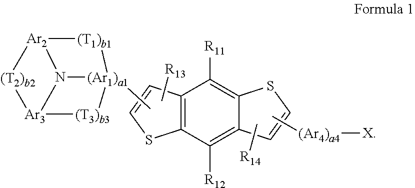
wherein, in Formula 1,
X is an electron-acceptor group,
Ar₁ to Ar₄ are each independently a C₃-C₆₀ carbocyclic group unsubstituted or substituted with at least one R₁₀ₐ or a C₁-C₆₀ heterocyclic group unsubstituted or substituted with at least one R₁₀ₐ,
T₁ to T₃ are each independently selected from a single bond, *—O—*′, *—S—*′, *—C(R₂₁)(R₂₂)—*′, *—Si(R₂₁)(R₂₂)—*′, and *—N(R₂₁)—*′,
each of a1, a4, and b1 to b3 is 0 or 1,
R₁₁ to R₁₄, R₂₁, and R₂₂ are each independently hydrogen, deuterium, —F, —Cl, —Br, —I, a hydroxyl group, a cyano group, a nitro group, a C₁-C₆₀ alkyl group unsubstituted or substituted with at least one R₁₀ₐ, a C₂-C₆₀ alkenyl group unsubstituted or substituted with at least one R₁₀ₐ, a C₂-C₆₀ alkynyl group unsubstituted or substituted with at least one R₁₀ₐ, a C₁-C₆ alkoxy group unsubstituted or substituted with at least one R₁₀ₐ, a C₃-C₆₀ carbocyclic group unsubstituted or substituted with at least one R₁₀ₐ, a C₁-C₆₀ heterocyclic group unsubstituted or substituted with at least one R₁₀ₐ, a C₆-C₆₀ aryloxy group unsubstituted or substituted with at least one R₁₀ₐ, a C₆-C₆₀ arylthio group unsubstituted or substituted with at least one R₁₀ₐ, a C₇-C₆₀ arylalkyl group unsubstituted or substituted with at least one R₁₀ₐ, a C₂-C₆₀ heteroarylalkyl group unsubstituted or substituted with at least one R₁₀ₐ, —C(Q₁)(Q₂)(Q₃), —Si(Q₁)(Q₂)(Q₃), —N(Q₁)(Q₂), —B(Q₁)(Q₂), —C(═O)(Q₁), —S(═O)₂(Q₁), or —P(═O)(Q₁)(Q₂),
R₁₀ₐ is:
deuterium, —F, —Cl, —Br, —I, a hydroxyl group, a cyano group, or a nitro group;
a C₁-C₆₀ alkyl group, a C₂-C₆₀ alkenyl group, a C₂-C₆₀ alkynyl group, or a C₁-C₆₀ alkoxy group, each unsubstituted or substituted with deuterium, —F, —Cl, —Br, —I, a hydroxyl group, a cyano group, a nitro group, a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, a C₂-C₆₀ heteroarylalkyl group, —Si(Q₁₁)(Q₁₂)(Q₁₃), —N(Q₁₁)(Q₁₂), —B(Q₁₁)(Q₁₂), —C(═O)(Q₁₁), —S(═O)₂(Q₁₁), —P(═O)(Q₁₁)(Q₁₂), or any combination thereof;
a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, or a C₂-C₆₀ heteroarylalkyl group, each unsubstituted or substituted with deuterium, —F, —Cl, —Br, —I, a hydroxyl group, a cyano group, a nitro group, a C₁-C₆₀ alkyl group, a C₂-C₆₀ alkenyl group, a C₂-C₆₀ alkynyl group, a C₁-C₆₀ alkoxy group, a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, a C₂-C₆₀ heteroarylalkyl group, —Si(Q₂₁)(Q₂₂)(Q₂₃), —N(Q₂₁)(Q₂₂), —B(Q₂₁)(Q₂₂), —C(═O)(Q₂₁), —S(═O)₂(Q₂₁), —P(═O)(Q₂₁)(Q₂₂), or any combination thereof; or
—Si(Q₃₁)(Q₃₂)(Q₃₃), —N(Q₃₁)(Q₃₂), —B(Q₃₁)(Q₃₂), —C(═O)(Q₃₁), —S(═O)₂(Q₃₁), or —P(═O)(Q₃₁)(Q₃₂), and
Q₁ to Q₃, Q₁₁ to Q₁₃, Q₂₁ to Q₂₃, and Q₃₁ to Q₃₃ are each independently:
hydrogen, deuterium, —F, —Cl, —Br, —I, a hydroxyl group, a cyano group, a nitro group, a C₁-C₆₀ alkyl group, a C₂-C₆₀ alkenyl group, a C₂-C₆₀ alkynyl group, or a C₁-C₆₀ alkoxy group; or
a C₃-C₆₀ carbocyclic group, a C₁-C₆₀ heterocyclic group, a C₇-C₆₀ arylalkyl group, or a C₂-C₆₀ heteroarylalkyl group, each unsubstituted or substituted with deuterium, —F, a cyano group, a C₁-C₆₀ alkyl group, a C₁-C₆₀ alkoxy group, a phenyl group, a biphenyl group, or any combination thereof.

2. The optoelectronic device of claim 1, further comprising a second compound represented by one selected from among Formulae 2-1 to 2-6: 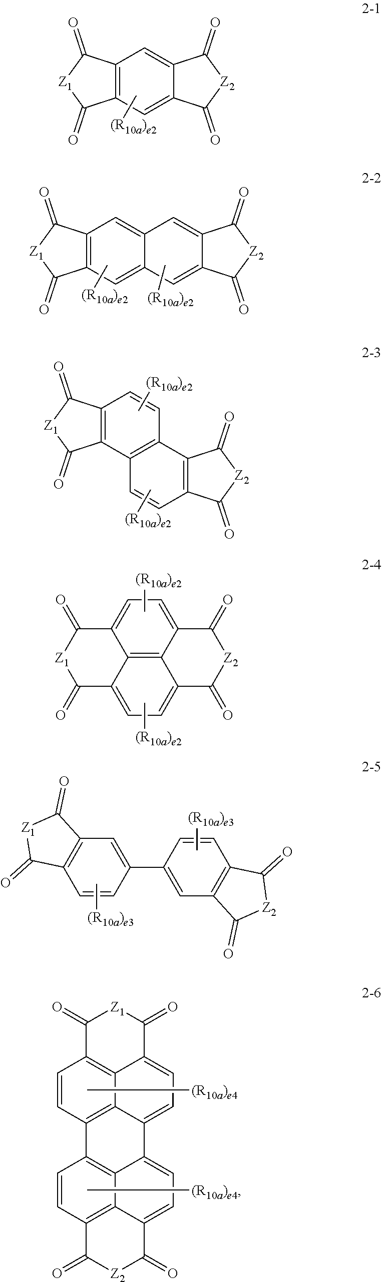
wherein, in Formulae 2-1 to 2-6,
Z₁ and Z₂ are each independently selected from O, S, N(R₁), C(R₁)(R₂), C(═O), C(═S), and C═C(R₁)(R₂),
R₁ and R₂ are each independently hydrogen, deuterium, —F, —Cl, —Br, —I, a hydroxyl group, a cyano group, a nitro group, a C₁-C₆₀ alkyl group unsubstituted or substituted with at least one R₁₀ₐ, a C₂-C₆₀ alkenyl group unsubstituted or substituted with at least one R₁₀ₐ, a C₂-C₆₀ alkynyl group unsubstituted or substituted with at least one R₁₀ₐ, a C₁-C₆₀ alkoxy group unsubstituted or substituted with at least one R₁₀ₐ, a C₃-C₆₀ carbocyclic group unsubstituted or substituted with at least one R₁₀ₐ, a C₁-C₆₀ heterocyclic group unsubstituted or substituted with at least one R₁₀ₐ, a C₆-C₆₀ aryloxy group unsubstituted or substituted with at least one R₁₀ₐ, a C₆-C₆₀ arylthio group unsubstituted or substituted with at least one R₁₀ₐ, a C₇-C₆₀ arylalkyl group unsubstituted or substituted with at least one R₁₀ₐ, a C₂-C₆₀ heteroarylalkyl group unsubstituted or substituted with at least one R₁₀ₐ, —C(Q₁)(Q₂)(Q₃), —Si(Q₁)(Q₂)(Q₃), —N(Q₁)(Q₂), —B(Q₁)(Q₂), —C(═O)(Q₁), —S(═O)₂(Q₁), or —P(═O)(Q₁)(Q₂),
Q₁ to Q₃ and R₁₀ₐ are each as defined in Formula 1,
e2 is an integer from 0 to 2,
e3 is an integer from 0 to 3, and
e4 is an integer from 0 to 4.

3. The optoelectronic device of claim 2, wherein R₁ and R₂ are each independently a C₁-C₆₀ alkyl group, a C₃-C₁₀ cycloalkyl group, a C₆-C₆₀ aryl group, or a C₁-C₆₀ heteroaryl group, each unsubstituted or substituted with deuterium, —F, —Cl, —Br, —I, a cyano group, a C₁-C₆₀ alkyl group, a C₁-C₆₀ alkoxy group, or any combination thereof.

4. The optoelectronic device of claim 2, wherein the second compound is one selected from among Compounds N1 to N43:

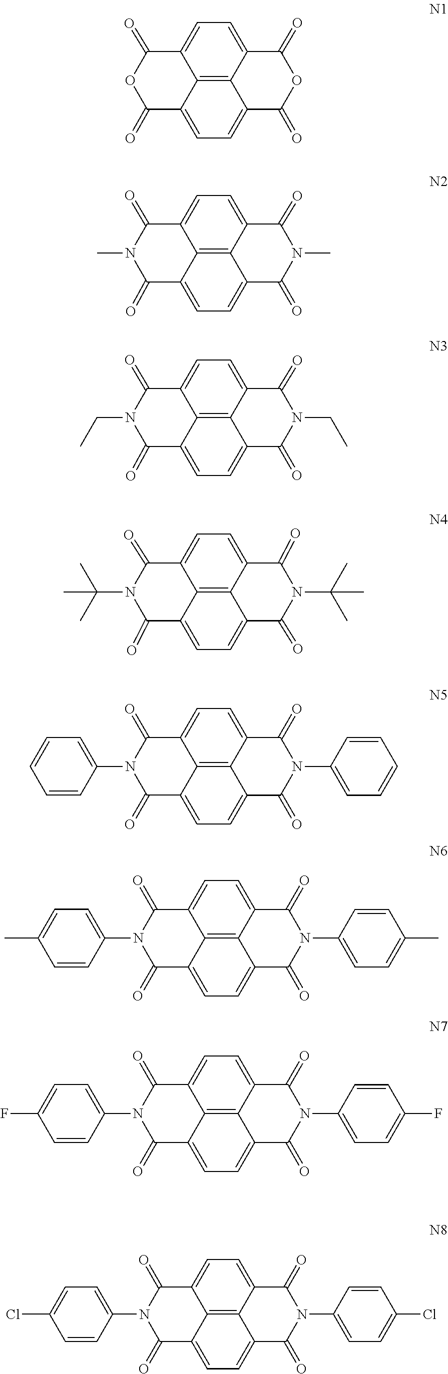

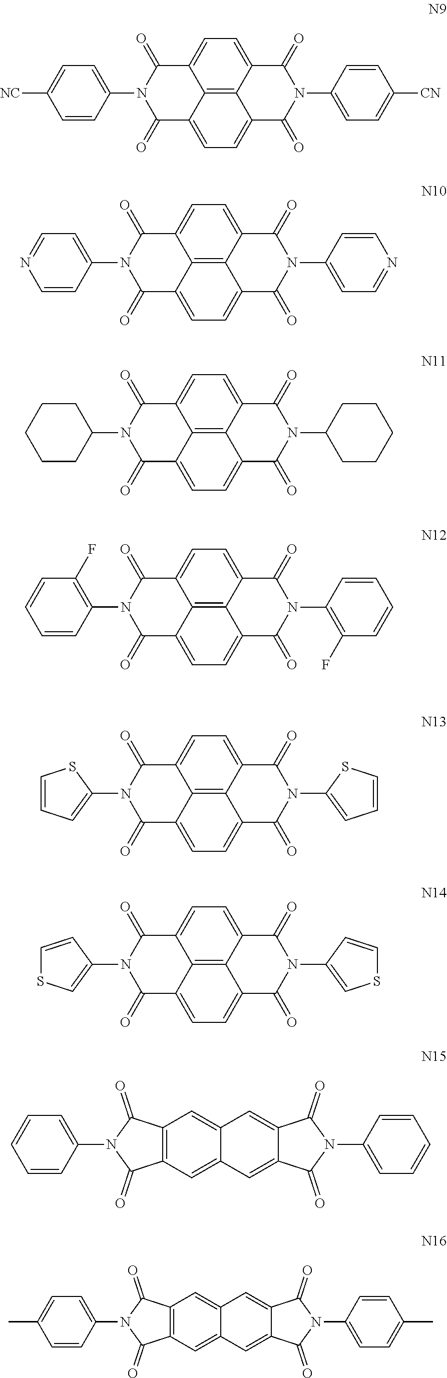

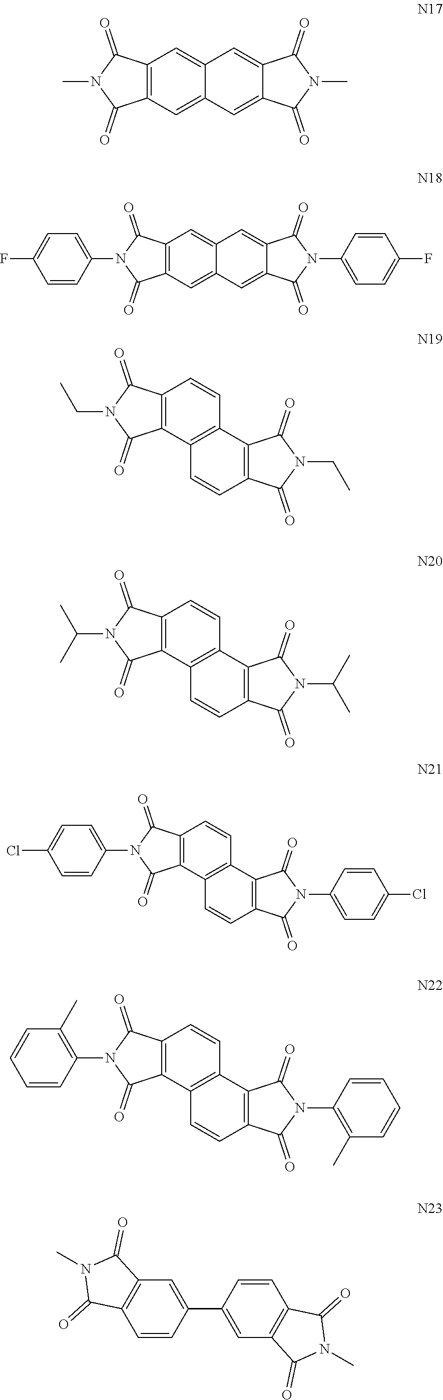

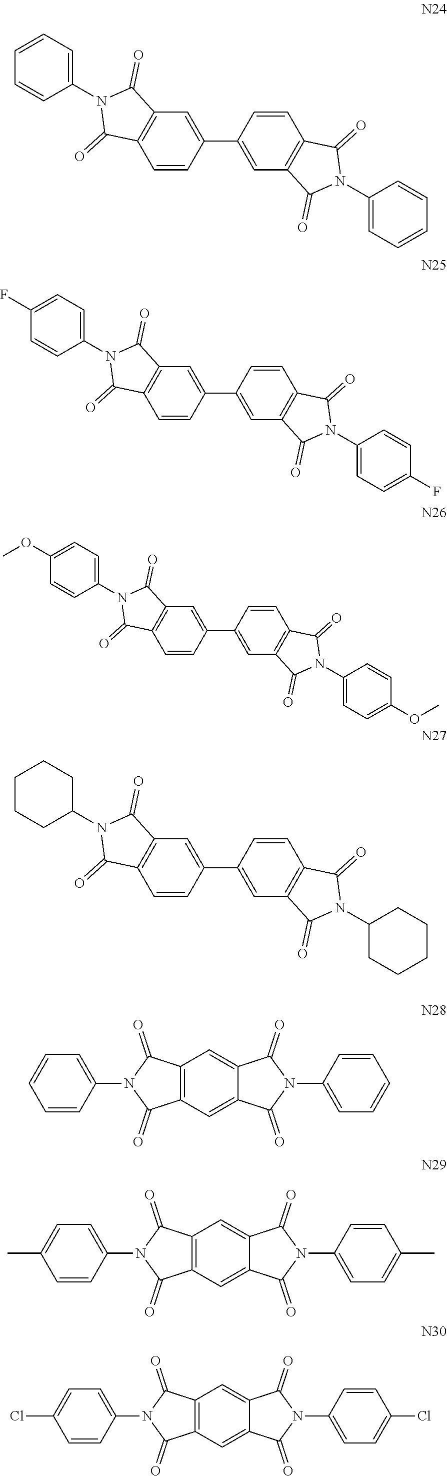

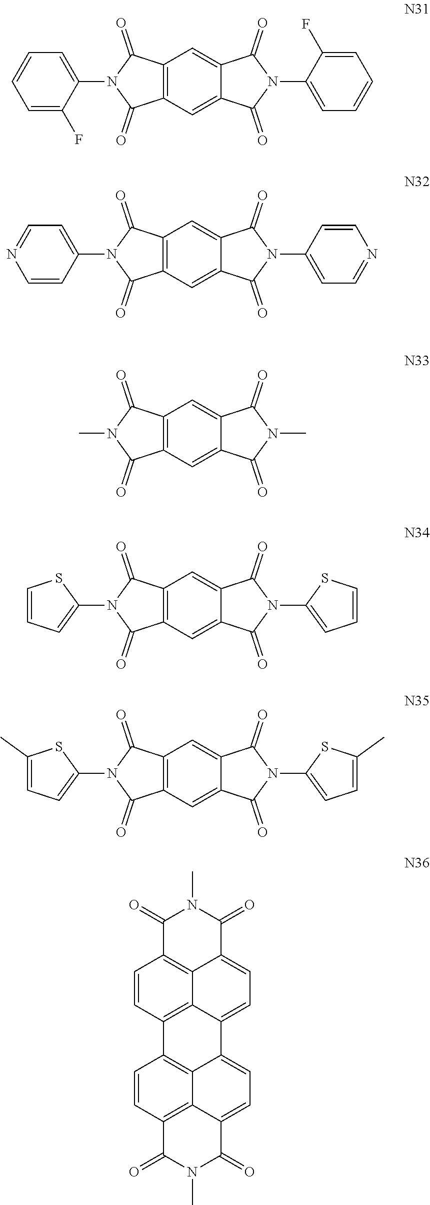

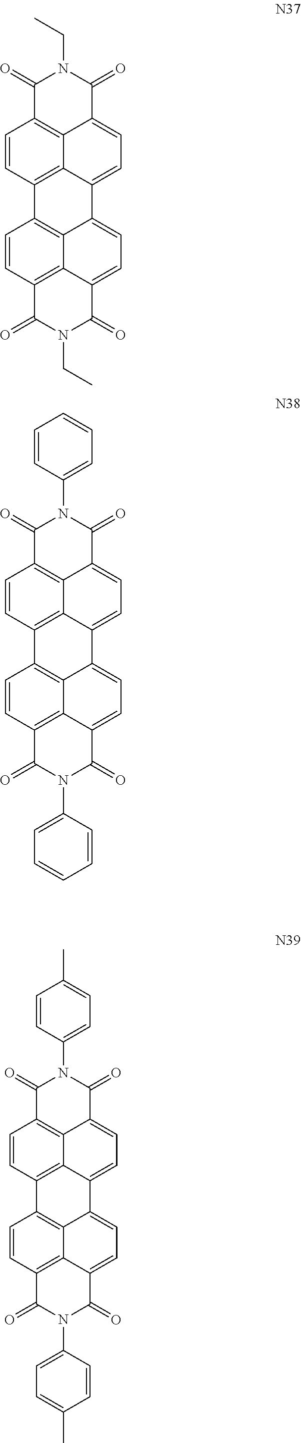

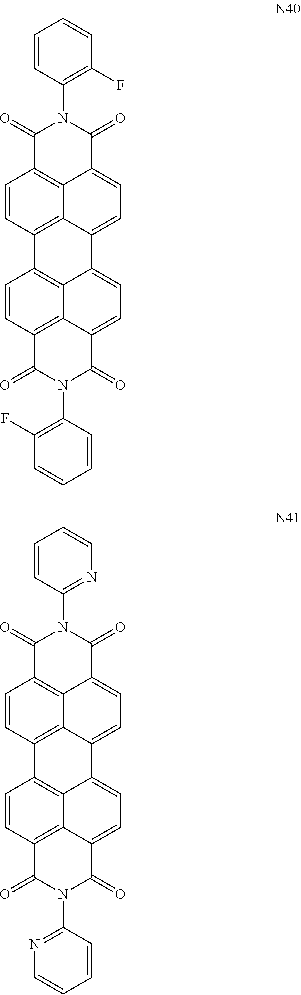

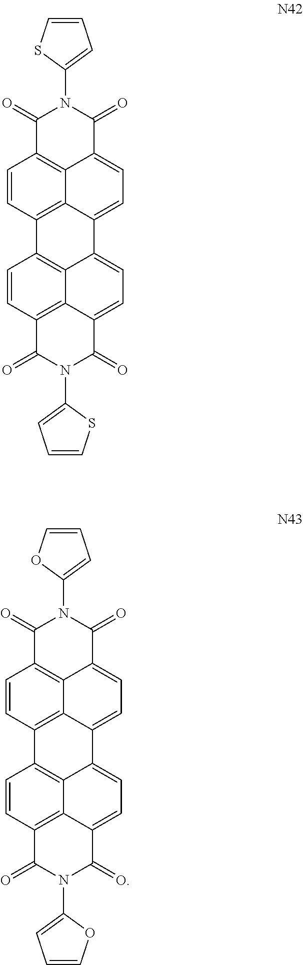 [SMILES: O=C1c2ccc3c4ccc5c6c(ccc(c7ccc(c2c37)C(=O)N1c1cccs1)c64)C(=O)N(c1cccs1)C5=O ; O=C1c2ccc3c4ccc5c6c(ccc(c7ccc(c2c37)C(=O)N1c1ccco1)c64)C(=O)N(c1ccco1)C5=O]

5. The optoelectronic device of claim 1, wherein the optical activation layer comprises the first compound.

6. The optoelectronic device of claim 1, wherein the optical activation layer comprises: a first layer adjacent to the first electrode; and a second layer adjacent to the second electrode.

7. The optoelectronic device of claim 6, wherein the first layer comprises the first compound.

8. An electronic apparatus comprising:
the optoelectronic device of claim 1;
a light-emitting device configured to not overlap the optoelectronic device; and
a color filter, a color conversion layer, a touch screen layer, a polarizing layer, or any combination thereof.

9. An electronic equipment comprising the optoelectronic device of claim 1, wherein the electronic equipment is at least one selected from among a flat panel display, a curved display, a computer monitor, a medical monitor, a television, a billboard, an indoor or outdoor light and/or light for signal, a head-up display, a fully or partially transparent display, a flexible display, a rollable display, a foldable display, a stretchable display, a laser printer, a telephone, a portable phone, a tablet personal computer, a phablet, a personal digital assistant (PDA), a wearable device, a laptop computer, a digital camera, a camcorder, a viewfinder, a micro display, a three-dimensional (3D) display, a virtual reality or augmented reality display, a vehicle, a video wall with multiple displays tiled together, a theater or stadium screen, a phototherapy device, a signboard, a vehicle sensor, a household sensor, a solar cell, and combinations thereof.

10. An organic compound represented by Formula 1: 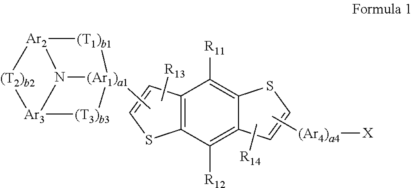
wherein, in Formula 1,
X is an electron-acceptor group,
Ar₁ to Ar₄ are each independently a C₃-C₆₀ carbocyclic group unsubstituted or substituted with at least one R₁₀ₐ or a C₁-C₆₀ heterocyclic group unsubstituted or substituted with at least one R₁₀ₐ,
T₁ to T₃ are each independently selected from a single bond, *—O—*′, *—S—*′, *—C(R₂₁)(R₂₂)—*′, *—Si(R₂₁)(R₂₂)—*′, and *—N(R₂₁)—*′,
each of a1, a4, and b1 to b3 is 0 or 1,
R₁₁ to R₁₄, R₂₁, and R₂₂ are each independently hydrogen, deuterium, —F, —Cl, —Br, —I, a hydroxyl group, a cyano group, a nitro group, a C₁-C₆₀ alkyl group unsubstituted or substituted with at least one R₁₀ₐ, a C₂-C₆₀ alkenyl group unsubstituted or substituted with at least one R₁₀ₐ, a C₂-C₆₀ alkynyl group unsubstituted or substituted with at least one R₁₀ₐ, a C₁-C₆₀ alkoxy group unsubstituted or substituted with at least one R₁₀ₐ, a C₃-C₆₀ carbocyclic group unsubstituted or substituted with at least one R₁₀ₐ, a C₁-C₆₀ heterocyclic group unsubstituted or substituted with at least one R₁₀ₐ, a C₆-C₆₀ aryloxy group unsubstituted or substituted with at least one R₁₀ₐ, a C₆-C₆₀ arylthio group unsubstituted or substituted with at least one R₁₀ₐ, a C₇-C₆₀ arylalkyl group unsubstituted or substituted with at least one R₁₀ₐ, a C₂-C₆₀ heteroarylalkyl group unsubstituted or substituted with at least one R₁₀ₐ, —C(Q₁)(Q₂)(Q₃), —Si(Q₁)(Q₂)(Q₃), —N(Q₁)(Q₂), —B(Q₁)(Q₂), —C(═O)(Q₁), —S(═O)₂(Q₁), or —P(═O)(Q₁)(Q₂),
R₁₀ₐ is:
deuterium, —F, —Cl, —Br, —I, a hydroxyl group, a cyano group, or a nitro group;
a C₁-C₆₀ alkyl group, a C₂-C₆₀ alkenyl group, a C₂-C₆₀ alkynyl group, or a C₁-C₆₀ alkoxy group, each unsubstituted or substituted with deuterium, —F, —Cl, —Br, —I, a hydroxyl group, a cyano group, a nitro group, a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, a C₂-C₆₀ heteroarylalkyl group, —Si(Q₁₁)(Q₁₂)(Q₁₃), —N(Q₁₁)(Q₁₂), —B(Q₁₁)(Q₁₂), —C(═O)(Q₁₁), —S(═O)₂(Q₁₁), —P(═O)(Q₁₁)(Q₁₂), or any combination thereof;
a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, or a C₂-C₆₀ heteroarylalkyl group, each unsubstituted or substituted with deuterium, —F, —Cl, —Br, —I, a hydroxyl group, a cyano group, a nitro group, a C₁-C₆₀ alkyl group, a C₂-C₆₀ alkenyl group, a C₂-C₆₀ alkynyl group, a C₁-C₆₀ alkoxy group, a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, a C₂-C₆₀ heteroarylalkyl group, —Si(Q₂₁)(Q₂₂)(Q₂₃), —N(Q₂₁)(Q₂₂), —B(Q₂₁)(Q₂₂), —C(═O)(Q₂₁), —S(═O)₂(Q₂₁), —P(═O)(Q₂₁)(Q₂₂), or any combination thereof; or
—Si(Q₃₁)(Q₃₂)(Q₃₃), —N(Q₃₁)(Q₃₂), —B(Q₃₁)(Q₃₂), —C(═O)(Q₃₁), —S(═O)₂(Q₃₁), or —P(═O)(Q₃₁)(Q₃₂), and
Q₁ to Q₃, Q₁₁ to Q₁₃, Q₂₁ to Q₂₃, and Q₃₁ to Q₃₃ are each independently:
hydrogen, deuterium, —F, —Cl, —Br, —I, a hydroxyl group, a cyano group, a nitro group, a C₁-C₆₀ alkyl group, a C₂-C₆₀ alkenyl group, a C₂-C₆₀ alkynyl group, or a C₁-C₆₀ alkoxy group; or
a C₃-C₆₀ carbocyclic group, a C₁-C₆₀ heterocyclic group, a C₇-C₆₀ arylalkyl group, or a C₂-C₆₀ heteroarylalkyl group, each unsubstituted or substituted with deuterium, —F, a cyano group, a C₁-C₆₀ alkyl group, a C₁-C₆₀ alkoxy group, a phenyl group, a biphenyl group, or any combination thereof.

11. The organic compound of claim 10, wherein a maximum absorption wavelength of the organic compound is about 490 nm to about 750 nm.

12. The organic compound of claim 10, wherein, in Formula 1, X is selected from among groups represented by Formulae 1-A, 1-B-1 to 1-B-5, and 1-C-1 to 1-C-6: 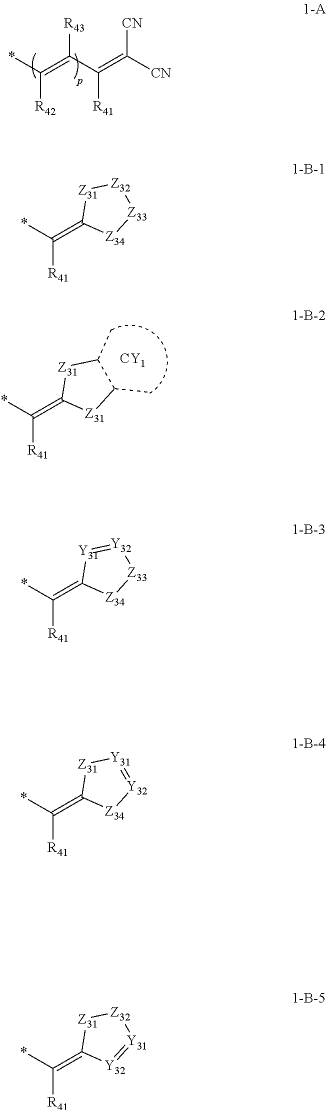 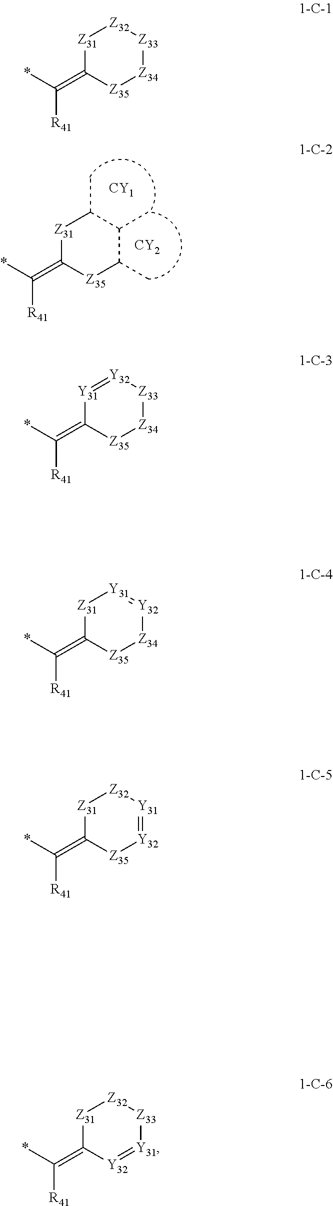
and
wherein, in Formulae 1-A, 1-B-1 to 1-B-5, and 1-C-1 to 1-C-6,
Z₃₁ to Z₃₅ are each independently selected from O, S, N(R₃₁), C(R₃₁)(R₃₂), C(═O), C(═S), and C═C(R₃₁)(R₃₂),
each of Y₃₁ and Y₃₂ is N or C(R₃₃),
R₃₁ to R₃₃ and R₄₁ to R₄₃ are each independently hydrogen, deuterium, —F, —Cl, —Br, —I, a hydroxyl group, a cyano group, a nitro group, a C₁-C₆₀ alkyl group unsubstituted or substituted with at least one R₁₀ₐ, a C₂-C₆₀ alkenyl group unsubstituted or substituted with at least one R₁₀ₐ, a C₂-C₆₀ alkynyl group unsubstituted or substituted with at least one R₁₀ₐ, a C₁-C₆₀ alkoxy group unsubstituted or substituted with at least one R₁₀ₐ, a C₃-C₆₀ carbocyclic group unsubstituted or substituted with at least one R₁₀ₐ, a C₁-C₆₀ heterocyclic group unsubstituted or substituted with at least one R₁₀ₐ, a C₆-C₆₀ aryloxy group unsubstituted or substituted with at least one R₁₀ₐ, a C₆-C₆₀ arylthio group unsubstituted or substituted with at least one R₁₀ₐ, a C₇-C₆₀ arylalkyl group unsubstituted or substituted with at least one R₁₀ₐ, a C₂-C₆₀ heteroarylalkyl group unsubstituted or substituted with at least one R₁₀ₐ, —C(Q₁)(Q₂)(Q₃), —Si(Q₁)(Q₂)(Q₃), —N(Q₁)(Q₂), —B(Q₁)(Q₂), —C(═O)(Q₁), —S(═O)₂(Q₁), or —P(═O)(Q₁)(Q₂),
CY₁ and CY₂ are each independently a C₃-C₆₀ carbocyclic group unsubstituted or substituted with at least one R₁₀ₐ or a C₁-C₆₀ heterocyclic group unsubstituted or substituted with at least one R₁₀ₐ,
p is an integer from 0 to 2,
Q₁ to Q₃ and R₁₀ₐ are each as defined in Formula 1, and
* indicates a binding site to a neighboring atom.

13. The organic compound of claim 10, wherein, in Formula 1, Ar₁ to Ar₄ are each independently a benzene group, a pyridine group, a pyrimidine group, a triazine group, a naphthalene group, an anthracene group, a phenanthrene group, a phenalene group, a thiophene group, a furan group, a benzothiophene group, a benzofuran group, a dibenzothiophene group, or a dibenzofuran group, each unsubstituted or substituted with at least one R₁₀ₐ.

14. The organic compound of claim 10, wherein, in Formula 1, a4 is 0.

15. The organic compound of claim 10, wherein, in Formula 1, R₁₁ to R₁₄ are each independently hydrogen, deuterium, —F, —Cl, —Br, —I, a cyano group, a C₁-C₆₀ alkyl group unsubstituted or substituted with at least one R₁₀ₐ, a C₁-C₆₀ alkoxy group unsubstituted or substituted with at least one R₁₀ₐ, or a C₃-C₆₀ aryl group unsubstituted or substituted with at least one R₁₀ₐ.

16. The organic compound of claim 10, wherein, in Formula 1, T₁ to T₃ are each independently selected from a single bond, *—O—*′, *—S—*′, and *—C(R₂₁)(R₂₂)—*′, and
R₂₁ and R₂₂ are each independently hydrogen, deuterium, —F, —Cl, —Br, —I, a cyano group, a C₁-C₆₀ alkyl group unsubstituted or substituted with at least one R₁₀ₐ, a C₁-C₆₀ alkoxy group unsubstituted or substituted with at least one R₁₀ₐ, a C₃-C₆₀ aryl group unsubstituted or substituted with at least one R₁₀ₐ, or a C₁-C₆₀ heteroaryl group unsubstituted or substituted with at least one R₁₀ₐ.

17. The organic compound of claim 10, wherein, in Formula 1, the sum of b1 to b3 is 0 or 1.

18. The organic compound of claim 10, wherein the organic compound is represented by Formula 1-1:

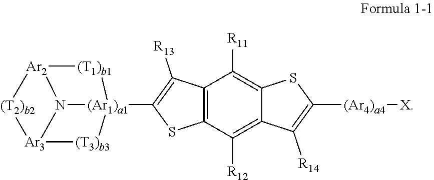

19. The organic compound of claim 10, wherein a group represented by 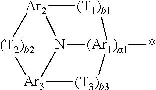
in Formula 1 is a group selected from among Formulae L1 to L26: 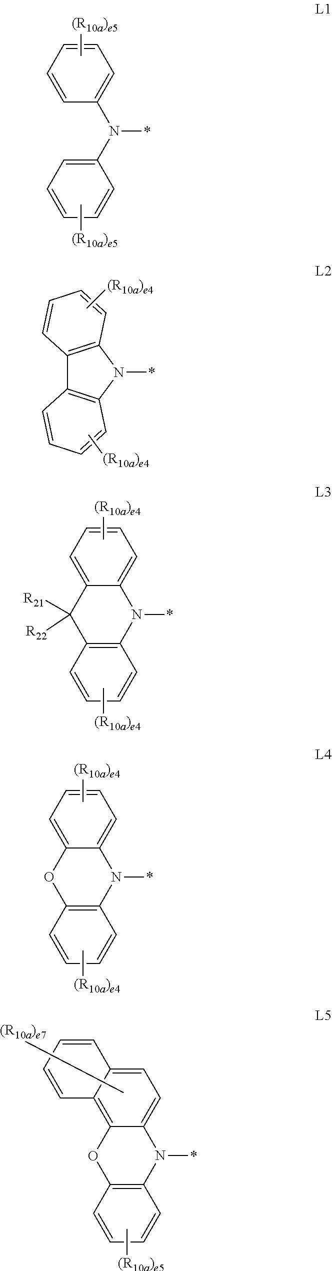 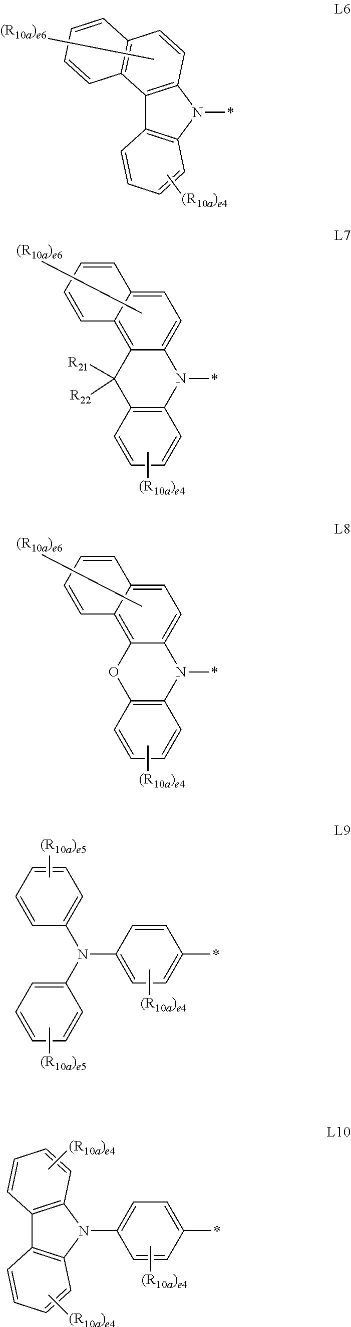 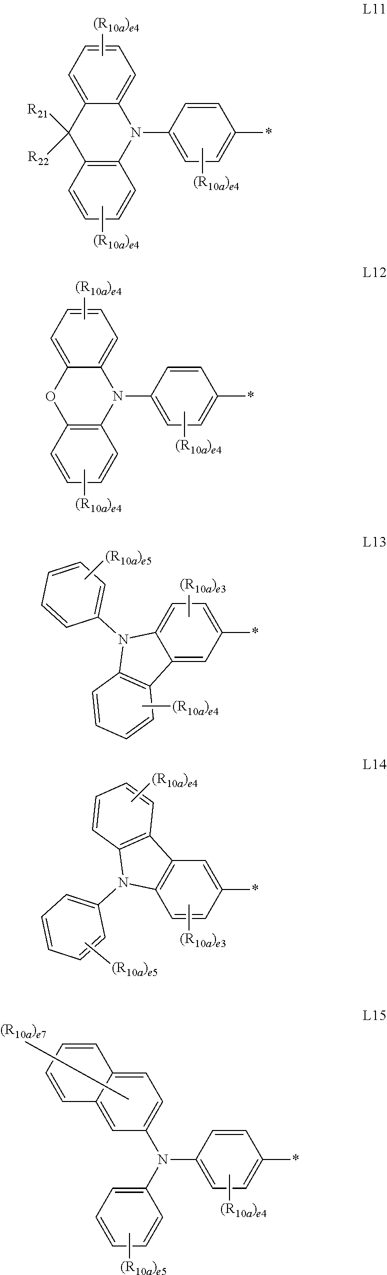 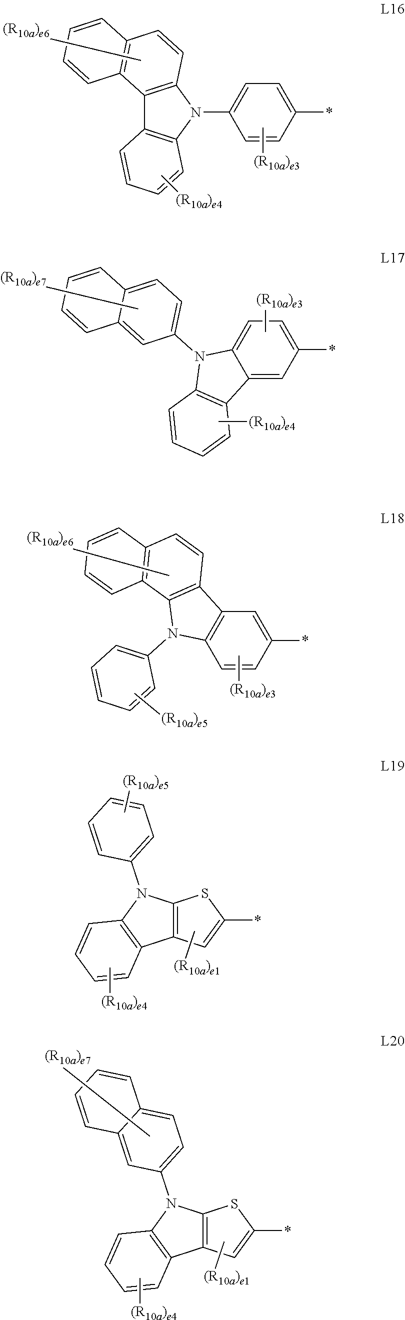 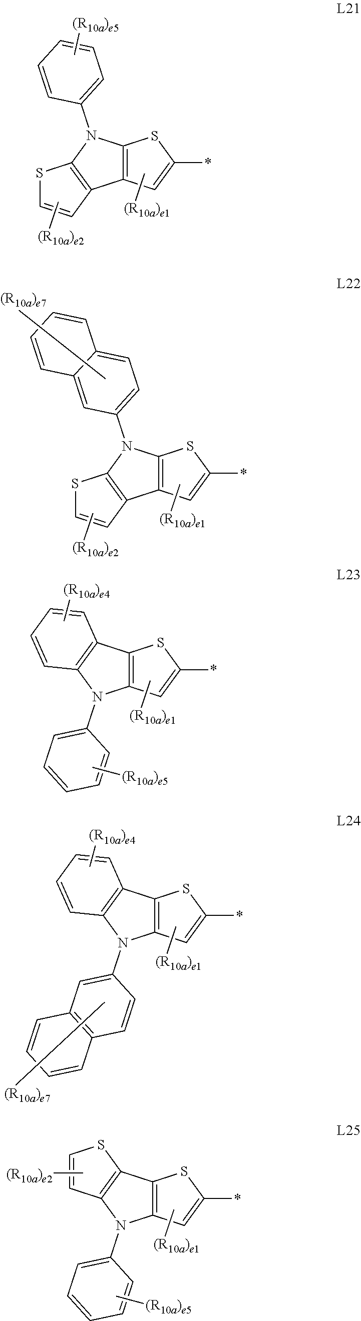 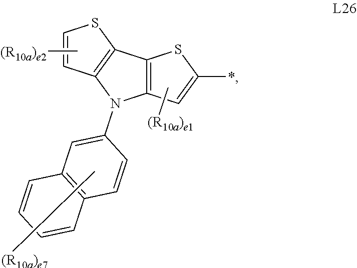
and
wherein, in Formulae L1 to L26,
R₁₀ₐ is as defined in Formula 1,
e1 is 0 or 1,
e2 is an integer from 0 to 2,
e3 is an integer from 0 to 3,
e4 is an integer from 0 to 4,
e5 is an integer from 0 to 5,
e6 is an integer from 0 to 6,
e7 is an integer from 0 to 7, and
* indicates a binding site to a neighboring atom.

20. The organic compound of claim 10, wherein the organic compound is one selected from among Compounds P1 to P35: 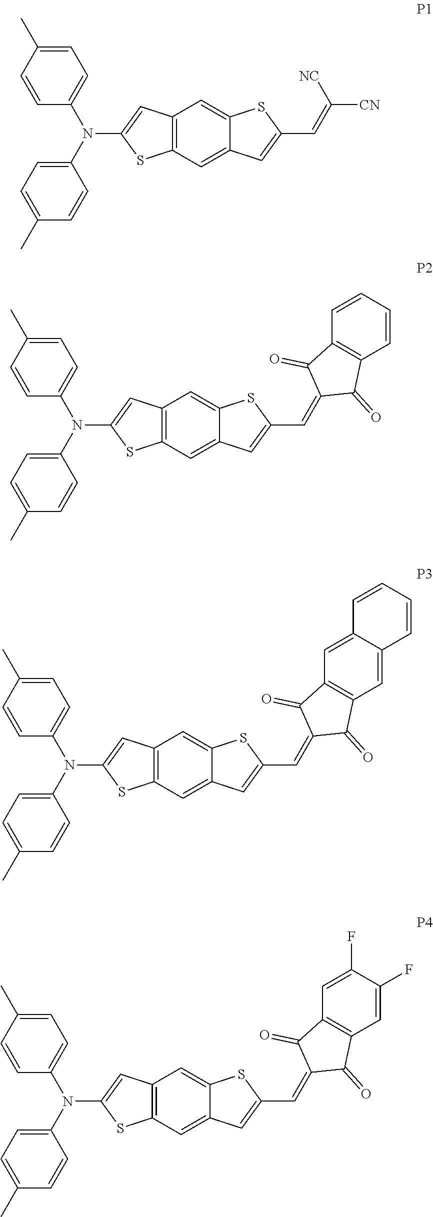 [SMILES: Cc1ccc(N(c2ccc(C)cc2)c2cc3cc4sc(C=C(C#N)C#N)cc4cc3s2)cc1 ; Cc1ccc(N(c2ccc(C)cc2)c2cc3cc4sc(C=C5C(=O)c6ccccc6C5=O)cc4cc3s2)cc1 ; Cc1ccc(N(c2ccc(C)cc2)c2cc3cc4sc(C=C5C(=O)c6cc7ccccc7cc6C5=O)cc4cc3s2)cc1 ; Cc1ccc(N(c2ccc(C)cc2)c2cc3cc4sc(C=C5C(=O)c6cc(F)c(F)cc6C5=O)cc4cc3s2)cc1] 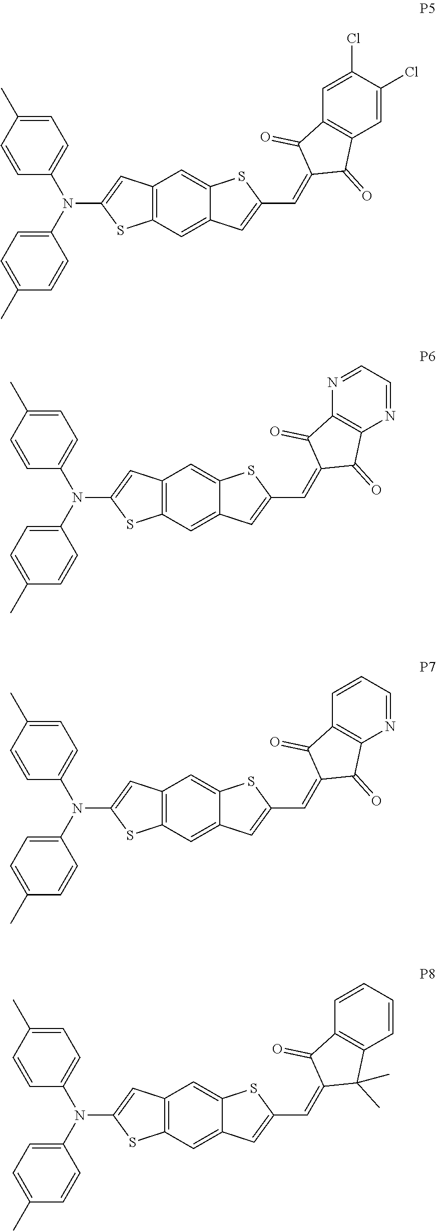 [SMILES: Cc1ccc(N(c2ccc(C)cc2)c2cc3cc4sc(C=C5C(=O)c6cc(Cl)c(Cl)cc6C5=O)cc4cc3s2)cc1 ; Cc1ccc(N(c2ccc(C)cc2)c2cc3cc4sc(C=C5C(=O)c6nccnc6C5=O)cc4cc3s2)cc1 ; Cc1ccc(N(c2ccc(C)cc2)c2cc3cc4sc(/C=C5\C(=O)c6cccnc6C5=O)cc4cc3s2)cc1 ; Cc1ccc(N(c2ccc(C)cc2)c2cc3cc4sc(/C=C5\C(=O)c6ccccc6C5(C)C)cc4cc3s2)cc1] 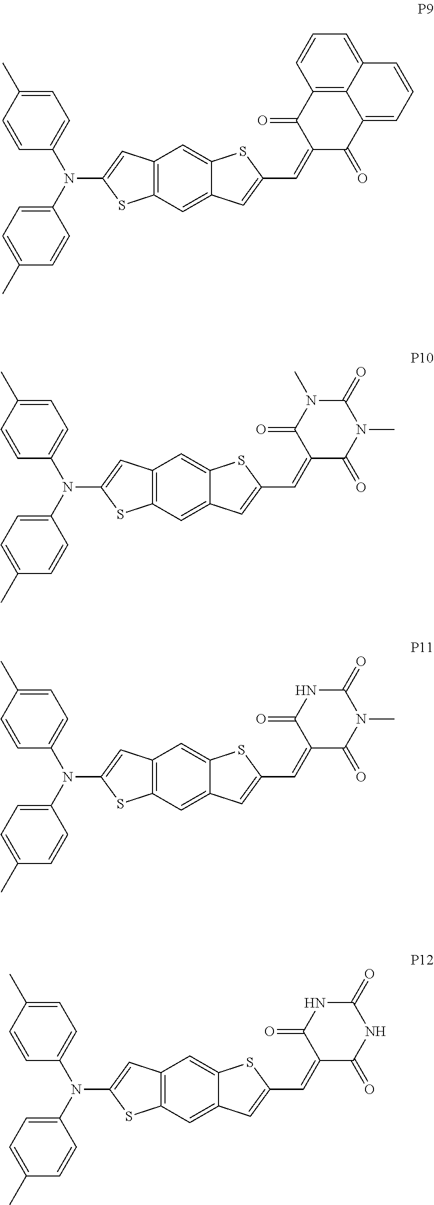 [SMILES: Cc1ccc(N(c2ccc(C)cc2)c2cc3cc4sc(C=c5c(=O)c6cccc7cccc(c5=O)c76)cc4cc3s2)cc1 ; Cc1ccc(N(c2ccc(C)cc2)c2cc3cc4sc(C=C5C(=O)N(C)C(=O)N(C)C5=O)cc4cc3s2)cc1 ; Cc1ccc(N(c2ccc(C)cc2)c2cc3cc4sc(/C=C5\C(=O)NC(=O)N(C)C5=O)cc4cc3s2)cc1 ; Cc1ccc(N(c2ccc(C)cc2)c2cc3cc4sc(C=C5C(=O)NC(=O)NC5=O)cc4cc3s2)cc1] 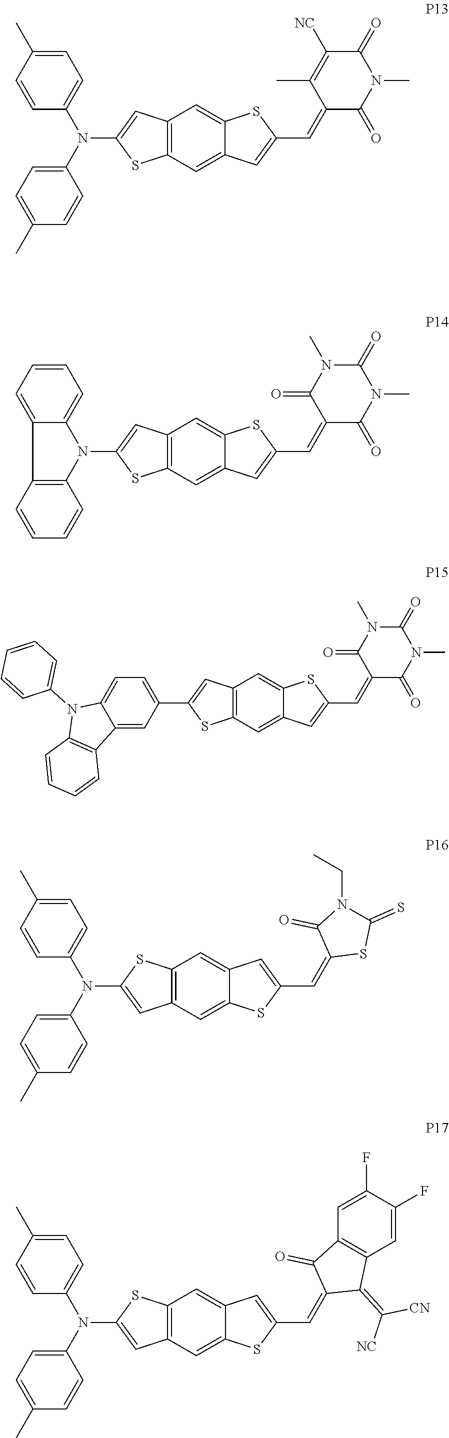 [SMILES: CC1=C(C#N)C(=O)N(C)C(=O)/C1=C/c1cc2cc3sc(N(c4ccc(C)cc4)c4ccc(C)cc4)cc3cc2s1 ; CN1C(=O)C(=Cc2cc3cc4sc(-n5c6ccccc6c6ccccc65)cc4cc3s2)C(=O)N(C)C1=O ; CN1C(=O)C(=Cc2cc3cc4sc(-c5ccc6c(c5)c5ccccc5n6-c5ccccc5)cc4cc3s2)C(=O)N(C)C1=O ; CCN1C(=O)/C(=C\c2cc3cc4sc(N(c5ccc(C)cc5)c5ccc(C)cc5)cc4cc3s2)SC1=S ; Cc1ccc(N(c2ccc(C)cc2)c2cc3cc4sc(/C=C5\C(=O)c6cc(F)c(F)cc6C5=C(C#N)C#N)cc4cc3s2)cc1] 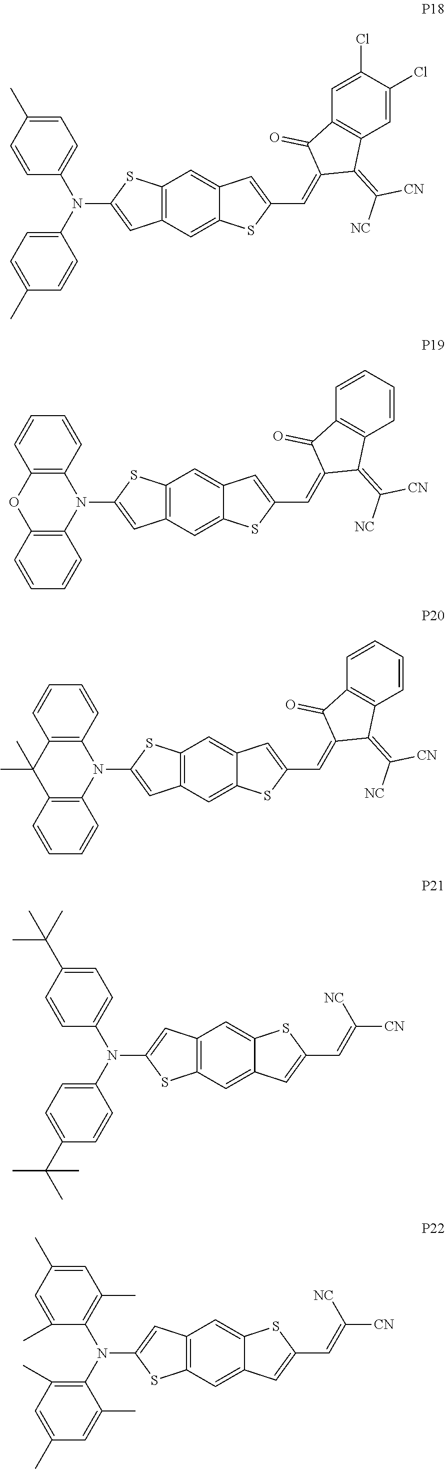 [SMILES: Cc1ccc(N(c2ccc(C)cc2)c2cc3cc4sc(/C=C5\C(=O)c6cc(Cl)c(Cl)cc6C5=C(C#N)C#N)cc4cc3s2)cc1 ; N#CC(C#N)=C1/C(=C/c2cc3cc4sc(N5c6ccccc6Oc6ccccc65)cc4cc3s2)C(=O)c2ccccc21 ; CC1(C)c2ccccc2N(c2cc3cc4sc(/C=C5\C(=O)c6ccccc6C5=C(C#N)C#N)cc4cc3s2)c2ccccc21 ; CC(C)(C)c1ccc(N(c2ccc(C(C)(C)C)cc2)c2cc3cc4sc(C=C(C#N)C#N)cc4cc3s2)cc1 ; Cc1cc(C)c(N(c2cc3cc4sc(C=C(C#N)C#N)cc4cc3s2)c2c(C)cc(C)cc2C)c(C)c1] 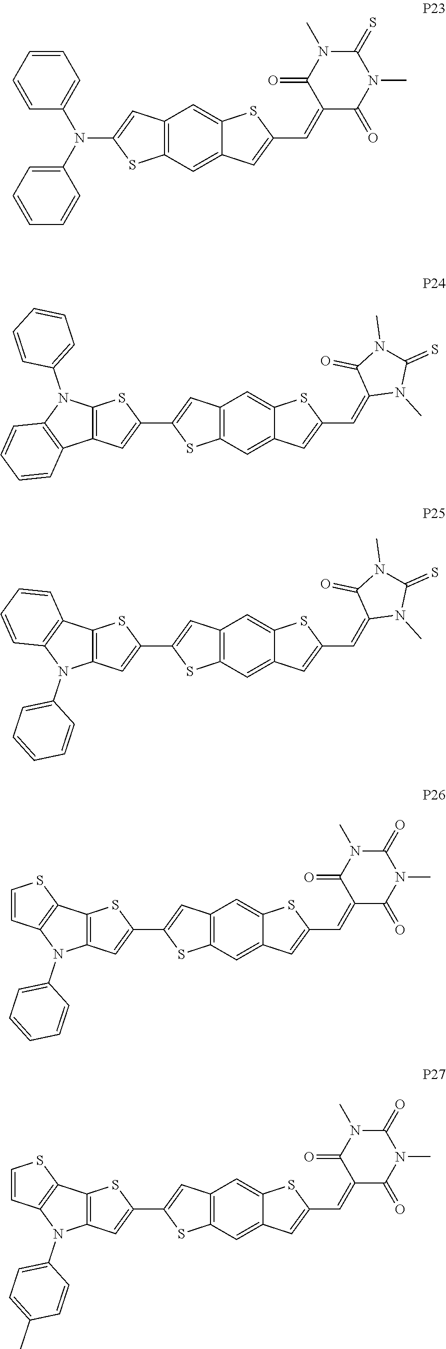 [SMILES: CN1C(=O)C(=Cc2cc3cc4sc(N(c5ccccc5)c5ccccc5)cc4cc3s2)C(=O)N(C)C1=S ; CN1C(=O)/C(=C\c2cc3cc4sc(-c5cc6c7ccccc7n(-c7ccccc7)c6s5)cc4cc3s2)N(C)C1=S ; CN1C(=O)/C(=C\c2cc3cc4sc(-c5cc6c(s5)c5ccccc5n6-c5ccccc5)cc4cc3s2)N(C)C1=S ; CN1C(=O)C(=Cc2cc3cc4sc(-c5cc6c(s5)c5sccc5n6-c5ccccc5)cc4cc3s2)C(=O)N(C)C1=O ; Cc1ccc(-n2c3ccsc3c3sc(-c4cc5cc6sc(C=C7C(=O)N(C)C(=O)N(C)C7=O)cc6cc5s4)cc32)cc1] 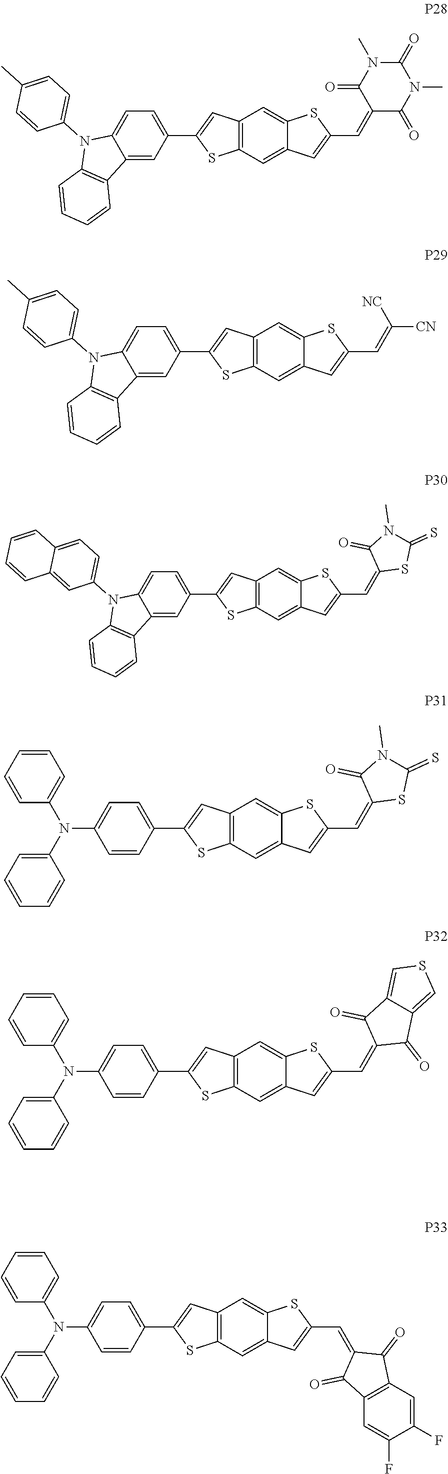 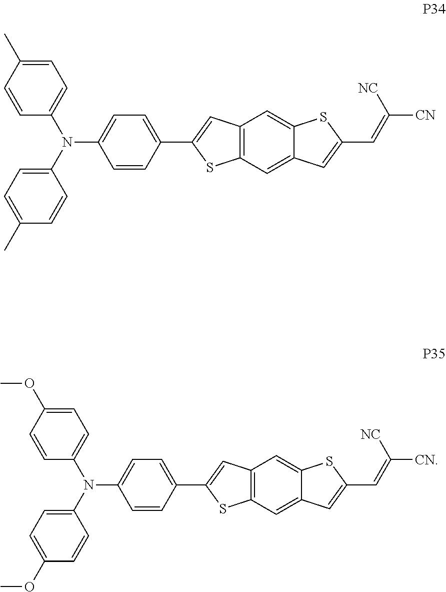 [SMILES: Cc1ccc(N(c2ccc(C)cc2)c2ccc(-c3cc4cc5sc(C=C(C#N)C#N)cc5cc4s3)cc2)cc1 ; COc1ccc(N(c2ccc(OC)cc2)c2ccc(-c3cc4cc5sc(C=C(C#N)C#N)cc5cc4s3)cc2)cc1]
